SEQUENCE LISTING (487 sequences)

SEQ ID NO: 1 - protein, 30 aa, synthetic construct
  REGION 1..30: note = PR004290 HFWR1 Kabat, PR005656 HFWR1 Kabat, PR005657 HFWR1 Kabat,PR005658 HFWR1 Kabat, PR005660 HFWR1 Kabat, PR005662 HFWR1 Kabat,PR006142 HFWR1 Kabat, PR006143 HFWR1 Kabat, PR006144 HFWR1 Kabat,PR
EVQLVESGGVLVQPGGSLRLSCAASGFTFS

SEQ ID NO: 2 - protein, 30 aa, synthetic construct
  REGION 1..30: note = PR006669 HFWR1 Kabat, PR006744 HFWR1 Kabat, PR006745 HFWR1 Kabat,PR006746 HFWR1 Kabat, PR302029 HFWR1 Kabat, PR302031 HFWR1 Kabat,PR302034 HFWR1 Kabat, PR302334-10 HFWR1 Kabat, PR302334-11 HFWR1Kab
EVQLVESGGGLVQPGGSLRLSCAASGFTFS

SEQ ID NO: 3 - protein, 30 aa, synthetic construct
  REGION 1..30: note = PR301306 HFWR1 Kabat
EVKLEESGGGLVQPGGSMKLSCVASGFTFS

SEQ ID NO: 4 - protein, 30 aa, synthetic construct
  REGION 1..30: note = PR301612 HFWR1 Kabat
EVKLEESGGGLVQPGGSIKLSCVASGFTFS

SEQ ID NO: 5 - protein, 30 aa, synthetic construct
  REGION 1..30: note = PR301777 HFWR1 Kabat, PR301788 HFWR1 Kabat, PR301790 HFWR1 Kabat
EMKLEESGGGLVQPGGSMKLSCVASGFTFS

SEQ ID NO: 6 - protein, 30 aa, synthetic construct
  REGION 1..30: note = PR301780 HFWR1 Kabat, PR302309-1 HFWR1 Kabat, PR302309-2 HFWR1Kabat, PR302309-3 HFWR1 Kabat, PR302309-4 HFWR1 Kabat, PR302309-5HFWR1 Kabat, PR302309-6 HFWR1 Kabat, PR302309 HFWR1 Kabat,PR302332 HFW
QIQLVQSGPELKKPGETVKISCKASGYTFT

SEQ ID NO: 7 - protein, 30 aa, synthetic construct
  REGION 1..30: note = PR301803 HFWR1 Kabat
EVKLEESGGGLVQPGGSMKLSCVASGFTFN

SEQ ID NO: 8 - protein, 30 aa, synthetic construct
  REGION 1..30: note = PR301806 HFWR1 Kabat
QVQLQQPGAELVKPGTSVKMSCKASGYTFT

SEQ ID NO: 9 - protein, 30 aa, synthetic construct
  REGION 1..30: note = PR302036 HFWR1 Kabat, PR302038 HFWR1 Kabat, PR302041 HFWR1 Kabat
EMQLVESGGGLVQPGGSLRLSCAASGFTFS

SEQ ID NO: 10 - protein, 30 aa, synthetic construct
  REGION 1..30: note = PR302043 HFWR1 Kabat, PR302045 HFWR1 Kabat, PR302048 HFWR1 Kabat
EVQLVESGGGLVQPGGSLKLSCAASGFTFS

SEQ ID NO: 11 - protein, 30 aa, synthetic construct
  REGION 1..30: note = PR302050 HFWR1 Kabat, PR302052 HFWR1 Kabat, PR302055 HFWR1 Kabat
EMQLVESGGGLVQPGGSLKLSCAASGFTFS

SEQ ID NO: 12 - protein, 30 aa, synthetic construct
  REGION 1..30: note = PR302064 HFWR1 Kabat, PR302066 HFWR1 Kabat, PR302069 HFWR1 Kabat
EMQLVESGGGLVKPGGSLRLSCAASGFTFS

SEQ ID NO: 13 - protein, 30 aa, synthetic construct
  REGION 1..30: note = PR302309-10 HFWR1 Kabat, PR302309-11 HFWR1 Kabat, PR302309-12HFWR1 Kabat, PR302309-14 HFWR1 Kabat, PR302309-7 HFWR1 Kabat,PR302309-8 HFWR1 Kabat, PR302309-9 HFWR1 Kabat
QIQLVQSGSELKKPGASVKVSCKASGYTFT

SEQ ID NO: 14 - protein, 30 aa, synthetic construct
  REGION 1..30: note = PR302309-13 HFWR1 Kabat, PR302309-17 HFWR1 Kabat
QVQLVQSGSELKKPGASVKVSCKASGYTFT

SEQ ID NO: 15 - protein, 30 aa, synthetic construct
  REGION 1..30: note = PR302309-15 HFWR1 Kabat, PR302309-18 HFWR1 Kabat, PR302341-10HFWR1 Kabat, PR302341-11 HFWR1 Kabat, PR302341-12 HFWR1 Kabat,PR302341-13 HFWR1 Kabat, PR302341-14 HFWR1 Kabat, PR302341-15HFWR1 Kabat,
QVQLVQSGAEVKKPGASVKVSCKASGYTFT

SEQ ID NO: 16 - protein, 30 aa, synthetic construct
  REGION 1..30: note = PR302309-16 HFWR1 Kabat, PR302309-19 HFWR1 Kabat, PR302309-20HFWR1 Kabat, PR302309-21 HFWR1 Kabat
QIQLVQSGAEVKKPGASVKVSCKASGYTFT

SEQ ID NO: 17 - protein, 30 aa, synthetic construct
  REGION 1..30: note = PR302312 HFWR1 Kabat, PR302324 HFWR1 Kabat, PR302341-1 HFWR1Kabat, PR302341-2 HFWR1 Kabat, PR302341-3 HFWR1 Kabat, PR302341-4HFWR1 Kabat, PR302341-5 HFWR1 Kabat, PR302341-6 HFWR1 Kabat,PR302341 HFW
EVQLQQSGPELVKPGASVKMSCKASGYTFT

SEQ ID NO: 18 - protein, 30 aa, synthetic construct
  REGION 1..30: note = PR302315 HFWR1 Kabat
DVQLVESGGGLMQPGGSRKLSCVASGFTFS

SEQ ID NO: 19 - protein, 30 aa, synthetic construct
  REGION 1..30: note = PR302328 HFWR1 Kabat, PR302331 HFWR1 Kabat, PR302334-1 HFWR1Kabat, PR302334-2 HFWR1 Kabat, PR302334-3 HFWR1 Kabat, PR302334-4HFWR1 Kabat, PR302334-5 HFWR1 Kabat, PR302334-6 HFWR1 Kabat,PR302334 HFW
EVKLVESGGGLVKPGGSLKLSCAASGFTFS

SEQ ID NO: 20 - protein, 30 aa, synthetic construct
  REGION 1..30: note = PR302334-15 HFWR1 Kabat, PR302334-16 HFWR1 Kabat, PR302334-24HFWR1 Kabat, PR302334-25 HFWR1 Kabat
EVQLVESGGGLVKPGGSLRLSCAASGFTFS

SEQ ID NO: 21 - protein, 30 aa, synthetic construct
  REGION 1..30: note = PR302335 HFWR1 Kabat
DVQLVESGGGLVQPGGSRKLSCAASGFTFS

SEQ ID NO: 22 - protein, 5 aa, synthetic construct
  REGION 1..5: note = PR004290 HCDR1 Kabat, PR005656 HCDR1 Kabat, PR005657 HCDR1 Kabat,PR005658 HCDR1 Kabat, PR005660 HCDR1 Kabat, PR005662 HCDR1 Kabat,PR006142 HCDR1 Kabat, PR006143 HCDR1 Kabat, PR006144 HCDR1 Kabat,PR
SYWMT

SEQ ID NO: 23 - protein, 5 aa, synthetic construct
  REGION 1..5: note = PR006669 HCDR1 Kabat, PR006744 HCDR1 Kabat, PR006745 HCDR1 Kabat,PR006746 HCDR1 Kabat
DNWMS

SEQ ID NO: 24 - protein, 5 aa, synthetic construct
  REGION 1..5: note = PR301306 HCDR1 Kabat, PR301777 HCDR1 Kabat, PR301788 HCDR1 Kabat,PR301790 HCDR1 Kabat, PR301803 HCDR1 Kabat, PR302029 HCDR1 Kabat,PR302031 HCDR1 Kabat, PR302034 HCDR1 Kabat, PR302036 HCDR1 Kabat,PR
NYWMN

SEQ ID NO: 25 - protein, 5 aa, synthetic construct
  REGION 1..5: note = PR301612 HCDR1 Kabat
GAWMA

SEQ ID NO: 26 - protein, 5 aa, synthetic construct
  REGION 1..5: note = PR301780 HCDR1 Kabat
NFGLN

SEQ ID NO: 27 - protein, 5 aa, synthetic construct
  REGION 1..5: note = PR301806 HCDR1 Kabat
SYWMH

SEQ ID NO: 28 - protein, 5 aa, synthetic construct
  REGION 1..5: note = PR302309-10 HCDR1 Kabat, PR302309-11 HCDR1 Kabat, PR302309-12HCDR1 Kabat, PR302309-13 HCDR1 Kabat, PR302309-14 HCDR1 Kabat,PR302309-15 HCDR1 Kabat, PR302309-16 HCDR1 Kabat, PR302309-17HCDR1 Kabat,
NYGMN

SEQ ID NO: 29 - protein, 5 aa, synthetic construct
  REGION 1..5: note = PR302312 HCDR1 Kabat, PR302324 HCDR1 Kabat, PR302341-10 HCDR1Kabat, PR302341-11 HCDR1 Kabat, PR302341-12 HCDR1 Kabat,PR302341-13 HCDR1 Kabat, PR302341-14 HCDR1 Kabat, PR302341-15HCDR1 Kabat, PR3023
DYFMK

SEQ ID NO: 30 - protein, 5 aa, synthetic construct
  REGION 1..5: note = PR302315 HCDR1 Kabat, PR302335 HCDR1 Kabat
SFGMH

SEQ ID NO: 31 - protein, 5 aa, synthetic construct
  REGION 1..5: note = PR302328 HCDR1 Kabat, PR302331 HCDR1 Kabat
SYAMS

SEQ ID NO: 32 - protein, 5 aa, synthetic construct
  REGION 1..5: note = PR302332 HCDR1 Kabat
NFGMH

SEQ ID NO: 33 - protein, 5 aa, synthetic construct
  REGION 1..5: note = PR302333 HCDR1 Kabat
NFGMN

SEQ ID NO: 34 - protein, 5 aa, synthetic construct
  REGION 1..5: note = PR302334-10 HCDR1 Kabat, PR302334-11 HCDR1 Kabat, PR302334-12HCDR1 Kabat, PR302334-14 HCDR1 Kabat, PR302334-15 HCDR1 Kabat,PR302334-16 HCDR1 Kabat, PR302334-17 HCDR1 Kabat, PR302334-18HCDR1 Kabat,
TYAMS

SEQ ID NO: 35 - protein, 5 aa, synthetic construct
  REGION 1..5: note = PR302339 HCDR1 Kabat
SYALS

SEQ ID NO: 36 - protein, 14 aa, synthetic construct
  REGION 1..14: note = PR004290 HFWR2 Kabat, PR005656 HFWR2 Kabat, PR005657 HFWR2 Kabat,PR005658 HFWR2 Kabat, PR005660 HFWR2 Kabat, PR005662 HFWR2 Kabat,PR006142 HFWR2 Kabat, PR006143 HFWR2 Kabat, PR006144 HFWR2 Kabat,PR
WVRQAPGKGLEWVA

SEQ ID NO: 37 - protein, 14 aa, synthetic construct
  REGION 1..14: note = PR301306 HFWR2 Kabat, PR301612 HFWR2 Kabat, PR301777 HFWR2 Kabat,PR301788 HFWR2 Kabat, PR301790 HFWR2 Kabat, PR301803 HFWR2 Kabat
WVRQSPEKGLEWVA

SEQ ID NO: 38 - protein, 14 aa, synthetic construct
  REGION 1..14: note = PR301780 HFWR2 Kabat
WVKQAPGKGLKWMG

SEQ ID NO: 39 - protein, 14 aa, synthetic construct
  REGION 1..14: note = PR301806 HFWR2 Kabat
WVKQRPGQGLEWIG

SEQ ID NO: 40 - protein, 14 aa, synthetic construct
  REGION 1..14: note = PR302029 HFWR2 Kabat, PR302031 HFWR2 Kabat, PR302034 HFWR2 Kabat
WVRQAPGKGLEWVG

SEQ ID NO: 41 - protein, 14 aa, synthetic construct
  REGION 1..14: note = PR302043 HFWR2 Kabat, PR302045 HFWR2 Kabat, PR302048 HFWR2 Kabat
WVRQASGKGLEWVG

SEQ ID NO: 42 - protein, 14 aa, synthetic construct
  REGION 1..14: note = PR302050 HFWR2 Kabat, PR302052 HFWR2 Kabat, PR302055 HFWR2 Kabat
WVRQASGKGLEWVA

SEQ ID NO: 43 - protein, 14 aa, synthetic construct
  REGION 1..14: note = PR302309-1 HFWR2 Kabat, PR302309-2 HFWR2 Kabat, PR302309-3 HFWR2Kabat, PR302309-4 HFWR2 Kabat, PR302309-5 HFWR2 Kabat, PR302309-6HFWR2 Kabat, PR302309 HFWR2 Kabat, PR302332 HFWR2 Kabat, PR302333HFW
WVKQAPGKVLKWMG

SEQ ID NO: 44 - protein, 14 aa, synthetic construct
  REGION 1..14: note = PR302309-10 HFWR2 Kabat, PR302309-11 HFWR2 Kabat, PR302309-12HFWR2 Kabat, PR302309-13 HFWR2 Kabat, PR302309-14 HFWR2 Kabat,PR302309-17 HFWR2 Kabat, PR302309-7 HFWR2 Kabat, PR302309-8 HFWR2Kabat, PR
WVRQAPGQGLEWMG

SEQ ID NO: 45 - protein, 14 aa, synthetic construct
  REGION 1..14: note = PR302309-15 HFWR2 Kabat, PR302309-16 HFWR2 Kabat, PR302309-18HFWR2 Kabat, PR302309-19 HFWR2 Kabat, PR302309-20 HFWR2 Kabat,PR302309-21 HFWR2 Kabat, PR302341-15 HFWR2 Kabat, PR302341-16HFWR2 Kabat,
WVRQAPGQRLEWMG

SEQ ID NO: 46 - protein, 14 aa, synthetic construct
  REGION 1..14: note = PR302312 HFWR2 Kabat
WMKQSHGKSLEWIG

SEQ ID NO: 47 - protein, 14 aa, synthetic construct
  REGION 1..14: note = PR302315 HFWR2 Kabat, PR302335 HFWR2 Kabat
WVRQAPEKGLEWVA

SEQ ID NO: 48 - protein, 14 aa, synthetic construct
  REGION 1..14: note = PR302324 HFWR2 Kabat
WVKQSHGGSLEWIG

SEQ ID NO: 49 - protein, 14 aa, synthetic construct
  REGION 1..14: note = PR302328 HFWR2 Kabat, PR302334-1 HFWR2 Kabat, PR302334-2 HFWR2Kabat, PR302334-3 HFWR2 Kabat, PR302334-4 HFWR2 Kabat, PR302334-5HFWR2 Kabat, PR302334-6 HFWR2 Kabat, PR302334 HFWR2 Kabat,PR302339 HFW
WVRQTPEKRLEWVA

SEQ ID NO: 50 - protein, 14 aa, synthetic construct
  REGION 1..14: note = PR302331 HFWR2 Kabat
WVRQSPEKRLEWVA

SEQ ID NO: 51 - protein, 14 aa, synthetic construct
  REGION 1..14: note = PR302334-15 HFWR2 Kabat, PR302334-17 HFWR2 Kabat, PR302334-22HFWR2 Kabat
WVRQAPGKGLEWVS

SEQ ID NO: 52 - protein, 14 aa, synthetic construct
  REGION 1..14: note = PR302341-1 HFWR2 Kabat, PR302341-2 HFWR2 Kabat, PR302341-3 HFWR2Kabat, PR302341-4 HFWR2 Kabat, PR302341-5 HFWR2 Kabat, PR302341-6HFWR2 Kabat, PR302341 HFWR2 Kabat
WVKQSHGESLEWIG

SEQ ID NO: 53 - protein, 17 aa, synthetic construct
  REGION 1..17: note = PR004290 HCDR2 Kabat, PR005658 HCDR2 Kabat
NIKQDGSEKYYVDSVKG

SEQ ID NO: 54 - protein, 17 aa, synthetic construct
  REGION 1..17: note = PR005656 HCDR2 Kabat, PR005660 HCDR2 Kabat, PR006142 HCDR2 Kabat,PR006143 HCDR2 Kabat, PR006144 HCDR2 Kabat, PR006145 HCDR2 Kabat
NIKQEGSEKYYVDSVKG

SEQ ID NO: 55 - protein, 17 aa, synthetic construct
  REGION 1..17: note = PR005657 HCDR2 Kabat, PR005662 HCDR2 Kabat, PR006146 HCDR2 Kabat,PR006147 HCDR2 Kabat, PR006148 HCDR2 Kabat, PR006149 HCDR2 Kabat
NIKQDASEKYYVDSVKG

SEQ ID NO: 56 - protein, 17 aa, synthetic construct
  REGION 1..17: note = PR006150 HCDR2 Kabat, PR006151 HCDR2 Kabat, PR006152 HCDR2 Kabat,PR006153 HCDR2 Kabat
NIKQDSSEKYYVDSVKG

SEQ ID NO: 57 - protein, 17 aa, synthetic construct
  REGION 1..17: note = PR006669 HCDR2 Kabat
NIKQDGSENYYVDSVKG

SEQ ID NO: 58 - protein, 17 aa, synthetic construct
  REGION 1..17: note = PR006744 HCDR2 Kabat
NIKQEGSENYYVDSVKG

SEQ ID NO: 59 - protein, 17 aa, synthetic construct
  REGION 1..17: note = PR006745 HCDR2 Kabat
NIKQDASENYYVDSVKG

SEQ ID NO: 60 - protein, 17 aa, synthetic construct
  REGION 1..17: note = PR006746 HCDR2 Kabat
NIKQDSSENYYVDSVKG

SEQ ID NO: 61 - protein, 19 aa, synthetic construct
  REGION 1..19: note = PR301306 HCDR2 Kabat
EIRLKSNNYATLYAESVKG

SEQ ID NO: 62 - protein, 19 aa, synthetic construct
  REGION 1..19: note = PR301612 HCDR2 Kabat
EIKNKANNHATFYAESVKG

SEQ ID NO: 63 - protein, 19 aa, synthetic construct
  REGION 1..19: note = PR301777 HCDR2 Kabat, PR301788 HCDR2 Kabat, PR301790 HCDR2 Kabat,PR302029 HCDR2 Kabat, PR302031 HCDR2 Kabat, PR302034 HCDR2 Kabat,PR302036 HCDR2 Kabat, PR302038 HCDR2 Kabat, PR302041 HCDR2 Kabat,PR
EIRLKSNNYATHYAESVKG

SEQ ID NO: 64 - protein, 17 aa, synthetic construct
  REGION 1..17: note = PR301780 HCDR2 Kabat
WINTYTGEPTYTDDFKG

SEQ ID NO: 65 - protein, 19 aa, synthetic construct
  REGION 1..19: note = PR301803 HCDR2 Kabat
EIRLKSNNYATHYAESLKG

SEQ ID NO: 66 - protein, 17 aa, synthetic construct
  REGION 1..17: note = PR301806 HCDR2 Kabat
DIYPGSDSTNYNEKFKS

SEQ ID NO: 67 - protein, 17 aa, synthetic construct
  REGION 1..17: note = PR302309-10 HCDR2 Kabat, PR302309-11 HCDR2 Kabat, PR302309-12HCDR2 Kabat, PR302309-13 HCDR2 Kabat, PR302309-14 HCDR2 Kabat,PR302309-15 HCDR2 Kabat, PR302309-16 HCDR2 Kabat, PR302309-17HCDR2 Kabat,
WINTYTGEPIYTDDFKG

SEQ ID NO: 68 - protein, 17 aa, synthetic construct
  REGION 1..17: note = PR302312 HCDR2 Kabat
VINPNNGDTFYNQNFKG

SEQ ID NO: 69 - protein, 17 aa, synthetic construct
  REGION 1..17: note = PR302315 HCDR2 Kabat
YISSGSSIIYYADTVKG

SEQ ID NO: 70 - protein, 17 aa, synthetic construct
  REGION 1..17: note = PR302324 HCDR2 Kabat
VISPNNGDTFYNQKFKG

SEQ ID NO: 71 - protein, 16 aa, synthetic construct
  REGION 1..16: note = PR302328 HCDR2 Kabat, PR302331 HCDR2 Kabat, PR302334-10 HCDR2Kabat, PR302334-11 HCDR2 Kabat, PR302334-12 HCDR2 Kabat,PR302334-14 HCDR2 Kabat, PR302334-15 HCDR2 Kabat, PR302334-16HCDR2 Kabat, PR3023
SIRSGGTTYYPDSVKG

SEQ ID NO: 72 - protein, 17 aa, synthetic construct
  REGION 1..17: note = PR302332 HCDR2 Kabat
WINTYTGEPIYAADFKG

SEQ ID NO: 73 - protein, 17 aa, synthetic construct
  REGION 1..17: note = PR302333 HCDR2 Kabat
WINTYTGEPIYADDFKG

SEQ ID NO: 74 - protein, 17 aa, synthetic construct
  REGION 1..17: note = PR302335 HCDR2 Kabat
YISSGSTIFYYADTVKG

SEQ ID NO: 75 - protein, 17 aa, synthetic construct
  REGION 1..17: note = PR302341-10 HCDR2 Kabat, PR302341-11 HCDR2 Kabat, PR302341-12HCDR2 Kabat, PR302341-13 HCDR2 Kabat, PR302341-14 HCDR2 Kabat,PR302341-15 HCDR2 Kabat, PR302341-16 HCDR2 Kabat, PR302341-17HCDR2 Kabat,
VINPNNGDIFYNQKFKG

SEQ ID NO: 76 - protein, 17 aa, synthetic construct
  REGION 1..17: note = PR302341-28 HCDR2 Kabat, PR302341-32 HCDR2 Kabat, PR302341-34HCDR2 Kabat, PR302341-38 HCDR2 Kabat, PR302341-45 HCDR2 Kabat,PR302341-48 HCDR2 Kabat, PR302341-51 HCDR2 Kabat, PR302341-54HCDR2 Kabat,
VINPNQGDIFYNQKFKG

SEQ ID NO: 77 - protein, 17 aa, synthetic construct
  REGION 1..17: note = PR302341-29 HCDR2 Kabat, PR302341-33 HCDR2 Kabat, PR302341-35HCDR2 Kabat, PR302341-39 HCDR2 Kabat, PR302341-43 HCDR2 Kabat,PR302341-46 HCDR2 Kabat, PR302341-49 HCDR2 Kabat, PR302341-52HCDR2 Kabat,
VINPNNADIFYNQKFKG

SEQ ID NO: 78 - protein, 17 aa, synthetic construct
  REGION 1..17: note = PR302341-36 HCDR2 Kabat, PR302341-37 HCDR2 Kabat, PR302341-40HCDR2 Kabat, PR302341-41 HCDR2 Kabat, PR302341-44 HCDR2 Kabat,PR302341-47 HCDR2 Kabat, PR302341-50 HCDR2 Kabat, PR302341-53HCDR2 Kabat,
VINPNSGDIFYNQKFKG

SEQ ID NO: 79 - protein, 32 aa, synthetic construct
  REGION 1..32: note = PR004290 HFWR3 Kabat, PR005656 HFWR3 Kabat, PR005657 HFWR3 Kabat,PR005658 HFWR3 Kabat, PR005660 HFWR3 Kabat, PR005662 HFWR3 Kabat,PR006142 HFWR3 Kabat, PR006143 HFWR3 Kabat, PR006144 HFWR3 Kabat,PR
RFTISRDNAKNSLFLQMNSLRAEDTAVYYCAR

SEQ ID NO: 80 - protein, 32 aa, synthetic construct
  REGION 1..32: note = PR006669 HFWR3 Kabat, PR006744 HFWR3 Kabat, PR006745 HFWR3 Kabat,PR006746 HFWR3 Kabat
RFTISRDNAKNSLYLQMNSLRAEDTAVYYCSR

SEQ ID NO: 81 - protein, 32 aa, synthetic construct
  REGION 1..32: note = PR301306 HFWR3 Kabat
RFTISRDDSKSSVYLQMNNLRAEDTGIYYCTR

SEQ ID NO: 82 - protein, 32 aa, synthetic construct
  REGION 1..32: note = PR301612 HFWR3 Kabat
RFIISRDDSKSSVYLQMNSLRAEDTGTYYCTL

SEQ ID NO: 83 - protein, 32 aa, synthetic construct
  REGION 1..32: note = PR301777 HFWR3 Kabat
RFTISRDDSKSGVYLQMNNLGAEDTGIYYCTV

SEQ ID NO: 84 - protein, 32 aa, synthetic construct
  REGION 1..32: note = PR301780 HFWR3 Kabat
RFAFSLETSASTAYLQINNLKKEDTATYFCAR

SEQ ID NO: 85 - protein, 32 aa, synthetic construct
  REGION 1..32: note = PR301788 HFWR3 Kabat, PR301790 HFWR3 Kabat
RFTISRDDSKSSVYLQMNNLRAEDTGIYYCTI

SEQ ID NO: 86 - protein, 32 aa, synthetic construct
  REGION 1..32: note = PR301803 HFWR3 Kabat
RFTISRDDSKSSVYLQMNNLRAEDTGIYYCTV

SEQ ID NO: 87 - protein, 32 aa, synthetic construct
  REGION 1..32: note = PR301806 HFWR3 Kabat
KASLTVDTSSSTAYMQLSSLTSEDSAVYYCAR

SEQ ID NO: 88 - protein, 32 aa, synthetic construct
  REGION 1..32: note = PR302029 HFWR3 Kabat, PR302031 HFWR3 Kabat, PR302034 HFWR3 Kabat
RFTISRDDSKNSLYLQMNSLKTEDTAVYYCTV

SEQ ID NO: 89 - protein, 32 aa, synthetic construct
  REGION 1..32: note = PR302036 HFWR3 Kabat, PR302038 HFWR3 Kabat, PR302041 HFWR3 Kabat
RFTISRDDSKSSVYLQMNSLKTEDTAVYYCTV

SEQ ID NO: 90 - protein, 32 aa, synthetic construct
  REGION 1..32: note = PR302043 HFWR3 Kabat, PR302045 HFWR3 Kabat, PR302048 HFWR3 Kabat
RFTISRDDSKNTAYLQMNSLKTEDTAVYYCTR

SEQ ID NO: 91 - protein, 32 aa, synthetic construct
  REGION 1..32: note = PR302050 HFWR3 Kabat, PR302052 HFWR3 Kabat, PR302055 HFWR3 Kabat,PR302064 HFWR3 Kabat, PR302066 HFWR3 Kabat, PR302069 HFWR3 Kabat
RFTISRDDSKSTVYLQMNSLKTEDTAVYYCTV

SEQ ID NO: 92 - protein, 32 aa, synthetic construct
  REGION 1..32: note = PR302309-1 HFWR3 Kabat, PR302309-2 HFWR3 Kabat, PR302309-3 HFWR3Kabat, PR302309-4 HFWR3 Kabat, PR302309-5 HFWR3 Kabat, PR302309-6HFWR3 Kabat, PR302309 HFWR3 Kabat, PR302332 HFWR3 Kabat
RFAFSLETSASTAYLQINNLKNEDTATYFCSR

SEQ ID NO: 93 - protein, 32 aa, synthetic construct
  REGION 1..32: note = PR302309-10 HFWR3 Kabat, PR302309-11 HFWR3 Kabat, PR302309-12HFWR3 Kabat, PR302309-14 HFWR3 Kabat, PR302309-7 HFWR3 Kabat,PR302309-8 HFWR3 Kabat, PR302309-9 HFWR3 Kabat
RFVFSLDTSVSTAYLQISSLKAEDTAVYYCSR

SEQ ID NO: 94 - protein, 32 aa, synthetic construct
  REGION 1..32: note = PR302309-13 HFWR3 Kabat, PR302309-17 HFWR3 Kabat
RFVFSLDTSVSTAYLQISSLKAEDTAVYYCAR

SEQ ID NO: 95 - protein, 32 aa, synthetic construct
  REGION 1..32: note = PR302309-15 HFWR3 Kabat, PR302309-18 HFWR3 Kabat, PR302341-15HFWR3 Kabat, PR302341-20 HFWR3 Kabat, PR302341-55 HFWR3 Kabat,PR302341-56 HFWR3 Kabat, PR302341-57 HFWR3 Kabat, PR302341-58HFWR3 Kabat,
RVTITRDTSASTAYMELSSLRSEDTAVYYCAR

SEQ ID NO: 96 - protein, 32 aa, synthetic construct
  REGION 1..32: note = PR302309-16 HFWR3 Kabat, PR302309-19 HFWR3 Kabat, PR302309-20HFWR3 Kabat, PR302309-21 HFWR3 Kabat
RVTITLDTSASTAYMELSSLRSEDTAVYYCSR

SEQ ID NO: 97 - protein, 32 aa, synthetic construct
  REGION 1..32: note = PR302312 HFWR3 Kabat
KATLTVDKSSSTAYMQLNSLTSEDSAVYYCTR

SEQ ID NO: 98 - protein, 32 aa, synthetic construct
  REGION 1..32: note = PR302315 HFWR3 Kabat
RFTISRDNPKNILFLQMTSLRSEDTAMYYCAR

SEQ ID NO: 99 - protein, 32 aa, synthetic construct
  REGION 1..32: note = PR302324 HFWR3 Kabat, PR302341-1 HFWR3 Kabat, PR302341-2 HFWR3Kabat, PR302341-3 HFWR3 Kabat, PR302341-4 HFWR3 Kabat, PR302341-5HFWR3 Kabat, PR302341-6 HFWR3 Kabat, PR302341 HFWR3 Kabat
KATLTVDKSSSTAFMQLNSLTSEDSAVYSCAR

SEQ ID NO: 100 - protein, 32 aa, synthetic construct
  REGION 1..32: note = PR302328 HFWR3 Kabat, PR302331 HFWR3 Kabat, PR302334-1 HFWR3Kabat, PR302334-2 HFWR3 Kabat, PR302334-3 HFWR3 Kabat, PR302334-4HFWR3 Kabat, PR302334-5 HFWR3 Kabat, PR302334-6 HFWR3 Kabat,PR302334 HFW
RFTISRDNARNILYLQMSSLRSEDTAIYYCTR

SEQ ID NO: 101 - protein, 32 aa, synthetic construct
  REGION 1..32: note = PR302333 HFWR3 Kabat
RFAFSLETSASAAYLQINNLKNEDTATYFCSR

SEQ ID NO: 102 - protein, 32 aa, synthetic construct
  REGION 1..32: note = PR302334-10 HFWR3 Kabat, PR302334-11 HFWR3 Kabat, PR302334-12HFWR3 Kabat, PR302334-14 HFWR3 Kabat, PR302334-7 HFWR3 Kabat,PR302334-8 HFWR3 Kabat, PR302334-9 HFWR3 Kabat
RFTISRDNSKNTLYLQMNSLRAEDTAVYYCTR

SEQ ID NO: 103 - protein, 32 aa, synthetic construct
  REGION 1..32: note = PR302334-15 HFWR3 Kabat
RFTISRDNAKNSLYLQMNSLRAEDTAVYYCAR

SEQ ID NO: 104 - protein, 32 aa, synthetic construct
  REGION 1..32: note = PR302334-16 HFWR3 Kabat, PR302334-24 HFWR3 Kabat, PR302334-25HFWR3 Kabat
RFTISRDNAKNSLYLQMNSLRAEDTAVYYCTR

SEQ ID NO: 105 - protein, 32 aa, synthetic construct
  REGION 1..32: note = PR302334-17 HFWR3 Kabat, PR302334-22 HFWR3 Kabat
RFTISRDNSKNSLYLQMNSLRAEDTAVYYCAR

SEQ ID NO: 106 - protein, 32 aa, synthetic construct
  REGION 1..32: note = PR302334-18 HFWR3 Kabat, PR302334-26 HFWR3 Kabat, PR302334-27HFWR3 Kabat
RFTISRDNSKNSLYLQMNSLRAEDTAVYYCTR

SEQ ID NO: 107 - protein, 32 aa, synthetic construct
  REGION 1..32: note = PR302335 HFWR3 Kabat
RFTISRDNPKNTLFLQMTSLRSEDTAMYYCAR

SEQ ID NO: 108 - protein, 32 aa, synthetic construct
  REGION 1..32: note = PR302341-10 HFWR3 Kabat, PR302341-11 HFWR3 Kabat, PR302341-12HFWR3 Kabat, PR302341-14 HFWR3 Kabat, PR302341-43 HFWR3 Kabat,PR302341-44 HFWR3 Kabat, PR302341-45 HFWR3 Kabat, PR302341-46HFWR3 Kabat,
RVTMTVDKSTSTAYMELSSLRSEDTAVYYCAR

SEQ ID NO: 109 - protein, 32 aa, synthetic construct
  REGION 1..32: note = PR302341-13 HFWR3 Kabat, PR302341-19 HFWR3 Kabat
RVTMTRDTSTSTVYMELSSLRSEDTAVYYCAR

SEQ ID NO: 110 - protein, 32 aa, synthetic construct
  REGION 1..32: note = PR302341-16 HFWR3 Kabat, PR302341-21 HFWR3 Kabat, PR302341-24HFWR3 Kabat, PR302341-25 HFWR3 Kabat
RVTITVDKSASTAYMELSSLRSEDTAVYYCAR

SEQ ID NO: 111 - protein, 32 aa, synthetic construct
  REGION 1..32: note = PR302341-17 HFWR3 Kabat, PR302341-22 HFWR3 Kabat
RVTMTRDTSISTAYMELSRLRSDDTAVYYCAR

SEQ ID NO: 112 - protein, 32 aa, synthetic construct
  REGION 1..32: note = PR302341-18 HFWR3 Kabat, PR302341-23 HFWR3 Kabat, PR302341-26HFWR3 Kabat, PR302341-27 HFWR3 Kabat, PR302341-28 HFWR3 Kabat,PR302341-29 HFWR3 Kabat, PR302341-30 HFWR3 Kabat, PR302341-31HFWR3 Kabat,
RVTMTVDKSISTAYMELSRLRSDDTAVYYCAR

SEQ ID NO: 113 - protein, 6 aa, synthetic construct
  REGION 1..6: note = PR004290 HCDR3 Kabat, PR005656 HCDR3 Kabat, PR005657 HCDR3 Kabat,PR005658 HCDR3 Kabat, PR005660 HCDR3 Kabat, PR005662 HCDR3 Kabat,PR006142 HCDR3 Kabat, PR006143 HCDR3 Kabat, PR006144 HCDR3 Kabat,PR
TLIFDY

SEQ ID NO: 114 - protein, 6 aa, synthetic construct
  REGION 1..6: note = PR006669 HCDR3 Kabat, PR006744 HCDR3 Kabat, PR006745 HCDR3 Kabat,PR006746 HCDR3 Kabat
GLALWG

SEQ ID NO: 115 - protein, 5 aa, synthetic construct
  REGION 1..5: note = PR301306 HCDR3 Kabat, PR301777 HCDR3 Kabat, PR301788 HCDR3 Kabat,PR301790 HCDR3 Kabat, PR301803 HCDR3 Kabat, PR302029 HCDR3 Kabat,PR302031 HCDR3 Kabat, PR302034 HCDR3 Kabat, PR302036 HCDR3 Kabat,PR
GTLVY

SEQ ID NO: 116 - protein, 12 aa, synthetic construct
  REGION 1..12: note = PR301780 HCDR3 Kabat
GTYRDDGAWFAY

SEQ ID NO: 117 - protein, 12 aa, synthetic construct
  REGION 1..12: note = PR301806 HCDR3 Kabat
SITTTTFWYFDV

SEQ ID NO: 118 - protein, 12 aa, synthetic construct
  REGION 1..12: note = PR302309-10 HCDR3 Kabat, PR302309-11 HCDR3 Kabat, PR302309-12HCDR3 Kabat, PR302309-13 HCDR3 Kabat, PR302309-14 HCDR3 Kabat,PR302309-15 HCDR3 Kabat, PR302309-16 HCDR3 Kabat, PR302309-17HCDR3 Kabat,
STMITTGGVFAY

SEQ ID NO: 119 - protein, 9 aa, synthetic construct
  REGION 1..9: note = PR302312 HCDR3 Kabat
GTARALFAY

SEQ ID NO: 120 - protein, 9 aa, synthetic construct
  REGION 1..9: note = PR302315 HCDR3 Kabat
DITTATFAY

SEQ ID NO: 121 - protein, 9 aa, synthetic construct
  REGION 1..9: note = PR302324 HCDR3 Kabat, PR302341-10 HCDR3 Kabat, PR302341-11 HCDR3Kabat, PR302341-12 HCDR3 Kabat, PR302341-13 HCDR3 Kabat,PR302341-14 HCDR3 Kabat, PR302341-15 HCDR3 Kabat, PR302341-16HCDR3 Kabat, PR3
GTDRALFAY

SEQ ID NO: 122 - protein, 9 aa, synthetic construct
  REGION 1..9: note = PR302335 HCDR3 Kabat
DITTATFAF

SEQ ID NO: 123 - protein, 11 aa, synthetic construct
  REGION 1..11: note = PR004290 HFWR4 Kabat, PR005656 HFWR4 Kabat, PR005657 HFWR4 Kabat,PR005658 HFWR4 Kabat, PR005660 HFWR4 Kabat, PR005662 HFWR4 Kabat,PR006142 HFWR4 Kabat, PR006143 HFWR4 Kabat, PR006144 HFWR4 Kabat,PR
WGQGTLVTVSS

SEQ ID NO: 124 - protein, 11 aa, synthetic construct
  REGION 1..11: note = PR301306 HFWR4 Kabat, PR301777 HFWR4 Kabat, PR301780 HFWR4 Kabat,PR301788 HFWR4 Kabat, PR301790 HFWR4 Kabat, PR301803 HFWR4 Kabat,PR302309-1 HFWR4 Kabat, PR302309-2 HFWR4 Kabat, PR302309-3 HFWR4Kab
WGQGTLVTVSA

SEQ ID NO: 125 - protein, 11 aa, synthetic construct
  REGION 1..11: note = PR301612 HFWR4 Kabat
WGTGTTVTVSS

SEQ ID NO: 126 - protein, 11 aa, synthetic construct
  REGION 1..11: note = PR301806 HFWR4 Kabat
WGAGTTVTVSS

SEQ ID NO: 127 - protein, 11 aa, synthetic construct
  REGION 1..11: note = PR302334-10 HFWR4 Kabat, PR302334-11 HFWR4 Kabat, PR302334-12HFWR4 Kabat, PR302334-14 HFWR4 Kabat, PR302334-15 HFWR4 Kabat,PR302334-16 HFWR4 Kabat, PR302334-17 HFWR4 Kabat, PR302334-18HFWR4 Kabat,
WGQGTTVTVSS

SEQ ID NO: 128 - protein, 11 aa, synthetic construct
  REGION 1..11: note = PR302341-1 HFWR4 Kabat, PR302341-2 HFWR4 Kabat, PR302341-3 HFWR4Kabat, PR302341-4 HFWR4 Kabat, PR302341-5 HFWR4 Kabat, PR302341-6HFWR4 Kabat, PR302341 HFWR4 Kabat
WGQGTLVAVSA

SEQ ID NO: 129 - protein, 23 aa, synthetic construct
  REGION 1..23: note = PR004290 LFWR1 Kabat, PR005656 LFWR1 Kabat, PR005657 LFWR1 Kabat,PR005658 LFWR1 Kabat, PR005660 LFWR1 Kabat, PR005662 LFWR1 Kabat,PR006142 LFWR1 Kabat, PR006143 LFWR1 Kabat, PR006144 LFWR1 Kabat,PR
DIVMTQSPLSLPVTPGEPASISC

SEQ ID NO: 130 - protein, 23 aa, synthetic construct
  REGION 1..23: note = PR006669 LFWR1 Kabat, PR006744 LFWR1 Kabat, PR006745 LFWR1 Kabat,PR006746 LFWR1 Kabat
DIVMTHSPLSLPVTPGDPASISC

SEQ ID NO: 131 - protein, 23 aa, synthetic construct
  REGION 1..23: note = PR301306 LFWR1 Kabat, PR301777 LFWR1 Kabat, PR301788 LFWR1 Kabat,PR301790 LFWR1 Kabat
DIVLTQSPASLAVSLGQRATISC

SEQ ID NO: 132 - protein, 23 aa, synthetic construct
  REGION 1..23: note = PR301612 LFWR1 Kabat
QIVLTQSPAIMSASPGERVTISC

SEQ ID NO: 133 - protein, 23 aa, synthetic construct
  REGION 1..23: note = PR301780 LFWR1 Kabat, PR301806 LFWR1 Kabat, PR302312 LFWR1 Kabat,PR302324 LFWR1 Kabat, PR302341-13 LFWR1 Kabat, PR302341-14 LFWR1Kabat, PR302341-15 LFWR1 Kabat, PR302341-16 LFWR1 Kabat,PR302341-17
DVLMTQTPLSLPVSLGDQASISC

SEQ ID NO: 134 - protein, 23 aa, synthetic construct
  REGION 1..23: note = PR301803 LFWR1 Kabat
DIVLTQSPASLVVSLGQRATISC

SEQ ID NO: 135 - protein, 23 aa, synthetic construct
  REGION 1..23: note = PR302029 LFWR1 Kabat, PR302036 LFWR1 Kabat, PR302043 LFWR1 Kabat,PR302050 LFWR1 Kabat, PR302064 LFWR1 Kabat
DIVLTQSPDSLAVSLGERATINC

SEQ ID NO: 136 - protein, 23 aa, synthetic construct
  REGION 1..23: note = PR302031 LFWR1 Kabat, PR302038 LFWR1 Kabat, PR302045 LFWR1 Kabat,PR302052 LFWR1 Kabat, PR302066 LFWR1 Kabat
EIVLTQSPATLSLSPGERATLSC

SEQ ID NO: 137 - protein, 23 aa, synthetic construct
  REGION 1..23: note = PR302034 LFWR1 Kabat, PR302041 LFWR1 Kabat, PR302048 LFWR1 Kabat,PR302055 LFWR1 Kabat, PR302069 LFWR1 Kabat
DIQLTQSPSSLSASVGDRVTITC

SEQ ID NO: 138 - protein, 23 aa, synthetic construct
  REGION 1..23: note = PR302309-10 LFWR1 Kabat, PR302309-20 LFWR1 Kabat, PR302309-4LFWR1 Kabat, PR302341-12 LFWR1 Kabat, PR302341-25 LFWR1 Kabat,PR302341-27 LFWR1 Kabat, PR302341-6 LFWR1 Kabat
DVVMTQTPLSLSVTPGQPASISC

SEQ ID NO: 139 - protein, 23 aa, synthetic construct
  REGION 1..23: note = PR302309-11 LFWR1 Kabat, PR302309-5 LFWR1 Kabat, PR302341-3 LFWR1Kabat, PR302341-9 LFWR1 Kabat
DIVMTQTPLSSPVTLGQPASISC

SEQ ID NO: 140 - protein, 23 aa, synthetic construct
  REGION 1..23: note = PR302309-12 LFWR1 Kabat, PR302309-21 LFWR1 Kabat, PR302309-6LFWR1 Kabat, PR302341-10 LFWR1 Kabat, PR302341-24 LFWR1 Kabat,PR302341-26 LFWR1 Kabat, PR302341-43 LFWR1 Kabat, PR302341-44LFWR1 Kabat, P
DVVMTQTPLSSPVTLGQPASISC

SEQ ID NO: 141 - protein, 23 aa, synthetic construct
  REGION 1..23: note = PR302309-13 LFWR1 Kabat, PR302309-14 LFWR1 Kabat, PR302309-15LFWR1 Kabat, PR302309-16 LFWR1 Kabat, PR302309 LFWR1 Kabat,PR302332 LFWR1 Kabat, PR302333 LFWR1 Kabat
DVLMAQTPLSLPVSLGDQASISC

SEQ ID NO: 142 - protein, 23 aa, synthetic construct
  REGION 1..23: note = PR302309-17 LFWR1 Kabat, PR302309-18 LFWR1 Kabat, PR302309-19LFWR1 Kabat, PR302309-1 LFWR1 Kabat, PR302309-2 LFWR1 Kabat,PR302309-7 LFWR1 Kabat, PR302309-8 LFWR1 Kabat, PR302341-19 LFWR1Kabat, PR30
DVVMTQSPLSLPVTLGQPASISC

SEQ ID NO: 143 - protein, 23 aa, synthetic construct
  REGION 1..23: note = PR302309-3 LFWR1 Kabat, PR302309-9 LFWR1 Kabat, PR302341-11 LFWR1Kabat, PR302341-5 LFWR1 Kabat
DIVMTQTPLSLSVTPGQPASISC

SEQ ID NO: 144 - protein, 23 aa, synthetic construct
  REGION 1..23: note = PR302315 LFWR1 Kabat
DVVMTQTPLSLPVSLGDQASISC

SEQ ID NO: 145 - protein, 23 aa, synthetic construct
  REGION 1..23: note = PR302328 LFWR1 Kabat, PR302339 LFWR1 Kabat
NIVMTQTPKFLLVSAGDRVTITC

SEQ ID NO: 146 - protein, 23 aa, synthetic construct
  REGION 1..23: note = PR302331 LFWR1 Kabat
NIVMTQTPKFLLVSAGDRVTIAC

SEQ ID NO: 147 - protein, 23 aa, synthetic construct
  REGION 1..23: note = PR302334-1 LFWR1 Kabat, PR302334-22 LFWR1 Kabat, PR302334-2 LFWR1Kabat, PR302334-7 LFWR1 Kabat, PR302334-8 LFWR1 Kabat
EIVMTQSPATLSVSPGERATLSC

SEQ ID NO: 148 - protein, 23 aa, synthetic construct
  REGION 1..23: note = PR302334-10 LFWR1 Kabat, PR302334-24 LFWR1 Kabat, PR302334-26LFWR1 Kabat, PR302334-3 LFWR1 Kabat, PR302334-4 LFWR1 Kabat,PR302334-9 LFWR1 Kabat
DIVMTQSPDSLAVSLGERATINC

SEQ ID NO: 149 - protein, 23 aa, synthetic construct
  REGION 1..23: note = PR302334-11 LFWR1 Kabat, PR302334-12 LFWR1 Kabat, PR302334-25LFWR1 Kabat, PR302334-27 LFWR1 Kabat, PR302334-5 LFWR1 Kabat,PR302334-6 LFWR1 Kabat
DIQMTQSSLSASVSLGDRVTITC

SEQ ID NO: 150 - protein, 23 aa, synthetic construct
  REGION 1..23: note = PR302334-14 LFWR1 Kabat, PR302334-15 LFWR1 Kabat, PR302334-16LFWR1 Kabat, PR302334-17 LFWR1 Kabat, PR302334-18 LFWR1 Kabat,PR302334 LFWR1 Kabat
NIVMTQTPKFLLVSAGDRITITC

SEQ ID NO: 151 - protein, 23 aa, synthetic construct
  REGION 1..23: note = PR302335 LFWR1 Kabat
DVVLTQTPLSLPVSLGDQVSISC

SEQ ID NO: 152 - protein, 16 aa, synthetic construct
  REGION 1..16: note = PR004290 LCDR1 Kabat, PR005656 LCDR1 Kabat, PR005657 LCDR1 Kabat
RSSQSLLHSTGYNSLE

SEQ ID NO: 153 - protein, 16 aa, synthetic construct
  REGION 1..16: note = PR005658 LCDR1 Kabat, PR005660 LCDR1 Kabat, PR005662 LCDR1 Kabat
RSSQSLLHSTGYQSLE

SEQ ID NO: 154 - protein, 16 aa, synthetic construct
  REGION 1..16: note = PR006142 LCDR1 Kabat, PR006146 LCDR1 Kabat, PR006150 LCDR1 Kabat
RSSQSLLHSTGYDSLE

SEQ ID NO: 155 - protein, 16 aa, synthetic construct
  REGION 1..16: note = PR006143 LCDR1 Kabat, PR006147 LCDR1 Kabat, PR006151 LCDR1 Kabat
RSSQSLLHSTGYHSLE

SEQ ID NO: 156 - protein, 16 aa, synthetic construct
  REGION 1..16: note = PR006144 LCDR1 Kabat, PR006148 LCDR1 Kabat, PR006152 LCDR1 Kabat
RSSQSLLHSTGYNPLE

SEQ ID NO: 157 - protein, 16 aa, synthetic construct
  REGION 1..16: note = PR006145 LCDR1 Kabat, PR006149 LCDR1 Kabat, PR006153 LCDR1 Kabat
RSSQSLLHSTGYSSLE

SEQ ID NO: 158 - protein, 16 aa, synthetic construct
  REGION 1..16: note = PR006669 LCDR1 Kabat, PR006744 LCDR1 Kabat, PR006745 LCDR1 Kabat,PR006746 LCDR1 Kabat
RSSQSLLHSTGYNYLD

SEQ ID NO: 159 - protein, 15 aa, synthetic construct
  REGION 1..15: note = PR301306 LCDR1 Kabat
RASQSVSTSDYNYMH

SEQ ID NO: 160 - protein, 10 aa, synthetic construct
  REGION 1..10: note = PR301612 LCDR1 Kabat
SASSSVSNMY

SEQ ID NO: 161 - protein, 15 aa, synthetic construct
  REGION 1..15: note = PR301777 LCDR1 Kabat, PR301788 LCDR1 Kabat, PR301803 LCDR1 Kabat,PR302029 LCDR1 Kabat, PR302031 LCDR1 Kabat, PR302034 LCDR1 Kabat,PR302036 LCDR1 Kabat, PR302038 LCDR1 Kabat, PR302041 LCDR1 Kabat,PR
RASQSVSTSTYNYMH

SEQ ID NO: 162 - protein, 16 aa, synthetic construct
  REGION 1..16: note = PR301780 LCDR1 Kabat
RSSQIIVHSNGNTYLE

SEQ ID NO: 163 - protein, 15 aa, synthetic construct
  REGION 1..15: note = PR301790 LCDR1 Kabat
RASQSVSTSSYNYMH

SEQ ID NO: 164 - protein, 16 aa, synthetic construct
  REGION 1..16: note = PR301806 LCDR1 Kabat
RSSQSFVYSNGNTYLQ

SEQ ID NO: 165 - protein, 16 aa, synthetic construct
  REGION 1..16: note = PR302309-10 LCDR1 Kabat, PR302309-11 LCDR1 Kabat, PR302309-12LCDR1 Kabat, PR302309-13 LCDR1 Kabat, PR302309-14 LCDR1 Kabat,PR302309-15 LCDR1 Kabat, PR302309-16 LCDR1 Kabat, PR302309-17LCDR1 Kabat,
RSSQTIVHSDGNTYLE

SEQ ID NO: 166 - protein, 16 aa, synthetic construct
  REGION 1..16: note = PR302312 LCDR1 Kabat, PR302324 LCDR1 Kabat, PR302332 LCDR1 Kabat
RSSQSIVHSDGNTYLE

SEQ ID NO: 167 - protein, 16 aa, synthetic construct
  REGION 1..16: note = PR302315 LCDR1 Kabat
RSSQSLVHSNGNTYLH

SEQ ID NO: 168 - protein, 11 aa, synthetic construct
  REGION 1..11: note = PR302328 LCDR1 Kabat, PR302331 LCDR1 Kabat, PR302334-10 LCDR1Kabat, PR302334-11 LCDR1 Kabat, PR302334-12 LCDR1 Kabat,PR302334-14 LCDR1 Kabat, PR302334-15 LCDR1 Kabat, PR302334-16LCDR1 Kabat, PR3023
KASQSVNNDVA

SEQ ID NO: 169 - protein, 16 aa, synthetic construct
  REGION 1..16: note = PR302333 LCDR1 Kabat
RSSQNIVHSDGNTYLE

SEQ ID NO: 170 - protein, 16 aa, synthetic construct
  REGION 1..16: note = PR302335 LCDR1 Kabat
RSSQNLLHNNGNTYLH

SEQ ID NO: 171 - protein, 16 aa, synthetic construct
  REGION 1..16: note = PR302341-10 LCDR1 Kabat, PR302341-11 LCDR1 Kabat, PR302341-12LCDR1 Kabat, PR302341-13 LCDR1 Kabat, PR302341-14 LCDR1 Kabat,PR302341-15 LCDR1 Kabat, PR302341-16 LCDR1 Kabat, PR302341-17LCDR1 Kabat,
RSGQSIVHSDGNTYLE

SEQ ID NO: 172 - protein, 16 aa, synthetic construct
  REGION 1..16: note = PR302341-30 LCDR1 Kabat, PR302341-32 LCDR1 Kabat, PR302341-35LCDR1 Kabat, PR302341-36 LCDR1 Kabat, PR302341-49 LCDR1 Kabat,PR302341-50 LCDR1 Kabat, PR302341-51 LCDR1 Kabat, PR302341-61LCDR1 Kabat,
RSGQSIVHSEGNTYLE

SEQ ID NO: 173 - protein, 16 aa, synthetic construct
  REGION 1..16: note = PR302341-31 LCDR1 Kabat, PR302341-33 LCDR1 Kabat, PR302341-34LCDR1 Kabat, PR302341-37 LCDR1 Kabat, PR302341-46 LCDR1 Kabat,PR302341-47 LCDR1 Kabat, PR302341-48 LCDR1 Kabat, PR302341-58LCDR1 Kabat,
RSGQSIVHSDANTYLE

SEQ ID NO: 174 - protein, 16 aa, synthetic construct
  REGION 1..16: note = PR302341-38 LCDR1 Kabat, PR302341-39 LCDR1 Kabat, PR302341-40LCDR1 Kabat, PR302341-42 LCDR1 Kabat, PR302341-52 LCDR1 Kabat,PR302341-53 LCDR1 Kabat, PR302341-54 LCDR1 Kabat, PR302341-64LCDR1 Kabat,
RSGQSIVHSSGNTYLE

SEQ ID NO: 175 - protein, 15 aa, synthetic construct
  REGION 1..15: note = PR004290 LFWR2 Kabat, PR005656 LFWR2 Kabat, PR005657 LFWR2 Kabat,PR005658 LFWR2 Kabat, PR005660 LFWR2 Kabat, PR005662 LFWR2 Kabat,PR006142 LFWR2 Kabat, PR006143 LFWR2 Kabat, PR006144 LFWR2 Kabat,PR
WYLQKPGQSPQLLIY

SEQ ID NO: 176 - protein, 15 aa, synthetic construct
  REGION 1..15: note = PR301306 LFWR2 Kabat, PR301790 LFWR2 Kabat, PR301803 LFWR2 Kabat
WYQQKPGQPPKLLIK

SEQ ID NO: 177 - protein, 15 aa, synthetic construct
  REGION 1..15: note = PR301612 LFWR2 Kabat
WYLQKPGSSPKPWIY

SEQ ID NO: 178 - protein, 15 aa, synthetic construct
  REGION 1..15: note = PR301777 LFWR2 Kabat, PR301788 LFWR2 Kabat, PR302029 LFWR2 Kabat,PR302036 LFWR2 Kabat, PR302043 LFWR2 Kabat, PR302050 LFWR2 Kabat,PR302064 LFWR2 Kabat
WFQQKPGQPPKLLIK

SEQ ID NO: 179 - protein, 15 aa, synthetic construct
  REGION 1..15: note = PR301780 LFWR2 Kabat, PR301806 LFWR2 Kabat, PR302309-13 LFWR2Kabat, PR302309-14 LFWR2 Kabat, PR302309-15 LFWR2 Kabat,PR302309-16 LFWR2 Kabat, PR302309 LFWR2 Kabat, PR302315 LFWR2Kabat, PR302332 LFW
WYLQKPGQSPKLLIY

SEQ ID NO: 180 - protein, 15 aa, synthetic construct
  REGION 1..15: note = PR302031 LFWR2 Kabat, PR302038 LFWR2 Kabat, PR302045 LFWR2 Kabat,PR302052 LFWR2 Kabat, PR302066 LFWR2 Kabat
WFQQKPGQAPRLLIK

SEQ ID NO: 181 - protein, 15 aa, synthetic construct
  REGION 1..15: note = PR302034 LFWR2 Kabat, PR302041 LFWR2 Kabat, PR302048 LFWR2 Kabat,PR302055 LFWR2 Kabat, PR302069 LFWR2 Kabat
WFQQKPGKAPKLLIK

SEQ ID NO: 182 - protein, 15 aa, synthetic construct
  REGION 1..15: note = PR302309-1 LFWR2 Kabat, PR302309-7 LFWR2 Kabat, PR302341-1 LFWR2Kabat, PR302341-7 LFWR2 Kabat
WFQQRPGQSPRRLIY

SEQ ID NO: 183 - protein, 15 aa, synthetic construct
  REGION 1..15: note = PR302309-11 LFWR2 Kabat, PR302309-5 LFWR2 Kabat, PR302341-3 LFWR2Kabat, PR302341-9 LFWR2 Kabat
WLQQRPGQPPRLLIY

SEQ ID NO: 184 - protein, 15 aa, synthetic construct
  REGION 1..15: note = PR302309-12 LFWR2 Kabat, PR302309-21 LFWR2 Kabat, PR302309-6LFWR2 Kabat, PR302341-10 LFWR2 Kabat, PR302341-24 LFWR2 Kabat,PR302341-26 LFWR2 Kabat, PR302341-43 LFWR2 Kabat, PR302341-44LFWR2 Kabat, P
WYQQRPGQPPRLLIY

SEQ ID NO: 185 - protein, 15 aa, synthetic construct
  REGION 1..15: note = PR302309-17 LFWR2 Kabat, PR302309-18 LFWR2 Kabat, PR302309-19LFWR2 Kabat, PR302309-2 LFWR2 Kabat, PR302309-8 LFWR2 Kabat,PR302341-19 LFWR2 Kabat, PR302341-20 LFWR2 Kabat, PR302341-21LFWR2 Kabat, PR
WYQQRPGQSPRLLIY

SEQ ID NO: 186 - protein, 15 aa, synthetic construct
  REGION 1..15: note = PR302312 LFWR2 Kabat, PR302324 LFWR2 Kabat
WYLQRPGQSPKLLIY

SEQ ID NO: 187 - protein, 15 aa, synthetic construct
  REGION 1..15: note = PR302328 LFWR2 Kabat, PR302331 LFWR2 Kabat, PR302334-14 LFWR2Kabat, PR302334-15 LFWR2 Kabat, PR302334-16 LFWR2 Kabat,PR302334-17 LFWR2 Kabat, PR302334-18 LFWR2 Kabat, PR302334 LFWR2Kabat, PR302339
WYQQKPGQSPKLLIY

SEQ ID NO: 188 - protein, 15 aa, synthetic construct
  REGION 1..15: note = PR302334-1 LFWR2 Kabat, PR302334-22 LFWR2 Kabat, PR302334-2 LFWR2Kabat, PR302334-7 LFWR2 Kabat, PR302334-8 LFWR2 Kabat
WYQQKPGQAPRLLIY

SEQ ID NO: 189 - protein, 15 aa, synthetic construct
  REGION 1..15: note = PR302334-10 LFWR2 Kabat, PR302334-24 LFWR2 Kabat, PR302334-26LFWR2 Kabat, PR302334-3 LFWR2 Kabat, PR302334-4 LFWR2 Kabat,PR302334-9 LFWR2 Kabat
WYQQKPGQPPRLLIY

SEQ ID NO: 190 - protein, 15 aa, synthetic construct
  REGION 1..15: note = PR302334-11 LFWR2 Kabat, PR302334-12 LFWR2 Kabat, PR302334-25LFWR2 Kabat, PR302334-27 LFWR2 Kabat, PR302334-5 LFWR2 Kabat,PR302334-6 LFWR2 Kabat
WYQQKPGKAPRLLIY

SEQ ID NO: 191 - protein, 15 aa, synthetic construct
  REGION 1..15: note = PR302335 LFWR2 Kabat
WYLQKPGQSPRLLIY

SEQ ID NO: 192 - protein, 15 aa, synthetic construct
  REGION 1..15: note = PR302341-13 LFWR2 Kabat, PR302341-14 LFWR2 Kabat, PR302341-15LFWR2 Kabat, PR302341-16 LFWR2 Kabat, PR302341-17 LFWR2 Kabat,PR302341-18 LFWR2 Kabat, PR302341 LFWR2 Kabat
WYLQRPGQSPRLLIY

SEQ ID NO: 193 - protein, 7 aa, synthetic construct
  REGION 1..7: note = PR004290 LCDR2 Kabat, PR005656 LCDR2 Kabat, PR005657 LCDR2 Kabat,PR005658 LCDR2 Kabat, PR005660 LCDR2 Kabat, PR005662 LCDR2 Kabat,PR006142 LCDR2 Kabat, PR006143 LCDR2 Kabat, PR006144 LCDR2 Kabat,PR
LGSNRAS

SEQ ID NO: 194 - protein, 7 aa, synthetic construct
  REGION 1..7: note = PR301306 LCDR2 Kabat
YASNLES

SEQ ID NO: 195 - protein, 7 aa, synthetic construct
  REGION 1..7: note = PR301612 LCDR2 Kabat
RTSNLAS

SEQ ID NO: 196 - protein, 7 aa, synthetic construct
  REGION 1..7: note = PR301777 LCDR2 Kabat, PR301788 LCDR2 Kabat, PR301790 LCDR2 Kabat,PR301803 LCDR2 Kabat, PR302029 LCDR2 Kabat, PR302031 LCDR2 Kabat,PR302034 LCDR2 Kabat, PR302036 LCDR2 Kabat, PR302038 LCDR2 Kabat,PR
FASNLES

SEQ ID NO: 197 - protein, 7 aa, synthetic construct
  REGION 1..7: note = PR301780 LCDR2 Kabat, PR301806 LCDR2 Kabat, PR302309-10 LCDR2Kabat, PR302309-11 LCDR2 Kabat, PR302309-12 LCDR2 Kabat,PR302309-13 LCDR2 Kabat, PR302309-14 LCDR2 Kabat, PR302309-15LCDR2 Kabat, PR3023
KVSNRFS

SEQ ID NO: 198 - protein, 7 aa, synthetic construct
  REGION 1..7: note = PR302328 LCDR2 Kabat, PR302331 LCDR2 Kabat, PR302334-10 LCDR2Kabat, PR302334-11 LCDR2 Kabat, PR302334-12 LCDR2 Kabat,PR302334-14 LCDR2 Kabat, PR302334-15 LCDR2 Kabat, PR302334-16LCDR2 Kabat, PR3023
YASNRYT

SEQ ID NO: 199 - protein, 32 aa, synthetic construct
  REGION 1..32: note = PR004290 LFWR3 Kabat, PR005656 LFWR3 Kabat, PR005657 LFWR3 Kabat,PR005658 LFWR3 Kabat, PR005660 LFWR3 Kabat, PR005662 LFWR3 Kabat,PR006142 LFWR3 Kabat, PR006143 LFWR3 Kabat, PR006144 LFWR3 Kabat,PR
GVPDRFSGSGSGTDFTLKISRVEAEDVGIYYC

SEQ ID NO: 200 - protein, 32 aa, synthetic construct
  REGION 1..32: note = PR006669 LFWR3 Kabat, PR006744 LFWR3 Kabat, PR006745 LFWR3 Kabat,PR006746 LFWR3 Kabat
GVPDRFSGSGSGTDFTLKFIRVEAEDVGVYYC

SEQ ID NO: 201 - protein, 32 aa, synthetic construct
  REGION 1..32: note = PR301306 LFWR3 Kabat
GVPARFSGSGSGTDFTLNIHPVEEEDTATYYC

SEQ ID NO: 202 - protein, 32 aa, synthetic construct
  REGION 1..32: note = PR301612 LFWR3 Kabat
GVPARFSGSGSGTSYSLTISSMEAEDVATYYC

SEQ ID NO: 203 - protein, 32 aa, synthetic construct
  REGION 1..32: note = PR301777 LFWR3 Kabat, PR301788 LFWR3 Kabat, PR301790 LFWR3 Kabat,PR301803 LFWR3 Kabat
GVPARFSGSGSGTDFTLNIHPVEEEDTATFYC

SEQ ID NO: 204 - protein, 32 aa, synthetic construct
  REGION 1..32: note = PR301780 LFWR3 Kabat
GVPDRFSGSGSGTDFTLKISRVEAEDLGVYHC

SEQ ID NO: 205 - protein, 32 aa, synthetic construct
  REGION 1..32: note = PR301806 LFWR3 Kabat, PR302309-10 LFWR3 Kabat, PR302309-13 LFWR3Kabat, PR302309-14 LFWR3 Kabat, PR302309-15 LFWR3 Kabat,PR302309-16 LFWR3 Kabat, PR302309-17 LFWR3 Kabat, PR302309-18LFWR3 Kabat, PR3
GVPDRFSGSGSGTDFTLKISRVEAEDLGVYYC

SEQ ID NO: 206 - protein, 32 aa, synthetic construct
  REGION 1..32: note = PR302029 LFWR3 Kabat, PR302036 LFWR3 Kabat, PR302043 LFWR3 Kabat,PR302050 LFWR3 Kabat, PR302064 LFWR3 Kabat
GVPDRFSGSGSGTDFTLTISSLQAEDTAVYYC

SEQ ID NO: 207 - protein, 32 aa, synthetic construct
  REGION 1..32: note = PR302031 LFWR3 Kabat, PR302038 LFWR3 Kabat, PR302045 LFWR3 Kabat,PR302052 LFWR3 Kabat, PR302066 LFWR3 Kabat
GIPARFSGSGSGTDFTLTISSLEPEDTAVYYC

SEQ ID NO: 208 - protein, 32 aa, synthetic construct
  REGION 1..32: note = PR302034 LFWR3 Kabat, PR302041 LFWR3 Kabat, PR302048 LFWR3 Kabat,PR302055 LFWR3 Kabat, PR302069 LFWR3 Kabat
GVPSRFSGSGSGTDFTLTISSLQPEDTATYYC

SEQ ID NO: 209 - protein, 32 aa, synthetic construct
  REGION 1..32: note = PR302309-1 LFWR3 Kabat, PR302309-3 LFWR3 Kabat, PR302309-7 LFWR3Kabat, PR302309-9 LFWR3 Kabat, PR302341-11 LFWR3 Kabat,PR302341-1 LFWR3 Kabat, PR302341-5 LFWR3 Kabat, PR302341-7 LFWR3Kabat
GVPDRFSGSGSGTDFTLKISRVEAEDVGVYYC

SEQ ID NO: 210 - protein, 32 aa, synthetic construct
  REGION 1..32: note = PR302309-11 LFWR3 Kabat, PR302309-5 LFWR3 Kabat, PR302341-3 LFWR3Kabat, PR302341-9 LFWR3 Kabat
GVPDRFSGSGAGTDFTLKISRVEAEDVGVYYC

SEQ ID NO: 211 - protein, 32 aa, synthetic construct
  REGION 1..32: note = PR302309-12 LFWR3 Kabat, PR302309-21 LFWR3 Kabat, PR302309-6LFWR3 Kabat, PR302341-10 LFWR3 Kabat, PR302341-24 LFWR3 Kabat,PR302341-26 LFWR3 Kabat, PR302341-43 LFWR3 Kabat, PR302341-44LFWR3 Kabat, P
GVPDRFSGSGAGTDFTLKISRVEAEDLGVYYC

SEQ ID NO: 212 - protein, 32 aa, synthetic construct
  REGION 1..32: note = PR302312 LFWR3 Kabat, PR302324 LFWR3 Kabat
GVPDRFSGSGSGTDFTLKISRVGAEDLGVYYC

SEQ ID NO: 213 - protein, 32 aa, synthetic construct
  REGION 1..32: note = PR302315 LFWR3 Kabat
GVPDRFSGSGSGTDFTLKISRVEAEDLGVYFC

SEQ ID NO: 214 - protein, 32 aa, synthetic construct
  REGION 1..32: note = PR302328 LFWR3 Kabat, PR302331 LFWR3 Kabat, PR302334-14 LFWR3Kabat, PR302334-15 LFWR3 Kabat, PR302334-16 LFWR3 Kabat,PR302334-17 LFWR3 Kabat, PR302334-18 LFWR3 Kabat, PR302334 LFWR3Kabat, PR302339
GVPDRFTGSGYGTDFTFTISTVQAEDLAVYFC

SEQ ID NO: 215 - protein, 32 aa, synthetic construct
  REGION 1..32: note = PR302334-1 LFWR3 Kabat, PR302334-7 LFWR3 Kabat
GIPARFSGSGSGTEFTLTISSLQSEDFAVYYC

SEQ ID NO: 216 - protein, 32 aa, synthetic construct
  REGION 1..32: note = PR302334-10 LFWR3 Kabat, PR302334-24 LFWR3 Kabat, PR302334-26LFWR3 Kabat, PR302334-4 LFWR3 Kabat
GVPDRFSGSGSGTDFTLTISSLQAEDLAVYFC

SEQ ID NO: 217 - protein, 32 aa, synthetic construct
  REGION 1..32: note = PR302334-11 LFWR3 Kabat, PR302334-5 LFWR3 Kabat
GVPSRFSGSGSGTDFTFTISSLQPEDIATYYC

SEQ ID NO: 218 - protein, 32 aa, synthetic construct
  REGION 1..32: note = PR302334-12 LFWR3 Kabat, PR302334-25 LFWR3 Kabat, PR302334-27LFWR3 Kabat, PR302334-6 LFWR3 Kabat
GVPSRFSGSGSGTDFTFTISSLQPEDLATYFC

SEQ ID NO: 219 - protein, 32 aa, synthetic construct
  REGION 1..32: note = PR302334-22 LFWR3 Kabat, PR302334-2 LFWR3 Kabat, PR302334-8 LFWR3Kabat
GIPARFSGSGSGTEFTLTISSLQSEDLAVYFC

SEQ ID NO: 220 - protein, 32 aa, synthetic construct
  REGION 1..32: note = PR302334-3 LFWR3 Kabat, PR302334-9 LFWR3 Kabat
GVPDRFSGSGSGTDFTLTISSLQAEDVAVYYC

SEQ ID NO: 221 - protein, 32 aa, synthetic construct
  REGION 1..32: note = PR302335 LFWR3 Kabat
GVPDRFSGSGSGTDFTLKITRVEAEDLGVYFC

SEQ ID NO: 222 - protein, 32 aa, synthetic construct
  REGION 1..32: note = PR302341-13 LFWR3 Kabat, PR302341-14 LFWR3 Kabat, PR302341-15LFWR3 Kabat, PR302341-16 LFWR3 Kabat, PR302341-17 LFWR3 Kabat,PR302341-18 LFWR3 Kabat, PR302341 LFWR3 Kabat
GVPDRFSGSGSGTDFTLKISRVGAEDLGIYYC

SEQ ID NO: 223 - protein, 9 aa, synthetic construct
  REGION 1..9: note = PR004290 LCDR3 Kabat, PR005656 LCDR3 Kabat, PR005657 LCDR3 Kabat,PR005658 LCDR3 Kabat, PR005660 LCDR3 Kabat, PR005662 LCDR3 Kabat,PR006142 LCDR3 Kabat, PR006143 LCDR3 Kabat, PR006144 LCDR3 Kabat,PR
MQTLQTPIT

SEQ ID NO: 224 - protein, 9 aa, synthetic construct
  REGION 1..9: note = PR006669 LCDR3 Kabat, PR006744 LCDR3 Kabat, PR006745 LCDR3 Kabat,PR006746 LCDR3 Kabat
MQALQTPYT

SEQ ID NO: 225 - protein, 9 aa, synthetic construct
  REGION 1..9: note = PR301306 LCDR3 Kabat, PR301777 LCDR3 Kabat, PR301788 LCDR3 Kabat,PR301790 LCDR3 Kabat, PR301803 LCDR3 Kabat, PR302029 LCDR3 Kabat,PR302031 LCDR3 Kabat, PR302034 LCDR3 Kabat, PR302036 LCDR3 Kabat,PR
QHSWEIPYT

SEQ ID NO: 226 - protein, 9 aa, synthetic construct
  REGION 1..9: note = PR301612 LCDR3 Kabat
QQYHSFPRT

SEQ ID NO: 227 - protein, 9 aa, synthetic construct
  REGION 1..9: note = PR301780 LCDR3 Kabat, PR302309-10 LCDR3 Kabat, PR302309-11 LCDR3Kabat, PR302309-12 LCDR3 Kabat, PR302309-13 LCDR3 Kabat,PR302309-14 LCDR3 Kabat, PR302309-15 LCDR3 Kabat, PR302309-16LCDR3 Kabat, PR3
FQGSHVPWT

SEQ ID NO: 228 - protein, 9 aa, synthetic construct
  REGION 1..9: note = PR301806 LCDR3 Kabat
FQGSHVPPT

SEQ ID NO: 229 - protein, 9 aa, synthetic construct
  REGION 1..9: note = PR302315 LCDR3 Kabat
SQSTHVPWT

SEQ ID NO: 230 - protein, 9 aa, synthetic construct
  REGION 1..9: note = PR302324 LCDR3 Kabat, PR302341-10 LCDR3 Kabat, PR302341-11 LCDR3Kabat, PR302341-12 LCDR3 Kabat, PR302341-13 LCDR3 Kabat,PR302341-14 LCDR3 Kabat, PR302341-15 LCDR3 Kabat, PR302341-16LCDR3 Kabat, PR3
FQGSHIPWT

SEQ ID NO: 231 - protein, 8 aa, synthetic construct
  REGION 1..8: note = PR302328 LCDR3 Kabat, PR302331 LCDR3 Kabat, PR302334-10 LCDR3Kabat, PR302334-11 LCDR3 Kabat, PR302334-12 LCDR3 Kabat,PR302334-14 LCDR3 Kabat, PR302334-15 LCDR3 Kabat, PR302334-16LCDR3 Kabat, PR3023
QQDYSSWT

SEQ ID NO: 232 - protein, 9 aa, synthetic construct
  REGION 1..9: note = PR302335 LCDR3 Kabat
SQSTHIPWT

SEQ ID NO: 233 - protein, 10 aa, synthetic construct
  REGION 1..10: note = PR004290 LFWR4 Kabat, PR005656 LFWR4 Kabat, PR005657 LFWR4 Kabat,PR005658 LFWR4 Kabat, PR005660 LFWR4 Kabat, PR005662 LFWR4 Kabat,PR006142 LFWR4 Kabat, PR006143 LFWR4 Kabat, PR006144 LFWR4 Kabat,PR
FGQGTRLEIK

SEQ ID NO: 234 - protein, 10 aa, synthetic construct
  REGION 1..10: note = PR006669 LFWR4 Kabat, PR006744 LFWR4 Kabat, PR006745 LFWR4 Kabat,PR006746 LFWR4 Kabat, PR302029 LFWR4 Kabat, PR302031 LFWR4 Kabat,PR302034 LFWR4 Kabat, PR302036 LFWR4 Kabat, PR302038 LFWR4 Kabat,PR
FGQGTKLEIK

SEQ ID NO: 235 - protein, 10 aa, synthetic construct
  REGION 1..10: note = PR301306 LFWR4 Kabat, PR301612 LFWR4 Kabat, PR301777 LFWR4 Kabat,PR301780 LFWR4 Kabat, PR301788 LFWR4 Kabat, PR301790 LFWR4 Kabat,PR301803 LFWR4 Kabat, PR302309-13 LFWR4 Kabat, PR302309-14 LFWR4Kab
FGGGTKLEIK

SEQ ID NO: 236 - protein, 10 aa, synthetic construct
  REGION 1..10: note = PR301806 LFWR4 Kabat
FGAGTKLELK

SEQ ID NO: 237 - protein, 10 aa, synthetic construct
  REGION 1..10: note = PR302309-10 LFWR4 Kabat, PR302309-11 LFWR4 Kabat, PR302309-12LFWR4 Kabat, PR302309-17 LFWR4 Kabat, PR302309-18 LFWR4 Kabat,PR302309-19 LFWR4 Kabat, PR302309-1 LFWR4 Kabat, PR302309-20LFWR4 Kabat, P
FGGGTKVEIK

SEQ ID NO: 238 - protein, 10 aa, synthetic construct
  REGION 1..10: note = PR302312 LFWR4 Kabat, PR302324 LFWR4 Kabat, PR302341-13 LFWR4Kabat, PR302341-14 LFWR4 Kabat, PR302341-15 LFWR4 Kabat,PR302341-16 LFWR4 Kabat, PR302341-17 LFWR4 Kabat, PR302341-18LFWR4 Kabat, PR3023
FGGGTKLEVK

SEQ ID NO: 239 - protein, 10 aa, synthetic construct
  REGION 1..10: note = PR302334-14 LFWR4 Kabat, PR302334-15 LFWR4 Kabat, PR302334-16LFWR4 Kabat, PR302334-17 LFWR4 Kabat, PR302334-18 LFWR4 Kabat,PR302334 LFWR4 Kabat
FGGGTNLEIK

SEQ ID NO: 240 - protein, 115 aa, synthetic construct
  REGION 1..115: note = PR004290 VH, PR005658 VH
EVQLVESGGVLVQPGGSLRLSCAASGFTFSSYWMTWVRQAPGKGLEWVANIKQDGSEKYYVDSVKGRFTISRDNAKNSLFLQMNSLRAEDTAVYYCARTLIFDYWGQGTLVTVSS

SEQ ID NO: 241 - protein, 115 aa, synthetic construct
  REGION 1..115: note = PR005656 VH, PR005660 VH, PR006142 VH, PR006143 VH, PR006144 VH,PR006145 VH
EVQLVESGGVLVQPGGSLRLSCAASGFTFSSYWMTWVRQAPGKGLEWVANIKQEGSEKYYVDSVKGRFTISRDNAKNSLFLQMNSLRAEDTAVYYCARTLIFDYWGQGTLVTVSS

SEQ ID NO: 242 - protein, 115 aa, synthetic construct
  REGION 1..115: note = PR005657 VH, PR005662 VH, PR006146 VH, PR006147 VH, PR006148 VH,PR006149 VH
EVQLVESGGVLVQPGGSLRLSCAASGFTFSSYWMTWVRQAPGKGLEWVANIKQDASEKYYVDSVKGRFTISRDNAKNSLFLQMNSLRAEDTAVYYCARTLIFDYWGQGTLVTVSS

SEQ ID NO: 243 - protein, 115 aa, synthetic construct
  REGION 1..115: note = PR006150 VH, PR006151 VH, PR006152 VH, PR006153 VH
EVQLVESGGVLVQPGGSLRLSCAASGFTFSSYWMTWVRQAPGKGLEWVANIKQDSSEKYYVDSVKGRFTISRDNAKNSLFLQMNSLRAEDTAVYYCARTLIFDYWGQGTLVTVSS

SEQ ID NO: 244 - protein, 115 aa, synthetic construct
  REGION 1..115: note = PR006669 VH
EVQLVESGGGLVQPGGSLRLSCAASGFTFSDNWMSWVRQAPGKGLEWVANIKQDGSENYYVDSVKGRFTISRDNAKNSLYLQMNSLRAEDTAVYYCSRGLALWGWGQGTLVTVSS

SEQ ID NO: 245 - protein, 115 aa, synthetic construct
  REGION 1..115: note = PR006744 VH
EVQLVESGGGLVQPGGSLRLSCAASGFTFSDNWMSWVRQAPGKGLEWVANIKQEGSENYYVDSVKGRFTISRDNAKNSLYLQMNSLRAEDTAVYYCSRGLALWGWGQGTLVTVSS

SEQ ID NO: 246 - protein, 115 aa, synthetic construct
  REGION 1..115: note = PR006745 VH
EVQLVESGGGLVQPGGSLRLSCAASGFTFSDNWMSWVRQAPGKGLEWVANIKQDASENYYVDSVKGRFTISRDNAKNSLYLQMNSLRAEDTAVYYCSRGLALWGWGQGTLVTVSS

SEQ ID NO: 247 - protein, 115 aa, synthetic construct
  REGION 1..115: note = PR006746 VH
EVQLVESGGGLVQPGGSLRLSCAASGFTFSDNWMSWVRQAPGKGLEWVANIKQDSSENYYVDSVKGRFTISRDNAKNSLYLQMNSLRAEDTAVYYCSRGLALWGWGQGTLVTVSS

SEQ ID NO: 248 - protein, 116 aa, synthetic construct
  REGION 1..116: note = PR301306 VH
EVKLEESGGGLVQPGGSMKLSCVASGFTFSNYWMNWVRQSPEKGLEWVAEIRLKSNNYATLYAESVKGRFTISRDDSKSSVYLQMNNLRAEDTGIYYCTRGTLVYWGQGTLVTVSA

SEQ ID NO: 249 - protein, 114 aa, synthetic construct
  REGION 1..114: note = PR301612 VH
EVKLEESGGGLVQPGGSIKLSCVASGFTFSGAWMAWVRQSPEKGLEWVAEIKNKANNHATFYAESVKGRFIISRDDSKSSVYLQMNSLRAEDTGTYYCTLPDVWGTGTTVTVSS

SEQ ID NO: 250 - protein, 116 aa, synthetic construct
  REGION 1..116: note = PR301777 VH
EMKLEESGGGLVQPGGSMKLSCVASGFTFSNYWMNWVRQSPEKGLEWVAEIRLKSNNYATHYAESVKGRFTISRDDSKSGVYLQMNNLGAEDTGIYYCTVGTLVYWGQGTLVTVSA

SEQ ID NO: 251 - protein, 121 aa, synthetic construct
  REGION 1..121: note = PR301780 VH
QIQLVQSGPELKKPGETVKISCKASGYTFTNFGLNWVKQAPGKGLKWMGWINTYTGEPTYTDDFKGRFAFSLETSASTAYLQINNLKKEDTATYFCARGTYRDDGAWFAYWGQGTLVTVSA

SEQ ID NO: 252 - protein, 116 aa, synthetic construct
  REGION 1..116: note = PR301788 VH, PR301790 VH
EMKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTIGTLVYWGQGTLVTVSA

SEQ ID NO: 253 - protein, 116 aa, synthetic construct
  REGION 1..116: note = PR301803 VH
EVKLEESGGGLVQPGGSMKLSCVASGFTFNNYWMNWVRQSPEKGLEWVAEIRLKSNNYATHYAESLKGRFTISRDDSKSSVYLQMNNLRAEDTGIYYCTVGTLVYWGQGTLVTVSA

SEQ ID NO: 254 - protein, 121 aa, synthetic construct
  REGION 1..121: note = PR301806 VH
QVQLQQPGAELVKPGTSVKMSCKASGYTFTSYWMHWVKQRPGQGLEWIGDIYPGSDSTNYNEKFKSKASLTVDTSSSTAYMQLSSLTSEDSAVYYCARSITTTTFWYFDVWGAGTTVTVSS

SEQ ID NO: 255 - protein, 116 aa, synthetic construct
  REGION 1..116: note = PR302029 VH, PR302031 VH, PR302034 VH
EVQLVESGGGLVQPGGSLRLSCAASGFTFSNYWMNWVRQAPGKGLEWVGEIRLKSNNYATHYAESVKGRFTISRDDSKNSLYLQMNSLKTEDTAVYYCTVGTLVYWGQGTLVTVSS

SEQ ID NO: 256 - protein, 116 aa, synthetic construct
  REGION 1..116: note = PR302036 VH, PR302038 VH, PR302041 VH
EMQLVESGGGLVQPGGSLRLSCAASGFTFSNYWMNWVRQAPGKGLEWVAEIRLKSNNYATHYAESVKGRFTISRDDSKSSVYLQMNSLKTEDTAVYYCTVGTLVYWGQGTLVTVSS

SEQ ID NO: 257 - protein, 116 aa, synthetic construct
  REGION 1..116: note = PR302043 VH, PR302045 VH, PR302048 VH
EVQLVESGGGLVQPGGSLKLSCAASGFTFSNYWMNWVRQASGKGLEWVGEIRLKSNNYATHYAESVKGRFTISRDDSKNTAYLQMNSLKTEDTAVYYCTRGTLVYWGQGTLVTVSS

SEQ ID NO: 258 - protein, 116 aa, synthetic construct
  REGION 1..116: note = PR302050 VH, PR302052 VH, PR302055 VH
EMQLVESGGGLVQPGGSLKLSCAASGFTFSNYWMNWVRQASGKGLEWVAEIRLKSNNYATHYAESVKGRFTISRDDSKSTVYLQMNSLKTEDTAVYYCTVGTLVYWGQGTLVTVSS

SEQ ID NO: 259 - protein, 116 aa, synthetic construct
  REGION 1..116: note = PR302064 VH, PR302066 VH, PR302069 VH
EMQLVESGGGLVKPGGSLRLSCAASGFTFSNYWMNWVRQAPGKGLEWVAEIRLKSNNYATHYAESVKGRFTISRDDSKSTVYLQMNSLKTEDTAVYYCTVGTLVYWGQGTLVTVSS

SEQ ID NO: 260 - protein, 121 aa, synthetic construct
  REGION 1..121: note = PR302309-1 VH, PR302309-2 VH, PR302309-3 VH, PR302309-4 VH,PR302309-5 VH, PR302309-6 VH, PR302309 VH
QIQLVQSGPELKKPGETVKISCKASGYTFTNYGMNWVKQAPGKVLKWMGWINTYTGEPIYTDDFKGRFAFSLETSASTAYLQINNLKNEDTATYFCSRSTMITTGGVFAYWGQGTLVTVSA

SEQ ID NO: 261 - protein, 121 aa, synthetic construct
  REGION 1..121: note = PR302309-10 VH, PR302309-11 VH, PR302309-12 VH, PR302309-14 VH,PR302309-7 VH, PR302309-8 VH, PR302309-9 VH
QIQLVQSGSELKKPGASVKVSCKASGYTFTNYGMNWVRQAPGQGLEWMGWINTYTGEPIYTDDFKGRFVFSLDTSVSTAYLQISSLKAEDTAVYYCSRSTMITTGGVFAYWGQGTLVTVSS

SEQ ID NO: 262 - protein, 121 aa, synthetic construct
  REGION 1..121: note = PR302309-13 VH, PR302309-17 VH
QVQLVQSGSELKKPGASVKVSCKASGYTFTNYGMNWVRQAPGQGLEWMGWINTYTGEPIYTDDFKGRFVFSLDTSVSTAYLQISSLKAEDTAVYYCARSTMITTGGVFAYWGQGTLVTVSS

SEQ ID NO: 263 - protein, 121 aa, synthetic construct
  REGION 1..121: note = PR302309-15 VH, PR302309-18 VH
QVQLVQSGAEVKKPGASVKVSCKASGYTFTNYGMNWVRQAPGQRLEWMGWINTYTGEPIYTDDFKGRVTITRDTSASTAYMELSSLRSEDTAVYYCARSTMITTGGVFAYWGQGTLVTVSS

SEQ ID NO: 264 - protein, 121 aa, synthetic construct
  REGION 1..121: note = PR302309-16 VH, PR302309-19 VH, PR302309-20 VH, PR302309-21 VH
QIQLVQSGAEVKKPGASVKVSCKASGYTFTNYGMNWVRQAPGQRLEWMGWINTYTGEPIYTDDFKGRVTITLDTSASTAYMELSSLRSEDTAVYYCSRSTMITTGGVFAYWGQGTLVTVSS

SEQ ID NO: 265 - protein, 118 aa, synthetic construct
  REGION 1..118: note = PR302312 VH
EVQLQQSGPELVKPGASVKMSCKASGYTFTDYFMKWMKQSHGKSLEWIGVINPNNGDTFYNQNFKGKATLTVDKSSSTAYMQLNSLTSEDSAVYYCTRGTARALFAYWGQGTLVTVSA

SEQ ID NO: 266 - protein, 118 aa, synthetic construct
  REGION 1..118: note = PR302315 VH
DVQLVESGGGLMQPGGSRKLSCVASGFTFSSFGMHWVRQAPEKGLEWVAYISSGSSIIYYADTVKGRFTISRDNPKNILFLQMTSLRSEDTAMYYCARDITTATFAYWGQGTLVTVSA

SEQ ID NO: 267 - protein, 118 aa, synthetic construct
  REGION 1..118: note = PR302324 VH
EVQLQQSGPELVKPGASVKMSCKASGYTFTDYFMKWVKQSHGGSLEWIGVISPNNGDTFYNQKFKGKATLTVDKSSSTAFMQLNSLTSEDSAVYSCARGTDRALFAYWGQGTLVTVSA

SEQ ID NO: 268 - protein, 111 aa, synthetic construct
  REGION 1..111: note = PR302328 VH
EVKLVESGGGLVKPGGSLKLSCAASGFTFSSYAMSWVRQTPEKRLEWVASIRSGGTTYYPDSVKGRFTISRDNARNILYLQMSSLRSEDTAIYYCTRGIDWGQGTLVTVSA

SEQ ID NO: 269 - protein, 111 aa, synthetic construct
  REGION 1..111: note = PR302331 VH
EVKLVESGGGLVKPGGSLKLSCAASGFTFSSYAMSWVRQSPEKRLEWVASIRSGGTTYYPDSVKGRFTISRDNARNILYLQMSSLRSEDTAIYYCTRGIDWGQGTLVTVSA

SEQ ID NO: 270 - protein, 121 aa, synthetic construct
  REGION 1..121: note = PR302332 VH
QIQLVQSGPELKKPGETVKISCKASGYTFTNFGMHWVKQAPGKVLKWMGWINTYTGEPIYAADFKGRFAFSLETSASTAYLQINNLKNEDTATYFCSRSTMITTGGVFAYWGQGTLVTVSA

SEQ ID NO: 271 - protein, 121 aa, synthetic construct
  REGION 1..121: note = PR302333 VH
QIQLVQSGPELKKPGETVKISCKASGYTFTNFGMNWVKQAPGKVLKWMGWINTYTGEPIYADDFKGRFAFSLETSASAAYLQINNLKNEDTATYFCSRSTMITTGGVFAYWGQGTLVTVSA

SEQ ID NO: 272 - protein, 111 aa, synthetic construct
  REGION 1..111: note = PR302334-1 VH, PR302334-2 VH, PR302334-3 VH, PR302334-4 VH,PR302334-5 VH, PR302334-6 VH, PR302334 VH
EVKLVESGGGLVKPGGSLKLSCAASGFTFSTYAMSWVRQTPEKRLEWVASIRSGGTTYYPDSVKGRFTISRDNARNILYLQMSSLRSEDTAIYYCTRGIDWGQGTLVTVSA

SEQ ID NO: 273 - protein, 111 aa, synthetic construct
  REGION 1..111: note = PR302334-10 VH, PR302334-11 VH, PR302334-12 VH, PR302334-14 VH,PR302334-7 VH, PR302334-8 VH, PR302334-9 VH
EVQLVESGGGLVQPGGSLRLSCAASGFTFSTYAMSWVRQAPGKGLEWVASIRSGGTTYYPDSVKGRFTISRDNSKNTLYLQMNSLRAEDTAVYYCTRGIDWGQGTTVTVSS

SEQ ID NO: 274 - protein, 111 aa, synthetic construct
  REGION 1..111: note = PR302334-15 VH
EVQLVESGGGLVKPGGSLRLSCAASGFTFSTYAMSWVRQAPGKGLEWVSSIRSGGTTYYPDSVKGRFTISRDNAKNSLYLQMNSLRAEDTAVYYCARGIDWGQGTTVTVSS

SEQ ID NO: 275 - protein, 111 aa, synthetic construct
  REGION 1..111: note = PR302334-16 VH, PR302334-24 VH, PR302334-25 VH
EVQLVESGGGLVKPGGSLRLSCAASGFTFSTYAMSWVRQAPGKGLEWVASIRSGGTTYYPDSVKGRFTISRDNAKNSLYLQMNSLRAEDTAVYYCTRGIDWGQGTTVTVSS

SEQ ID NO: 276 - protein, 111 aa, synthetic construct
  REGION 1..111: note = PR302334-17 VH, PR302334-22 VH
EVQLVESGGGLVQPGGSLRLSCAASGFTFSTYAMSWVRQAPGKGLEWVSSIRSGGTTYYPDSVKGRFTISRDNSKNSLYLQMNSLRAEDTAVYYCARGIDWGQGTTVTVSS

SEQ ID NO: 277 - protein, 111 aa, synthetic construct
  REGION 1..111: note = PR302334-18 VH, PR302334-26 VH, PR302334-27 VH
EVQLVESGGGLVQPGGSLRLSCAASGFTFSTYAMSWVRQAPGKGLEWVASIRSGGTTYYPDSVKGRFTISRDNSKNSLYLQMNSLRAEDTAVYYCTRGIDWGQGTTVTVSS

SEQ ID NO: 278 - protein, 118 aa, synthetic construct
  REGION 1..118: note = PR302335 VH
DVQLVESGGGLVQPGGSRKLSCAASGFTFSSFGMHWVRQAPEKGLEWVAYISSGSTIFYYADTVKGRFTISRDNPKNTLFLQMTSLRSEDTAMYYCARDITTATFAFWGQGTLVTVSA

SEQ ID NO: 279 - protein, 111 aa, synthetic construct
  REGION 1..111: note = PR302339 VH
EVKLVESGGGLVKPGGSLKLSCAASGFTFSSYALSWVRQTPEKRLEWVASIRSGGTTYYPDSVKGRFTISRDNARNILYLQMSSLRSEDTAIYYCTRGIDWGQGTLVTVSA

SEQ ID NO: 280 - protein, 118 aa, synthetic construct
  REGION 1..118: note = PR302341-1 VH, PR302341-2 VH, PR302341-3 VH, PR302341-4 VH,PR302341-5 VH, PR302341-6 VH, PR302341 VH
EVQLQQSGPELVKPGASVKMSCKASGYTFTDYFMKWVKQSHGESLEWIGVINPNNGDIFYNQKFKGKATLTVDKSSSTAFMQLNSLTSEDSAVYSCARGTDRALFAYWGQGTLVAVSA

SEQ ID NO: 281 - protein, 118 aa, synthetic construct
  REGION 1..118: note = PR302341-10 VH, PR302341-11 VH, PR302341-12 VH, PR302341-14 VH,PR302341-67 VH, PR302341-68 VH, PR302341-69 VH, PR302341-7 VH,PR302341-8 VH, PR302341-9 VH
QVQLVQSGAEVKKPGASVKVSCKASGYTFTDYFMKWVRQAPGQGLEWMGVINPNNGDIFYNQKFKGRVTMTVDKSTSTAYMELSSLRSEDTAVYYCARGTDRALFAYWGQGTLVTVSS

SEQ ID NO: 282 - protein, 118 aa, synthetic construct
  REGION 1..118: note = PR302341-13 VH, PR302341-19 VH
QVQLVQSGAEVKKPGASVKVSCKASGYTFTDYFMKWVRQAPGQGLEWMGVINPNNGDIFYNQKFKGRVTMTRDTSTSTVYMELSSLRSEDTAVYYCARGTDRALFAYWGQGTLVTVSS

SEQ ID NO: 283 - protein, 118 aa, synthetic construct
  REGION 1..118: note = PR302341-15 VH, PR302341-20 VH, PR302341-70 VH, PR302341-71 VH,PR302341-72 VH
QVQLVQSGAEVKKPGASVKVSCKASGYTFTDYFMKWVRQAPGQRLEWMGVINPNNGDIFYNQKFKGRVTITRDTSASTAYMELSSLRSEDTAVYYCARGTDRALFAYWGQGTLVTVSS

SEQ ID NO: 284 - protein, 118 aa, synthetic construct
  REGION 1..118: note = PR302341-16 VH, PR302341-21 VH, PR302341-24 VH, PR302341-25 VH
QVQLVQSGAEVKKPGASVKVSCKASGYTFTDYFMKWVRQAPGQRLEWMGVINPNNGDIFYNQKFKGRVTITVDKSASTAYMELSSLRSEDTAVYYCARGTDRALFAYWGQGTLVTVSS

SEQ ID NO: 285 - protein, 118 aa, synthetic construct
  REGION 1..118: note = PR302341-17 VH, PR302341-22 VH
QVQLVQSGAEVKKPGASVKVSCKASGYTFTDYFMKWVRQAPGQGLEWMGVINPNNGDIFYNQKFKGRVTMTRDTSISTAYMELSRLRSDDTAVYYCARGTDRALFAYWGQGTLVTVSS

SEQ ID NO: 286 - protein, 118 aa, synthetic construct
  REGION 1..118: note = PR302341-18 VH, PR302341-23 VH, PR302341-26 VH, PR302341-27 VH,PR302341-30 VH, PR302341-31 VH, PR302341-42 VH
QVQLVQSGAEVKKPGASVKVSCKASGYTFTDYFMKWVRQAPGQGLEWMGVINPNNGDIFYNQKFKGRVTMTVDKSISTAYMELSRLRSDDTAVYYCARGTDRALFAYWGQGTLVTVSS

SEQ ID NO: 287 - protein, 118 aa, synthetic construct
  REGION 1..118: note = PR302341-28 VH, PR302341-32 VH, PR302341-34 VH, PR302341-38 VH
QVQLVQSGAEVKKPGASVKVSCKASGYTFTDYFMKWVRQAPGQGLEWMGVINPNQGDIFYNQKFKGRVTMTVDKSISTAYMELSRLRSDDTAVYYCARGTDRALFAYWGQGTLVTVSS

SEQ ID NO: 288 - protein, 118 aa, synthetic construct
  REGION 1..118: note = PR302341-29 VH, PR302341-33 VH, PR302341-35 VH, PR302341-39 VH
QVQLVQSGAEVKKPGASVKVSCKASGYTFTDYFMKWVRQAPGQGLEWMGVINPNNADIFYNQKFKGRVTMTVDKSISTAYMELSRLRSDDTAVYYCARGTDRALFAYWGQGTLVTVSS

SEQ ID NO: 289 - protein, 118 aa, synthetic construct
  REGION 1..118: note = PR302341-36 VH, PR302341-37 VH, PR302341-40 VH, PR302341-41 VH
QVQLVQSGAEVKKPGASVKVSCKASGYTFTDYFMKWVRQAPGQGLEWMGVINPNSGDIFYNQKFKGRVTMTVDKSISTAYMELSRLRSDDTAVYYCARGTDRALFAYWGQGTLVTVSS

SEQ ID NO: 290 - protein, 118 aa, synthetic construct
  REGION 1..118: note = PR302341-43 VH, PR302341-46 VH, PR302341-49 VH, PR302341-52 VH
QVQLVQSGAEVKKPGASVKVSCKASGYTFTDYFMKWVRQAPGQGLEWMGVINPNNADIFYNQKFKGRVTMTVDKSTSTAYMELSSLRSEDTAVYYCARGTDRALFAYWGQGTLVTVSS

SEQ ID NO: 291 - protein, 118 aa, synthetic construct
  REGION 1..118: note = PR302341-44 VH, PR302341-47 VH, PR302341-50 VH, PR302341-53 VH
QVQLVQSGAEVKKPGASVKVSCKASGYTFTDYFMKWVRQAPGQGLEWMGVINPNSGDIFYNQKFKGRVTMTVDKSTSTAYMELSSLRSEDTAVYYCARGTDRALFAYWGQGTLVTVSS

SEQ ID NO: 292 - protein, 118 aa, synthetic construct
  REGION 1..118: note = PR302341-45 VH, PR302341-48 VH, PR302341-51 VH, PR302341-54 VH
QVQLVQSGAEVKKPGASVKVSCKASGYTFTDYFMKWVRQAPGQGLEWMGVINPNQGDIFYNQKFKGRVTMTVDKSTSTAYMELSSLRSEDTAVYYCARGTDRALFAYWGQGTLVTVSS

SEQ ID NO: 293 - protein, 118 aa, synthetic construct
  REGION 1..118: note = PR302341-55 VH, PR302341-58 VH, PR302341-61 VH, PR302341-64 VH
QVQLVQSGAEVKKPGASVKVSCKASGYTFTDYFMKWVRQAPGQRLEWMGVINPNNADIFYNQKFKGRVTITRDTSASTAYMELSSLRSEDTAVYYCARGTDRALFAYWGQGTLVTVSS

SEQ ID NO: 294 - protein, 118 aa, synthetic construct
  REGION 1..118: note = PR302341-56 VH, PR302341-59 VH, PR302341-62 VH, PR302341-65 VH
QVQLVQSGAEVKKPGASVKVSCKASGYTFTDYFMKWVRQAPGQRLEWMGVINPNSGDIFYNQKFKGRVTITRDTSASTAYMELSSLRSEDTAVYYCARGTDRALFAYWGQGTLVTVSS

SEQ ID NO: 295 - protein, 118 aa, synthetic construct
  REGION 1..118: note = PR302341-57 VH, PR302341-60 VH, PR302341-63 VH, PR302341-66 VH
QVQLVQSGAEVKKPGASVKVSCKASGYTFTDYFMKWVRQAPGQRLEWMGVINPNQGDIFYNQKFKGRVTITRDTSASTAYMELSSLRSEDTAVYYCARGTDRALFAYWGQGTLVTVSS

SEQ ID NO: 296 - protein, 112 aa, synthetic construct
  REGION 1..112: note = PR004290 VL, PR005656 VL, PR005657 VL
DIVMTQSPLSLPVTPGEPASISCRSSQSLLHSTGYNSLEWYLQKPGQSPQLLIYLGSNRASGVPDRFSGSGSGTDFTLKISRVEAEDVGIYYCMQTLQTPITFGQGTRLEIK

SEQ ID NO: 297 - protein, 112 aa, synthetic construct
  REGION 1..112: note = PR005658 VL, PR005660 VL, PR005662 VL
DIVMTQSPLSLPVTPGEPASISCRSSQSLLHSTGYQSLEWYLQKPGQSPQLLIYLGSNRASGVPDRFSGSGSGTDFTLKISRVEAEDVGIYYCMQTLQTPITFGQGTRLEIK

SEQ ID NO: 298 - protein, 112 aa, synthetic construct
  REGION 1..112: note = PR006142 VL, PR006146 VL, PR006150 VL
DIVMTQSPLSLPVTPGEPASISCRSSQSLLHSTGYDSLEWYLQKPGQSPQLLIYLGSNRASGVPDRFSGSGSGTDFTLKISRVEAEDVGIYYCMQTLQTPITFGQGTRLEIK

SEQ ID NO: 299 - protein, 112 aa, synthetic construct
  REGION 1..112: note = PR006143 VL, PR006147 VL, PR006151 VL
DIVMTQSPLSLPVTPGEPASISCRSSQSLLHSTGYHSLEWYLQKPGQSPQLLIYLGSNRASGVPDRFSGSGSGTDFTLKISRVEAEDVGIYYCMQTLQTPITFGQGTRLEIK

SEQ ID NO: 300 - protein, 112 aa, synthetic construct
  REGION 1..112: note = PR006144 VL, PR006148 VL, PR006152 VL
DIVMTQSPLSLPVTPGEPASISCRSSQSLLHSTGYNPLEWYLQKPGQSPQLLIYLGSNRASGVPDRFSGSGSGTDFTLKISRVEAEDVGIYYCMQTLQTPITFGQGTRLEIK

SEQ ID NO: 301 - protein, 112 aa, synthetic construct
  REGION 1..112: note = PR006145 VL, PR006149 VL, PR006153 VL
DIVMTQSPLSLPVTPGEPASISCRSSQSLLHSTGYSSLEWYLQKPGQSPQLLIYLGSNRASGVPDRFSGSGSGTDFTLKISRVEAEDVGIYYCMQTLQTPITFGQGTRLEIK

SEQ ID NO: 302 - protein, 112 aa, synthetic construct
  REGION 1..112: note = PR006669 VL, PR006744 VL, PR006745 VL, PR006746 VL
DIVMTHSPLSLPVTPGDPASISCRSSQSLLHSTGYNYLDWYLQKPGQSPQLLIYLGSNRASGVPDRFSGSGSGTDFTLKFIRVEAEDVGVYYCMQALQTPYTFGQGTKLEIK

SEQ ID NO: 303 - protein, 111 aa, synthetic construct
  REGION 1..111: note = PR301306 VL
DIVLTQSPASLAVSLGQRATISCRASQSVSTSDYNYMHWYQQKPGQPPKLLIKYASNLESGVPARFSGSGSGTDFTLNIHPVEEEDTATYYCQHSWEIPYTFGGGTKLEIK

SEQ ID NO: 304 - protein, 106 aa, synthetic construct
  REGION 1..106: note = PR301612 VL
QIVLTQSPAIMSASPGERVTISCSASSSVSNMYWYLQKPGSSPKPWIYRTSNLASGVPARFSGSGSGTSYSLTISSMEAEDVATYYCQQYHSFPRTFGGGTKLEIK

SEQ ID NO: 305 - protein, 111 aa, synthetic construct
  REGION 1..111: note = PR301777 VL, PR301788 VL
DIVLTQSPASLAVSLGQRATISCRASQSVSTSTYNYMHWFQQKPGQPPKLLIKFASNLESGVPARFSGSGSGTDFTLNIHPVEEEDTATFYCQHSWEIPYTFGGGTKLEIK

SEQ ID NO: 306 - protein, 112 aa, synthetic construct
  REGION 1..112: note = PR301780 VL
DVLMTQTPLSLPVSLGDQASISCRSSQIIVHSNGNTYLEWYLQKPGQSPKLLIYKVSNRFSGVPDRFSGSGSGTDFTLKISRVEAEDLGVYHCFQGSHVPWTFGGGTKLEIK

SEQ ID NO: 307 - protein, 111 aa, synthetic construct
  REGION 1..111: note = PR301790 VL
DIVLTQSPASLAVSLGQRATISCRASQSVSTSSYNYMHWYQQKPGQPPKLLIKFASNLESGVPARFSGSGSGTDFTLNIHPVEEEDTATFYCQHSWEIPYTFGGGTKLEIK

SEQ ID NO: 308 - protein, 111 aa, synthetic construct
  REGION 1..111: note = PR301803 VL
DIVLTQSPASLVVSLGQRATISCRASQSVSTSTYNYMHWYQQKPGQPPKLLIKFASNLESGVPARFSGSGSGTDFTLNIHPVEEEDTATFYCQHSWEIPYTFGGGTKLEIK

SEQ ID NO: 309 - protein, 112 aa, synthetic construct
  REGION 1..112: note = PR301806 VL
DVLMTQTPLSLPVSLGDQASISCRSSQSFVYSNGNTYLQWYLQKPGQSPKLLIYKVSNRFSGVPDRFSGSGSGTDFTLKISRVEAEDLGVYYCFQGSHVPPTFGAGTKLELK

SEQ ID NO: 310 - protein, 111 aa, synthetic construct
  REGION 1..111: note = PR302029 VL, PR302036 VL, PR302043 VL, PR302050 VL, PR302064 VL
DIVLTQSPDSLAVSLGERATINCRASQSVSTSTYNYMHWFQQKPGQPPKLLIKFASNLESGVPDRFSGSGSGTDFTLTISSLQAEDTAVYYCQHSWEIPYTFGQGTKLEIK

SEQ ID NO: 311 - protein, 111 aa, synthetic construct
  REGION 1..111: note = PR302031 VL, PR302038 VL, PR302045 VL, PR302052 VL, PR302066 VL
EIVLTQSPATLSLSPGERATLSCRASQSVSTSTYNYMHWFQQKPGQAPRLLIKFASNLESGIPARFSGSGSGTDFTLTISSLEPEDTAVYYCQHSWEIPYTFGQGTKLEIK

SEQ ID NO: 312 - protein, 111 aa, synthetic construct
  REGION 1..111: note = PR302034 VL, PR302041 VL, PR302048 VL, PR302055 VL, PR302069 VL
DIQLTQSPSSLSASVGDRVTITCRASQSVSTSTYNYMHWFQQKPGKAPKLLIKFASNLESGVPSRFSGSGSGTDFTLTISSLQPEDTATYYCQHSWEIPYTFGQGTKLEIK

SEQ ID NO: 313 - protein, 112 aa, synthetic construct
  REGION 1..112: note = PR302309-1 VL, PR302309-7 VL
DVVMTQSPLSLPVTLGQPASISCRSSQTIVHSDGNTYLEWFQQRPGQSPRRLIYKVSNRFSGVPDRFSGSGSGTDFTLKISRVEAEDVGVYYCFQGSHVPWTFGGGTKVEIK

SEQ ID NO: 314 - protein, 112 aa, synthetic construct
  REGION 1..112: note = PR302309-10 VL, PR302309-20 VL, PR302309-4 VL
DVVMTQTPLSLSVTPGQPASISCRSSQTIVHSDGNTYLEWYLQKPGQSPQLLIYKVSNRFSGVPDRFSGSGSGTDFTLKISRVEAEDLGVYYCFQGSHVPWTFGGGTKVEIK

SEQ ID NO: 315 - protein, 112 aa, synthetic construct
  REGION 1..112: note = PR302309-11 VL, PR302309-5 VL
DIVMTQTPLSSPVTLGQPASISCRSSQTIVHSDGNTYLEWLQQRPGQPPRLLIYKVSNRFSGVPDRFSGSGAGTDFTLKISRVEAEDVGVYYCFQGSHVPWTFGGGTKVEIK

SEQ ID NO: 316 - protein, 112 aa, synthetic construct
  REGION 1..112: note = PR302309-12 VL, PR302309-21 VL, PR302309-6 VL
DVVMTQTPLSSPVTLGQPASISCRSSQTIVHSDGNTYLEWYQQRPGQPPRLLIYKVSNRFSGVPDRFSGSGAGTDFTLKISRVEAEDLGVYYCFQGSHVPWTFGGGTKVEIK

SEQ ID NO: 317 - protein, 112 aa, synthetic construct
  REGION 1..112: note = PR302309-13 VL, PR302309-14 VL, PR302309-15 VL, PR302309-16 VL,PR302309 VL
DVLMAQTPLSLPVSLGDQASISCRSSQTIVHSDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 318 - protein, 112 aa, synthetic construct
  REGION 1..112: note = PR302309-17 VL, PR302309-18 VL, PR302309-19 VL, PR302309-2 VL,PR302309-8 VL
DVVMTQSPLSLPVTLGQPASISCRSSQTIVHSDGNTYLEWYQQRPGQSPRLLIYKVSNRFSGVPDRFSGSGSGTDFTLKISRVEAEDLGVYYCFQGSHVPWTFGGGTKVEIK

SEQ ID NO: 319 - protein, 112 aa, synthetic construct
  REGION 1..112: note = PR302309-3 VL, PR302309-9 VL
DIVMTQTPLSLSVTPGQPASISCRSSQTIVHSDGNTYLEWYLQKPGQSPQLLIYKVSNRFSGVPDRFSGSGSGTDFTLKISRVEAEDVGVYYCFQGSHVPWTFGGGTKVEIK

SEQ ID NO: 320 - protein, 112 aa, synthetic construct
  REGION 1..112: note = PR302312 VL
DVLMTQTPLSLPVSLGDQASISCRSSQSIVHSDGNTYLEWYLQRPGQSPKLLIYKVSNRFSGVPDRFSGSGSGTDFTLKISRVGAEDLGVYYCFQGSHVPWTFGGGTKLEVK

SEQ ID NO: 321 - protein, 112 aa, synthetic construct
  REGION 1..112: note = PR302315 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 322 - protein, 112 aa, synthetic construct
  REGION 1..112: note = PR302324 VL
DVLMTQTPLSLPVSLGDQASISCRSSQSIVHSDGNTYLEWYLQRPGQSPKLLIYKVSNRFSGVPDRFSGSGSGTDFTLKISRVGAEDLGVYYCFQGSHIPWTFGGGTKLEVK

SEQ ID NO: 323 - protein, 106 aa, synthetic construct
  REGION 1..106: note = PR302328 VL, PR302339 VL
NIVMTQTPKFLLVSAGDRVTITCKASQSVNNDVAWYQQKPGQSPKLLIYYASNRYTGVPDRFTGSGYGTDFTFTISTVQAEDLAVYFCQQDYSSWTFGGGTKLEIK

SEQ ID NO: 324 - protein, 106 aa, synthetic construct
  REGION 1..106: note = PR302331 VL
NIVMTQTPKFLLVSAGDRVTIACKASQSVNNDVAWYQQKPGQSPKLLIYYASNRYTGVPDRFTGSGYGTDFTFTISTVQAEDLAVYFCQQDYSSWTFGGGTKLEIK

SEQ ID NO: 325 - protein, 112 aa, synthetic construct
  REGION 1..112: note = PR302332 VL
DVLMAQTPLSLPVSLGDQASISCRSSQSIVHSDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 326 - protein, 112 aa, synthetic construct
  REGION 1..112: note = PR302333 VL
DVLMAQTPLSLPVSLGDQASISCRSSQNIVHSDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 327 - protein, 106 aa, synthetic construct
  REGION 1..106: note = PR302334-1 VL, PR302334-7 VL
EIVMTQSPATLSVSPGERATLSCKASQSVNNDVAWYQQKPGQAPRLLIYYASNRYTGIPARFSGSGSGTEFTLTISSLQSEDFAVYYCQQDYSSWTFGGGTKVEIK

SEQ ID NO: 328 - protein, 106 aa, synthetic construct
  REGION 1..106: note = PR302334-10 VL, PR302334-24 VL, PR302334-26 VL, PR302334-4 VL
DIVMTQSPDSLAVSLGERATINCKASQSVNNDVAWYQQKPGQPPRLLIYYASNRYTGVPDRFSGSGSGTDFTLTISSLQAEDLAVYFCQQDYSSWTFGGGTKVEIK

SEQ ID NO: 329 - protein, 106 aa, synthetic construct
  REGION 1..106: note = PR302334-11 VL, PR302334-5 VL
DIQMTQSSLSASVSLGDRVTITCKASQSVNNDVAWYQQKPGKAPRLLIYYASNRYTGVPSRFSGSGSGTDFTFTISSLQPEDIATYYCQQDYSSWTFGGGTKVEIK

SEQ ID NO: 330 - protein, 106 aa, synthetic construct
  REGION 1..106: note = PR302334-12 VL, PR302334-25 VL, PR302334-27 VL, PR302334-6 VL
DIQMTQSSLSASVSLGDRVTITCKASQSVNNDVAWYQQKPGKAPRLLIYYASNRYTGVPSRFSGSGSGTDFTFTISSLQPEDLATYFCQQDYSSWTFGGGTKVEIK

SEQ ID NO: 331 - protein, 106 aa, synthetic construct
  REGION 1..106: note = PR302334-14 VL, PR302334-15 VL, PR302334-16 VL, PR302334-17 VL,PR302334-18 VL, PR302334 VL
NIVMTQTPKFLLVSAGDRITITCKASQSVNNDVAWYQQKPGQSPKLLIYYASNRYTGVPDRFTGSGYGTDFTFTISTVQAEDLAVYFCQQDYSSWTFGGGTNLEIK

SEQ ID NO: 332 - protein, 106 aa, synthetic construct
  REGION 1..106: note = PR302334-22 VL, PR302334-2 VL, PR302334-8 VL
EIVMTQSPATLSVSPGERATLSCKASQSVNNDVAWYQQKPGQAPRLLIYYASNRYTGIPARFSGSGSGTEFTLTISSLQSEDLAVYFCQQDYSSWTFGGGTKVEIK

SEQ ID NO: 333 - protein, 106 aa, synthetic construct
  REGION 1..106: note = PR302334-3 VL, PR302334-9 VL
DIVMTQSPDSLAVSLGERATINCKASQSVNNDVAWYQQKPGQPPRLLIYYASNRYTGVPDRFSGSGSGTDFTLTISSLQAEDVAVYYCQQDYSSWTFGGGTKVEIK

SEQ ID NO: 334 - protein, 112 aa, synthetic construct
  REGION 1..112: note = PR302335 VL
DVVLTQTPLSLPVSLGDQVSISCRSSQNLLHNNGNTYLHWYLQKPGQSPRLLIYKVSNRFSGVPDRFSGSGSGTDFTLKITRVEAEDLGVYFCSQSTHIPWTFGGGTKLEIK

SEQ ID NO: 335 - protein, 112 aa, synthetic construct
  REGION 1..112: note = PR302341-1 VL, PR302341-7 VL
DVVMTQSPLSLPVTLGQPASISCRSGQSIVHSDGNTYLEWFQQRPGQSPRRLIYKVSNRFSGVPDRFSGSGSGTDFTLKISRVEAEDVGVYYCFQGSHIPWTFGGGTKVEIK

SEQ ID NO: 336 - protein, 112 aa, synthetic construct
  REGION 1..112: note = PR302341-10 VL, PR302341-24 VL, PR302341-26 VL, PR302341-43 VL,PR302341-44 VL, PR302341-45 VL, PR302341-4 VL
DVVMTQTPLSSPVTLGQPASISCRSGQSIVHSDGNTYLEWYQQRPGQPPRLLIYKVSNRFSGVPDRFSGSGAGTDFTLKISRVEAEDLGVYYCFQGSHIPWTFGGGTKVEIK

SEQ ID NO: 337 - protein, 112 aa, synthetic construct
  REGION 1..112: note = PR302341-11 VL, PR302341-5 VL
DIVMTQTPLSLSVTPGQPASISCRSGQSIVHSDGNTYLEWYLQKPGQSPQLLIYKVSNRFSGVPDRFSGSGSGTDFTLKISRVEAEDVGVYYCFQGSHIPWTFGGGTKVEIK

SEQ ID NO: 338 - protein, 112 aa, synthetic construct
  REGION 1..112: note = PR302341-12 VL, PR302341-25 VL, PR302341-27 VL, PR302341-6 VL
DVVMTQTPLSLSVTPGQPASISCRSGQSIVHSDGNTYLEWYLQKPGQSPQLLIYKVSNRFSGVPDRFSGSGSGTDFTLKISRVEAEDLGVYYCFQGSHIPWTFGGGTKVEIK

SEQ ID NO: 339 - protein, 112 aa, synthetic construct
  REGION 1..112: note = PR302341-13 VL, PR302341-14 VL, PR302341-15 VL, PR302341-16 VL,PR302341-17 VL, PR302341-18 VL, PR302341 VL
DVLMTQTPLSLPVSLGDQASISCRSGQSIVHSDGNTYLEWYLQRPGQSPRLLIYKVSNRFSGVPDRFSGSGSGTDFTLKISRVGAEDLGIYYCFQGSHIPWTFGGGTKLEVK

SEQ ID NO: 340 - protein, 112 aa, synthetic construct
  REGION 1..112: note = PR302341-19 VL, PR302341-20 VL, PR302341-21 VL, PR302341-22 VL,PR302341-23 VL, PR302341-28 VL, PR302341-29 VL, PR302341-2 VL,PR302341-41 VL, PR302341-55 VL, PR302341-56 VL, PR302341-57 VL,PR302341-
DVVMTQSPLSLPVTLGQPASISCRSGQSIVHSDGNTYLEWYQQRPGQSPRLLIYKVSNRFSGVPDRFSGSGSGTDFTLKISRVEAEDLGVYYCFQGSHIPWTFGGGTKVEIK

SEQ ID NO: 341 - protein, 112 aa, synthetic construct
  REGION 1..112: note = PR302341-3 VL, PR302341-9 VL
DIVMTQTPLSSPVTLGQPASISCRSGQSIVHSDGNTYLEWLQQRPGQPPRLLIYKVSNRFSGVPDRFSGSGAGTDFTLKISRVEAEDVGVYYCFQGSHIPWTFGGGTKVEIK

SEQ ID NO: 342 - protein, 112 aa, synthetic construct
  REGION 1..112: note = PR302341-30 VL, PR302341-32 VL, PR302341-35 VL, PR302341-36 VL,PR302341-61 VL, PR302341-62 VL, PR302341-63 VL, PR302341-71 VL
DVVMTQSPLSLPVTLGQPASISCRSGQSIVHSEGNTYLEWYQQRPGQSPRLLIYKVSNRFSGVPDRFSGSGSGTDFTLKISRVEAEDLGVYYCFQGSHIPWTFGGGTKVEIK

SEQ ID NO: 343 - protein, 112 aa, synthetic construct
  REGION 1..112: note = PR302341-31 VL, PR302341-33 VL, PR302341-34 VL, PR302341-37 VL,PR302341-58 VL, PR302341-59 VL, PR302341-60 VL, PR302341-70 VL
DVVMTQSPLSLPVTLGQPASISCRSGQSIVHSDANTYLEWYQQRPGQSPRLLIYKVSNRFSGVPDRFSGSGSGTDFTLKISRVEAEDLGVYYCFQGSHIPWTFGGGTKVEIK

SEQ ID NO: 344 - protein, 112 aa, synthetic construct
  REGION 1..112: note = PR302341-38 VL, PR302341-39 VL, PR302341-40 VL, PR302341-42 VL,PR302341-64 VL, PR302341-65 VL, PR302341-66 VL, PR302341-72 VL
DVVMTQSPLSLPVTLGQPASISCRSGQSIVHSSGNTYLEWYQQRPGQSPRLLIYKVSNRFSGVPDRFSGSGSGTDFTLKISRVEAEDLGVYYCFQGSHIPWTFGGGTKVEIK

SEQ ID NO: 345 - protein, 112 aa, synthetic construct
  REGION 1..112: note = PR302341-46 VL, PR302341-47 VL, PR302341-48 VL, PR302341-67 VL
DVVMTQTPLSSPVTLGQPASISCRSGQSIVHSDANTYLEWYQQRPGQPPRLLIYKVSNRFSGVPDRFSGSGAGTDFTLKISRVEAEDLGVYYCFQGSHIPWTFGGGTKVEIK

SEQ ID NO: 346 - protein, 112 aa, synthetic construct
  REGION 1..112: note = PR302341-49 VL, PR302341-50 VL, PR302341-51 VL, PR302341-68 VL
DVVMTQTPLSSPVTLGQPASISCRSGQSIVHSEGNTYLEWYQQRPGQPPRLLIYKVSNRFSGVPDRFSGSGAGTDFTLKISRVEAEDLGVYYCFQGSHIPWTFGGGTKVEIK

SEQ ID NO: 347 - protein, 112 aa, synthetic construct
  REGION 1..112: note = PR302341-52 VL, PR302341-53 VL, PR302341-54 VL, PR302341-69 VL
DVVMTQTPLSSPVTLGQPASISCRSGQSIVHSSGNTYLEWYQQRPGQPPRLLIYKVSNRFSGVPDRFSGSGAGTDFTLKISRVEAEDLGVYYCFQGSHIPWTFGGGTKVEIK

SEQ ID NO: 348 - protein, 445 aa, synthetic construct
  REGION 1..445: note = PR004290 HC, PR005658 HC
EVQLVESGGVLVQPGGSLRLSCAASGFTFSSYWMTWVRQAPGKGLEWVANIKQDGSEKYYVDSVKGRFTISRDNAKNSLFLQMNSLRAEDTAVYYCARTLIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 445 aa, synthetic construct
  REGION 1..445: note = PR005656 HC, PR005660 HC, PR006142 HC, PR006143 HC, PR006144 HC,PR006145 HC
EVQLVESGGVLVQPGGSLRLSCAASGFTFSSYWMTWVRQAPGKGLEWVANIKQEGSEKYYVDSVKGRFTISRDNAKNSLFLQMNSLRAEDTAVYYCARTLIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 445 aa, synthetic construct
  REGION 1..445: note = PR005657 HC, PR005662 HC, PR006146 HC, PR006147 HC, PR006148 HC,PR006149 HC
EVQLVESGGVLVQPGGSLRLSCAASGFTFSSYWMTWVRQAPGKGLEWVANIKQDASEKYYVDSVKGRFTISRDNAKNSLFLQMNSLRAEDTAVYYCARTLIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 445 aa, synthetic construct
  REGION 1..445: note = PR006150 HC, PR006151 HC, PR006152 HC, PR006153 HC
EVQLVESGGVLVQPGGSLRLSCAASGFTFSSYWMTWVRQAPGKGLEWVANIKQDSSEKYYVDSVKGRFTISRDNAKNSLFLQMNSLRAEDTAVYYCARTLIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 445 aa, synthetic construct
  REGION 1..445: note = PR006669 HC
EVQLVESGGGLVQPGGSLRLSCAASGFTFSDNWMSWVRQAPGKGLEWVANIKQDGSENYYVDSVKGRFTISRDNAKNSLYLQMNSLRAEDTAVYYCSRGLALWGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 445 aa, synthetic construct
  REGION 1..445: note = PR006744 HC
EVQLVESGGGLVQPGGSLRLSCAASGFTFSDNWMSWVRQAPGKGLEWVANIKQEGSENYYVDSVKGRFTISRDNAKNSLYLQMNSLRAEDTAVYYCSRGLALWGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 445 aa, synthetic construct
  REGION 1..445: note = PR006745 HC
EVQLVESGGGLVQPGGSLRLSCAASGFTFSDNWMSWVRQAPGKGLEWVANIKQDASENYYVDSVKGRFTISRDNAKNSLYLQMNSLRAEDTAVYYCSRGLALWGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 445 aa, synthetic construct
  REGION 1..445: note = PR006746 HC
EVQLVESGGGLVQPGGSLRLSCAASGFTFSDNWMSWVRQAPGKGLEWVANIKQDSSENYYVDSVKGRFTISRDNAKNSLYLQMNSLRAEDTAVYYCSRGLALWGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 446 aa, synthetic construct
  REGION 1..446: note = PR301306 HC
EVKLEESGGGLVQPGGSMKLSCVASGFTFSNYWMNWVRQSPEKGLEWVAEIRLKSNNYATLYAESVKGRFTISRDDSKSSVYLQMNNLRAEDTGIYYCTRGTLVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 444 aa, synthetic construct
  REGION 1..444: note = PR301612 HC
EVKLEESGGGLVQPGGSIKLSCVASGFTFSGAWMAWVRQSPEKGLEWVAEIKNKANNHATFYAESVKGRFIISRDDSKSSVYLQMNSLRAEDTGTYYCTLPDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 446 aa, synthetic construct
  REGION 1..446: note = PR301777 HC
EMKLEESGGGLVQPGGSMKLSCVASGFTFSNYWMNWVRQSPEKGLEWVAEIRLKSNNYATHYAESVKGRFTISRDDSKSGVYLQMNNLGAEDTGIYYCTVGTLVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 451 aa, synthetic construct
  REGION 1..451: note = PR301780 HC
QIQLVQSGPELKKPGETVKISCKASGYTFTNFGLNWVKQAPGKGLKWMGWINTYTGEPTYTDDFKGRFAFSLETSASTAYLQINNLKKEDTATYFCARGTYRDDGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 446 aa, synthetic construct
  REGION 1..446: note = PR301788 HC, PR301790 HC
EMKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTIGTLVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 446 aa, synthetic construct
  REGION 1..446: note = PR301803 HC
EVKLEESGGGLVQPGGSMKLSCVASGFTFNNYWMNWVRQSPEKGLEWVAEIRLKSNNYATHYAESLKGRFTISRDDSKSSVYLQMNNLRAEDTGIYYCTVGTLVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 451 aa, synthetic construct
  REGION 1..451: note = PR301806 HC
QVQLQQPGAELVKPGTSVKMSCKASGYTFTSYWMHWVKQRPGQGLEWIGDIYPGSDSTNYNEKFKSKASLTVDTSSSTAYMQLSSLTSEDSAVYYCARSITTTTFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 446 aa, synthetic construct
  REGION 1..446: note = PR302029 HC, PR302031 HC, PR302034 HC
EVQLVESGGGLVQPGGSLRLSCAASGFTFSNYWMNWVRQAPGKGLEWVGEIRLKSNNYATHYAESVKGRFTISRDDSKNSLYLQMNSLKTEDTAVYYCTVGTLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 446 aa, synthetic construct
  REGION 1..446: note = PR302036 HC, PR302038 HC, PR302041 HC
EMQLVESGGGLVQPGGSLRLSCAASGFTFSNYWMNWVRQAPGKGLEWVAEIRLKSNNYATHYAESVKGRFTISRDDSKSSVYLQMNSLKTEDTAVYYCTVGTLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 446 aa, synthetic construct
  REGION 1..446: note = PR302043 HC, PR302045 HC, PR302048 HC
EVQLVESGGGLVQPGGSLKLSCAASGFTFSNYWMNWVRQASGKGLEWVGEIRLKSNNYATHYAESVKGRFTISRDDSKNTAYLQMNSLKTEDTAVYYCTRGTLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 446 aa, synthetic construct
  REGION 1..446: note = PR302050 HC, PR302052 HC, PR302055 HC
EMQLVESGGGLVQPGGSLKLSCAASGFTFSNYWMNWVRQASGKGLEWVAEIRLKSNNYATHYAESVKGRFTISRDDSKSTVYLQMNSLKTEDTAVYYCTVGTLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 446 aa, synthetic construct
  REGION 1..446: note = PR302064 HC, PR302066 HC, PR302069 HC
EMQLVESGGGLVKPGGSLRLSCAASGFTFSNYWMNWVRQAPGKGLEWVAEIRLKSNNYATHYAESVKGRFTISRDDSKSTVYLQMNSLKTEDTAVYYCTVGTLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 451 aa, synthetic construct
  REGION 1..451: note = PR302309-1 HC, PR302309-2 HC, PR302309-3 HC, PR302309-4 HC,PR302309-5 HC, PR302309-6 HC, PR302309 HC
QIQLVQSGPELKKPGETVKISCKASGYTFTNYGMNWVKQAPGKVLKWMGWINTYTGEPIYTDDFKGRFAFSLETSASTAYLQINNLKNEDTATYFCSRSTMITTGGVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 451 aa, synthetic construct
  REGION 1..451: note = PR302309-10 HC, PR302309-11 HC, PR302309-12 HC, PR302309-14 HC,PR302309-7 HC, PR302309-8 HC, PR302309-9 HC
QIQLVQSGSELKKPGASVKVSCKASGYTFTNYGMNWVRQAPGQGLEWMGWINTYTGEPIYTDDFKGRFVFSLDTSVSTAYLQISSLKAEDTAVYYCSRSTMITTGGVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 451 aa, synthetic construct
  REGION 1..451: note = PR302309-13 HC, PR302309-17 HC
QVQLVQSGSELKKPGASVKVSCKASGYTFTNYGMNWVRQAPGQGLEWMGWINTYTGEPIYTDDFKGRFVFSLDTSVSTAYLQISSLKAEDTAVYYCARSTMITTGGVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 451 aa, synthetic construct
  REGION 1..451: note = PR302309-15 HC, PR302309-18 HC
QVQLVQSGAEVKKPGASVKVSCKASGYTFTNYGMNWVRQAPGQRLEWMGWINTYTGEPIYTDDFKGRVTITRDTSASTAYMELSSLRSEDTAVYYCARSTMITTGGVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 451 aa, synthetic construct
  REGION 1..451: note = PR302309-16 HC, PR302309-19 HC, PR302309-20 HC, PR302309-21 HC
QIQLVQSGAEVKKPGASVKVSCKASGYTFTNYGMNWVRQAPGQRLEWMGWINTYTGEPIYTDDFKGRVTITLDTSASTAYMELSSLRSEDTAVYYCSRSTMITTGGVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 448 aa, synthetic construct
  REGION 1..448: note = PR302312 HC
EVQLQQSGPELVKPGASVKMSCKASGYTFTDYFMKWMKQSHGKSLEWIGVINPNNGDTFYNQNFKGKATLTVDKSSSTAYMQLNSLTSEDSAVYYCTRGTARALFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 448 aa, synthetic construct
  REGION 1..448: note = PR302315 HC
DVQLVESGGGLMQPGGSRKLSCVASGFTFSSFGMHWVRQAPEKGLEWVAYISSGSSIIYYADTVKGRFTISRDNPKNILFLQMTSLRSEDTAMYYCARDITTATFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 448 aa, synthetic construct
  REGION 1..448: note = PR302324 HC
EVQLQQSGPELVKPGASVKMSCKASGYTFTDYFMKWVKQSHGGSLEWIGVISPNNGDTFYNQKFKGKATLTVDKSSSTAFMQLNSLTSEDSAVYSCARGTDRALFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 441 aa, synthetic construct
  REGION 1..441: note = PR302328 HC
EVKLVESGGGLVKPGGSLKLSCAASGFTFSSYAMSWVRQTPEKRLEWVASIRSGGTTYYPDSVKGRFTISRDNARNILYLQMSSLRSEDTAIYYCTRGIDWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 441 aa, synthetic construct
  REGION 1..441: note = PR302331 HC
EVKLVESGGGLVKPGGSLKLSCAASGFTFSSYAMSWVRQSPEKRLEWVASIRSGGTTYYPDSVKGRFTISRDNARNILYLQMSSLRSEDTAIYYCTRGIDWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 451 aa, synthetic construct
  REGION 1..451: note = PR302332 HC
QIQLVQSGPELKKPGETVKISCKASGYTFTNFGMHWVKQAPGKVLKWMGWINTYTGEPIYAADFKGRFAFSLETSASTAYLQINNLKNEDTATYFCSRSTMITTGGVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 451 aa, synthetic construct
  REGION 1..451: note = PR302333 HC
QIQLVQSGPELKKPGETVKISCKASGYTFTNFGMNWVKQAPGKVLKWMGWINTYTGEPIYADDFKGRFAFSLETSASAAYLQINNLKNEDTATYFCSRSTMITTGGVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 441 aa, synthetic construct
  REGION 1..441: note = PR302334-1 HC, PR302334-2 HC, PR302334-3 HC, PR302334-4 HC,PR302334-5 HC, PR302334-6 HC, PR302334 HC
EVKLVESGGGLVKPGGSLKLSCAASGFTFSTYAMSWVRQTPEKRLEWVASIRSGGTTYYPDSVKGRFTISRDNARNILYLQMSSLRSEDTAIYYCTRGIDWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 441 aa, synthetic construct
  REGION 1..441: note = PR302334-10 HC, PR302334-11 HC, PR302334-12 HC, PR302334-14 HC,PR302334-7 HC, PR302334-8 HC, PR302334-9 HC
EVQLVESGGGLVQPGGSLRLSCAASGFTFSTYAMSWVRQAPGKGLEWVASIRSGGTTYYPDSVKGRFTISRDNSKNTLYLQMNSLRAEDTAVYYCTRGIDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 441 aa, synthetic construct
  REGION 1..441: note = PR302334-15 HC
EVQLVESGGGLVKPGGSLRLSCAASGFTFSTYAMSWVRQAPGKGLEWVSSIRSGGTTYYPDSVKGRFTISRDNAKNSLYLQMNSLRAEDTAVYYCARGIDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 441 aa, synthetic construct
  REGION 1..441: note = PR302334-16 HC, PR302334-24 HC, PR302334-25 HC
EVQLVESGGGLVKPGGSLRLSCAASGFTFSTYAMSWVRQAPGKGLEWVASIRSGGTTYYPDSVKGRFTISRDNAKNSLYLQMNSLRAEDTAVYYCTRGIDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 384 - protein, 441 aa, synthetic construct
  REGION 1..441: note = PR302334-17 HC, PR302334-22 HC
EVQLVESGGGLVQPGGSLRLSCAASGFTFSTYAMSWVRQAPGKGLEWVSSIRSGGTTYYPDSVKGRFTISRDNSKNSLYLQMNSLRAEDTAVYYCARGIDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 441 aa, synthetic construct
  REGION 1..441: note = PR302334-18 HC, PR302334-26 HC, PR302334-27 HC
EVQLVESGGGLVQPGGSLRLSCAASGFTFSTYAMSWVRQAPGKGLEWVASIRSGGTTYYPDSVKGRFTISRDNSKNSLYLQMNSLRAEDTAVYYCTRGIDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 448 aa, synthetic construct
  REGION 1..448: note = PR302335 HC
DVQLVESGGGLVQPGGSRKLSCAASGFTFSSFGMHWVRQAPEKGLEWVAYISSGSTIFYYADTVKGRFTISRDNPKNTLFLQMTSLRSEDTAMYYCARDITTATFAFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 441 aa, synthetic construct
  REGION 1..441: note = PR302339 HC
EVKLVESGGGLVKPGGSLKLSCAASGFTFSSYALSWVRQTPEKRLEWVASIRSGGTTYYPDSVKGRFTISRDNARNILYLQMSSLRSEDTAIYYCTRGIDWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 388 - protein, 448 aa, synthetic construct
  REGION 1..448: note = PR302341-1 HC, PR302341-2 HC, PR302341-3 HC, PR302341-4 HC,PR302341-5 HC, PR302341-6 HC, PR302341 HC
EVQLQQSGPELVKPGASVKMSCKASGYTFTDYFMKWVKQSHGESLEWIGVINPNNGDIFYNQKFKGKATLTVDKSSSTAFMQLNSLTSEDSAVYSCARGTDRALFAYWGQGTLVAVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 389 - protein, 448 aa, synthetic construct
  REGION 1..448: note = PR302341-10 HC, PR302341-11 HC, PR302341-12 HC, PR302341-14 HC,PR302341-67 HC, PR302341-68 HC, PR302341-69 HC, PR302341-7 HC,PR302341-8 HC, PR302341-9 HC
QVQLVQSGAEVKKPGASVKVSCKASGYTFTDYFMKWVRQAPGQGLEWMGVINPNNGDIFYNQKFKGRVTMTVDKSTSTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 390 - protein, 448 aa, synthetic construct
  REGION 1..448: note = PR302341-13 HC, PR302341-19 HC
QVQLVQSGAEVKKPGASVKVSCKASGYTFTDYFMKWVRQAPGQGLEWMGVINPNNGDIFYNQKFKGRVTMTRDTSTSTVYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 448 aa, synthetic construct
  REGION 1..448: note = PR302341-15 HC, PR302341-20 HC, PR302341-70 HC, PR302341-71 HC,PR302341-72 HC
QVQLVQSGAEVKKPGASVKVSCKASGYTFTDYFMKWVRQAPGQRLEWMGVINPNNGDIFYNQKFKGRVTITRDTSASTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 448 aa, synthetic construct
  REGION 1..448: note = PR302341-16 HC, PR302341-21 HC, PR302341-24 HC, PR302341-25 HC
QVQLVQSGAEVKKPGASVKVSCKASGYTFTDYFMKWVRQAPGQRLEWMGVINPNNGDIFYNQKFKGRVTITVDKSASTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 448 aa, synthetic construct
  REGION 1..448: note = PR302341-17 HC, PR302341-22 HC
QVQLVQSGAEVKKPGASVKVSCKASGYTFTDYFMKWVRQAPGQGLEWMGVINPNNGDIFYNQKFKGRVTMTRDTSISTAYMELSRLRSDDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - protein, 448 aa, synthetic construct
  REGION 1..448: note = PR302341-18 HC, PR302341-23 HC, PR302341-26 HC, PR302341-27 HC,PR302341-30 HC, PR302341-31 HC, PR302341-42 HC
QVQLVQSGAEVKKPGASVKVSCKASGYTFTDYFMKWVRQAPGQGLEWMGVINPNNGDIFYNQKFKGRVTMTVDKSISTAYMELSRLRSDDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 395 - protein, 448 aa, synthetic construct
  REGION 1..448: note = PR302341-28 HC, PR302341-32 HC, PR302341-34 HC, PR302341-38 HC
QVQLVQSGAEVKKPGASVKVSCKASGYTFTDYFMKWVRQAPGQGLEWMGVINPNQGDIFYNQKFKGRVTMTVDKSISTAYMELSRLRSDDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 396 - protein, 448 aa, synthetic construct
  REGION 1..448: note = PR302341-29 HC, PR302341-33 HC, PR302341-35 HC, PR302341-39 HC
QVQLVQSGAEVKKPGASVKVSCKASGYTFTDYFMKWVRQAPGQGLEWMGVINPNNADIFYNQKFKGRVTMTVDKSISTAYMELSRLRSDDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 397 - protein, 448 aa, synthetic construct
  REGION 1..448: note = PR302341-36 HC, PR302341-37 HC, PR302341-40 HC, PR302341-41 HC
QVQLVQSGAEVKKPGASVKVSCKASGYTFTDYFMKWVRQAPGQGLEWMGVINPNSGDIFYNQKFKGRVTMTVDKSISTAYMELSRLRSDDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - protein, 448 aa, synthetic construct
  REGION 1..448: note = PR302341-43 HC, PR302341-46 HC, PR302341-49 HC, PR302341-52 HC
QVQLVQSGAEVKKPGASVKVSCKASGYTFTDYFMKWVRQAPGQGLEWMGVINPNNADIFYNQKFKGRVTMTVDKSTSTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 399 - protein, 448 aa, synthetic construct
  REGION 1..448: note = PR302341-44 HC, PR302341-47 HC, PR302341-50 HC, PR302341-53 HC
QVQLVQSGAEVKKPGASVKVSCKASGYTFTDYFMKWVRQAPGQGLEWMGVINPNSGDIFYNQKFKGRVTMTVDKSTSTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 400 - protein, 448 aa, synthetic construct
  REGION 1..448: note = PR302341-45 HC, PR302341-48 HC, PR302341-51 HC, PR302341-54 HC
QVQLVQSGAEVKKPGASVKVSCKASGYTFTDYFMKWVRQAPGQGLEWMGVINPNQGDIFYNQKFKGRVTMTVDKSTSTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 401 - protein, 448 aa, synthetic construct
  REGION 1..448: note = PR302341-55 HC, PR302341-58 HC, PR302341-61 HC, PR302341-64 HC
QVQLVQSGAEVKKPGASVKVSCKASGYTFTDYFMKWVRQAPGQRLEWMGVINPNNADIFYNQKFKGRVTITRDTSASTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 402 - protein, 448 aa, synthetic construct
  REGION 1..448: note = PR302341-56 HC, PR302341-59 HC, PR302341-62 HC, PR302341-65 HC
QVQLVQSGAEVKKPGASVKVSCKASGYTFTDYFMKWVRQAPGQRLEWMGVINPNSGDIFYNQKFKGRVTITRDTSASTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - protein, 448 aa, synthetic construct
  REGION 1..448: note = PR302341-57 HC, PR302341-60 HC, PR302341-63 HC, PR302341-66 HC
QVQLVQSGAEVKKPGASVKVSCKASGYTFTDYFMKWVRQAPGQRLEWMGVINPNQGDIFYNQKFKGRVTITRDTSASTAYMELSSLRSEDTAVYYCARGTDRALFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 404 - protein, 219 aa, synthetic construct
  REGION 1..219: note = PR004290 LC, PR005656 LC, PR005657 LC
DIVMTQSPLSLPVTPGEPASISCRSSQSLLHSTGYNSLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 405 - protein, 219 aa, synthetic construct
  REGION 1..219: note = PR005658 LC, PR005660 LC, PR005662 LC
DIVMTQSPLSLPVTPGEPASISCRSSQSLLHSTGYQSLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 406 - protein, 219 aa, synthetic construct
  REGION 1..219: note = PR006142 LC, PR006146 LC, PR006150 LC
DIVMTQSPLSLPVTPGEPASISCRSSQSLLHSTGYDSLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 407 - protein, 219 aa, synthetic construct
  REGION 1..219: note = PR006143 LC, PR006147 LC, PR006151 LC
DIVMTQSPLSLPVTPGEPASISCRSSQSLLHSTGYHSLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 408 - protein, 219 aa, synthetic construct
  REGION 1..219: note = PR006144 LC, PR006148 LC, PR006152 LC
DIVMTQSPLSLPVTPGEPASISCRSSQSLLHSTGYNPLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409 - protein, 219 aa, synthetic construct
  REGION 1..219: note = PR006145 LC, PR006149 LC, PR006153 LC
DIVMTQSPLSLPVTPGEPASISCRSSQSLLHSTGYSSLEWYLQKPGQSPQLLIYLGSNRASGVPDRFSGSGSGTDFTLKISRVEAEDVGIYYCMQTLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 410 - protein, 219 aa, synthetic construct
  REGION 1..219: note = PR006669 LC, PR006744 LC, PR006745 LC, PR006746 LC
DIVMTHSPLSLPVTPGDPASISCRSSQSLLHSTGYNYLDWYLQKPGQSPQLLIYLGSNRASGVPDRFSGSGSGTDFTLKFIRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 411 - protein, 218 aa, synthetic construct
  REGION 1..218: note = PR301306 LC
DIVLTQSPASLAVSLGQRATISCRASQSVSTSDYNYMHWYQQKPGQPPKLLIKYASNLESGVPARFSGSGSGTDFTLNIHPVEEEDTATYYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 213 aa, synthetic construct
  REGION 1..213: note = PR301612 LC
QIVLTQSPAIMSASPGERVTISCSASSSVSNMYWYLQKPGSSPKPWIYRTSNLASGVPARFSGSGSGTSYSLTISSMEAEDVATYYCQQYHSFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 413 - protein, 218 aa, synthetic construct
  REGION 1..218: note = PR301777 LC, PR301788 LC
DIVLTQSPASLAVSLGQRATISCRASQSVSTSTYNYMHWFQQKPGQPPKLLIKFASNLESGVPARFSGSGSGTDFTLNIHPVEEEDTATFYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 414 - protein, 219 aa, synthetic construct
  REGION 1..219: note = PR301780 LC
DVLMTQTPLSLPVSLGDQASISCRSSQIIVHSNGNTYLEWYLQKPGQSPKLLIYKVSNRFSGVPDRFSGSGSGTDFTLKISRVEAEDLGVYHCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 415 - protein, 218 aa, synthetic construct
  REGION 1..218: note = PR301790 LC
DIVLTQSPASLAVSLGQRATISCRASQSVSTSSYNYMHWYQQKPGQPPKLLIKFASNLESGVPARFSGSGSGTDFTLNIHPVEEEDTATFYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 416 - protein, 218 aa, synthetic construct
  REGION 1..218: note = PR301803 LC
DIVLTQSPASLVVSLGQRATISCRASQSVSTSTYNYMHWYQQKPGQPPKLLIKFASNLESGVPARFSGSGSGTDFTLNIHPVEEEDTATFYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 417 - protein, 219 aa, synthetic construct
  REGION 1..219: note = PR301806 LC
DVLMTQTPLSLPVSLGDQASISCRSSQSFVYSNGNTYLQWYLQKPGQSPKLLIYKVSNRFSGVPDRFSGSGSGTDFTLKISRVEAEDLGVYYCFQGSHVPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - protein, 218 aa, synthetic construct
  REGION 1..218: note = PR302029 LC, PR302036 LC, PR302043 LC, PR302050 LC, PR302064 LC
DIVLTQSPDSLAVSLGERATINCRASQSVSTSTYNYMHWFQQKPGQPPKLLIKFASNLESGVPDRFSGSGSGTDFTLTISSLQAEDTAVYYCQHSWEIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 419 - protein, 218 aa, synthetic construct
  REGION 1..218: note = PR302031 LC, PR302038 LC, PR302045 LC, PR302052 LC, PR302066 LC
EIVLTQSPATLSLSPGERATLSCRASQSVSTSTYNYMHWFQQKPGQAPRLLIKFASNLESGIPARFSGSGSGTDFTLTISSLEPEDTAVYYCQHSWEIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 218 aa, synthetic construct
  REGION 1..218: note = PR302034 LC, PR302041 LC, PR302048 LC, PR302055 LC, PR302069 LC
DIQLTQSPSSLSASVGDRVTITCRASQSVSTSTYNYMHWFQQKPGKAPKLLIKFASNLESGVPSRFSGSGSGTDFTLTISSLQPEDTATYYCQHSWEIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 219 aa, synthetic construct
  REGION 1..219: note = PR302309-1 LC, PR302309-7 LC
DVVMTQSPLSLPVTLGQPASISCRSSQTIVHSDGNTYLEWFQQRPGQSPRRLIYKVSNRFSGVPDRFSGSGSGTDFTLKISRVEAEDVGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 219 aa, synthetic construct
  REGION 1..219: note = PR302309-10 LC, PR302309-20 LC, PR302309-4 LC
DVVMTQTPLSLSVTPGQPASISCRSSQTIVHSDGNTYLEWYLQKPGQSPQLLIYKVSNRFSGVPDRFSGSGSGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 423 - protein, 219 aa, synthetic construct
  REGION 1..219: note = PR302309-11 LC, PR302309-5 LC
DIVMTQTPLSSPVTLGQPASISCRSSQTIVHSDGNTYLEWLQQRPGQPPRLLIYKVSNRFSGVPDRFSGSGAGTDFTLKISRVEAEDVGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 424 - protein, 219 aa, synthetic construct
  REGION 1..219: note = PR302309-12 LC, PR302309-21 LC, PR302309-6 LC
DVVMTQTPLSSPVTLGQPASISCRSSQTIVHSDGNTYLEWYQQRPGQPPRLLIYKVSNRFSGVPDRFSGSGAGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 425 - protein, 219 aa, synthetic construct
  REGION 1..219: note = PR302309-13 LC, PR302309-14 LC, PR302309-15 LC, PR302309-16 LC,PR302309 LC
DVLMAQTPLSLPVSLGDQASISCRSSQTIVHSDGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 426 - protein, 219 aa, synthetic construct
  REGION 1..219: note = PR302309-17 LC, PR302309-18 LC, PR302309-19 LC, PR302309-2 LC,PR302309-8 LC
DVVMTQSPLSLPVTLGQPASISCRSSQTIVHSDGNTYLEWYQQRPGQSPRLLIYKVSNRFSGVPDRFSGSGSGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 427 - protein, 219 aa, synthetic construct
  REGION 1..219: note = PR302309-3 LC, PR302309-9 LC
DIVMTQTPLSLSVTPGQPASISCRSSQTIVHSDGNTYLEWYLQKPGQSPQLLIYKVSNRFSGVPDRFSGSGSGTDFTLKISRVEAEDVGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 428 - protein, 219 aa, synthetic construct
  REGION 1..219: note = PR302312 LC
DVLMTQTPLSLPVSLGDQASISCRSSQSIVHSDGNTYLEWYLQRPGQSPKLLIYKVSNRFSGVPDRFSGSGSGTDFTLKISRVGAEDLGVYYCFQGSHVPWTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 429 - protein, 219 aa, synthetic construct
  REGION 1..219: note = PR302315 LC
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 430 - protein, 219 aa, synthetic construct
  REGION 1..219: note = PR302324 LC
DVLMTQTPLSLPVSLGDQASISCRSSQSIVHSDGNTYLEWYLQRPGQSPKLLIYKVSNRFSGVPDRFSGSGSGTDFTLKISRVGAEDLGVYYCFQGSHIPWTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 431 - protein, 213 aa, synthetic construct
  REGION 1..213: note = PR302328 LC, PR302339 LC
NIVMTQTPKFLLVSAGDRVTITCKASQSVNNDVAWYQQKPGQSPKLLIYYASNRYTGVPDRFTGSGYGTDFTFTISTVQAEDLAVYFCQQDYSSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 432 - protein, 213 aa, synthetic construct
  REGION 1..213: note = PR302331 LC
NIVMTQTPKFLLVSAGDRVTIACKASQSVNNDVAWYQQKPGQSPKLLIYYASNRYTGVPDRFTGSGYGTDFTFTISTVQAEDLAVYFCQQDYSSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 433 - protein, 219 aa, synthetic construct
  REGION 1..219: note = PR302332 LC
DVLMAQTPLSLPVSLGDQASISCRSSQSIVHSDGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 434 - protein, 219 aa, synthetic construct
  REGION 1..219: note = PR302333 LC
DVLMAQTPLSLPVSLGDQASISCRSSQNIVHSDGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - protein, 213 aa, synthetic construct
  REGION 1..213: note = PR302334-1 LC, PR302334-7 LC
EIVMTQSPATLSVSPGERATLSCKASQSVNNDVAWYQQKPGQAPRLLIYYASNRYTGIPARFSGSGSGTEFTLTISSLQSEDFAVYYCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 436 - protein, 213 aa, synthetic construct
  REGION 1..213: note = PR302334-10 LC, PR302334-24 LC, PR302334-26 LC, PR302334-4 LC
DIVMTQSPDSLAVSLGERATINCKASQSVNNDVAWYQQKPGQPPRLLIYYASNRYTGVPDRFSGSGSGTDFTLTISSLQAEDLAVYFCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 437 - protein, 213 aa, synthetic construct
  REGION 1..213: note = PR302334-11 LC, PR302334-5 LC
DIQMTQSSLSASVSLGDRVTITCKASQSVNNDVAWYQQKPGKAPRLLIYYASNRYTGVPSRFSGSGSGTDFTFTISSLQPEDIATYYCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 438 - protein, 213 aa, synthetic construct
  REGION 1..213: note = PR302334-12 LC, PR302334-25 LC, PR302334-27 LC, PR302334-6 LC
DIQMTQSSLSASVSLGDRVTITCKASQSVNNDVAWYQQKPGKAPRLLIYYASNRYTGVPSRFSGSGSGTDFTFTISSLQPEDLATYFCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 439 - protein, 213 aa, synthetic construct
  REGION 1..213: note = PR302334-14 LC, PR302334-15 LC, PR302334-16 LC, PR302334-17 LC,PR302334-18 LC, PR302334 LC
NIVMTQTPKFLLVSAGDRITITCKASQSVNNDVAWYQQKPGQSPKLLIYYASNRYTGVPDRFTGSGYGTDFTFTISTVQAEDLAVYFCQQDYSSWTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 440 - protein, 213 aa, synthetic construct
  REGION 1..213: note = PR302334-22 LC, PR302334-2 LC, PR302334-8 LC
EIVMTQSPATLSVSPGERATLSCKASQSVNNDVAWYQQKPGQAPRLLIYYASNRYTGIPARFSGSGSGTEFTLTISSLQSEDLAVYFCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 441 - protein, 213 aa, synthetic construct
  REGION 1..213: note = PR302334-3 LC, PR302334-9 LC
DIVMTQSPDSLAVSLGERATINCKASQSVNNDVAWYQQKPGQPPRLLIYYASNRYTGVPDRFSGSGSGTDFTLTISSLQAEDVAVYYCQQDYSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 442 - protein, 219 aa, synthetic construct
  REGION 1..219: note = PR302335 LC
DVVLTQTPLSLPVSLGDQVSISCRSSQNLLHNNGNTYLHWYLQKPGQSPRLLIYKVSNRFSGVPDRFSGSGSGTDFTLKITRVEAEDLGVYFCSQSTHIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - protein, 219 aa, synthetic construct
  REGION 1..219: note = PR302341-1 LC, PR302341-7 LC
DVVMTQSPLSLPVTLGQPASISCRSGQSIVHSDGNTYLEWFQQRPGQSPRRLIYKVSNRFSGVPDRFSGSGSGTDFTLKISRVEAEDVGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 219 aa, synthetic construct
  REGION 1..219: note = PR302341-10 LC, PR302341-24 LC, PR302341-26 LC, PR302341-43 LC,PR302341-44 LC, PR302341-45 LC, PR302341-4 LC
DVVMTQTPLSSPVTLGQPASISCRSGQSIVHSDGNTYLEWYQQRPGQPPRLLIYKVSNRFSGVPDRFSGSGAGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 219 aa, synthetic construct
  REGION 1..219: note = PR302341-11 LC, PR302341-5 LC
DIVMTQTPLSLSVTPGQPASISCRSGQSIVHSDGNTYLEWYLQKPGQSPQLLIYKVSNRFSGVPDRFSGSGSGTDFTLKISRVEAEDVGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 219 aa, synthetic construct
  REGION 1..219: note = PR302341-12 LC, PR302341-25 LC, PR302341-27 LC, PR302341-6 LC
DVVMTQTPLSLSVTPGQPASISCRSGQSIVHSDGNTYLEWYLQKPGQSPQLLIYKVSNRFSGVPDRFSGSGSGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 447 - protein, 219 aa, synthetic construct
  REGION 1..219: note = PR302341-13 LC, PR302341-14 LC, PR302341-15 LC, PR302341-16 LC,PR302341-17 LC, PR302341-18 LC, PR302341 LC
DVLMTQTPLSLPVSLGDQASISCRSGQSIVHSDGNTYLEWYLQRPGQSPRLLIYKVSNRFSGVPDRFSGSGSGTDFTLKISRVGAEDLGIYYCFQGSHIPWTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - protein, 219 aa, synthetic construct
  REGION 1..219: note = PR302341-19 LC, PR302341-20 LC, PR302341-21 LC, PR302341-22 LC,PR302341-23 LC, PR302341-28 LC, PR302341-29 LC, PR302341-2 LC,PR302341-41 LC, PR302341-55 LC, PR302341-56 LC, PR302341-57 LC,PR302341-
DVVMTQSPLSLPVTLGQPASISCRSGQSIVHSDGNTYLEWYQQRPGQSPRLLIYKVSNRFSGVPDRFSGSGSGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 449 - protein, 219 aa, synthetic construct
  REGION 1..219: note = PR302341-3 LC, PR302341-9 LC
DIVMTQTPLSSPVTLGQPASISCRSGQSIVHSDGNTYLEWLQQRPGQPPRLLIYKVSNRFSGVPDRFSGSGAGTDFTLKISRVEAEDVGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 450 - protein, 219 aa, synthetic construct
  REGION 1..219: note = PR302341-30 LC, PR302341-32 LC, PR302341-35 LC, PR302341-36 LC,PR302341-61 LC, PR302341-62 LC, PR302341-63 LC, PR302341-71 LC
DVVMTQSPLSLPVTLGQPASISCRSGQSIVHSEGNTYLEWYQQRPGQSPRLLIYKVSNRFSGVPDRFSGSGSGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 219 aa, synthetic construct
  REGION 1..219: note = PR302341-31 LC, PR302341-33 LC, PR302341-34 LC, PR302341-37 LC,PR302341-58 LC, PR302341-59 LC, PR302341-60 LC, PR302341-70 LC
DVVMTQSPLSLPVTLGQPASISCRSGQSIVHSDANTYLEWYQQRPGQSPRLLIYKVSNRFSGVPDRFSGSGSGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 452 - protein, 219 aa, synthetic construct
  REGION 1..219: note = PR302341-38 LC, PR302341-39 LC, PR302341-40 LC, PR302341-42 LC,PR302341-64 LC, PR302341-65 LC, PR302341-66 LC, PR302341-72 LC
DVVMTQSPLSLPVTLGQPASISCRSGQSIVHSSGNTYLEWYQQRPGQSPRLLIYKVSNRFSGVPDRFSGSGSGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 453 - protein, 219 aa, synthetic construct
  REGION 1..219: note = PR302341-46 LC, PR302341-47 LC, PR302341-48 LC, PR302341-67 LC
DVVMTQTPLSSPVTLGQPASISCRSGQSIVHSDANTYLEWYQQRPGQPPRLLIYKVSNRFSGVPDRFSGSGAGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 219 aa, synthetic construct
  REGION 1..219: note = PR302341-49 LC, PR302341-50 LC, PR302341-51 LC, PR302341-68 LC
DVVMTQTPLSSPVTLGQPASISCRSGQSIVHSEGNTYLEWYQQRPGQPPRLLIYKVSNRFSGVPDRFSGSGAGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 455 - protein, 219 aa, synthetic construct
  REGION 1..219: note = PR302341-52 LC, PR302341-53 LC, PR302341-54 LC, PR302341-69 LC
DVVMTQTPLSSPVTLGQPASISCRSGQSIVHSSGNTYLEWYQQRPGQPPRLLIYKVSNRFSGVPDRFSGSGAGTDFTLKISRVEAEDLGVYYCFQGSHIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 456 - protein, 107 aa, synthetic construct [sequence omitted]

SEQ ID NO: 457 - protein, 330 aa, synthetic construct
  REGION 1..330: note = human IgG1 constant region (include CH1, Hinge, CH2, CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - protein, 30 aa, synthetic construct
  REGION 1..30: note = PR004289 HFWR1 Kabat
EVQLVESGGVLVQPGGSLRLSCAASGFIFS

SEQ ID NO: 459 - protein, 115 aa, synthetic construct
  REGION 1..115: note = PR004289 VH
EVQLVESGGVLVQPGGSLRLSCAASGFIFSSYWMTWVRQAPGKGLEWVANIKQDGSEKYYVDSVKGRFTISRDNAKNSLFLQMNSLRAEDTAVYYCARTLIFDYWGQGTLVTVSS

SEQ ID NO: 460 - protein, 445 aa, synthetic construct
  REGION 1..445: note = PR004289 HC
EVQLVESGGVLVQPGGSLRLSCAASGFIFSSYWMTWVRQAPGKGLEWVANIKQDGSEKYYVDSVKGRFTISRDNAKNSLFLQMNSLRAEDTAVYYCARTLIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 461 - protein, 113 aa, synthetic construct
  REGION 1..113: note = CTRND05_VH
EVQLQQSGPELVKPGVSMKISCKASGYSFTDSTMNWVKQSHGKNLEWIGLIHPDNGGTIYNQKFKGKATLTVHKSSSTAYMELLSLTSEDSAVYYCANGFAYWGQGTLVTVSA

SEQ ID NO: 462 - protein, 112 aa, synthetic construct
  REGION 1..112: note = CTRND05_VL
DVVMTQTPLSLPVSLGDQASISCRSSQSLLHSNGNTYLHWYLQKPGQSPELLIYKVSNRFSGVPDRFSGSGSGTDFTLKISRVEAEDLGVYFCSQSTHVPLTFGAGTKLELK

SEQ ID NO: 463 - protein, 443 aa, synthetic construct
  REGION 1..443: note = PR303394_HC
EVQLQQSGPELVKPGVSMKISCKASGYSFTDSTMNWVKQSHGKNLEWIGLIHPDNGGTIYNQKFKGKATLTVHKSSSTAYMELLSLTSEDSAVYYCANGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 464 - protein, 219 aa, synthetic construct
  REGION 1..219: note = PR303394_LC
DVVMTQTPLSLPVSLGDQASISCRSSQSLLHSNGNTYLHWYLQKPGQSPELLIYKVSNRFSGVPDRFSGSGSGTDFTLKISRVEAEDLGVYFCSQSTHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 465 - DNA, 11 bp, synthetic construct
  source 1..11: mol_type = other DNA
  misc_feature 1..11: note = Base pair deletion
cagccggttct

SEQ ID NO: 466 - protein, 41 aa, synthetic construct
  REGION 1..41: note = human and mouse CRH (UniProtKB - P06850; Genbank Accession No.EAW86897.1
SEEPPISLDLTFHLLREVLEMARAEQLAQQAHSNRKLMEII

SEQ ID NO: 467 - protein, 41 aa, synthetic construct; identical to SEQ ID NO: 466
  REGION 1..41: note = CRH-KLH conjugates

SEQ ID NO: 468 - protein, 41 aa, synthetic construct; identical to SEQ ID NO: 466
  REGION 1..41: note = Peptide 1

SEQ ID NO: 469 - protein, 41 aa, synthetic construct; identical to SEQ ID NO: 466
  REGION 1..41: note = Peptide 2

SEQ ID NO: 470 - protein, 41 aa, synthetic construct; identical to SEQ ID NO: 466
  REGION 1..41: note = Peptide 3

SEQ ID NO: 471 - protein, 41 aa, synthetic construct; identical to SEQ ID NO: 466
  REGION 1..41: note = Peptide 4

SEQ ID NO: 472 - protein, 41 aa, synthetic construct; identical to SEQ ID NO: 466
  REGION 1..41: note = Biotin-CRH

SEQ ID NO: 473 - DNA, 21 bp, synthetic construct
  source 1..21: mol_type = other DNA
  misc_feature 1..21: note = Wild type allele of exon 1 of MRAP1: forward primer
gcgtctcttagtagcctttgg

SEQ ID NO: 474 - DNA, 20 bp, synthetic construct
  source 1..20: mol_type = other DNA
  misc_feature 1..20: note = Wild type allele of exon 1 of MRAP1: reverse primer
cttgttggctttcagcttct

SEQ ID NO: 475 - DNA, 29 bp, synthetic construct
  source 1..29: mol_type = other DNA
  misc_feature 1..29: note = Mutant allele of exon 1 of MRAP1: forward primer
gaacttgcctggtcaactgttaaaaggac

SEQ ID NO: 476 - DNA, 27 bp, synthetic construct
  source 1..27: mol_type = other DNA
  misc_feature 1..27: note = Mutant allele of exon 1 of MRAP1: reverse primer
tctttaggctcacttcctccactgacc

SEQ ID NO: 477 - DNA, 23 bp, synthetic construct
  source 1..23: mol_type = other DNA
  misc_feature 1..23: note = CRHbp70 forward sequence
gccctgctcagcaggggatccgt

SEQ ID NO: 478 - DNA, 23 bp, synthetic construct
  source 1..23: mol_type = other DNA
  misc_feature 1..23: note = CRHbp215R reverse sequence
ctgttgagattccccaggcggag

SEQ ID NO: 479 - DNA, 21 bp, synthetic construct
  source 1..21: mol_type = other DNA
  misc_feature 1..21: note = CRHbp208F Forward sequence
ctcaacagaagtcccgctcgg

SEQ ID NO: 480 - DNA, 23 bp, synthetic construct
  source 1..23: mol_type = other DNA
  misc_feature 1..23: note = CRHbp310 Reverse sequence
ggaaaaagttagccgcagcctgg

SEQ ID NO: 481 - protein, 41 aa, synthetic construct; identical to SEQ ID NO: 466
  REGION 1..41: note = Full Length CRH

SEQ ID NO: 482 - protein, 31 aa, synthetic construct
  REGION 1..31: note = N-terminal CRH
SEEPPISLDLTFHLLREVLEMARAEQLAQQA

SEQ ID NO: 483 - protein, 24 aa, synthetic construct
  REGION 1..24: note = C-Terminal CRH
VLEMARAEQLAQQAHSNRKLMEII

SEQ ID NO: 484 - protein, 20 aa, synthetic construct
  REGION 1..20: note = Central CRH
LREVLEMARAEQLAQQAHSN

SEQ ID NO: 485 - protein, 20 aa, synthetic construct
  REGION 1..20: note = Central CRH
LREVLEMARAEQLAQQAHSN

SEQ ID NO: 486 - DNA, 564 bp, synthetic construct
  source 1..564: mol_type = other DNA
  misc_feature 1..564: note = corticotropin-releasing hormone
atgcggctgcggctgctggtgtccgcgggcatgctgctggtggctctgtcgtcctgcctgccttgcagggccctgctcagcaggggatccgtcccccgagcgccgcgggccccgcagcccttgaatttcttgcagccggagcagccccagcaacctgatccgcatggcagccggttctgtgaagaatacttcctccgcctggggaatctcaacagaagtcccgctgctcggctgtcccccaactccacgcccctcaccgcgggtcgcggcagccgcccctcgcacgaccaggctgcggctaactttttccgcgtgttgctgcagcagctgcagatgcctcagcgctcgctcgacagccgcgcggagccggccgaacgcggcgccgaggatgccctcggtggccaccagggggcgctggagagggagaggcggtcggaggagccgcccatctctctggatctcaccttccaccttctgcgggaagtcttggaaatggcccgggcagagcagttagctcagcaagctcacagcaacaggaaactgatggagattatcgggaaatga

SEQ ID NO: 487 - DNA, 553 bp, synthetic construct
  misc_feature 1..553: note = corticotrophin-releasing hormone (CRH) frame shift knock out
  source 1..553: mol_type = other DNA
atgcggctgcggctgctggtgtccgcgggcatgctgctggtggctctgtcgtcctgcctgccttgcagggccctgctcagcaggggatccgtcccccgagcgccgcgggccccgcagcccttgaatttcttgcagccggagcagccccagcaacctgatccgcatgggtgaagaatacttcctccgcctggggaatctcaacagaagtcccgctgctcggctgtcccccaactccacgcccctcaccgcgggtcgcggcagccgcccctcgcacgaccaggctgcggctaactttttccgcgtgttgctgcagcagctgcagatgcctcagcgctcgctcgacagccgcgcggagccggccgaacgcggcgccgaggatgccctcggtggccaccagggggcgctggagagggagaggcggtcggaggagccgcccatctctctggatctcaccttccaccttctgcgggaagtcttggaaatggcccgggcagagcagttagctcagcaagctcacagcaacaggaaactgatggagattatcgggaaatga

(19) United States
(12) Patent Application Publication
(10) Pub. No.: US 2025/0197490 A1
(43) Pub. Date: Jun. 19, 2025

(54) ANTI-CORTICOTROPIN-RELEASING HORMONE ANTIBODIES AND USE IN CONGENITAL ADRENAL HYPERPLASIA

(71) Applicant: HBM ALPHA THERAPEUTICS, INC., Wilmington, DE (US)

(72) Inventors: Joseph A. MAJZOUB, Rockport, MA (US); Lile LIU, Suzhou (CN); Hongjie PAN, West Roxbury, MA (US); Qiang LV, Suzhou (CN); Frank GROSVELD, Rotterdam (NL); Dubravka DRABEK, Rotterdam (NL); Rien VAN HAPEREN, Prinsenbeek (NL); Xiaoxiao WANG, Suzhou (CN); Yun HE, Shanghai (CN); Yongqiang WANG, Shanghai (CN); Jin HUANG, Boston, MA (US); Jung LEE, McLean, VA (US); Jiuqiao ZHAO, Hockessin, DE (US)

(73) Assignee: HBM ALPHA THERAPEUTICS, INC., Wilmington, DE (US)

(21) Appl. No.: 18/681,163

(22) PCT Filed: Aug. 5, 2022
(86) PCT No.: PCT/US2022/074609

§ 371 Date: Feb. 5, 2024

Related U.S. Application Data
(60) Provisional application No. 63/234,130, filed on Aug. 17, 2021.

(51) Int. Cl. C07K 16/26; A61K 39/00; A61P 5/38

(52) U.S. Cl. CPC C07K 16/26; A61P 5/38; A61K 2039/505; C07K 2317/24; C07K 2317/34; C07K 2317/40; C07K 2317/76; C07K 2317/92

(57) ABSTRACT

Disclosed herein are antibodies and antigen-binding fragments thereof that specifically bind to corticotropin-releasing hormone (CRH). Also disclosed are methods and pharmaceutical compositions containing such antibodies and antigen-binding fragments, and the use of said anti-corticotropin-releasing hormone (CRH) antibodies and antigen-binding fragments thereof in treating congenital adrenal hyperplasia (CAH) and related disorders.

### CROSS REFERENCE TO RELATED APPLICATIONS

This application is the U.S. national phase entry of Int'l Appl. No. PCT/US2022/074609, filed Aug. 5, 2022, which claims the priority benefit of U.S. Provisional Application No. 63/234,130, filed Aug. 17, 2021, which are incorporated herein by reference in their entireties.

### REFERENCE TO SEQUENCE LISTING SUBMITTED ELECTRONICALLY

The content of the electronically submitted sequence listing (Name: 4857_0010002_Seqlisting_ST26; Size: 686,229 bytes; and Date of Creation: Jul. 12, 2024) is herein incorporated by reference in its entirety.

### FIELD

The present disclosure relates to antibodies and antigen-binding fragments thereof that specifically bind to corticotropin-releasing hormone (CRH) and block the binding of CRH to its receptors. The present disclosure also relates to pharmaceutical compositions containing such antibodies and antigen binding fragments, and the use of anti-CRH antibodies and antigen binding fragments thereof in treating congenital adrenal hyperplasia and other related disorders and conditions.

### BACKGROUND

Congenital adrenal hyperplasia (CAH) is a group of rare inherited autosomal recessive disorders characterized by a deficiency of one of the enzymes needed for hormone production in the adrenal glands. CAH affects the adrenal glands located at the top of each kidney. Normally, the adrenal glands are responsible for producing three different hormones: (i) corticosteroids, which control the body's response to illness or injury, (ii) mineralocorticoids, which regulate salt and water levels, and (iii) androgens, which are male sex hormones. An enzyme deficiency associated with CAH makes the adrenal gland unable to produce one or more of these hormones, which in turn results in the overproduction of another type of hormone precursor in response to the loss.

The most common cause of CAH is the absence of the enzyme 21-hydroxylase. CAH due to 21-hydroxylase deficiency is responsible for 95% of all cases of CAH and is broken down further into two subcategories: (i) classic CAH, which can be subdivided into the salt-losing form and the simple-virilizing form, and (ii) non-classic CAH. Classic CAH is the more severe form and can result in adrenal crisis and death if not detected and treated. Non-classic CAH is milder, and may or may not present symptoms. The absence of 21-hydroxylase interferes with these individuals' ability to make the hormone cortisol and, in the case of salt-losing CAH, aldosterone. In addition, the body produces more androgens which cause a variety of symptoms such as abnormal genital development in infant girls. There are also other much rarer forms of CAH as well, including 11-Beta hydroxylase deficiency, 17α-hydroxylase deficiency, 3-Beta-hydroxysteroid dehydrogenase deficiency, congenital lipoid adrenal hyperplasia, and p450 oxidoreductase deficiency.

Classic CAH is a disease that includes a group of autosomal recessive disorders that result in an enzyme deficiency that alters the production of adrenal steroids due to 21-hydroxylase deficiency, a condition that results in little or no cortisol biosynthesis. One clinical manifestation of the absence of cortisol is the lack of feedback inhibition of pituitary adrenocorticotropic hormone (ACTH) secretion. Increased ACTH levels cause adrenal hyperplasia and the enzyme mutation causes a shunting of cortisol precursor steroids to alternate pathways. Most notably, shunting into androgens leads to virilization and other developmental complications in females and high ACTH concentrations are associated with the formation of testicular adrenal rest tumors (TARTs) in males. In addition, since 21-hydroxylase is used in the pathway for the biosynthesis of the mineralocorticoids, a number of these patients suffer from aldosterone deficiency which can result in dehydration and death due to salt-wasting. The prevalence of classic 21-hydroxylase deficiency CAH in the U.S. general population, based on newborn screening, has been documented as 1:10,000 to 1:20, 800 (Trakakis et al., Gynecol Endocrinol (2010) 26 (1): 63-71; Hertzberg et al., Pediatr. (2011) 159 (4): 555-560).

Pediatric patients from birth through adolescence, and females in particular, appear to be the most vulnerable population of CAH sufferers and represent the subgroup of patients with the greatest unmet medical need (Cheng and Speiser, Adv. Pediatr. (2012) 59 (1): 269-281; Merke and Poppas, Lancet Diabetes Endocrinol. (2013) 1 (4): 341-352). Excessive androgen production in these younger patients results in early onset puberty and adrenarche, changes in skeletal maturation patterns, short stature caused by premature growth plate fusion, as well as significant hirsutism and acne problems. While survival is properly ensured through steroid replacement strategies based on physiologic dosing of glucocorticoids (e.g., hydrocortisone) and mineralocorticoids (e.g., fludrocortisone), these doses are often inadequate to suppress the accumulating ACTH and overproduction of progestogens and androgens (e.g., 17-hydroxyprogesterone [17-OHP], testosterone, free testosterone, androstenedione, an 11-oxygenated androgen, or a combination thereof). The uncontrolled symptoms of androgen excess have a substantial impact on the day-to-day functioning and development of these patients.

Currently, exogenous corticosteroids are the standard of care for treating patients with classic CAH. This treatment is used to correct the cortisol deficiency and reduce the excessive ACTH levels and androgen excess. However, the dose and duration of steroid use required to suppress ACTH are typically well above the normal physiological level used for cortisol replacement alone. This increased exposure to glucocorticoids can lead to, for example, iatrogenic Cushing syndrome, increased cardiovascular risk factors, glucose intolerance, reduced growth velocity, and decreased bone mineral density in CAH patients (Elnecave et al., J. Pediatr. Endocrinol. Metab. (2008) 21 (12): 1155-1162; King et al., J. Clin. Endocrinol Metab. (2006) 91 (3): 865-869; Migeon and Wisniewski, Endocrinol. Metab. Clin. North Am. (2001) 30 (1): 193-206). Therefore, there is a current need in the art for improved therapeutics for CAH.

### BRIEF SUMMARY

Provided herein is an antibody or antigen-binding fragment thereof that specifically binds to corticotropin-releasing hormone (CRH), comprising: (a) a heavy chain variable domain (VH) CDR1 comprising an amino acid sequence of any one of SEQ ID NOs: 22-35, a VH CDR2 comprising an amino acid sequence of any one of SEQ ID NOs: 53-78, and a VH CDR3 comprising an amino acid sequence of any one of SEQ ID NOs: 113-122 or the amino acid sequence of PDV or GID; and (b) a light chain variable domain (VL) CDR1 comprising an amino acid sequence of any one of SEQ ID NOs: 152-174, a VL CDR2 comprising the amino acid sequence of any one of SEQ ID NOs: 193-198, and a VL CDR3 comprising an amino acid sequence of any one of SEQ ID NOs: 223-232.

In one aspect, the antibody comprises: (a) the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223; (b) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223; (c) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223; (d) the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223; (e) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223; (f) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223; (g) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 154, the VL CDR2 comprises the amino acid sequence of SEQ ID NO: 193, and the VL CDR3 comprises the amino acid sequence of SEQ ID NO: 223; (h) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223; (i) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 156, the VL CDR2 comprises the amino acid sequence of SEQ ID NO: 193, and the VL CDR3 comprises the amino acid sequence of SEQ ID NO: 223; (j) the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 157, the VL CDR2 comprises the amino acid sequence of SEQ ID NO: 193, and the VL CDR3 comprises the amino acid sequence of SEQ ID NO: 223; (k) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 154, the VL CDR2 comprises the amino acid sequence of SEQ ID NO: 193, and the VL CDR3 comprises the amino acid sequence of SEQ ID NO: 223; (1) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223; (m) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 156, the VL CDR2 comprises the amino acid sequence of SEQ ID NO: 193, and the VL CDR3 comprises the amino acid sequence of SEQ ID NO: 223; (n) the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 157, the VL CDR2 comprises the amino acid sequence of SEQ ID NO: 193, and the VL CDR3 comprises the amino acid sequence of SEQ ID NO: 223; (0) the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 154, the VL CDR2 comprises the amino acid sequence of SEQ ID NO: 193, and the VL CDR3 comprises the amino acid sequence of SEQ ID NO: 223; (p) the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223; (q) the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 156, the VL CDR2 comprises the amino acid sequence of SEQ ID NO: 193, and the VL CDR3 comprises the amino acid sequence of SEQ ID NO: 223; (r) the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 157, the VL CDR2 comprises the amino acid sequence of SEQ ID NO: 193, and the VL CDR3 comprises the amino acid sequence of SEQ ID NO: 223; (s) the VH CDR1 comprises the amino acid sequence of SEQ ID NO: 23, the VH CDR2 comprises the amino acid sequence of SEQ ID NO: 57, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224; (t) the VH CDR1 comprises the amino acid sequence of SEQ ID NO: 23, the VH CDR2 comprises the amino acid sequence of SEQ ID NO: 58, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224; (u) the VH CDR1 comprises the amino acid sequence of SEQ ID NO: 23, the VH CDR2 comprises the amino acid sequence of SEQ ID NO: 59, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224; (v) the VH CDR1 comprises the amino acid sequence of SEQ ID NO: 23, the VH CDR2 comprises the amino acid sequence of SEQ ID NO: 60, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224; (w) the VH CDR1 comprises the amino acid sequence of SEQ ID NO: 24, the VH CDR2 comprises the amino acid sequence of SEQ ID NO: 61, the VH CDR3 comprises the amino acid sequence of SEQ ID NO: 115, the VL CDR1 comprises the amino acid sequence of SEQ ID NO: 159, the VL CDR2 comprises the amino acid sequence of SEQ ID NO: 194, and the VL CDR3 comprises the amino acid sequence of SEQ ID NO: 225; (x) the VH CDR1 comprises the amino acid sequence of SEQ ID NO: 25, the VH CDR2 comprises the amino acid sequence of SEQ ID NO: 62, the VH CDR3 comprises the amino acid sequence of PDV, the VL CDR1 comprises the amino acid sequence of SEQ ID NO: 160, the VL CDR2 comprises the amino acid sequence of SEQ ID NO: 195, and the VL CDR3 comprises the amino acid sequence of SEQ ID NO: 226; (y) the VH CDR1 comprises the amino acid sequence of SEQ ID NO: 24, the VH CDR2 comprises the amino acid sequence of SEQ ID NO: 63, the VH CDR3 comprises the amino acid sequence of SEQ ID NO: 115, the VL CDR1 comprises the amino acid sequence of SEQ ID NO: 161, the VL CDR2 comprises the amino acid sequence of SEQ ID NO: 196, and the VL CDR3 comprises the amino acid sequence of SEQ ID NO: 225; (z) the VH CDR1 comprises the amino acid sequence of SEQ ID NO: 26, the VH CDR2 comprises the amino acid sequence of SEQ ID NO: 64, the VH CDR3 comprises the amino acid sequence of SEQ ID NO: 116, the VL CDR1 comprises the amino acid sequence of SEQ ID NO: 162, the VL CDR2 comprises the amino acid sequence of SEQ ID NO: 197, and the VL CDR3 comprises the amino acid sequence of SEQ ID NO: 227; (aa) the VH CDR1 comprises the amino acid sequence of SEQ ID NO: 24, the VH CDR2 comprises the amino acid sequence of SEQ ID NO: 63, the VH CDR3 comprises the amino acid sequence of SEQ ID NO: 115, the VL CDR1 comprises the amino acid sequence of SEQ ID NO: 163, the VL CDR2 comprises the amino acid sequence of SEQ ID NO: 196, and the VL CDR3 comprises the amino acid sequence of SEQ ID NO: 225; (bb) the VH CDR1 comprises the amino acid sequence of SEQ ID NO: 24, the VH CDR2 comprises the amino acid sequence of SEQ ID NO: 65, the VH CDR3 comprises the amino acid sequence of SEQ ID NO: 115, the VL CDR1 comprises the amino acid sequence of SEQ ID NO: 161, the VL CDR2 comprises the amino acid sequence of SEQ ID NO: 196, and the VL CDR3 comprises the amino acid sequence of SEQ ID NO: 225; (cc) the VH CDR1 comprises the amino acid sequence of SEQ ID NO: 27, the VH CDR2 comprises the amino acid sequence of SEQ ID NO: 66, the VH CDR3 comprises the amino acid sequence of SEQ ID NO: 117, the VL CDR1 comprises the amino acid sequence of SEQ ID NO: 164, the VL CDR2 comprises the amino acid sequence of SEQ ID NO: 197, and the VL CDR3 comprises the amino acid sequence of SEQ ID NO: 228; (dd) the VH CDR1 comprises the amino acid sequence of SEQ ID NO: 28, the VH CDR2 comprises the amino acid sequence of SEQ ID NO: 67, the VH CDR3 comprises the amino acid sequence of SEQ ID NO: 118, the VL CDR1 comprises the amino acid sequence of SEQ ID NO: 165, the VL CDR2 comprises the amino acid sequence of SEQ ID NO: 197, and the VL CDR3 comprises the amino acid sequence of SEQ ID NO: 227; (ee) the VH CDR1 comprises the amino acid sequence of SEQ ID NO: 29, the VH CDR2 comprises the amino acid sequence of SEQ ID NO: 68, the VH CDR3 comprises the amino acid sequence of SEQ ID NO: 119, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 227; (ff) the VH CDR1 comprises the amino acid sequence of SEQ ID NO: 30, the VH CDR2 comprises the amino acid sequence of SEQ ID NO: 69, the VH CDR3 comprises the amino acid sequence of SEQ ID NO: 120, the VL CDR1 comprises the amino acid sequence of SEQ ID NO: 167, the VL CDR2 comprises the amino acid sequence of SEQ ID NO: 197, and the VL CDR3 comprises the amino acid sequence of SEQ ID NO: 229; (gg) the VH CDR1 comprises the amino acid sequence of SEQ ID NO: 29, the VH CDR2 comprises the amino acid sequence of SEQ ID NO: 70, the VH CDR3 comprises the amino acid sequence of SEQ ID NO: 121, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 230; (hh) the VH CDR1 comprises the amino acid sequence of SEQ ID NO: 31, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231; (ii) the VH CDR1 comprises the amino acid sequence of SEQ ID NO: 32, the VH CDR2 comprises the amino acid sequence of SEQ ID NO: 72, the VH CDR3 comprises the amino acid sequence of SEQ ID NO: 118, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 227; (jj) the VH CDR1 comprises the amino acid sequence of SEQ ID NO: 33, the VH CDR2 comprises the amino acid sequence of SEQ ID NO: 73, the VH CDR3 comprises the amino acid sequence of SEQ ID NO: 118, the VL CDR1 comprises the amino acid sequence of SEQ ID NO: 169, the VL CDR2 comprises the amino acid sequence of SEQ ID NO: 197, and the VL CDR3 comprises the amino acid sequence of SEQ ID NO: 227; (kk) the VH CDR1 comprises the amino acid sequence of SEQ ID NO: 34, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231; (ll) the VH CDR1 comprises the amino acid sequence of SEQ ID NO: 30, the VH CDR2 comprises the amino acid sequence of SEQ ID NO: 74, the VH CDR3 comprises the amino acid sequence of SEQ ID NO: 122, the VL CDR1 comprises the amino acid sequence of SEQ ID NO: 170, the VL CDR2 comprises the amino acid sequence of SEQ ID NO: 197, and the VL CDR3 comprises the amino acid sequence of SEQ ID NO: 232; (mm) the VH CDR1 comprises the amino acid sequence of SEQ ID NO: 35, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231; (nn) the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230; (oo) the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230; (pp) the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230; (qq) the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230; (rr) the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230; (ss) the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230; (tt) the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230; (uu) the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230; (vv) the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230; (ww) the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230; (xx) the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230; (yy) the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230; (zz) the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230; (aaa) the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230; (bbb) the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230; (ccc) the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230; or (ddd) the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223.

In another aspect, the invention relates to an antibody or antigen-binding fragment thereof that comprises (a) a VH comprising an amino acid sequence at least 90%, 95%, 99%, or 100% identical to any one of SEQ ID NOs: 240-295, 459 and 461; and/or (b) a VL comprising an amino acid sequence at least 90%, 95%, 99%, or 100% identical to any one of SEQ ID NOs: 296-347 and 462. In one aspect, the antibody or antigen-binding fragment thereof comprises an amino acid sequence at least 90%, 95%, 99%, or 100% identical to the: (a) VH amino acid sequence of SEQ ID NO: 240, and/or the VL amino acid sequence of SEQ ID NO: 296; (b) VH amino acid sequence of SEQ ID NO: 241, and/or the VL amino acid sequence of SEQ ID NO: 296; (c) the VH amino acid sequence of SEQ ID NO: 242, and/or the VL amino acid sequence of SEQ ID NO: 296; (d) the VH amino acid sequence of SEQ ID NO: 240, and/or the VL amino acid sequence of SEQ ID NO: 297; (e) the VH amino acid sequence of SEQ ID NO: 241, and/or the VL amino acid sequence of SEQ ID NO: 297; (f) the VH amino acid sequence of SEQ ID NO: 242, and/or the VL amino acid sequence of SEQ ID NO: 297; (g) the VH amino acid sequence of SEQ ID NO: 241, and/or the VL amino acid sequence of SEQ ID NO: 298; (h) the VH amino acid sequence of SEQ ID NO: 241, and the VL amino acid sequence of SEQ ID NO: 299; (i) the VH amino acid sequence of SEQ ID NO: 241, and the VL amino acid sequence of SEQ ID NO: 300; (j) the VH amino acid sequence of SEQ ID NO: 241, and/or the VL amino acid sequence of SEQ ID NO: 301; (k) the VH amino acid sequence of SEQ ID NO: 242, and/or the VL amino acid sequence of SEQ ID NO: 298; (1) the VH amino acid sequence of SEQ ID NO: 242, and/or the VL amino acid sequence of SEQ ID NO: 299; (m) VH amino acid sequence of SEQ ID NO: 242, and/or the VL amino acid sequence of SEQ ID NO: 300; (n) VH amino acid sequence of SEQ ID NO: 242, and/or the VL amino acid sequence of SEQ ID NO: 301; (o) VH amino acid sequence of SEQ ID NO: 243, and/or the VL amino acid sequence of SEQ ID NO: 298; (p) VH amino acid sequence of SEQ ID NO: 243, and/or the VL amino acid sequence of SEQ ID NO: 299; (q) VH amino acid sequence of SEQ ID NO: 243, and/or the VL amino acid sequence of SEQ ID NO: 300; (r) the VH amino acid sequence of SEQ ID NO: 243, and/or the VL amino acid sequence of SEQ ID NO: 301; (s) the VH amino acid sequence of SEQ ID NO: 244, and/or the VL amino acid sequence of SEQ ID NO: 302; (t) the VH amino acid sequence of SEQ ID NO: 245, and/or the VL amino acid sequence of SEQ ID NO: 302; (u) the VH amino acid sequence of SEQ ID NO: 246, and/or the VL amino acid sequence of SEQ ID NO: 302; (v) the VH amino acid sequence of SEQ ID NO: 247, and/or the VL amino acid sequence of SEQ ID NO: 302; (w) the VH amino acid sequence of SEQ ID NO: 248, and/or the VL amino acid sequence of SEQ ID NO: 303; (x) VH amino acid sequence of SEQ ID NO: 249, and/or the VL amino acid sequence of SEQ ID NO: 304; (y) the VH amino acid sequence of SEQ ID NO: 250, and/or the VL amino acid sequence of SEQ ID NO: 305; (z) the VH amino acid sequence of SEQ ID NO: 251, and/or the VL amino acid sequence of SEQ ID NO: 306; (aa) the VH amino acid sequence of SEQ ID NO: 252, and/or the VL amino acid sequence of SEQ ID NO: 305; (bb) the VH amino acid sequence of SEQ ID NO: 252, and/or the VL amino acid sequence of SEQ ID NO: 307; (cc) the VH amino acid sequence of SEQ ID NO: 253 and/or the VL amino acid sequence of SEQ ID NO: 308; (dd) VH amino acid sequence of SEQ ID NO: 254, and/or the VL amino acid sequence of SEQ ID NO: 309; (ee) the VH amino acid sequence of SEQ ID NO: 255, and/or the VL amino acid sequence of SEQ ID NO: 310; (ff) the VH amino acid sequence of SEQ ID NO: 255, and/or the VL amino acid sequence of SEQ ID NO: 311; (gg) the VH amino acid sequence of SEQ ID NO: 255, and/or the VL amino acid sequence of SEQ ID NO: 312; (hh) the VH amino acid sequence of SEQ ID NO: 256, and/or the VL amino acid sequence of SEQ ID NO: 310; (ii) the VH amino acid sequence of SEQ ID NO: 256, and/or the VL amino acid sequence of SEQ ID NO: 311; (jj) the VH amino acid sequence of SEQ ID NO: 256, and/or the VL amino acid sequence of SEQ ID NO: 312; (kk) the VH amino acid sequence of SEQ ID NO: 257, and/or the VL amino acid sequence of SEQ ID NO: 310; (11) the VH amino acid sequence of SEQ ID NO: 257, and/or the VL amino acid sequence of SEQ ID NO: 311; (mm) the VH amino acid sequence of SEQ ID NO: 257, and/or the VL amino acid sequence of SEQ ID NO: 312; (nn) the VH amino acid sequence of SEQ ID NO: 258, and/or the VL amino acid sequence of SEQ ID NO: 310; (00) the VH amino acid sequence of SEQ ID NO: 258, and/or the VL amino acid sequence of SEQ ID NO: 311; (pp) the VH amino acid sequence of SEQ ID NO: 258, and/or the VL amino acid sequence of SEQ ID NO: 312; (qq) the VH amino acid sequence of SEQ ID NO: 2, and/or the VL amino acid sequence of SEQ ID NO: 310; (rr) the VH amino acid sequence of SEQ ID NO: 2, and/or the VL amino acid sequence of SEQ ID NO: 311; (ss) the VH amino acid sequence of SEQ ID NO: 2, and/or the VL amino acid sequence of SEQ ID NO: 312; (tt) the VH amino acid sequence of SEQ ID NO: 260, and/or the VL amino acid sequence of SEQ ID NO: 317; (uu) the VH amino acid sequence of SEQ ID NO: 260, and/or the VL amino acid sequence of SEQ ID NO: 313; (vv) the VH amino acid sequence of SEQ ID NO: 261, and/or the VL amino acid sequence of SEQ ID NO: 314; (ww) the VH amino acid sequence of SEQ ID NO: 261, and/or the VL amino acid sequence of SEQ ID NO: 315; (xx) the VH amino acid sequence of SEQ ID NO: 261, and/or the VL amino acid sequence of SEQ ID NO: 316; (yy) the VH amino acid sequence of SEQ ID NO: 262, and/or the VL amino acid sequence of SEQ ID NO: 317; (zz) the VH amino acid sequence of SEQ ID NO: 261, and/or the VL amino acid sequence of SEQ ID NO: 317; (aaa) the VH amino acid sequence of SEQ ID NO: 263, and/or the VL amino acid sequence of SEQ ID NO: 317; (bbb) the VH amino acid sequence of SEQ ID NO: 264, and/or the VL amino acid sequence of SEQ ID NO: 317; (ccc) the VH amino acid sequence of SEQ ID NO: 262, and/or the VL amino acid sequence of SEQ ID NO: 318; (ddd) the VH amino acid sequence of SEQ ID NO: 263, and/or the VL amino acid sequence of SEQ ID NO: 318; (eee) the VH amino acid sequence of SEQ ID NO: 264, and/or the VL amino acid sequence of SEQ ID NO: 318; (fff) the VH amino acid sequence of SEQ ID NO: 260, and/or the VL amino acid sequence of SEQ ID NO: 318; (ggg) the VH amino acid sequence of SEQ ID NO: 264, and/or the VL amino acid sequence of SEQ ID NO: 314; (hhh) the VH amino acid sequence of SEQ ID NO: 264, and/or the VL amino acid sequence of SEQ ID NO: 316; (iii) the VH amino acid sequence of SEQ ID NO: 260, and/or the VL amino acid sequence of SEQ ID NO: 319; (jjj) the VH amino acid sequence of SEQ ID NO: 260, and/or the VL amino acid sequence of SEQ ID NO: 314; (kkk) the VH amino acid sequence of SEQ ID NO: 260, and/or the VL amino acid sequence of SEQ ID NO: 315; (111) the VH amino acid sequence of SEQ ID NO: 260, and/or the VL amino acid sequence of SEQ ID NO: 316; (mmm) the VH amino acid sequence of SEQ ID NO: 261, and/or the VL amino acid sequence of SEQ ID NO: 313; (nnn) the VH amino acid sequence of SEQ ID NO: 261, and/or the VL amino acid sequence of SEQ ID NO: 318; (000) the VH amino acid sequence of SEQ ID NO: 261, and/or the VL amino acid sequence of SEQ ID NO: 319; (ppp) the VH amino acid sequence of SEQ ID NO: 265, and/or the VL amino acid sequence of SEQ ID NO: 320; (qqq) the VH amino acid sequence of SEQ ID NO: 266, and/or the VL amino acid sequence of SEQ ID NO: 321; (rrr) the VH amino acid sequence of SEQ ID NO: 267, and/or the VL amino acid sequence of SEQ ID NO: 322; (sss) the VH amino acid sequence of SEQ ID NO: 268, and/or the VL amino acid sequence of SEQ ID NO: 323; (ttt) the VH amino acid sequence of SEQ ID NO: 269, and/or the VL amino acid sequence of SEQ ID NO: 324; (uuu) the VH amino acid sequence of SEQ ID NO: 270, and/or the VL amino acid sequence of SEQ ID NO: 325; (vvv) the VH amino acid sequence of SEQ ID NO: 271, and/or the VL amino acid sequence of SEQ ID NO: 326; (www) the VH amino acid sequence of SEQ ID NO: 272, and/or the VL amino acid sequence of SEQ ID NO: 331; (xxx) the VH amino acid sequence of SEQ ID NO: 272, and/or the VL amino acid sequence of SEQ ID NO: 327; (yyy) the VH amino acid sequence of SEQ ID NO: 273, and/or the VL amino acid sequence of SEQ ID NO: 328; (zzz) the VH amino acid sequence of SEQ ID NO: 273, and/or the VL amino acid sequence of SEQ ID NO: 329; (aaaa) the VH amino acid sequence of SEQ ID NO: 273, and/or the VL amino acid sequence of SEQ ID NO: 330; (bbbb) the VH amino acid sequence of SEQ ID NO: 273, and/or the VL amino acid sequence of SEQ ID NO: 331; (cccc) the VH amino acid sequence of SEQ ID NO: 274, and/or the VL amino acid sequence of SEQ ID NO: 331; (dddd) the VH amino acid sequence of SEQ ID NO: 275, and/or the VL amino acid sequence of SEQ ID NO: 331; (eeee) the VH amino acid sequence of SEQ ID NO: 276, and/or the VL amino acid sequence of SEQ ID NO: 331; (ffff) the VH amino acid sequence of SEQ ID NO: 277, and/or the VL amino acid sequence of SEQ ID NO: 331; (gggg) the VH amino acid sequence of SEQ ID NO: 272, and/or the VL amino acid sequence of SEQ ID NO: 332; (hhhh) the VH amino acid sequence of SEQ ID NO: 276, and/or the VL amino acid sequence of SEQ ID NO: 332; (iiii) the VH amino acid sequence of SEQ ID NO: 275, and/or the VL amino acid sequence of SEQ ID NO: 328; (jjjj) the VH amino acid sequence of SEQ ID NO: 275, and/or the VL amino acid sequence of SEQ ID NO: 330; (kkkk) the VH amino acid sequence of SEQ ID NO: 277, and/or the VL amino acid sequence of SEQ ID NO: 328; (1111) the VH amino acid sequence of SEQ ID NO: 277, and/or the VL amino acid sequence of SEQ ID NO: 330; (mmmm) the VH amino acid sequence of SEQ ID NO: 272, and/or the VL amino acid sequence of SEQ ID NO: 333; (nnnn) the VH amino acid sequence of SEQ ID NO: 272, and/or the VL amino acid sequence of SEQ ID NO: 328; (oooo) the VH amino acid sequence of SEQ ID NO: 272, and/or the VL amino acid sequence of SEQ ID NO: 329; (pppp) the VH amino acid sequence of SEQ ID NO: 272 and/or the VL amino acid sequence of SEQ ID NO: 330; (qqqq) the VH amino acid sequence of SEQ ID NO: 273, and/or the VL amino acid sequence of SEQ ID NO: 327; (rrrr) the VH amino acid sequence of SEQ ID NO: 273, and/or the VL amino acid sequence of SEQ ID NO: 332; (ssss) the VH amino acid sequence of SEQ ID NO: 273, and/or the VL amino acid sequence of SEQ ID NO: 333; (tttt) the VH amino acid sequence of SEQ ID NO: 278, and/or the VL amino acid sequence of SEQ ID NO: 334; (uuuu) the VH amino acid sequence of SEQ ID NO: 279, and/or the VL amino acid sequence of SEQ ID NO: 323; (vvvv) the VH amino acid sequence of SEQ ID NO: 280, and/or the VL amino acid sequence of SEQ ID NO: 339; (wwww) the VH amino acid sequence of SEQ ID NO: 280, and/or the VL amino acid sequence of SEQ ID NO: 335; (xxxx) the VH amino acid sequence of SEQ ID NO: 281, and/or the VL amino acid sequence of SEQ ID NO: 336; (yyyy) the VH amino acid sequence of SEQ ID NO: 281, and/or the VL amino acid sequence of SEQ ID NO: 337; (zzzz) the VH amino acid sequence of SEQ ID NO: 281, and/or the VL amino acid sequence of SEQ ID NO: 338; (aaaaa) the VH amino acid sequence of SEQ ID NO: 282, and/or the VL amino acid sequence of SEQ ID NO: 339; (bbbbb) the VH amino acid sequence of SEQ ID NO: 281, and/or the VL amino acid sequence of SEQ ID NO: 339; (ccccc) the VH amino acid sequence of SEQ ID NO: 283, and/or the VL amino acid sequence of SEQ ID NO: 339; (ddddd) the VH amino acid sequence of SEQ ID NO: 284, and/or the VL amino acid sequence of SEQ ID NO: 339; (eeeee) the VH amino acid sequence of SEQ ID NO: 285, and/or the VL amino acid sequence of SEQ ID NO: 339; (fffff) the VH amino acid sequence of SEQ ID NO: 286, and/or the VL amino acid sequence of SEQ ID NO: 339; (ggggg) the VH amino acid sequence of SEQ ID NO: 282, and/or the VL amino acid sequence of SEQ ID NO: 340; (hhhhh) the VH amino acid sequence of SEQ ID NO: 280, and/or the VL amino acid sequence of SEQ ID NO: 340; (iiiii) the VH amino acid sequence of SEQ ID NO: 283, and/or the VL amino acid sequence of SEQ ID NO: 340; (jjjjj) the VH amino acid sequence of SEQ ID NO: 284, and/or the VL amino acid sequence of SEQ ID NO: 340; (kkkkk) the VH amino acid sequence of SEQ ID NO: 285, and/or the VL amino acid sequence of SEQ ID NO: 340; (lllll) the VH amino acid sequence of SEQ ID NO: 286, and/or the VL amino acid sequence of SEQ ID NO: 340; (mmmmm) the VH amino acid sequence of SEQ ID NO: 284, and/or the VL amino acid sequence of SEQ ID NO: 336; (nnnnn) the VH amino acid sequence of SEQ ID NO: 284, and/or the VL amino acid sequence of SEQ ID NO: 338; (ooooo) the VH amino acid sequence of SEQ ID NO: 286, and/or the VL amino acid sequence of SEQ ID NO: 336; (ppppp) the VH amino acid sequence of SEQ ID NO: 286, and/or the VL amino acid sequence of SEQ ID NO: 338; (qqqqq) the VH amino acid sequence of SEQ ID NO: 287, and/or the VL amino acid sequence of SEQ ID NO: 340; (rrrrr) the VH amino acid sequence of SEQ ID NO: 288, and/or the VL amino acid sequence of SEQ ID NO: 340; (sssss) the VH amino acid sequence of SEQ ID NO: 280, and/or the VL amino acid sequence of SEQ ID NO: 341; (ttttt) the VH amino acid sequence of SEQ ID NO: 286, and/or the VL amino acid sequence of SEQ ID NO: 342; (uuuuu) the VH amino acid sequence of SEQ ID NO: 286, and/or the VL amino acid sequence of SEQ ID NO: 343; (vvvvv) the VH amino acid sequence of SEQ ID NO: 287, and/or the VL amino acid sequence of SEQ ID NO: 342; (wwwww) the VH amino acid sequence of SEQ ID NO: 288, and/or the VL amino acid sequence of SEQ ID NO: 343; (xxxxx) the VH amino acid sequence of SEQ ID NO: 287, and/or the VL amino acid sequence of SEQ ID NO: 343; (yyyyy) the VH amino acid sequence of SEQ ID NO: 288, and/or the VL amino acid sequence of SEQ ID NO: 342; (zzzzz) the VH amino acid sequence of SEQ ID NO: 289, and/or the VL amino acid sequence of SEQ ID NO: 342; (aaaaaa) the VH amino acid sequence of SEQ ID NO: 289, and/or the VL amino acid sequence of SEQ ID NO: 343; (bbbbbb) the VH amino acid sequence of SEQ ID NO: 287, and/or the VL amino acid sequence of SEQ ID NO: 344; (cccccc) the VH amino acid sequence of SEQ ID NO: 288, and/or the VL amino acid sequence of SEQ ID NO: 344; (dddddd) the VH amino acid sequence of SEQ ID NO: 280, and/or the VL amino acid sequence of SEQ ID NO: 336: (eeeeee) the VH amino acid sequence of SEQ ID NO: 289, and/or the VL amino acid sequence of SEQ ID NO: 344; (ffffff) the VH amino acid sequence of SEQ ID NO: 289, and/or the VL amino acid sequence of SEQ ID NO: 340; (gggggg) the VH amino acid sequence of SEQ ID NO: 286, and/or the VL amino acid sequence of SEQ ID NO: 344; (hhhhhh) the VH amino acid sequence of SEQ ID NO: 290, and/or the VL amino acid sequence of SEQ ID NO: 336; (irmi) the VH amino acid sequence of SEQ ID NO: 291, and/or the VL amino acid sequence of SEQ ID NO: 336: (jjjjjj) the VH amino acid sequence of SEQ ID NO: 292, and/or the VL amino acid sequence of SEQ ID NO: 336; (kkkkkk) the VH amino acid sequence of SEQ ID NO: 290, and/or the VL amino acid sequence of SEQ ID NO: 345; (llllll) the VH amino acid sequence of SEQ ID NO: 291, and/or the VL amino acid sequence of SEQ ID NO: 345; (mmmmmm) the VH amino acid sequence of SEQ ID NO: 292, and/or the VL amino acid sequence of SEQ ID NO: 345; (nnnnnn) the VH amino acid sequence of SEQ ID NO: 290, and/or the VL amino acid sequence of SEQ ID NO: 346; (oooooo) the VH amino acid sequence of SEQ ID NO: 280, and/or the VL amino acid sequence of SEQ ID NO: 337; (pppppp) the VH amino acid sequence of SEQ ID NO: 291, and/or the VL amino acid sequence of SEQ ID NO: 346; (qqqqqq) the VH amino acid sequence of SEQ ID NO: 292, and/or the VL amino acid sequence of SEQ ID NO: 346; (rrrrrr) the VH amino acid sequence of SEQ ID NO: 290, and/or the VL amino acid sequence of SEQ ID NO: 347; (ssssss) the VH amino acid sequence of SEQ ID NO: 291, and/or the VL amino acid sequence of SEQ ID NO: 347; (tttttt) the VH amino acid sequence of SEQ ID NO: 292, and/or the VL amino acid sequence of SEQ ID NO: 347; (uuuuuu) the VH amino acid sequence of SEQ ID NO: 293, and/or the VL amino acid sequence of SEQ ID NO: 340; (vvVVVV) the VH amino acid sequence of SEQ ID NO: 294, and/or the VL amino acid sequence of SEQ ID NO: 340; (wwwwww) the VH amino acid sequence of SEQ ID NO: 295, and/or the VL amino acid sequence of SEQ ID NO: 340; (xxxxxx) the VH amino acid sequence of SEQ ID NO: 293, and/or the VL amino acid sequence of SEQ ID NO: 343; (yyyyyy) the VH amino acid sequence of SEQ ID NO: 294, and/or the VL amino acid sequence of SEQ ID NO: 343; (zzzzzz) the VH amino acid sequence of SEQ ID NO: 280, and/or the VL amino acid sequence of SEQ ID NO: 338; (aaaaaaa) the VH amino acid sequence of SEQ ID NO: 295, and/or the VL amino acid sequence of SEQ ID NO: 343; (bbbbbbb) the VH amino acid sequence of SEQ ID NO: 293, and/or the VL amino acid sequence of SEQ ID NO: 342; (ccccccc) the VH amino acid sequence of SEQ ID NO: 294, and/or the VL amino acid sequence of SEQ ID NO: 342; (ddddddd) the VH amino acid sequence of SEQ ID NO: 295 and/or the VL amino acid sequence of SEQ ID NO: 342; (eeeeeee) the VH amino acid sequence of SEQ ID NO: 293, and/or the VL amino acid sequence of SEQ ID NO: 344; (fffffff) the VH amino acid sequence of SEQ ID NO: 294, and/or the VL amino acid sequence of SEQ ID NO: 344; (ggggggg) the VH amino acid sequence of SEQ ID NO: 295, and/or the VL amino acid sequence of SEQ ID NO: 344; (hhhhhhh) the VH amino acid sequence of SEQ ID NO: 281, and/or the VL amino acid sequence of SEQ ID NO: 345; (irmii) the VH amino acid sequence of SEQ ID NO: 281, and/or the VL amino acid sequence of SEQ ID NO: 346; (jjjjjjj) the VH amino acid sequence of SEQ ID NO: 281, and/or the VL amino acid sequence of SEQ ID NO: 347; (kkkkkkk) the VH amino acid sequence of SEQ ID NO: 281, and/or the VL amino acid sequence of SEQ ID NO: 335; (lllllll) the VH amino acid sequence of SEQ ID NO: 283, and/or the VL amino acid sequence of SEQ ID NO: 343; (mmmmmmm) the VH amino acid sequence of SEQ ID NO: 283, and/or the VL amino acid sequence of SEQ ID NO: 342; (nnnnnnn) the VH amino acid sequence of SEQ ID NO: 283, and/or the VL amino acid sequence of SEQ ID NO: 344; (ooooooo) the VH amino acid sequence of SEQ ID NO: 281, and/or the VL amino acid sequence of SEQ ID NO: 340; (ppppppp) the VH amino acid sequence of SEQ ID NO: 281, and/or the VL amino acid sequence of SEQ ID NO: 341, (qqqqqqq) the VH amino acid sequence of SEQ ID NO: 461, and/or the VL amino acid sequence of SEQ ID NO: 462, or (rrrrrrr) the VH amino acid sequence of SEQ ID NO: 459, and/or the VL amino acid sequence of SEQ ID NO: 296.

In another aspect, the invention relates to an antibody or antigen-binding fragment thereof that comprises (a) a heavy chain comprising an amino acid sequence at least 90%, 95%, 99%, or 100% identical to any one of SEQ ID NOs: 348-403, 460 and 463; and/or (b) a light chain comprising an amino acid sequence at least 90%, 95%, 99%, or 100% identical to any one of SEQ ID NOs: 404-455 and 464. In one aspect, the antibody or antigen-binding fragment thereof comprises an amino acid sequence at least 90%, 95%, 99%, or 100% identical to: (a) the heavy chain amino acid sequence of SEQ ID NO: 348, and/or the light chain amino acid sequence of SEQ ID NO: 404; (b) the heavy chain amino acid sequence of SEQ ID NO: 349, and/or the light chain amino acid sequence of SEQ ID NO: 404; (c) the heavy chain amino acid sequence of SEQ ID NO: 350, and/or the light chain amino acid sequence of SEQ ID NO: 404; (d) the heavy chain amino acid sequence of SEQ ID NO: 348, and/or the light chain amino acid sequence of SEQ ID NO: 405; (e) the heavy chain amino acid sequence of SEQ ID NO: 349, and/or the light chain amino acid sequence of SEQ ID NO: 405; (f) the heavy chain amino acid sequence of SEQ ID NO: 350, and/or the light chain amino acid sequence of SEQ ID NO: 405; (g) the heavy chain amino acid sequence of SEQ ID NO: 349, and/or the light chain amino acid sequence of SEQ ID NO: 406; (h) the heavy chain amino acid sequence of SEQ ID NO: 349, and/or the light chain amino acid sequence of SEQ ID NO: 407; (i) the heavy chain amino acid sequence of SEQ ID NO: 349, and/or the light chain amino acid sequence of SEQ ID NO: 408; (j) the heavy chain amino acid sequence of SEQ ID NO: 349, and/or the light chain amino acid sequence of SEQ ID NO: 409; (k) the heavy chain amino acid sequence of SEQ ID NO: 350, and/or the light chain amino acid sequence of SEQ ID NO: 406; (1) the heavy chain amino acid sequence of SEQ ID NO: 350, and/or the light chain amino acid sequence of SEQ ID NO: 407; (m) the heavy chain amino acid sequence of SEQ ID NO: 350, and/or the light chain amino acid sequence of SEQ ID NO: 408; (n) the heavy chain amino acid sequence of SEQ ID NO: 350, and/or the light chain amino acid sequence of SEQ ID NO: 409; (o) the heavy chain amino acid sequence of SEQ ID NO: 351, and/or the light chain amino acid sequence of SEQ ID NO: 406; (p) the heavy chain amino acid sequence of SEQ ID NO: 351, and/or the light chain amino acid sequence of SEQ ID NO: 407; (q) the heavy chain amino acid sequence of SEQ ID NO: 351, and/or the light chain amino acid sequence of SEQ ID NO: 408; (r) the heavy chain amino acid sequence of SEQ ID NO: 351, and/or the light chain amino acid sequence of SEQ ID NO: 409; (s) the heavy chain amino acid sequence of SEQ ID NO: 352, and/or the light chain amino acid sequence of SEQ ID NO: 410; (t) the heavy chain amino acid sequence of SEQ ID NO: 353, and/or the light chain amino acid sequence of SEQ ID NO: 410; (u) the heavy chain amino acid sequence of SEQ ID NO: 354, and/or the light chain amino acid sequence of SEQ ID NO: 410; (v) the heavy chain amino acid sequence of SEQ ID NO: 355, and/or the light chain amino acid sequence of SEQ ID NO: 410; (w) the heavy chain amino acid sequence of SEQ ID NO: 356, and/or the light chain amino acid sequence of SEQ ID NO: 411; (x) the heavy chain amino acid sequence of SEQ ID NO: 357, and/or the light chain amino acid sequence of SEQ ID NO: 412; (y) the heavy chain amino acid sequence of SEQ ID NO: 358, and/or the light chain amino acid sequence of SEQ ID NO: 413; (z) the heavy chain amino acid sequence of SEQ ID NO: 359, and/or the light chain amino acid sequence of SEQ ID NO: 414; (aa) the heavy chain amino acid sequence of SEQ ID NO: 360, and/or the light chain amino acid sequence of SEQ ID NO: 413; (bb) the heavy chain amino acid sequence of SEQ ID NO: 360, and/or the light chain amino acid sequence of SEQ ID NO: 415; (cc) the heavy chain amino acid sequence of SEQ ID NO: 361, and/or the light chain amino acid sequence of SEQ ID NO: 416: (dd) the heavy chain amino acid sequence of SEQ ID NO: 362, and/or the light chain amino acid sequence of SEQ ID NO: 417; (ee) the heavy chain amino acid sequence of SEQ ID NO: 363, and/or the light chain amino acid sequence of SEQ ID NO: 418; (ff) the heavy chain amino acid sequence of SEQ ID NO: 363, and/or the light chain amino acid sequence of SEQ ID NO: 419; (gg) the heavy chain amino acid sequence of SEQ ID NO: 363, and/or the light chain amino acid sequence of SEQ ID NO: 420; (hh) the heavy chain amino acid sequence of SEQ ID NO: 364, and/or the light chain amino acid sequence of SEQ ID NO: 418; (ii) the heavy chain amino acid sequence of SEQ ID NO: 364, and/or the light chain amino acid sequence of SEQ ID NO: 419; (jj) the heavy chain amino acid sequence of SEQ ID NO: 364, and/or the light chain amino acid sequence of SEQ ID NO: 420; (kk) the heavy chain amino acid sequence of SEQ ID NO: 365, and/or the light chain amino acid sequence of SEQ ID NO: 418; (11) the heavy chain amino acid sequence of SEQ ID NO: 365, and/or the light chain amino acid sequence of SEQ ID NO: 419; (mm) the heavy chain amino acid sequence of SEQ ID NO: 365, and/or the light chain amino acid sequence of SEQ ID NO: 420; (nn) the heavy chain amino acid sequence of SEQ ID NO: 366, and/or the light chain amino acid sequence of SEQ ID NO: 418; (00) the heavy chain amino acid sequence of SEQ ID NO: 366, and/or the light chain amino acid sequence of SEQ ID NO: 419; (pp) the heavy chain amino acid sequence of SEQ ID NO: 366, and/or the light chain amino acid sequence of SEQ ID NO: 420; (qq) the heavy chain amino acid sequence of SEQ ID NO: 367, and/or the light chain amino acid sequence of SEQ ID NO: 418; (rr) the heavy chain amino acid sequence of SEQ ID NO: 367, and/or the light chain amino acid sequence of SEQ ID NO: 419; (ss) the heavy chain amino acid sequence of SEQ ID NO: 367, and/or the light chain amino acid sequence of SEQ ID NO: 420; (tt) the heavy chain amino acid sequence of SEQ ID NO: 368, and/or the light chain amino acid sequence of SEQ ID NO: 425; (uu) the heavy chain amino acid sequence of SEQ ID NO: 368, and/or the light chain amino acid sequence of SEQ ID NO: 421; (vv) the heavy chain amino acid sequence of SEQ ID NO: 369, and/or the light chain amino acid sequence of SEQ ID NO: 422; (ww) the heavy chain amino acid sequence of SEQ ID NO: 369, and/or the light chain amino acid sequence of SEQ ID NO: 423; (xx) the heavy chain amino acid sequence of SEQ ID NO: 369, and/or the light chain amino acid sequence of SEQ ID NO: 424; (yy) the heavy chain amino acid sequence of SEQ ID NO: 370, and/or the light chain amino acid sequence of SEQ ID NO: 425; (zz) the heavy chain amino acid sequence of SEQ ID NO: 369, and/or the light chain amino acid sequence of SEQ ID NO: 425; (aaa) the heavy chain amino acid sequence of SEQ ID NO: 371, and/or the light chain amino acid sequence of SEQ ID NO: 425; (bbb) the heavy chain amino acid sequence of SEQ ID NO: 372, and/or the light chain amino acid sequence of SEQ ID NO: 425; (ccc) the heavy chain amino acid sequence of SEQ ID NO: 370, and/or the light chain amino acid sequence of SEQ ID NO: 426; (ddd) the heavy chain amino acid sequence of SEQ ID NO: 371, and/or the light chain amino acid sequence of SEQ ID NO: 426; (eee) the heavy chain amino acid sequence of SEQ ID NO: 372, and/or the light chain amino acid sequence of SEQ ID NO: 426; (fff) the heavy chain amino acid sequence of SEQ ID NO: 368, and/or the light chain amino acid sequence of SEQ ID NO: 426; (ggg) the heavy chain amino acid sequence of SEQ ID NO: 372, and/or the light chain amino acid sequence of SEQ ID NO: 422; (hhh) the heavy chain amino acid sequence of SEQ ID NO: 372, and/or the light chain amino acid sequence of SEQ ID NO: 424; (iii) the heavy chain amino acid sequence of SEQ ID NO: 368, and/or the light chain amino acid sequence of SEQ ID NO: 427; (jjj) the heavy chain amino acid sequence of SEQ ID NO: 368, and/or the light chain amino acid sequence of SEQ ID NO: 422; (kkk) the heavy chain amino acid sequence of SEQ ID NO: 368, and/or the light chain amino acid sequence of SEQ ID NO: 423; (111) the heavy chain amino acid sequence of SEQ ID NO: 368, and/or the light chain amino acid sequence of SEQ ID NO: 424; (mmm) the heavy chain amino acid sequence of SEQ ID NO: 369, and/or the light chain amino acid sequence of SEQ ID NO: 421; (nnn) the heavy chain amino acid sequence of SEQ ID NO: 369, and/or the light chain amino acid sequence of SEQ ID NO: 426; (000) the heavy chain amino acid sequence of SEQ ID NO: 369, and/or the light chain amino acid sequence of SEQ ID NO: 427; (ppp) the heavy chain amino acid sequence of SEQ ID NO: 373, and/or the light chain amino acid sequence of SEQ ID NO: 428; (qqq) the heavy chain amino acid sequence of SEQ ID NO: 374, and/or the light chain amino acid sequence of SEQ ID NO: 429; (rrr) the heavy chain amino acid sequence of SEQ ID NO: 375, and/or the light chain amino acid sequence of SEQ ID NO: 430; (sss) the heavy chain amino acid sequence of SEQ ID NO: 376, and/or the light chain amino acid sequence of SEQ ID NO: 431; (ttt) the heavy chain amino acid sequence of SEQ ID NO: 377, and/or the light chain amino acid sequence of SEQ ID NO: 432; (uuu) the heavy chain amino acid sequence of SEQ ID NO: 378, and/or the light chain amino acid sequence of SEQ ID NO: 433; (vvv) the heavy chain amino acid sequence of SEQ ID NO: 379, and/or the light chain amino acid sequence of SEQ ID NO: 434; (www) the heavy chain amino acid sequence of SEQ ID NO: 380, and/or the light chain amino acid sequence of SEQ ID NO: 439; (xxx) the heavy chain amino acid sequence of SEQ ID NO: 380, and/or the light chain amino acid sequence of SEQ ID NO: 435; (yyy) the heavy chain amino acid sequence of SEQ ID NO: 381, and/or the light chain amino acid sequence of SEQ ID NO: 436; (zzz) the heavy chain amino acid sequence of SEQ ID NO: 381, and/or the light chain amino acid sequence of SEQ ID NO: 437; (aaaa) the heavy chain amino acid sequence of SEQ ID NO: 381, and/or the light chain amino acid sequence of SEQ ID NO: 438; (bbbb) the heavy chain amino acid sequence of SEQ ID NO: 381, and/or the light chain amino acid sequence of SEQ ID NO: 439; (cccc) the heavy chain amino acid sequence of SEQ ID NO: 382, and/or the light chain amino acid sequence of SEQ ID NO: 439; (dddd) the heavy chain amino acid sequence of SEQ ID NO: 383, and/or the light chain amino acid sequence of SEQ ID NO: 439; (eeee) the heavy chain amino acid sequence of SEQ ID NO: 384, and/or the light chain amino acid sequence of SEQ ID NO: 439; (ffff) the heavy chain amino acid sequence of SEQ ID NO: 385, and/or the light chain amino acid sequence of SEQ ID NO: 439; (gggg) the heavy chain amino acid sequence of SEQ ID NO: 380, and/or the light chain amino acid sequence of SEQ ID NO: 440; (hhhh) the heavy chain amino acid sequence of SEQ ID NO: 384, and/or the light chain amino acid sequence of SEQ ID NO: 440; (iiii) the heavy chain amino acid sequence of SEQ ID NO: 383, and/or the light chain amino acid sequence of SEQ ID NO: 436; (jjjj) the heavy chain amino acid sequence of SEQ ID NO: 383, and/or the light chain amino acid sequence of SEQ ID NO: 438; (kkkk) the heavy chain amino acid sequence of SEQ ID NO: 385, and/or the light chain amino acid sequence of SEQ ID NO: 436; (III) the heavy chain amino acid sequence of SEQ ID NO: 385, and/or the light chain amino acid sequence of SEQ ID NO: 438; (mmmm) the heavy chain amino acid sequence of SEQ ID NO: 380, and/or the light chain amino acid sequence of SEQ ID NO: 441; (nnnn) the heavy chain amino acid sequence of SEQ ID NO: 380, and/or the light chain amino acid sequence of SEQ ID NO: 436; (0000) the heavy chain amino acid sequence of SEQ ID NO: 380, and/or the light chain amino acid sequence of SEQ ID NO: 437; (pppp) the heavy chain amino acid sequence of SEQ ID NO: 380, and/or the light chain amino acid sequence of SEQ ID NO: 438; (qqqq) the heavy chain amino acid sequence of SEQ ID NO: 381, and/or the light chain amino acid sequence of SEQ ID NO: 435; (rrrr) the heavy chain amino acid sequence of SEQ ID NO: 381, and/or the light chain amino acid sequence of SEQ ID NO: 440; (ssss) the heavy chain amino acid sequence of SEQ ID NO: 381, and/or the light chain amino acid sequence of SEQ ID NO: 441; (tttt) the heavy chain amino acid sequence of SEQ ID NO: 386, and/or the light chain amino acid sequence of SEQ ID NO: 442; (uuuu) the heavy chain amino acid sequence of SEQ ID NO: 387, and/or the light chain amino acid sequence of SEQ ID NO: 431; (vvvv) the heavy chain amino acid sequence of SEQ ID NO: 388, and/or the light chain amino acid sequence of SEQ ID NO: 447; (wwww) the heavy chain amino acid sequence of SEQ ID NO: 388, and/or the light chain amino acid sequence of SEQ ID NO: 443; (xxxx) the heavy chain amino acid sequence of SEQ ID NO: 389, and/or the light chain amino acid sequence of SEQ ID NO: 444; (yyyy) the heavy chain amino acid sequence of SEQ ID NO: 389, and/or the light chain amino acid sequence of SEQ ID NO: 445; (zzzz) the heavy chain amino acid sequence of SEQ ID NO: 389, and/or the light chain amino acid sequence of SEQ ID NO: 446; (aaaaa) the heavy chain amino acid sequence of SEQ ID NO: 390, and/or the light chain amino acid sequence of SEQ ID NO: 447; (bbbbb) the heavy chain amino acid sequence of SEQ ID NO: 389, and/or the light chain amino acid sequence of SEQ ID NO: 447; (ccccc) the heavy chain amino acid sequence of SEQ ID NO: 391, and/or the light chain amino acid sequence of SEQ ID NO: 447; (ddddd) the heavy chain amino acid sequence of SEQ ID NO: 392, and/or the light chain amino acid sequence of SEQ ID NO: 447; (eeeee) the heavy chain amino acid sequence of SEQ ID NO: 393, and/or the light chain amino acid sequence of SEQ ID NO: 447; (fffff) the heavy chain amino acid sequence of SEQ ID NO: 394, and/or the light chain amino acid sequence of SEQ ID NO: 447; (ggggg) the heavy chain amino acid sequence of SEQ ID NO: 390, and/or the light chain amino acid sequence of SEQ ID NO: 448; (hhhhh) the heavy chain amino acid sequence of SEQ ID NO: 388, and/or the light chain amino acid sequence of SEQ ID NO: 448; (iiiii) the heavy chain amino acid sequence of SEQ ID NO: 391, and/or the light chain amino acid sequence of SEQ ID NO: 448; (jjjjj) the heavy chain amino acid sequence of SEQ ID NO: 392, and/or the light chain amino acid sequence of SEQ ID NO: 448; (kkkkk) the heavy chain amino acid sequence of SEQ ID NO: 393, and/or the light chain amino acid sequence of SEQ ID NO: 448; (lllll) the heavy chain amino acid sequence of SEQ ID NO: 394, and/or the light chain amino acid sequence of SEQ ID NO: 448; (mmmmm) the heavy chain amino acid sequence of SEQ ID NO: 392, and/or the light chain amino acid sequence of SEQ ID NO: 444; (nnnnn) the heavy chain amino acid sequence of SEQ ID NO: 392, and/or the light chain amino acid sequence of SEQ ID NO: 446; (00000) the heavy chain amino acid sequence of SEQ ID NO: 394, and/or the light chain amino acid sequence of SEQ ID NO: 444; (ppppp) the heavy chain amino acid sequence of SEQ ID NO: 394, and/or the light chain amino acid sequence of SEQ ID NO: 446; (qqqqq) the heavy chain amino acid sequence of SEQ ID NO: 395, and/or the light chain amino acid sequence of SEQ ID NO: 448; (rrrrr) the heavy chain amino acid sequence of SEQ ID NO: 396, and/or the light chain amino acid sequence of SEQ ID NO: 448; (sssss) the heavy chain amino acid sequence of SEQ ID NO: 388, and/or the light chain amino acid sequence of SEQ ID NO: 449; (ttttt) the heavy chain amino acid sequence of SEQ ID NO: 394, and/or the light chain amino acid sequence of SEQ ID NO: 450; (uuuuu) the heavy chain amino acid sequence of SEQ ID NO: 394, and/or the light chain amino acid sequence of SEQ ID NO: 451; (vvvvv) the heavy chain amino acid sequence of SEQ ID NO: 395, and/or the light chain amino acid sequence of SEQ ID NO: 450; (wwwww) the heavy chain amino acid sequence of SEQ ID NO: 396, and/or the light chain amino acid sequence of SEQ ID NO: 451; (xxxxx) the heavy chain amino acid sequence of SEQ ID NO: 395, and/or the light chain amino acid sequence of SEQ ID NO: 451; (yyyyy) the heavy chain amino acid sequence of SEQ ID NO: 396, and/or the light chain amino acid sequence of SEQ ID NO: 450; (zzzzz) the heavy chain amino acid sequence of SEQ ID NO: 397, and/or the light chain amino acid sequence of SEQ ID NO: 450; (aaaaaa) the heavy chain amino acid sequence of SEQ ID NO: 397, and/or the light chain amino acid sequence of SEQ ID NO: 451; (bbbbbb) the heavy chain amino acid sequence of SEQ ID NO: 395, and/or the light chain amino acid sequence of SEQ ID NO: 452; (cccccc) the heavy chain amino acid sequence of SEQ ID NO: 396, and/or the light chain amino acid sequence of SEQ ID NO: 452; (dddddd) the heavy chain amino acid sequence of SEQ ID NO: 388, and/or the light chain amino acid sequence of SEQ ID NO: 444; (eeeeee) the heavy chain amino acid sequence of SEQ ID NO: 397, and/or the light chain amino acid sequence of SEQ ID NO: 452; (ffffff) the heavy chain amino acid sequence of SEQ ID NO: 397, and/or the light chain amino acid sequence of SEQ ID NO: 448; (gggggg) the heavy chain amino acid sequence of SEQ ID NO: 394, and/or the light chain amino acid sequence of SEQ ID NO: 452; (hhhhhh) the heavy chain amino acid sequence of SEQ ID NO: 398, and/or the light chain amino acid sequence of SEQ ID NO: 444; (iiiiii) the heavy chain amino acid sequence of SEQ ID NO: 399, and/or the light chain amino acid sequence of SEQ ID NO: 444; (jjjjjj) the heavy chain amino acid sequence of SEQ ID NO: 400, and/or the light chain amino acid sequence of SEQ ID NO: 444; (kkkkkk) the heavy chain amino acid sequence of SEQ ID NO: 398, and/or the light chain amino acid sequence of SEQ ID NO: 453; (llllll) the heavy chain amino acid sequence of SEQ ID NO: 399, and/or the light chain amino acid sequence of SEQ ID NO: 453; (mmmmmm) the heavy chain amino acid sequence of SEQ ID NO: 400, and/or the light chain amino acid sequence of SEQ ID NO: 453; (nnnnnn) the heavy chain amino acid sequence of SEQ ID NO: 398, and/or the light chain amino acid sequence of SEQ ID NO: 454; (oooooo) the heavy chain amino acid sequence of SEQ ID NO: 388, and/or the light chain amino acid sequence of SEQ ID NO: 445; (pppppp) the heavy chain amino acid sequence of SEQ ID NO: 399, and/or the light chain amino acid sequence of SEQ ID NO: 454; (qqqqqq) the heavy chain amino acid sequence of SEQ ID NO: 400, and/or the light chain amino acid sequence of SEQ ID NO: 454; (rrrrrr) the heavy chain amino acid sequence of SEQ ID NO: 398, and/or the light chain amino acid sequence of SEQ ID NO: 455; (ssssss) the heavy chain amino acid sequence of SEQ ID NO: 399, and/or the light chain amino acid sequence of SEQ ID NO: 455; (tttttt) the heavy chain amino acid sequence of SEQ ID NO: 400, and/or the light chain amino acid sequence of SEQ ID NO: 455; (uuuuuu) the heavy chain amino acid sequence of SEQ ID NO: 401, and/or the light chain amino acid sequence of SEQ ID NO: 448; (vvvvvv) the heavy chain amino acid sequence of SEQ ID NO: 402, and/or the light chain amino acid sequence of SEQ ID NO: 448; (wwwwww) the heavy chain amino acid sequence of SEQ ID NO: 403, and/or the light chain amino acid sequence of SEQ ID NO: 448; (xxxxxx) the heavy chain amino acid sequence of SEQ ID NO: 401, and/or the light chain amino acid sequence of SEQ ID NO: 451; (yyyyyy) the heavy chain amino acid sequence of SEQ ID NO: 402, and/or the light chain amino acid sequence of SEQ ID NO: 451; (zzzzzz) the heavy chain amino acid sequence of SEQ ID NO: 388, and/or the light chain amino acid sequence of SEQ ID NO: 446; (aaaaaaa) the heavy chain amino acid sequence of SEQ ID NO: 403, and/or the light chain amino acid sequence of SEQ ID NO: 451; (bbbbbbb) the heavy chain amino acid sequence of SEQ ID NO: 401, and/or the light chain amino acid sequence of SEQ ID NO: 450; (ccccccc) the heavy chain amino acid sequence of SEQ ID NO: 402, and/or the light chain amino acid sequence of SEQ ID NO: 450; (ddddddd) the heavy chain amino acid sequence of SEQ ID NO: 403, and/or the light chain amino acid sequence of SEQ ID NO: 450; (eeeeeee) the heavy chain amino acid sequence of SEQ ID NO: 401, and/or the light chain amino acid sequence of SEQ ID NO: 452; (fffffff) the heavy chain amino acid sequence of SEQ ID NO: 402, and/or the light chain amino acid sequence of SEQ ID NO: 452; (ggggggg) the heavy chain amino acid sequence of SEQ ID NO: 403, and/or the light chain amino acid sequence of SEQ ID NO: 452; (hhhhhhh) the heavy chain amino acid sequence of SEQ ID NO: 389, and/or the light chain amino acid sequence of SEQ ID NO: 453; (iiiiiii) the heavy chain amino acid sequence of SEQ ID NO: 389, and/or the light chain amino acid sequence of SEQ ID NO: 454; (jjjjjjj) the heavy chain amino acid sequence of SEQ ID NO: 389, and/or the light chain amino acid sequence of SEQ ID NO: 455; (kkkkkkk) the heavy chain amino acid sequence of SEQ ID NO: 389, and/or the light chain amino acid sequence of SEQ ID NO: 443; (lllllll) the heavy chain amino acid sequence of SEQ ID NO: 391, and/or the light chain amino acid sequence of SEQ ID NO: 451; (mmmmmmm) the heavy chain amino acid sequence of SEQ ID NO: 391, and/or the light chain amino acid sequence of SEQ ID NO: 450; (nnnnnnn) the heavy chain amino acid sequence of SEQ ID NO: 391, and/or the light chain amino acid sequence of SEQ ID NO: 452; (ooooooo) the heavy chain amino acid sequence of SEQ ID NO: 389, and/or the light chain amino acid sequence of SEQ ID NO: 448; (ppppppp) the heavy chain amino acid sequence of SEQ ID NO: 389, and/or the light chain amino acid sequence of SEQ ID NO: 449, (qqqqqqq) the heavy chain amino acid sequence of SEQ ID NO: 463, and/or the light chain amino acid sequence of SEQ ID NO: 464, or (rrrrrrr) the heavy chain amino acid sequence of SEQ ID NO: 460, and/or the light chain amino acid sequence of SEQ ID NO: 404.

In one aspect, the antigen-binding fragment is a Fab, scFab, Fab′, F(ab′)2, Fv, scFv, diabody, or triabody.

In another aspect, the disclosure provides a pharmaceutical composition, comprising an antibody or antigen-binding fragment thereof disclosed herein and a pharmaceutically acceptable carrier.

The disclosure is also directed to a method of treating congenital adrenal hyperplasia (CAH) in a subject in need thereof, comprising administering to the subject an antibody or antigen-binding fragment thereof that specifically binds to CRH. In one aspect, the disclosure provides a method of treating CAH in a subject in need thereof, comprising administering to the subject an antibody or antigen-binding fragment thereof disclosed herein.

The disclosure is also directed to a method of treating CAH in a subject in need thereof, comprising administering to the subject (i) a mineralocorticoid and/or a glucocorticoid, and (ii) an antibody or antigen-binding fragment thereof that specifically binds to CRH. The disclosure is also directed to a method of treating CAH in a subject in need thereof, comprising administering to the subject (i) a mineralocorticoid and/or a glucocorticoid, and (ii) an antibody or antigen-binding fragment thereof disclosed herein.

The disclosure is also directed to a method of reducing androgen concentration in a subject in need thereof, comprising administering to the subject an antibody or antigen-binding fragment disclosed herein.

The disclosure is also directed to a method of reducing androgen concentration in a subject having CAH, comprising administering to the subject (i) a mineralocorticoid and/or a glucocorticoid, and (ii) an antibody or antigen-binding fragment thereof that specifically binds to CRH. The disclosure is also directed to a method of reducing androgen concentration in a subject having CAH, comprising administering to the subject an antibody or antigen-binding fragment disclosed herein.

The disclosure is also directed to a method of reducing androgen concentration in a subject having CAH, comprising administering to the subject (i) a mineralocorticoid and/or a glucocorticoid, and (ii) an antibody or antigen-binding fragment thereof disclosed herein.

In one aspect, the androgen is testosterone, free testosterone, androstenedione, an 11-oxygenated androgen, or a combination thereof. In another aspect, the 11-oxygenated androgen is 11-hydroxyandrostenedione (11OHA4), 11-hydroxytestosterone (11OHT), 11-ketoandrostenedione (11KA4), 11-ketotestosterone (11KT), or a combination thereof.

The disclosure is also directed to a method for reducing the severity of one or more symptoms or signs in a subject having CAH, comprising administering to the subject an antibody or antigen-binding fragment thereof that specifically binds to CRH. The disclosure is also directed to a method for reducing the severity of one or more symptoms or signs in a subject having CAH, comprising administering to the subject an antibody or antigen-binding fragment thereof disclosed herein.

The disclosure is also directed to a method for reducing the severity of one or more symptoms or signs in a subject having CAH, comprising administering to the subject (i) a mineralocorticoid and/or a glucocorticoid, and (ii) an antibody or antigen-binding fragment thereof that specifically binds to CRH. The disclosure is also directed to a method for reducing the severity of one or more symptoms or signs in a subject having CAH, comprising administering to the subject (i) a mineralocorticoid and/or a glucocorticoid, and (ii) an antibody or antigen-binding fragment thereof disclosed herein. In one aspect, the one or more symptoms or signs is virilization, acne, oily skin or hair, bone age advancement, precocious puberty, short height, hirsutism, male-pattern baldness, enlarged thyroid cartilage, fusion of the labia minora, fusion of the labia majora, hyperpigmentation of the labia majora or minora, rugation of the labia majora, clitoralmegaly, testicular adrenal rest tumors, fertility problems, or irregular or absent menstrual periods.

The disclosure is also directed to a method of treating CAH in a subject in need thereof, comprising administering to the subject (i) a glucocorticoid, and (ii) an antibody or antigen-binding fragment thereof that specifically binds to CRH, wherein the dose of glucocorticoid administered is reduced compared to a glucocorticoid monotherapy. The disclosure is also directed to a method of treating CAH in a subject in need thereof, comprising administering to the subject (i) a glucocorticoid, and (ii) an antibody or antigen-binding fragment thereof disclosed herein, wherein the dose of glucocorticoid administered is reduced compared to a glucocorticoid monotherapy.

The disclosure is also directed to a method of treating CAH in a subject in need thereof, comprising administering to the subject (i) a glucocorticoid, and (ii) an antibody or antigen-binding fragment thereof that specifically binds to CRH, wherein the dose of glucocorticoid administered is reduced compared to a combination therapy comprising a glucocorticoid and a small molecule inhibitor of CRH. The disclosure is also directed to a method of treating CAH in a subject in need thereof, comprising administering to the subject (i) a glucocorticoid, and (ii) an antibody or antigen-binding fragment thereof disclosed herein, wherein the dose of glucocorticoid administered is reduced compared to a combination therapy comprising a glucocorticoid and a small molecule inhibitor of CRH. In one aspect, the severity of one or more undesirable side effects of the glucocorticoid is reduced compared to a glucocorticoid monotherapy. In another aspect, the severity of one or more undesirable side effects of the glucocorticoid is reduced compared to a combination therapy comprising a glucocorticoid and a small molecule inhibitor of CRH. In another aspect, the one or more undesirable side effects is osteoporosis, hyperglycemia, diabetes mellitus, dyslipidemia, increased appetite, weight gain, Cushing syndrome, Cushingoid features, growth suppression, adrenal suppression, gastritis, peptic ulcer, gastrointestinal bleeding, hypertension, mood changes, irritability, anxiety, or infection.

The disclosure is also directed to a method for reducing the levels of one or more biomarkers of CAH in a subject having CAH, comprising administering to the subject an antibody or antigen-binding fragment thereof that specifically binds to CRH. The disclosure is also directed to a method for reducing the levels of one or more biomarkers of CAH in a subject having CAH, comprising administering to the subject an antibody or antigen-binding fragment thereof disclosed herein.

The disclosure is also directed to a method for reducing the levels of one or more biomarkers of CAH in a subject having CAH, comprising administering to the subject (i) a mineralocorticoid and/or a glucocorticoid, and (ii) an antibody or antigen-binding fragment thereof that specifically binds to CRH. The disclosure is also directed to a method for reducing the levels of one or more biomarkers of CAH in a subject having CAH, comprising administering to the subject (i) a mineralocorticoid and/or a glucocorticoid, and (ii) an antibody or antigen-binding fragment thereof disclosed herein. In one aspect, the one or more biomarkers of CAH is 17-hydroxyprogesterone (17-OHP), adrenocorticotropic hormone (ACTH), or androstenedione.

The disclosure is also directed to a method of inhibiting the hypothalamic pituitary adrenal (HPA) axis in a subject in need thereof, comprising administering to the subject an antibody or antigen-binding fragment thereof disclosed herein. In one aspect, the mineralocorticoid is fludrocortisone. In another aspect, the glucocorticoid is beclomethasone, betamethasone, budesonide, cortisone, dexamethasone, hydrocortisone, methylprednisolone, prednisolone, prednisone, or triamcinolone.

In one aspect of the disclosure, the CAH is classic CAH. In another aspect, the CAH is nonclassic CAH.

In one aspect of the disclosure, the treatment methods comprise administering to the subject (i) a glucocorticoid, (ii) an antibody or antigen-binding fragment thereof that specifically binds to CRH, and (iii) a CRHR1 antagonist. In one aspect, the method comprises administering to the subject (i) a glucocorticoid, (ii) an antibody or antigen-binding fragment thereof disclosed herein, and (iii) a CRHR1 antagonist. In one aspect, the method comprises administering to the subject a mineralocorticoid. In one aspect, the method comprises administering to the subject a glucocorticoid. In one aspect, the method comprises administering to the subject a mineralocorticoid and a glucocorticoid.

In one aspect, the CAH is associated with a mutation or deletion in the 21-hydroxylase gene (CYP21A2). In another aspect, the CAH is associated with a mutation or deletion in the 11-beta-hydroxylase gene (CYP11B1). In another aspect, the CAH is associated with a mutation or deletion in the 3-beta-hydroxysteroid dehydrogenase gene (HSD3B2).

In one aspect, the subject is human. In another aspect, the subject is male or female.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 shows the coding sequence open reading frame of mouse CRH (GenBank NM_205769), along with the CRISPR targeting gRNAs and PCR primers used in Example 1.

FIGS. 2A-2B show the identification of CRH knockout founder mice by PCR. The expected PCR DNA fragment sizes from wildtype mice are 146 bp and 103 bp. PCR fragments smaller than these expected sizes indicated deletion in founder mice. PCR Primers CRHbp70F and CRHbp215R flanking the gRNA1 were used to amplify an expected size of 146 bp from wildtype H2L2 Harbour Mice® (FIG. 2A, left panel). PCR primers CRHbp208F and CRHbp310R were used to amplify an expected size of 103 bp from wildtype H2L2 Harbour Mice® (FIG. 2A, middle panel). PCR Primers CRHbp70F and CRHbp310R resulted in PCR fragment size of 241 bp (FIG. 2A, right panel). PCR product smaller than these expected sizes indicated mutations. Founder Mice 1, 2, 4 and 5 were identified as carrying mutations. Founder Mouse 5 from litter 12198 in FIG. 2B carried the 11 bp deletion (5′-CAGCCGGTTCT-3′) (SEQ ID NO: 465), which was used for subsequent breedings.

FIG. 3 shows the location of an 11 bp deletion (5′-CAGCCGGTTCT-3′) (SEQ ID NO: 465) in CRH gene coding region 157-167, that leads to a downstream reading frame shift and premature stop codon. This defective CRH open reading frame is not able to synthesize CRH peptide. A founder having this deletion was chosen for further breeding to homozygosity, rescue and immunization.

FIGS. 4A-4N show CRH ELISA binding activities of the indicated antibodies in PR numbers (FIGS. 4A, 4B, 4D, 4E, 4G, 4H, 4J, 4K, 4L and 4M) and human CRHR1 (hCRHR1) luciferase reporter blocking activities (FIGS. 4C, 4F, 4I and 4N) of anti-CRH mAbs. An unrelated human IgG1 was used as a negative control.

FIGS. 5A-5L show CRH ELISA binding activities (FIGS. 5A-5D) and human CRHR1 (hCRHR1), human CRHR2 (hCRHR2), mouse CRHR1 (mCRHR1), and mouse CRHR2 (mCRHR2) luciferase reporter blocking activities (FIGS. 5E-5L) of Series 1 mAb PR004290 and its ΔPTM variants.

FIGS. 6A-6D show human CRHR1, human CRHR2, mouse CRHR1, and mouse CRHR2 luciferase reporter blocking activities of Series 2 mAb PR006669 and its ΔPTM variants.

FIGS. 7A-7L show CRH ELISA binding activities (FIGS. 7A-7D) and human CRHR1, human CRHR2, mouse CRHR1, and mouse CRHR2 luciferase reporter blocking activities (FIGS. 7E-7L) of Series 5 mAb PR301777 and its humanized variants.

FIGS. 8A-8D show human CRHR1 luciferase reporter blocking activities of Series 7 mAb PR302309 and its humanized variants.

FIGS. 9A-9F show human CRHR1, human CRHR2, mouse CRHR1, and mouse CRHR2 luciferase reporter blocking activities of Series 9 mAb PR302334 and its humanized variants.

FIGS. 10A-10G show human CRHR1, human CRHR2, mouse CRHR1, and mouse CRHR2 luciferase reporter blocking activities of Series 10 mAb PR302341 and its humanized variants.

FIG. 11A-11L show human CRHR1, human CRHR2, mouse CRHR1, and mouse CRHR2 luciferase reporter blocking activities of Series 10 humanized mAbs PR302341-10, PR302341-20, and PR302341-23 and their ΔPTM variants.

FIGS. 12A-12B show that some anti-CRH mAbs bind to human UCN1, but not human UCN2 or human UCN3 by ELISA.

FIGS. 13A-13D show ELISA binding of selected mAbs to CRH and human UCN1.

FIGS. 14A-14H show inhibition of human UCN1-mediated human CRHR1 reporter activity and human CRHR2 reporter activity.

FIGS. 15A-15D show that anti-CRH mAbs bind to the N-terminal region of CRH peptide.

FIG. 16 is a diagram of an epitope binding experiment using the Octet platform.

FIG. 17 shows anti-CRH mAb PR302050 reduces restraint stress-induced ACTH and corticosterone concentrations in wildtype mice.

FIG. 18 shows anti-CRH mAb PR302050 reduces constitutively high ACTH in Mrap1 knockout mice.

FIGS. 19A-19C show anti-CRH mAbs reduce constitutively high ACTH in Mrap1 knockout mice.

FIGS. 20A-20B show additional anti-CRH mAbs that reduce constitutively high ACTH in Mrap1 knockout mice.

FIGS. 21A-21B show anti-CRH mAbs PR302038, PR005660, and PR302050 reduced plasma ACTH concentrations in Mrap1 KO mice, in a dose dependent manner, at 20 mg/kg and 5 mg/kg doses of antibody. FIG. 21A shows results at day 2 after mAb dosing. FIG. 21B shows results at day 16 after mAb dosing.

FIGS. 22A-22B show anti-CRH mAbs PR302334-24 and PR005660 at 20 mg/kg significantly reduced plasma ACTH concentrations in Mrap1 KO mice, whereas PR301429, an anti-CRH mAb with comparable binding affinity, but a non-blocker in vitro, did not inhibit ACTH production. PR303394, an analog of CTRND05, was used as a comparator. FIG. 22A shows results at day 2 after mAb dosing. FIG. 22B shows results at day 16 after mAb dosing.

FIGS. 23A-23B show anti-CRH mAb PR302064 is more efficacious than SSR125543A at inhibiting restraint-induced ACTH (FIG. 23A) and restraint-induced corticosterone (FIG. 23B) release.

FIGS. 24A-24B show anti-CRH mAbs PR302064 and PR302334-24 had superior efficacy to SSR125543A in the Mrap1 KO mice constitutively high ACTH model. ACTH levels at day 2 (FIG. 24A) and day 14 (FIG. 24B) after mAb dosing are shown.

FIGS. 25A-25B show anti-CRH mAb PR302334-24 had superior efficacy to SSR125543A in the wildtype mice restraint stress induced high ACTH and corticosterone model. Induced ACTH (FIG. 25A) and corticosterone (FIG. 25B) plasma levels after mAb dosing are shown.

FIG. 26A is a diagram of a method to test the pharmacokinetics of anti-CRH mAbs. Specifically, anti-CRH mAbs in human IgG1-WT-Fc present in mouse serum were captured by goat anti-human Fc polyclonal antibody and then detected with goat anti-human (H+L) secondary antibody conjugated with HRP.

FIGS. 26B-26D show mean serum concentration time profiles of anti-CRH mAbs administered in a single intravenous dose of 5 mg/kg in female C57BL/6 wildtype mice.

### DETAILED DESCRIPTION OF THE INVENTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

### 1. Definitions

The term “antibody” (Ab) includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibodies,” as used herein, refers to antibodies that are produced by a single clone of B-cells and bind to the same epitope. In contrast, the term “polyclonal antibodies” refers to a population of antibodies that are produced by different B-cells and bind to different epitopes of the same antigen. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

An “antigen-binding fragment” of an antibody refers to one or more fragments or portions of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody described herein, include: The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some aspects, an antibody is an antigen-binding fragment.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a mammalian variable region, e.g., a human or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (a), delta (8), epsilon (¿), gamma (Y), and mu (u), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of kₒₙ/k_{off}. Kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as Octet BLI, BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

“Subject,” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate. In some aspects, the subject is a human.

In some aspects, the subject has experienced and/or exhibited at least one symptom of the disease or disorder to be treated and/or prevented. In some aspects, the subject has been identified or diagnosed as having congenital adrenal hyperplasia (CAH). In some aspects, the subject is suspected of having CAH. In some aspects, the subject has a clinical record indicating that the subject has CAH (and optionally the clinical record indicates that the subject should be treated with the antibodies or antigen binding fragments provided herein).

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “preventing,” as used herein, means the prevention of the onset, recurrence or spread, in whole or in part, of the disease or condition as described herein, or a symptom thereof.

As used herein, “therapeutically effective amount” is an amount of an antibody or antigen binding fragment thereof disclosed herein which is sufficient to achieve the desired effect and can vary according to the nature and severity of the disease condition, and the potency of the antibody/fragment. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage).

In some aspects, the antibodies and antigen-binding fragments described herein comprises a monoclonal antibody, an antibody or an antigen-binding fragment thereof, directed against corticotropin-releasing hormone (CRH), also called corticotropin-releasing factor (CRF) or CRH1. CRH is a neuropeptide hormone that activates the synthesis and release of adrenocorticotropic hormone (ACTH) from the pituitary gland. CRH regulates various neuroendocrine, sympathetic, and behavioral functions, including crucial roles in controlling stress response, anxiety and depression, arousal, feeding behavior, energy metabolism, and digestive and cardiovascular function. CRH is a 41-amino acid peptide derived by enzymatic cleavage from a 196-amino acid preprohormone, and is secreted from the paraventricular nucleus (PVN) of the hypothalamus. The amino acid sequence of human and mouse CRH (UniProtKB-P06850; Genbank Accession No. EAW86897.1) is: SEEPPISLDLTFHLLREVLEMARAEQLAQQAHSNRKLMEII (SEQ ID NO: 466).

CRH acts via two distinct G protein-coupled receptors, CRHR1 and CRHR2. CRHR1 expression is prevalent in brain areas responsible for sensory and motor control, such as the cortical mantle, olfactory bulb, hippocampus, amygdala, basal ganglia, medial and lateral hypothalamic nuclei, and cerebellum. In contrast, CRHR2 is predominant in subcortical regions, including the lateral septum, bed nucleus of the stria terminalis, ventromedial hypothalamic nucleus, and medial and cortical nuclei of the amygdala. In the anterior pituitary, CRHR1 mediates the release of ACTH in response to CRH.

In response to stress, the hypothalamus releases CRH and triggers the release of ACTH from the anterior pituitary into the circulation. Subsequently, ACTH binds to its receptor on the adrenal cortex and triggers the release of stress hormones such as cortisol. This entire system is known as the hypothalamic-pituitary-adrenal (HPA) axis, which plays a crucial role in modulating fight-or-flight responses to stress.

The term “small molecule inhibitor of CRH,” as used herein, refers to chemical compounds or molecules having a molecular weight of approximately 2000 daltons or less that inhibit corticotropin-releasing hormone (CRH). A small molecule inhibitor of CRH includes, but is not limited to, an antagonist of CRH receptor 1 (CRHR1) (also known as corticotropin-releasing factor type-1 (CRF1) receptor). More specific examples of such inhibitors include, but are not limited to, Crinecerfont (SSR125543A or NBI-74788, e.g., Int'l Pub. No. WO 2020/115555), Antalarmin (N-butyl-N-ethyl-(2,5,6-trimethyl-7-(2,4,6-trimethylphenyl)-7H-pyrrolo (2,3-d)pyrimidin-4-yl)amine; U.S. App. Pub. No. 2017/0020877), and Tildacerfont (3-(4-Chloro-2-morpholin-4-yl-thiazol-5-yl)-7-(1-ethyl-propyl)-2,5-dimethyl-pyrazolo[1,5-a]pyrimidine; Int'l Pub. No. WO 2008/036579).

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects are described in further detail in the following sections.

### 2. Anti-CRH Antibodies or Antigen-Binding Fragments Thereof

In some aspects of the present disclosure, the antibody or antigen-binding fragment thereof described herein specifically binds to human CRH. In some aspects, the antibody or antigen-binding fragment thereof comprises a heavy chain variable domain (VH) CDR1 comprising an amino acid sequence of any one of SEQ ID NOs: 22-35, a VH CDR2 comprising an amino acid sequence of any one of SEQ ID NOs: 53-78, and a VH CDR3 comprising an amino acid sequence of any one of SEQ ID NOs: 113-122 or the amino acid sequence of PDV or GID; and/or a light chain variable domain (VL) CDR1 comprising an amino acid sequence of any one of SEQ ID NOs: 152-174, a VL CDR2 comprising the amino acid sequence of any one of SEQ ID NOs: 193-198, and a VL CDR3 comprising an amino acid sequence of any one of SEQ ID NOs: 223-232.

In some aspects, the antibody or antigen-binding fragment thereof comprises a VH comprising an amino acid sequence at least 90%, 95%, or 99% identical to any one of SEQ ID NOs: 240-295, 459 and 461; and/or a VL comprising an amino acid sequence at least 90%, 95%, or 99% identical to any one of SEQ ID NOs: 296-347 and 462. In some aspects, the antibody or antigen-binding fragment comprises a VH comprising an amino acid sequence of any one of SEQ ID NOs: 240-295, 459 and 461; and/or a VL comprising an amino acid sequence of any one of SEQ ID NOs: 296-347 and 462.

In some aspects, the antibody or antigen-binding fragment thereof comprises a heavy chain comprising an amino acid sequence at least 90%, 95%, or 99% identical to any one of SEQ ID NOs: 348-403, 460 and 463; and/or a light chain comprising an amino acid sequence least 90%, 95%, or 99% identical to any one of SEQ ID NOs: 404-455 and 464. In some aspects, the antibody or antigen-binding fragment thereof comprises a heavy chain comprising an amino acid sequence of any one of SEQ ID NOs: 348-403, 460 and 463; and/or a light chain comprising an amino acid sequence of any one of SEQ ID NOs: 404-455 and 464.

In some aspects, the antibody or antigen-binding fragment is an antibody or antigen binding-fragment thereof of Table 1.

TABLE 1

| | VH CDR1 | VH CDR2 | VH CDR3 | VL CDR1 | VL CDR2 | VL CDR3 | VH | VL | H Chain | L Chain |
|---|---|---|---|---|---|---|---|---|---|---|
| | SEQ ID NO: | SEQ ID NO: | SEQ ID NO: | SEQ ID NO: | SEQ ID NO: | SEQ ID NO: | SEQ ID | SEQ ID | SEQ ID | SEQ ID |
| mAb ID | Sequence | Sequence | Sequence | Sequence | SEQ ID NO: | Sequence | NO: | NO: | NO: | NO: |
| PR303394 | | | | | | | 461 | 462 | 463 | 464 |
| PR004289 | 22 | 53 | 113 | 152 | 193 | 223 | 459 | 296 | 460 | 404 |
| | SYWMT | NIKQDGSEKYYVDSVKG | TLIFDY | RSSQSLLHSTGYNSLE | LGSNRAS | MQTLQTPIT | | | | |
| PR004290 | 22 | 53 | 113 | 152 | 193 | 223 | 240 | 296 | 348 | 404 |
| | SYWMT | NIKQDGSEKYYVDSVKG | TLIFDY | RSSQSLLHSTGYNSLE | LGSNRAS | MQTLQTPIT | | | | |
| PR005656 | 22 | 54 | 113 | 152 | 193 | 223 | 241 | 296 | 349 | 404 |
| | SYWMT | NIKQEGSEKYYVDSVKG | TLIFDY | RSSQSLLHSTGYNSLE | LGSNRAS | MQTLQTPIT | | | | |
| PR005657 | 22 | 55 | 113 | 152 | 193 | 223 | 242 | 296 | 350 | 404 |
| | SYWMT | NIKQDASEKYYVDSVKG | TLIFDY | RSSQSLLHSTGYNSLE | LGSNRAS | MQTLQTPIT | | | | |
| PR005658 | 22 | 53 | 113 | 153 | 193 | 223 | 240 | 297 | 348 | 405 |
| | SYWMT | NIKQDGSEKYYVDSVKG | TLIFDY | RSSQSLLHSTGYQSLE | LGSNRAS | MQTLQTPIT | | | | |
| PR005660 | 22 | 54 | 113 | 153 | 193 | 223 | 241 | 297 | 349 | 405 |
| | SYWMT | NIKQEGSEKYYVDSVKG | TLIFDY | RSSQSLLHSTGYQSLE | LGSNRAS | MQTLQTPIT | | | | |
| PR005662 | 22 | 55 | 113 | 153 | 193 | 223 | 242 | 297 | 350 | 405 |
| | SYWMT | NIKQDASEKYYVDSVKG | TLIFDY | RSSQSLLHSTGYQSLE | LGSNRAS | MQTLQTPIT | | | | |
| PR006142 | 22 | 54 | 113 | 154 | 193 | 223 | 241 | 298 | 349 | 406 |
| | SYWMT | NIKQEGSEKYYVDSVKG | TLIFDY | RSSQSLLHSTGYDSLE | LGSNRAS | MQTLQTPIT | | | | |
| PR006143 | 22 | 54 | 113 | 155 | 193 | 223 | 241 | 299 | 349 | 407 |
| | SYWMT | NIKQEGSEKYYVDSVKG | TLIFDY | RSSQSLLHSTGYHSLE | LGSNRAS | MQTLQTPIT | | | | |
| PR006144 | 22 | 54 | 113 | 156 | 193 | 223 | 241 | 300 | 349 | 408 |
| | SYWMT | NIKQEGSEKYYVDSVKG | TLIFDY | RSSQSLLHSTGYNPLE | LGSNRAS | MQTLQTPIT | | | | |
| PR006145 | 22 | 54 | 113 | 157 | 193 | 223 | 241 | 301 | 349 | 409 |
| | SYWMT | NIKQEGSEKYYVDSVKG | TLIFDY | RSSQSLLHSTGYSSLE | LGSNRAS | MQTLQTPIT | | | | |
| PR006146 | 22 | 55 | 113 | 154 | 193 | 223 | 242 | 298 | 350 | 406 |
| | SYWMT | NIKQDASEKYYVDSVKG | TLIFDY | RSSQSLLHSTGYDSLE | LGSNRAS | MQTLQTPIT | | | | |
| PR006147 | 22 | 55 | 113 | 155 | 193 | 223 | 242 | 299 | 350 | 407 |
| | SYWMT | NIKQDASEKYYVDSVKG | TLIFDY | RSSQSLLHSTGYHSLE | LGSNRAS | MQTLQTPIT | | | | |
| PR006148 | 22 | 55 | 113 | 156 | 193 | 223 | 242 | 300 | 350 | 408 |
| | SYWMT | NIKQDASEKYYVDSVKG | TLIFDY | RSSQSLLHSTGYNPLE | LGSNRAS | MQTLQTPIT | | | | |
| PR006149 | 22 | 55 | 113 | 157 | 193 | 223 | 242 | 301 | 350 | 409 |
| | SYWMT | NIKQDASEKYYVDSVKG | TLIFDY | RSSQSLLHSTGYSSLE | LGSNRAS | MQTLQTPIT | | | | |
| PR006150 | 22 | 56 | 113 | 154 | 193 | 223 | 243 | 298 | 351 | 406 |
| | SYWMT | NIKQDSSEKYYVDSVKG | TLIFDY | RSSQSLLHSTGYDSLE | LGSNRAS | MQTLQTPIT | | | | |
| PR006151 | 22 | 56 | 113 | 155 | 193 | 223 | 243 | 299 | 351 | 407 |
| | SYWMT | NIKQDSSEKYYVDSVKG | TLIFDY | RSSQSLLHSTGYHSLE | LGSNRAS | MQTLQTPIT | | | | |
| PR006152 | 22 | 56 | 113 | 156 | 193 | 223 | 243 | 300 | 351 | 408 |
| | SYWMT | NIKQDSSEKYYVDSVKG | TLIFDY | RSSQSLLHSTGYNPLE | LGSNRAS | MQTLQTPIT | | | | |
| PR006153 | 22 | 56 | 113 | 157 | 193 | 223 | 243 | 301 | 351 | 409 |
| | SYWMT | NIKQDSSEKYYVDSVKG | TLIFDY | RSSQSLLHSTGYSSLE | LGSNRAS | MQTLQTPIT | | | | |
| PR006669 | 23 | 57 | 114 | 158 | 193 | 224 | 244 | 302 | 352 | 410 |
| | DNWMS | NIKQDGSENYYVDSVKG | GLALWG | RSSQSLLHSTGYNYLD | LGSNRAS | MQALQTPYT | | | | |
| PR006744 | 23 | 58 | 114 | 158 | 193 | 224 | 245 | 302 | 353 | 410 |
| | DNWMS | NIKQEGSENYYVDSVKG | GLALWG | RSSQSLLHSTGYNYLD | LGSNRAS | MQALQTPYT | | | | |
| PR006745 | 23 | 59 | 114 | 158 | 193 | 224 | 246 | 302 | 354 | 410 |
| | DNWMS | NIKQDASENYYVDSVKG | GLALWG | RSSQSLLHSTGYNYLD | LGSNRAS | MQALQTPYT | | | | |
| PR006746 | 23 | 60 | 114 | 158 | 193 | 224 | 247 | 302 | 355 | 410 |
| | DNWMS | NIKQDSSENYYVDSVKG | GLALWG | RSSQSLLHSTGYNYLD | LGSNRAS | MQALQTPYT | | | | |
| PR301306 | 24 | 61 | 115 | 159 | 194 | 225 | 248 | 303 | 356 | 411 |
| | NYWMN | EIRLKSNNYATLYAESVKG | GTLVY | RASQSVSTSDYNYMH | YASNLES | QHSWEIPYT | | | | |
| PR301612 | 25 | 62 | N/A | 160 | 195 | 226 | 249 | 304 | 357 | 412 |
| | GAWMA | EIKNKANNHATFYAESVKG | PDV | SASSSVSNMY | RTSNLAS | QQYHSFPRT | | | | |
| PR301777 | 24 | 63 | 115 | 161 | 196 | 225 | 250 | 305 | 358 | 413 |
| | NYWMN | EIRLKSNNYATHYAESVKG | GTLVY | RASQSVSTSTYNYMH | FASNLES | QHSWEIPYT | | | | |
| PR301780 | 26 | 64 | 116 | 162 | 197 | 227 | 251 | 306 | 359 | 414 |
| | NFGLN | WINTYTGEPTYTDDFKG | GTYRDDGAWFAY | RSSQIIVHSNGNTYLE | KVSNRFS | FQGSHVPWT | | | | |
| PR301788 | 24 | 63 | 115 | 161 | 196 | 225 | 252 | 305 | 360 | 413 |
| | NYWMN | EIRLKSNNYATHYAESVKG | GTLVY | RASQSVSTSTYNYMH | FASNLES | QHSWEIPYT | | | | |
| PR301790 | 24 | 63 | 115 | 163 | 196 | 225 | 252 | 307 | 360 | 415 |
| | NYWMN | EIRLKSNNYATHYAESVKG | GTLVY | RASQSVSTSSYNYMH | FASNLES | QHSWEIPYT | | | | |
| PR301803 | 24 | 65 | 115 | 161 | 196 | 225 | 253 | 308 | 361 | 416 |
| | NYWMN | EIRLKSNNYATHYAESLKG | GTLVY | RASQSVSTSTYNYMH | FASNLES | QHSWEIPYT | | | | |
| PR301806 | 27 | 66 | 117 | 164 | 197 | 228 | 254 | 309 | 362 | 417 |
| | SYWMH | DIYPGSDSTNYNEKFKS | SITTTTFWYFDV | RSSQSFVYSNGNTYLQ | KVSNRFS | FQGSHVPPT | | | | |
| PR302029 | 24 | 63 | 115 | 161 | 196 | 225 | 255 | 310 | 363 | 418 |
| | NYWMN | EIRLKSNNYATHYAESVKG | GTLVY | RASQSVSTSTYNYMH | FASNLES | QHSWEIPYT | | | | |
| PR302031 | 24 | 63 | 115 | 161 | 196 | 225 | 255 | 311 | 363 | 419 |
| | NYWMN | EIRLKSNNYATHYAESVKG | GTLVY | RASQSVSTSTYNYMH | FASNLES | QHSWEIPYT | | | | |
| PR302034 | 24 | 63 | 115 | 161 | 196 | 225 | 255 | 312 | 363 | 420 |
| | NYWMN | EIRLKSNNYATHYAESVKG | GTLVY | RASQSVSTSTYNYMH | FASNLES | QHSWEIPYT | | | | |
| PR302036 | 24 | 63 | 115 | 161 | 196 | 225 | 256 | 310 | 364 | 418 |
| | NYWMN | EIRLKSNNYATHYAESVKG | GTLVY | RASQSVSTSTYNYMH | FASNLES | QHSWEIPYT | | | | |
| PR302038 | 24 | 63 | 115 | 161 | 196 | 225 | 256 | 311 | 364 | 419 |
| | NYWMN | EIRLKSNNYATHYAESVKG | GTLVY | RASQSVSTSTYNYMH | FASNLES | QHSWEIPYT | | | | |
| PR302041 | 24 | 63 | 115 | 161 | 196 | 225 | 256 | 312 | 364 | 420 |
| | NYWMN | EIRLKSNNYATHYAESVKG | GTLVY | RASQSVSTSTYNYMH | FASNLES | QHSWEIPYT | | | | |
| PR302043 | 24 | 63 | 115 | 161 | 196 | 225 | 257 | 310 | 365 | 418 |
| | NYWMN | EIRLKSNNYATHYAESVKG | GTLVY | RASQSVSTSTYNYMH | FASNLES | QHSWEIPYT | | | | |
| PR302045 | 24 | 63 | 115 | 161 | 196 | 225 | 257 | 311 | 365 | 419 |
| | NYWMN | EIRLKSNNYATHYAESVKG | GTLVY | RASQSVSTSTYNYMH | FASNLES | QHSWEIPYT | | | | |
| PR302048 | 24 | 63 | 115 | 161 | 196 | 225 | 257 | 312 | 365 | 420 |
| | NYWMN | EIRLKSNNYATHYAESVKG | GTLVY | RASQSVSTSTYNYMH | FASNLES | QHSWEIPYT | | | | |
| PR302050 | 24 | 63 | 115 | 161 | 196 | 225 | 258 | 310 | 366 | 418 |
| | NYWMN | EIRLKSNNYATHYAESVKG | GTLVY | RASQSVSTSTYNYMH | FASNLES | QHSWEIPYT | | | | |
| PR302052 | 24 | 63 | 115 | 161 | 196 | 225 | 258 | 311 | 366 | 419 |
| | NYWMN | EIRLKSNNYATHYAESVKG | GTLVY | RASQSVSTSTYNYMH | FASNLES | QHSWEIPYT | | | | |
| PR302055 | 24 | 63 | 115 | 161 | 196 | 225 | 258 | 312 | 366 | 420 |
| | NYWMN | EIRLKSNNYATHYAESVKG | GTLVY | RASQSVSTSTYNYMH | FASNLES | QHSWEIPYT | | | | |
| PR302064 | 24 | 63 | 115 | 161 | 196 | 225 | 259 | 310 | 367 | 418 |
| | NYWMN | EIRLKSNNYATHYAESVKG | GTLVY | RASQSVSTSTYNYMH | FASNLES | QHSWEIPYT | | | | |
| PR302066 | 24 | 63 | 115 | 161 | 196 | 225 | 259 | 311 | 367 | 419 |
| | NYWMN | EIRLKSNNYATHYAESVKG | GTLVY | RASQSVSTSTYNYMH | FASNLES | QHSWEIPYT | | | | |
| PR302069 | 24 | 63 | 115 | 161 | 196 | 225 | 259 | 312 | 367 | 420 |
| | NYWMN | EIRLKSNNYATHYAESVKG | GTLVY | RASQSVSTSTYNYMH | FASNLES | QHSWEIPYT | | | | |
| PR302309 | 28 | 67 | 118 | 165 | 197 | 227 | 260 | 317 | 368 | 425 |
| | NYGMN | WINTYTGEPIYTDDFKG | STMITTGGVFAY | RSSQTIVHSDGNTYLE | KVSNRFS | FQGSHVPWT | | | | |
| PR302309-1 | 28 | 67 | 118 | 165 | 197 | 227 | 260 | 313 | 368 | 421 |
| | NYGMN | WINTYTGEPIYTDDFKG | STMITTGGVFAY | RSSQTIVHSDGNTYLE | KVSNRFS | FQGSHVPWT | | | | |
| PR302309-10 | 28 | 67 | 118 | 165 | 197 | 227 | 261 | 314 | 369 | 422 |
| | NYGMN | WINTYTGEPIYTDDFKG | STMITTGGVFAY | RSSQTIVHSDGNTYLE | KVSNRFS | FQGSHVPWT | | | | |
| PR302309-11 | 28 | 67 | 118 | 165 | 197 | 227 | 261 | 315 | 369 | 423 |
| | NYGMN | WINTYTGEPIYTDDFKG | STMITTGGVFAY | RSSQTIVHSDGNTYLE | KVSNRFS | FQGSHVPWT | | | | |
| PR302309-12 | 28 | 67 | 118 | 165 | 197 | 227 | 261 | 316 | 369 | 424 |
| | NYGMN | WINTYTGEPIYTDDFKG | STMITTGGVFAY | RSSQTIVHSDGNTYLE | KVSNRFS | FQGSHVPWT | | | | |
| PR302309-13 | 28 | 67 | 118 | 165 | 197 | 227 | 262 | 317 | 370 | 425 |
| | NYGMN | WINTYTGEPIYTDDFKG | STMITTGGVFAY | RSSQTIVHSDGNTYLE | KVSNRFS | FQGSHVPWT | | | | |
| PR302309-14 | 28 | 67 | 118 | 165 | 197 | 227 | 261 | 317 | 369 | 425 |
| | NYGMN | WINTYTGEPIYTDDFKG | STMITTGGVFAY | RSSQTIVHSDGNTYLE | KVSNRFS | FQGSHVPWT | | | | |
| PR302309-15 | 28 | 67 | 118 | 165 | 197 | 227 | 263 | 317 | 371 | 425 |
| | NYGMN | WINTYTGEPIYTDDFKG | STMITTGGVFAY | RSSQTIVHSDGNTYLE | KVSNRFS | FQGSHVPWT | | | | |
| PR302309-16 | 28 | 67 | 118 | 165 | 197 | 227 | 264 | 317 | 372 | 425 |
| | NYGMN | WINTYTGEPIYTDDFKG | STMITTGGVFAY | RSSQTIVHSDGNTYLE | KVSNRFS | FQGSHVPWT | | | | |
| PR302309-17 | 28 | 67 | 118 | 165 | 197 | 227 | 262 | 318 | 370 | 426 |
| | NYGMN | WINTYTGEPIYTDDFKG | STMITTGGVFAY | RSSQTIVHSDGNTYLE | KVSNRFS | FQGSHVPWT | | | | |
| PR302309-18 | 28 | 67 | 118 | 165 | 197 | 227 | 263 | 318 | 371 | 426 |
| | NYGMN | WINTYTGEPIYTDDFKG | STMITTGGVFAY | RSSQTIVHSDGNTYLE | KVSNRFS | FQGSHVPWT | | | | |
| PR302309-19 | 28 | 67 | 118 | 165 | 197 | 227 | 264 | 318 | 372 | 426 |
| | NYGMN | WINTYTGEPIYTDDFKG | STMITTGGVFAY | RSSQTIVHSDGNTYLE | KVSNRFS | FQGSHVPWT | | | | |
| PR302309-2 | 28 | 67 | 118 | 165 | 197 | 227 | 260 | 318 | 368 | 426 |
| | NYGMN | WINTYTGEPIYTDDFKG | STMITTGGVFAY | RSSQTIVHSDGNTYLE | KVSNRFS | FQGSHVPWT | | | | |
| PR302309-20 | 28 | 67 | 118 | 165 | 197 | 227 | 264 | 314 | 372 | 422 |
| | NYGMN | WINTYTGEPIYTDDFKG | STMITTGGVFAY | RSSQTIVHSDGNTYLE | KVSNRFS | FQGSHVPWT | | | | |
| PR302309-21 | 28 | 67 | 118 | 165 | 197 | 227 | 264 | 316 | 372 | 424 |
| | NYGMN | WINTYTGEPIYTDDFKG | STMITTGGVFAY | RSSQTIVHSDGNTYLE | KVSNRFS | FQGSHVPWT | | | | |
| PR302309-3 | 28 | 67 | 118 | 165 | 197 | 227 | 260 | 319 | 368 | 427 |
| | NYGMN | WINTYTGEPIYTDDFKG | STMITTGGVFAY | RSSQTIVHSDGNTYLE | KVSNRFS | FQGSHVPWT | | | | |
| PR302309-4 | 28 | 67 | 118 | 165 | 197 | 227 | 260 | 314 | 368 | 422 |
| | NYGMN | WINTYTGEPIYTDDFKG | STMITTGGVFAY | RSSQTIVHSDGNTYLE | KVSNRFS | FQGSHVPWT | | | | |
| PR302309-5 | 28 | 67 | 118 | 165 | 197 | 227 | 260 | 315 | 368 | 423 |
| | NYGMN | WINTYTGEPIYTDDFKG | STMITTGGVFAY | RSSQTIVHSDGNTYLE | KVSNRFS | FQGSHVPWT | | | | |
| PR302309-6 | 28 | 67 | 118 | 165 | 197 | 227 | 260 | 316 | 368 | 424 |
| | NYGMN | WINTYTGEPIYTDDFKG | STMITTGGVFAY | RSSQTIVHSDGNTYLE | KVSNRFS | FQGSHVPWT | | | | |
| PR302309-7 | 28 | 67 | 118 | 165 | 197 | 227 | 261 | 313 | 369 | 421 |
| | NYGMN | WINTYTGEPIYTDDFKG | STMITTGGVFAY | RSSQTIVHSDGNTYLE | KVSNRFS | FQGSHVPWT | | | | |
| PR302309-8 | 28 | 67 | 118 | 165 | 197 | 227 | 261 | 318 | 369 | 426 |
| | NYGMN | WINTYTGEPIYTDDFKG | STMITTGGVFAY | RSSQTIVHSDGNTYLE | KVSNRFS | FQGSHVPWT | | | | |
| PR302309-9 | 28 | 67 | 118 | 165 | 197 | 227 | 261 | 319 | 369 | 427 |
| | NYGMN | WINTYTGEPIYTDDFKG | STMITTGGVFAY | RSSQTIVHSDGNTYLE | KVSNRFS | FQGSHVPWT | | | | |
| PR302312 | 29 | 68 | 119 | 166 | 197 | 227 | 265 | 320 | 373 | 428 |
| | DYFMK | VINPNNGDTFYNQNFKG | GTARALFAY | RSSQSIVHSDGNTYLE | KVSNRFS | FQGSHVPWT | | | | |
| PR302315 | 30 | 69 | 120 | 167 | 197 | 229 | 266 | 321 | 374 | 429 |
| | SFGMH | YISSGSSIIYYADTVKG | DITTATFAY | RSSQSLVHSNGNTYLH | KVSNRFS | SQSTHVPWT | | | | |
| PR302324 | 29 | 70 | 121 | 166 | 197 | 230 | 267 | 322 | 375 | 430 |
| | DYFMK | VISPNNGDTFYNQKFKG | GTDRALFAY | RSSQSIVHSDGNTYLE | KVSNRFS | FQGSHIPWT | | | | |
| PR302328 | 31 | 71 | N/A | 168 | 198 | 231 | 268 | 323 | 376 | 431 |
| | SYAMS | SIRSGGTTYYPDSVKG | GID | KASQSVNNDVA | YASNRYT | QQDYSSWT | | | | |
| PR302331 | 31 | 71 | N/A | 168 | 198 | 231 | 269 | 324 | 377 | 432 |
| | SYAMS | SIRSGGTTYYPDSVKG | GID | KASQSVNNDVA | YASNRYT | QQDYSSWT | | | | |
| PR302332 | 32 | 72 | 118 | 166 | 197 | 227 | 270 | 325 | 378 | 433 |
| | NFGMH | WINTYTGEPIYAADFKG | STMITTGGVFAY | RSSQSIVHSDGNTYLE | KVSNRFS | FQGSHVPWT | | | | |
| PR302333 | 33 | 73 | 118 | 169 | 197 | 227 | 271 | 326 | 379 | 434 |
| | NFGMN | WINTYTGEPIYADDFKG | STMITTGGVFAY | RSSQNIVHSDGNTYLE | KVSNRFS | FQGSHVPWT | | | | |
| PR302334 | 34 | 71 | N/A | 168 | 198 | 231 | 272 | 331 | 380 | 439 |
| | TYAMS | SIRSGGTTYYPDSVKG | GID | KASQSVNNDVA | YASNRYT | QQDYSSWT | | | | |
| PR302334-1 | 34 | 71 | N/A | 168 | 198 | 231 | 272 | 327 | 380 | 435 |
| | TYAMS | SIRSGGTTYYPDSVKG | GID | KASQSVNNDVA | YASNRYT | QQDYSSWT | | | | |
| PR302334-10 | 34 | 71 | N/A | 168 | 198 | 231 | 273 | 328 | 381 | 436 |
| | TYAMS | SIRSGGTTYYPDSVKG | GID | KASQSVNNDVA | YASNRYT | QQDYSSWT | | | | |
| PR302334-11 | 34 | 71 | N/A | 168 | 198 | 231 | 273 | 329 | 381 | 437 |
| | TYAMS | SIRSGGTTYYPDSVKG | GID | KASQSVNNDVA | YASNRYT | QQDYSSWT | | | | |
| PR302334-12 | 34 | 71 | N/A | 168 | 198 | 231 | 273 | 330 | 381 | 438 |
| | TYAMS | SIRSGGTTYYPDSVKG | GID | KASQSVNNDVA | YASNRYT | QQDYSSWT | | | | |
| PR302334-14 | 34 | 71 | N/A | 168 | 198 | 231 | 273 | 331 | 381 | 439 |
| | TYAMS | SIRSGGTTYYPDSVKG | GID | KASQSVNNDVA | YASNRYT | QQDYSSWT | | | | |
| PR302334-15 | 34 | 71 | N/A | 168 | 198 | 231 | 274 | 331 | 382 | 439 |
| | TYAMS | SIRSGGTTYYPDSVKG | GID | KASQSVNNDVA | YASNRYT | QQDYSSWT | | | | |
| PR302334-16 | 34 | 71 | N/A | 168 | 198 | 231 | 275 | 331 | 383 | 439 |
| | TYAMS | SIRSGGTTYYPDSVKG | GID | KASQSVNNDVA | YASNRYT | QQDYSSWT | | | | |
| PR302334-17 | 34 | 71 | N/A | 168 | 198 | 231 | 276 | 331 | 384 | 439 |
| | TYAMS | SIRSGGTTYYPDSVKG | GID | KASQSVNNDVA | YASNRYT | QQDYSSWT | | | | |
| PR302334-18 | 34 | 71 | N/A | 168 | 198 | 231 | 277 | 331 | 385 | 439 |
| | TYAMS | SIRSGGTTYYPDSVKG | GID | KASQSVNNDVA | YASNRYT | QQDYSSWT | | | | |
| PR302334-2 | 34 | 71 | N/A | 168 | 198 | 231 | 272 | 332 | 380 | 440 |
| | TYAMS | SIRSGGTTYYPDSVKG | GID | KASQSVNNDVA | YASNRYT | QQDYSSWT | | | | |
| PR302334-22 | 34 | 71 | N/A | 168 | 198 | 231 | 276 | 332 | 384 | 440 |
| | TYAMS | SIRSGGTTYYPDSVKG | GID | KASQSVNNDVA | YASNRYT | QQDYSSWT | | | | |
| PR302334-24 | 34 | 71 | N/A | 168 | 198 | 231 | 275 | 328 | 383 | 436 |
| | TYAMS | SIRSGGTTYYPDSVKG | GID | KASQSVNNDVA | YASNRYT | QQDYSSWT | | | | |
| PR302334-25 | 34 | 71 | N/A | 168 | 198 | 231 | 275 | 330 | 383 | 438 |
| | TYAMS | SIRSGGTTYYPDSVKG | GID | KASQSVNNDVA | YASNRYT | QQDYSSWT | | | | |
| PR302334-26 | 34 | 71 | N/A | 168 | 198 | 231 | 277 | 328 | 385 | 436 |
| | TYAMS | SIRSGGTTYYPDSVKG | GID | KASQSVNNDVA | YASNRYT | QQDYSSWT | | | | |
| PR302334-27 | 34 | 71 | N/A | 168 | 198 | 231 | 277 | 330 | 385 | 438 |
| | TYAMS | SIRSGGTTYYPDSVKG | GID | KASQSVNNDVA | YASNRYT | QQDYSSWT | | | | |
| PR302334-3 | 34 | 71 | N/A | 168 | 198 | 231 | 272 | 333 | 380 | 441 |
| | TYAMS | SIRSGGTTYYPDSVKG | GID | KASQSVNNDVA | YASNRYT | QQDYSSWT | | | | |
| PR302334-4 | 34 | 71 | N/A | 168 | 198 | 231 | 272 | 328 | 380 | 436 |
| | TYAMS | SIRSGGTTYYPDSVKG | GID | KASQSVNNDVA | YASNRYT | QQDYSSWT | | | | |
| PR302334-5 | 34 | 71 | N/A | 168 | 198 | 231 | 272 | 329 | 380 | 437 |
| | TYAMS | SIRSGGTTYYPDSVKG | GID | KASQSVNNDVA | YASNRYT | QQDYSSWT | | | | |
| PR302334-6 | 34 | 71 | N/A | 168 | 198 | 231 | 272 | 330 | 380 | 438 |
| | TYAMS | SIRSGGTTYYPDSVKG | GID | KASQSVNNDVA | YASNRYT | QQDYSSWT | | | | |
| PR302334-7 | 34 | 71 | N/A | 168 | 198 | 231 | 273 | 327 | 381 | 435 |
| | TYAMS | SIRSGGTTYYPDSVKG | GID | KASQSVNNDVA | YASNRYT | QQDYSSWT | | | | |
| PR302334-8 | 34 | 71 | N/A | 168 | 198 | 231 | 273 | 332 | 381 | 440 |
| | TYAMS | SIRSGGTTYYPDSVKG | GID | KASQSVNNDVA | YASNRYT | QQDYSSWT | | | | |
| PR302334-9 | 34 | 71 | N/A | 168 | 198 | 231 | 273 | 333 | 381 | 441 |
| | TYAMS | SIRSGGTTYYPDSVKG | GID | KASQSVNNDVA | YASNRYT | QQDYSSWT | | | | |
| PR302335 | 30 | 74 | 122 | 170 | 197 | 232 | 278 | 334 | 386 | 442 |
| | SFGMH | YISSGSTIFYYADTVKG | DITTATFAF | RSSQNLLHNNGNTYLH | KVSNRFS | SQSTHIPWT | | | | |
| PR302339 | 35 | 71 | N/A | 168 | 198 | 231 | 279 | 323 | 387 | 431 |
| | SYALS | SIRSGGTTYYPDSVKG | GID | KASQSVNNDVA | YASNRYT | QQDYSSWT | | | | |
| PR302341 | 29 | 75 | 121 | 171 | 197 | 230 | 280 | 339 | 388 | 447 |
| | DYFMK | VINPNNGDIFYNQKFKG | GTDRALFAY | RSGQSIVHSDGNTYLE | KVSNRFS | FQGSHIPWT | | | | |
| PR302341-1 | 29 | 75 | 121 | 171 | 197 | 230 | 280 | 335 | 388 | 443 |
| | DYFMK | VINPNNGDIFYNQKFKG | GTDRALFAY | RSGQSIVHSDGNTYLE | KVSNRFS | FQGSHIPWT | | | | |
| PR302341-10 | 29 | 75 | 121 | 171 | 197 | 230 | 281 | 336 | 389 | 444 |
| | DYFMK | VINPNNGDIFYNQKFKG | GTDRALFAY | RSGQSIVHSDGNTYLE | KVSNRFS | FQGSHIPWT | | | | |
| PR302341-11 | 29 | 75 | 121 | 171 | 197 | 230 | 281 | 337 | 389 | 445 |
| | DYFMK | VINPNNGDIFYNQKFKG | GTDRALFAY | RSGQSIVHSDGNTYLE | KVSNRFS | FQGSHIPWT | | | | |
| PR302341-12 | 29 | 75 | 121 | 171 | 197 | 230 | 281 | 338 | 389 | 446 |
| | DYFMK | VINPNNGDIFYNQKFKG | GTDRALFAY | RSGQSIVHSDGNTYLE | KVSNRFS | FQGSHIPWT | | | | |
| PR302341-13 | 29 | 75 | 121 | 171 | 197 | 230 | 282 | 339 | 390 | 447 |
| | DYFMK | VINPNNGDIFYNQKFKG | GTDRALFAY | RSGQSIVHSDGNTYLE | KVSNRFS | FQGSHIPWT | | | | |
| PR302341-14 | 29 | 75 | 121 | 171 | 197 | 230 | 281 | 339 | 389 | 447 |
| | DYFMK | VINPNNGDIFYNQKFKG | GTDRALFAY | RSGQSIVHSDGNTYLE | KVSNRFS | FQGSHIPWT | | | | |
| PR302341-15 | 29 | 75 | 121 | 171 | 197 | 230 | 283 | 339 | 391 | 447 |
| | DYFMK | VINPNNGDIFYNQKFKG | GTDRALFAY | RSGQSIVHSDGNTYLE | KVSNRFS | FQGSHIPWT | | | | |
| PR302341-16 | 29 | 75 | 121 | 171 | 197 | 230 | 284 | 339 | 392 | 447 |
| | DYFMK | VINPNNGDIFYNQKFKG | GTDRALFAY | RSGQSIVHSDGNTYLE | KVSNRFS | FQGSHIPWT | | | | |
| PR302341-17 | 29 | 75 | 121 | 171 | 197 | 230 | 285 | 339 | 393 | 447 |
| | DYFMK | VINPNNGDIFYNQKFKG | GTDRALFAY | RSGQSIVHSDGNTYLE | KVSNRFS | FQGSHIPWT | | | | |
| PR302341-18 | 29 | 75 | 121 | 171 | 197 | 230 | 286 | 339 | 394 | 447 |
| | DYFMK | VINPNNGDIFYNQKFKG | GTDRALFAY | RSGQSIVHSDGNTYLE | KVSNRFS | FQGSHIPWT | | | | |
| PR302341-19 | 29 | 75 | 121 | 171 | 197 | 230 | 282 | 340 | 390 | 448 |
| | DYFMK | VINPNNGDIFYNQKFKG | GTDRALFAY | RSGQSIVHSDGNTYLE | KVSNRFS | FQGSHIPWT | | | | |
| PR302341-2 | 29 | 75 | 121 | 171 | 197 | 230 | 280 | 340 | 388 | 448 |
| | DYFMK | VINPNNGDIFYNQKFKG | GTDRALFAY | RSGQSIVHSDGNTYLE | KVSNRFS | FQGSHIPWT | | | | |
| PR302341-20 | 29 | 75 | 121 | 171 | 197 | 230 | 283 | 340 | 391 | 448 |
| | DYFMK | VINPNNGDIFYNQKFKG | GTDRALFAY | RSGQSIVHSDGNTYLE | KVSNRFS | FQGSHIPWT | | | | |
| PR302341-21 | 29 | 75 | 121 | 171 | 197 | 230 | 284 | 340 | 392 | 448 |
| | DYFMK | VINPNNGDIFYNQKFKG | GTDRALFAY | RSGQSIVHSDGNTYLE | KVSNRFS | FQGSHIPWT | | | | |
| PR302341-22 | 29 | 75 | 121 | 171 | 197 | 230 | 285 | 340 | 393 | 448 |
| | DYFMK | VINPNNGDIFYNQKFKG | GTDRALFAY | RSGQSIVHSDGNTYLE | KVSNRFS | FQGSHIPWT | | | | |
| PR302341-23 | 29 | 75 | 121 | 171 | 197 | 230 | 286 | 340 | 394 | 448 |
| | DYFMK | VINPNNGDIFYNQKFKG | GTDRALFAY | RSGQSIVHSDGNTYLE | KVSNRFS | FQGSHIPWT | | | | |
| PR302341-24 | 29 | 75 | 121 | 171 | 197 | 230 | 284 | 336 | 392 | 444 |
| | DYFMK | VINPNNGDIFYNQKFKG | GTDRALFAY | RSGQSIVHSDGNTYLE | KVSNRFS | FQGSHIPWT | | | | |
| PR302341-25 | 29 | 75 | 121 | 171 | 197 | 230 | 284 | 338 | 392 | 446 |
| | DYFMK | VINPNNGDIFYNQKFKG | GTDRALFAY | RSGQSIVHSDGNTYLE | KVSNRFS | FQGSHIPWT | | | | |
| PR302341-26 | 29 | 75 | 121 | 171 | 197 | 230 | 286 | 336 | 394 | 444 |
| | DYFMK | VINPNNGDIFYNQKFKG | GTDRALFAY | RSGQSIVHSDGNTYLE | KVSNRFS | FQGSHIPWT | | | | |
| PR302341-27 | 29 | 75 | 121 | 171 | 197 | 230 | 286 | 338 | 394 | 446 |
| | DYFMK | VINPNNGDIFYNQKFKG | GTDRALFAY | RSGQSIVHSDGNTYLE | KVSNRFS | FQGSHIPWT | | | | |
| PR302341-28 | 29 | 76 | 121 | 171 | 197 | 230 | 287 | 340 | 395 | 448 |
| | DYFMK | VINPNQGDIFYNQKFKG | GTDRALFAY | RSGQSIVHSDGNTYLE | KVSNRFS | FQGSHIPWT | | | | |
| PR302341-29 | 29 | 77 | 121 | 171 | 197 | 230 | 288 | 340 | 396 | 448 |
| | DYFMK | VINPNNADIFYNQKFKG | GTDRALFAY | RSGQSIVHSDGNTYLE | KVSNRFS | FQGSHIPWT | | | | |
| PR302341-3 | 29 | 75 | 121 | 171 | 197 | 230 | 280 | 341 | 388 | 449 |
| | DYFMK | VINPNNGDIFYNQKFKG | GTDRALFAY | RSGQSIVHSDGNTYLE | KVSNRFS | FQGSHIPWT | | | | |
| PR302341-30 | 29 | 75 | 121 | 172 | 197 | 230 | 286 | 342 | 394 | 450 |
| | DYFMK | VINPNNGDIFYNQKFKG | GTDRALFAY | RSGQSIVHSEGNTYLE | KVSNRFS | FQGSHIPWT | | | | |
| PR302341-31 | 29 | 75 | 121 | 173 | 197 | 230 | 286 | 343 | 394 | 451 |
| | DYFMK | VINPNNGDIFYNQKFKG | GTDRALFAY | RSGQSIVHSDANTYLE | KVSNRFS | FQGSHIPWT | | | | |
| PR302341-32 | 29 | 76 | 121 | 172 | 197 | 230 | 287 | 342 | 395 | 450 |
| | DYFMK | VINPNQGDIFYNQKFKG | GTDRALFAY | RSGQSIVHSEGNTYLE | KVSNRFS | FQGSHIPWT | | | | |
| PR302341-33 | 29 | 77 | 121 | 173 | 197 | 230 | 288 | 343 | 396 | 451 |
| | DYFMK | VINPNNADIFYNQKFKG | GTDRALFAY | RSGQSIVHSDANTYLE | KVSNRFS | FQGSHIPWT | | | | |
| PR302341-34 | 29 | 76 | 121 | 173 | 197 | 230 | 287 | 343 | 395 | 451 |
| | DYFMK | VINPNQGDIFYNQKFKG | GTDRALFAY | RSGQSIVHSDANTYLE | KVSNRFS | FQGSHIPWT | | | | |
| PR302341-35 | 29 | 77 | 121 | 172 | 197 | 230 | 288 | 342 | 396 | 450 |
| | DYFMK | VINPNNADIFYNQKFKG | GTDRALFAY | RSGQSIVHSEGNTYLE | KVSNRFS | FQGSHIPWT | | | | |
| PR302341-36 | 29 | 78 | 121 | 172 | 197 | 230 | 289 | 342 | 397 | 450 |
| | DYFMK | VINPNSGDIFYNQKFKG | GTDRALFAY | RSGQSIVHSEGNTYLE | KVSNRFS | FQGSHIPWT | | | | |
| PR302341-37 | 29 | 78 | 121 | 173 | 197 | 230 | 289 | 343 | 397 | 451 |
| | DYFMK | VINPNSGDIFYNQKFKG | GTDRALFAY | RSGQSIVHSDANTYLE | KVSNRFS | FQGSHIPWT | | | | |
| PR302341-38 | 29 | 76 | 121 | 174 | 197 | 230 | 287 | 344 | 395 | 452 |
| | DYFMK | VINPNQGDIFYNQKFKG | GTDRALFAY | RSGQSIVHSSGNTYLE | KVSNRFS | FQGSHIPWT | | | | |
| PR302341-39 | 29 | 77 | 121 | 174 | 197 | 230 | 288 | 344 | 396 | 452 |
| | DYFMK | VINPNNADIFYNQKFKG | GTDRALFAY | RSGQSIVHSSGNTYLE | KVSNRFS | FQGSHIPWT | | | | |
| PR302341-4 | 29 | 75 | 121 | 171 | 197 | 230 | 280 | 336 | 388 | 444 |
| | DYFMK | VINPNNGDIFYNQKFKG | GTDRALFAY | RSGQSIVHSDGNTYLE | KVSNRFS | FQGSHIPWT | | | | |
| PR302341-40 | 29 | 78 | 121 | 174 | 197 | 230 | 289 | 344 | 397 | 452 |
| | DYFMK | VINPNSGDIFYNQKFKG | GTDRALFAY | RSGQSIVHSSGNTYLE | KVSNRFS | FQGSHIPWT | | | | |
| PR302341-41 | 29 | 78 | 121 | 171 | 197 | 230 | 289 | 340 | 397 | 448 |
| | DYFMK | VINPNSGDIFYNQKFKG | GTDRALFAY | RSGQSIVHSDGNTYLE | KVSNRFS | FQGSHIPWT | | | | |
| PR302341-42 | 29 | 75 | 121 | 174 | 197 | 230 | 286 | 344 | 394 | 452 |
| | DYFMK | VINPNNGDIFYNQKFKG | GTDRALFAY | RSGQSIVHSSGNTYLE | KVSNRFS | FQGSHIPWT | | | | |
| PR302341-43 | 29 | 77 | 121 | 171 | 197 | 230 | 290 | 336 | 398 | 444 |
| | DYFMK | VINPNNADIFYNQKFKG | GTDRALFAY | RSGQSIVHSDGNTYLE | KVSNRFS | FQGSHIPWT | | | | |
| PR302341-44 | 29 | 78 | 121 | 171 | 197 | 230 | 291 | 336 | 399 | 444 |
| | DYFMK | VINPNSGDIFYNQKFKG | GTDRALFAY | RSGQSIVHSDGNTYLE | KVSNRFS | FQGSHIPWT | | | | |
| PR302341-45 | 29 | 76 | 121 | 171 | 197 | 230 | 292 | 336 | 400 | 444 |
| | DYFMK | VINPNQGDIFYNQKFKG | GTDRALFAY | RSGQSIVHSDGNTYLE | KVSNRFS | FQGSHIPWT | | | | |
| PR302341-46 | 29 | 77 | 121 | 173 | 197 | 230 | 290 | 345 | 398 | 453 |
| | DYFMK | VINPNNADIFYNQKFKG | GTDRALFAY | RSGQSIVHSDANTYLE | KVSNRFS | FQGSHIPWT | | | | |
| PR302341-47 | 29 | 78 | 121 | 173 | 197 | 230 | 291 | 345 | 399 | 453 |
| | DYFMK | VINPNSGDIFYNQKFKG | GTDRALFAY | RSGQSIVHSDANTYLE | KVSNRFS | FQGSHIPWT | | | | |
| PR302341-48 | 29 | 76 | 121 | 173 | 197 | 230 | 292 | 345 | 400 | 453 |
| | DYFMK | VINPNQGDIFYNQKFKG | GTDRALFAY | RSGQSIVHSDANTYLE | KVSNRFS | FQGSHIPWT | | | | |
| PR302341-49 | 29 | 77 | 121 | 172 | 197 | 230 | 290 | 346 | 398 | 454 |
| | DYFMK | VINPNNADIFYNQKFKG | GTDRALFAY | RSGQSIVHSEGNTYLE | KVSNRFS | FQGSHIPWT | | | | |
| PR302341-5 | 29 | 75 | 121 | 171 | 197 | 230 | 280 | 337 | 388 | 445 |
| | DYFMK | VINPNNGDIFYNQKFKG | GTDRALFAY | RSGQSIVHSDGNTYLE | KVSNRFS | FQGSHIPWT | | | | |
| PR302341-50 | 29 | 78 | 121 | 172 | 197 | 230 | 291 | 346 | 399 | 454 |
| | DYFMK | VINPNSGDIFYNQKFKG | GTDRALFAY | RSGQSIVHSEGNTYLE | KVSNRFS | FQGSHIPWT | | | | |
| PR302341-51 | 29 | 76 | 121 | 172 | 197 | 230 | 292 | 346 | 400 | 454 |
| | DYFMK | VINPNQGDIFYNQKFKG | GTDRALFAY | RSGQSIVHSEGNTYLE | KVSNRFS | FQGSHIPWT | | | | |
| PR302341-52 | 29 | 77 | 121 | 174 | 197 | 230 | 290 | 347 | 398 | 455 |
| | DYFMK | VINPNNADIFYNQKFKG | GTDRALFAY | RSGQSIVHSSGNTYLE | KVSNRFS | FQGSHIPWT | | | | |
| PR302341-53 | 29 | 78 | 121 | 174 | 197 | 230 | 291 | 347 | 399 | 455 |
| | DYFMK | VINPNSGDIFYNQKFKG | GTDRALFAY | RSGQSIVHSSGNTYLE | KVSNRFS | FQGSHIPWT | | | | |
| PR302341-54 | 29 | 76 | 121 | 174 | 197 | 230 | 292 | 347 | 400 | 455 |
| | DYFMK | VINPNQGDIFYNQKFKG | GTDRALFAY | RSGQSIVHSSGNTYLE | KVSNRFS | FQGSHIPWT | | | | |
| PR302341-55 | 29 | 77 | 121 | 171 | 197 | 230 | 293 | 340 | 401 | 448 |
| | DYFMK | VINPNNADIFYNQKFKG | GTDRALFAY | RSGQSIVHSDGNTYLE | KVSNRFS | FQGSHIPWT | | | | |
| PR302341-56 | 29 | 78 | 121 | 171 | 197 | 230 | 294 | 340 | 402 | 448 |
| | DYFMK | VINPNSGDIFYNQKFKG | GTDRALFAY | RSGQSIVHSDGNTYLE | KVSNRFS | FQGSHIPWT | | | | |
| PR302341-57 | 29 | 76 | 121 | 171 | 197 | 230 | 295 | 340 | 403 | 448 |
| | DYFMK | VINPNQGDIFYNQKFKG | GTDRALFAY | RSGQSIVHSDGNTYLE | KVSNRFS | FQGSHIPWT | | | | |
| PR302341-58 | 29 | 77 | 121 | 173 | 197 | 230 | 293 | 343 | 401 | 451 |
| | DYFMK | VINPNNADIFYNQKFKG | GTDRALFAY | RSGQSIVHSDANTYLE | KVSNRFS | FQGSHIPWT | | | | |
| PR302341-59 | 29 | 78 | 121 | 173 | 197 | 230 | 294 | 343 | 402 | 451 |
| | DYFMK | VINPNSGDIFYNQKFKG | GTDRALFAY | RSGQSIVHSDANTYLE | KVSNRFS | FQGSHIPWT | | | | |
| PR302341-6 | 29 | 75 | 121 | 171 | 197 | 230 | 280 | 338 | 388 | 446 |
| | DYFMK | VINPNNGDIFYNQKFKG | GTDRALFAY | RSGQSIVHSDGNTYLE | KVSNRFS | FQGSHIPWT | | | | |
| PR302341-60 | 29 | 76 | 121 | 173 | 197 | 230 | 295 | 343 | 403 | 451 |
| | DYFMK | VINPNQGDIFYNQKFKG | GTDRALFAY | RSGQSIVHSDANTYLE | KVSNRFS | FQGSHIPWT | | | | |
| PR302341-61 | 29 | 77 | 121 | 172 | 197 | 230 | 293 | 342 | 401 | 450 |
| | DYFMK | VINPNNADIFYNQKFKG | GTDRALFAY | RSGQSIVHSEGNTYLE | KVSNRFS | FQGSHIPWT | | | | |
| PR302341-62 | 29 | 78 | 121 | 172 | 197 | 230 | 294 | 342 | 402 | 450 |
| | DYFMK | VINPNSGDIFYNQKFKG | GTDRALFAY | RSGQSIVHSEGNTYLE | KVSNRFS | FQGSHIPWT | | | | |
| PR302341-63 | 29 | 76 | 121 | 172 | 197 | 230 | 295 | 342 | 403 | 450 |
| | DYFMK | VINPNQGDIFYNQKFKG | GTDRALFAY | RSGQSIVHSEGNTYLE | KVSNRFS | FQGSHIPWT | | | | |
| PR302341-64 | 29 | 77 | 121 | 174 | 197 | 230 | 293 | 344 | 401 | 452 |
| | DYFMK | VINPNNADIFYNQKFKG | GTDRALFAY | RSGQSIVHSSGNTYLE | KVSNRFS | FQGSHIPWT | | | | |
| PR302341-65 | 29 | 78 | 121 | 174 | 197 | 230 | 294 | 344 | 402 | 452 |
| | DYFMK | VINPNSGDIFYNQKFKG | GTDRALFAY | RSGQSIVHSSGNTYLE | KVSNRFS | FQGSHIPWT | | | | |
| PR302341-66 | 29 | 76 | 121 | 174 | 197 | 230 | 295 | 344 | 403 | 452 |
| | DYFMK | VINPNQGDIFYNQKFKG | GTDRALFAY | RSGQSIVHSSGNTYLE | KVSNRFS | FQGSHIPWT | | | | |
| PR302341-67 | 29 | 75 | 121 | 173 | 197 | 230 | 281 | 345 | 389 | 453 |
| | DYFMK | VINPNNGDIFYNQKFKG | GTDRALFAY | RSGQSIVHSDANTYLE | KVSNRFS | FQGSHIPWT | | | | |
| PR302341-68 | 29 | 75 | 121 | 172 | 197 | 230 | 281 | 346 | 389 | 454 |
| | DYFMK | VINPNNGDIFYNQKFKG | GTDRALFAY | RSGQSIVHSEGNTYLE | KVSNRFS | FQGSHIPWT | | | | |
| PR302341-69 | 29 | 75 | 121 | 174 | 197 | 230 | 281 | 347 | 389 | 455 |
| | DYFMK | VINPNNGDIFYNQKFKG | GTDRALFAY | RSGQSIVHSSGNTYLE | KVSNRFS | FQGSHIPWT | | | | |
| PR302341-7 | 29 | 75 | 121 | 171 | 197 | 230 | 281 | 335 | 389 | 443 |
| | DYFMK | VINPNNGDIFYNQKFKG | GTDRALFAY | RSGQSIVHSDGNTYLE | KVSNRFS | FQGSHIPWT | | | | |
| PR302341-70 | 29 | 75 | 121 | 173 | 197 | 230 | 283 | 343 | 391 | 451 |
| | DYFMK | VINPNNGDIFYNQKFKG | GTDRALFAY | RSGQSIVHSDANTYLE | KVSNRFS | FQGSHIPWT | | | | |
| PR302341-71 | 29 | 75 | 121 | 172 | 197 | 230 | 283 | 342 | 391 | 450 |
| | DYFMK | VINPNNGDIFYNQKFKG | GTDRALFAY | RSGQSIVHSEGNTYLE | KVSNRFS | FQGSHIPWT | | | | |
| PR302341-72 | 29 | 75 | 121 | 174 | 197 | 230 | 283 | 344 | 391 | 452 |
| | DYFMK | VINPNNGDIFYNQKFKG | GTDRALFAY | RSGQSIVHSSGNTYLE | KVSNRFS | FQGSHIPWT | | | | |
| PR302341-8 | 29 | 75 | 121 | 171 | 197 | 230 | 281 | 340 | 389 | 448 |
| | DYFMK | VINPNNGDIFYNQKFKG | GTDRALFAY | RSGQSIVHSDGNTYLE | KVSNRFS | FQGSHIPWT | | | | |
| PR302341-9 | 29 | 75 | 121 | 171 | 197 | 230 | 281 | 341 | 389 | 449 |
| | DYFMK | VINPNNGDIFYNQKFKG | GTDRALFAY | RSGQSIVHSDGNTYLE | KVSNRFS | FQGSHIPWT | | | | |

In some aspects, provided herein are polynucleotides comprising a nucleotide sequence encoding an antibody or antigen-binding fragment thereof described herein or a domain thereof (e.g., a variable light chain region and/or variable heavy chain region) that binds to human CRH, and vectors, e.g., vectors comprising such polynucleotides for recombinant expression in host cells (e.g., E. coli and mammalian cells).

In some aspects, provided herein are polynucleotides comprising a nucleic acid molecule encoding a VH comprising an amino acid sequence at least 90%, 95%, or 99% identical to any one of SEQ ID NOs: 240-295; and/or a VL comprising an amino acid sequence at least 90%, 95%, or 99% identical to any one of SEQ ID NOs: 296-347. In some aspects, the polynucleotide encodes an antibody or antigen-binding fragment comprising a VH comprising an amino acid sequence of any one of SEQ ID NOs: 240-295; and/or a VL comprising an amino acid sequence of any one of SEQ ID NOs: 296-347.

In some aspects, the polynucleotide encodes an antibody or antigen-binding fragment thereof comprising a heavy chain comprising an amino acid sequence at least 90%, 95%, or 99% identical to any one of SEQ ID NOs: 348-403; and/or a light chain comprising an amino acid sequence least 90%, 95%, or 99% identical to any one of SEQ ID NOs: 404-455. In some aspects, the polynucleotide encodes an antibody or antigen-binding fragment thereof comprising a heavy chain comprising an amino acid sequence of any one of SEQ ID NOs: 348-403; and/or a light chain comprising an amino acid sequence of any one of SEQ ID NOs: 404-455.

A polynucleotide encoding an antibody or antigen-binding fragment thereof described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof described herein. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate antibodies or antigen-binding fragments thereof.

The polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

In some aspects, provided herein are vectors (e.g., expression vectors) comprising polynucleotides encoding the amino acid sequences disclosed herein. In some aspects, provided herein are vectors (e.g., expression vectors) comprising polynucleotides encoding the variable heavy chain (VH) complementarity determining regions (CDRs), the variable light chain (VL) CDRs, the variable heavy chain (VH), the variable light chain (VL), the heavy chain (HC), and the light chain (LC) disclosed herein.

Also provided herein are cells, e.g. host cells, comprising such vectors for recombinantly expressing an anti-CRH antibody or antigen-biding fragment thereof.

In some aspects, provided herein are vectors (e.g., expression vectors) comprising polynucleotides comprising nucleotide sequences encoding antibodies and antigen-binding fragments thereof that specifically bind to human CRH.

Methods which are well known to those skilled in the art can be used to construct expression vectors containing protein- or antibody or antigen-binding fragment thereof or domain thereof (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a protein or antibody or antigen-binding fragment thereof described herein, a heavy or light chain, a heavy or light chain variable domain, or a heavy or light chain CDR, operably linked to a promoter.

An expression vector can be transferred to a cell (e.g., host cell) by conventional techniques and the resulting cells can then be cultured by conventional techniques to produce a protein or an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of an antibody disclosed herein) or a domain thereof (e.g., the VH, the VL, the VH and the VL, the heavy chain, or the light chain of an antibody disclosed herein). Thus, provided herein are host cells containing a polynucleotide encoding a protein or an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of an antibody disclosed herein) or a domain thereof (e.g., the VH, the VL, the VH and the VL, the heavy chain, or the light chain of an antibody disclosed herein), operably linked to a promoter for expression of such sequences in the host cell. In some aspects, for the expression of double-chained antibodies or antigen-binding fragments thereof, vectors encoding both the heavy and light chains, individually, can be co-expressed in the host cell for expression of the entire immunoglobulin. In some aspects, a host cell contains a vector comprising a polynucleotide encoding both the heavy chain and light chain of an antibody described herein, or a domain thereof. In some aspects, a host cell contains two different vectors, a first vector comprising a polynucleotide encoding a heavy chain or a heavy chain variable region of an antibody or antigen-binding fragment thereof described herein, and a second vector comprising a polynucleotide encoding a light chain or a light chain variable region of an antibody described herein, or a domain thereof. In some aspects, a first host cell comprises a first vector comprising a polynucleotide encoding a heavy chain or a heavy chain variable region of an antibody or antigen-binding fragment thereof described herein, and a second host cell comprises a second vector comprising a polynucleotide encoding a light chain or a light chain variable region of an antibody or antigen-binding fragment thereof described herein. In some aspects, a heavy chain/heavy chain variable region expressed by a first cell associated with a light chain/light chain variable region of a second cell to form an antibody or antigen-binding fragment thereof described herein. In some aspects, provided herein is a population of host cells comprising such first host cell and such second host cell.

In some aspects, provided herein is a population of vectors comprising a first vector comprising a polynucleotide encoding a light chain/light chain variable region of an antibody or antigen-binding fragment thereof described herein, and a second vector comprising a polynucleotide encoding a heavy chain/heavy chain variable region of an antibody or antigen-binding fragment thereof described herein. Alternatively, a single vector can be used which encodes, and is capable of expressing, both heavy and light chain polypeptides.

A variety of host-expression vector systems can be utilized to express proteins and antibodies and antigen-binding fragments thereof described herein. Such host-expression systems represent vehicles by which the coding sequences of interest can be produced and subsequently purified, but also represent cells which can, when transformed or transfected with the appropriate nucleotide coding sequences, express a protein or an antibody or antigen-binding fragment thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing proteins or antibodies and antigen-binding fragments thereof described herein are CHO cells, for example CHO cells from the CHO GS System™ (Lonza). In some aspects, cells for expressing proteins or antibodies and antigen-binding fragments thereof described herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In some aspects, proteins or antibodies or antigen-binding fragments thereof described herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a protein or an antibody or antigen-binding fragment thereof described herein has been produced by recombinant expression, it can be purified by any method known in the art for purification of a protein or an immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the proteins or antibodies or antigen-binding fragments thereof described herein can be fused to heterologous polypeptide sequences described herein or otherwise known in the art to facilitate purification.

In some aspects, an antibody or antigen-binding fragment thereof described herein is isolated or purified. Generally, an isolated protein or antibody or antigen-binding fragment thereof is one that is substantially free of other proteins or antibodies or antigen-binding fragments thereof with different antigenic specificities than the isolated antibody or antigen-binding fragment thereof. For example, in some aspects, a preparation of an antibody or antigen-binding fragment thereof described herein is substantially free of cellular material and/or chemical precursors.

### 3. Treatment Methods

Treatment of CAH is based on normalization of hormone and steroid levels using a variety of medications from diagnosis in infancy through adulthood. Glucocorticoids are the current standard treatment in CAH and are used both to correct the endogenous cortisol deficiency and for reducing the elevated ACTH levels from the pituitary, which drives increased androgen production. Unlike the treatment of Addison's disease (adrenal insufficiency), in which physiological cortisol replacement is sufficient to reduce ACTH concentrations towards normal, the treatment of CAH must also reduce ACTH production in order to control the concomittent androgen excess. Thus, the goals of glucocorticoid treatment include cortisol replacement and suppression of ACTH to reduce accelerated skeletal maturation and subsequent short stature in both sexes, reduce virilization and menstrual disturbances in women, and to inhibit the development of testicular adrenal rest tumors in men. Mineralocorticoid replacement is needed to achieve normal plasma renin activity for maintenance of normal blood pressure, electrolyte balance, and volume status in those patients with the salt-wasting form of CAH.

The regimen of glucocorticoid treatment must support normal physiology and also ensure that sufficient cortisol is available during events that may elicit a strong stress response (e.g., intercurrent illness, exercise, hypotension). Careful monitoring is also necessary to avoid the development of iatrogenic Cushing syndrome due to glucocorticoid overtreatment in an effort to adequately suppress androgen production, or Addisonian syndrome due to undertreatment.

Overtreatment with mineralocorticoids may cause hypertension while undertreatment may lead to low blood pressure, salt loss, fatigue and increased requirements for glucocorticoids. Typical laboratory tests for monitoring treatment efficacy include measurement of plasma concentrations of 17-OHP, testosterone, free testosterone, androstenedione, an 11-oxygenated androgen, renin activity, electrolytes, or a combination thereof.

Adult patients with CAH have an increased prevalence of risk factors for cardiovascular disease including obesity, hypertension, and insulin resistance (see, e.g., Kim et al., Semin. Reprod. Med. 27 (4): 316-21 (2009)). A study of a large cohort of pediatric and adult CAH patients (n=244) demonstrated that patients are prescribed a variety of glucocorticoid treatment regimens yet frequently suffer from poor hormonal control and the aforementioned adverse outcomes (see, e.g., Finkielstain et al., J. Clin. Endocrinol Meted). 97 (12): 4429-38 (2012)).

Treatment of CAH includes efforts to normalize the cortisol deficiency with glucocorticoids (usually hydrocortisone in children but often more potent agents with narrow therapeutic indices, such as dexamethasone, in adults) and, if necessary for salt-wasting, mineralocorticoids (usually fludrocortisone). The glucocorticoid doses required to achieve sufficient suppression of excess androgens, however, are usually well above the normal physiologic dose used for cortisol replacement alone as in patients with Addison disease. This increased exposure to glucocorticoids can lead to iatrogenic Cushing syndrome, increased cardiovascular risk factors, glucose intolerance, and decreased bone mineral density in CAH patients (see, e.g., Elnecave et al., J. Pediatr. Endocrinol. Metab. 21:1155-62 (2008); King et al., J. Clin. Endocrinol. Metab. 91 (3): 8656-59 (2006); Migeon et al., Endocrinol. Metab. Clin, North Am. 30:193-206 (2001)). Recently, best practices for the clinical management of congenital adrenal hyperplasia were published in the Journal of Cliniced Endocrinology and Metabolism (Speiser, P. W., et al. J. Clin. Endocrinol Metab. November 2018, 103 (11): 1-46). This article is incorporated by reference in its entirety.

Corticotropin-releasing factor (CRF) (also known as corticotropin-releasing hormone (CRH)) has been found to produce profound alterations in endocrine, nervous, and immune system function. CRF is believed to be the major physiological regulator of the basal and stress-induced release of adrenocorticotropic hormone (“ACTH”), b-endorphin, and other proopiomelanocortin (“POMC”)-derived peptides from the anterior pituitary (see, e.g., Vale et al., Science 273:1394-1397, 1981). Secretion of CRF causes release of ACTH from corticotrophs in the anterior pituitary via binding to the CRFi receptor, a member of the class B family of G-protein coupled receptors.

The pituitary hormone ACTH, under the control of hypothalamic CRF, stimulates uptake of cholesterol and drives the synthesis of pregnenolone initiating steroidogenesis in the adrenal gland. The adrenal cortex is comprised of three zones, which produce distinct classes of hormones many of which are driven by ACTH mobilizing cholesterol through this pathway. Deficiencies in these enzymes as a result of mutation or deletion cause the substrate concentrations to increase.

In the most common form of CAH resulting from mutations or deletions in the 21-hydroxylase gene (CYP21A2), potent androgens are produced by the adrenal because of the accumulation of the steroid precursors, progesterone and 17-hydroxyprogesterone (17-OHP). Plasma levels of 17-OHP can reach 10-1000 times the normal concentration in these cases. These increases result in the overproduction of androgens, specifically testosterone, free testosterone, androstenedione, an 11-oxygenated androgen, or a combination thereof, causing virilization in females. In addition, 21-hydroxylase deficiency in CAH causes insufficient biosynthesis of glucocorticoids and mineralocorticoids, specifically cortisol and aldosterone. Cortisol is a critical negative feedback regulator of hypothalamic CRF secretion and pituitary ACTH release. The lack of glucocorticoid synthesis and release eliminates the restraint on the hypothalamus and pituitary, which causes ACTH levels to increase. The excessive ACTH stimulation causes hypertrophy of the zona fasciculata and zona reticularis resulting in adrenal hyperplasia.

Patients with CAH are unable to synthesize and secrete glucocorticoid and cortisol in normal amounts due to loss-of-function mutations in CYP21A2. Reduced glucocorticoid concentrations result in decreased negative feedback upon the pituitary and brain, causing an increase in blood ACTH concentration, which drives increased adrenal androgen secretion. The increase in ACTH concentration due to decreased negative feedback of cortisol requires CRH, without which cortisol does not rise, as shown in mice with genetic deficiency of CRH (Muglia et al., Journal of Clinical Investigation, 105 (9): 1269-1277, 2000). This indicates that blockade of CRH action should reduce the elevated blood concentration of ACTH in CAH patients, resulting in a reduction in androgen secretion in these patients.

In addition, a mutation or deletion in the CYP11B1 gene can be associated with CAH. The CYP11B1 gene encodes the enzyme, 11-beta-hydroxylase. This enzyme is found in the adrenal glands, where it helps produce cortisol and corticosterone.

A mutation or deletion in the HSD3B2 gene can also be associated with CAH. The HSD3B2 gene encodes the enzyme, 3-beta-hydroxysteroid dehydrogenase. 3-beta-hydroxysteroid dehydrogenase is found in the gonads and adrenal glands and is involved in the production of many hormones, including cortisol, aldosterone, androgens and estrogen.

In some aspects of any of the methods provided herein, the CAH is associated with one or more mutations or deletions in a gene selected from the group consisting of CYP21A2, CYP11B1 and HSD3B2. In some aspects of any of the methods provided herein, the CAH is associated with a mutation or deletion in CYP21A2. In some aspects of any of the methods provided herein, the CAH is associated with a mutation or deletion in CYP11B1. In some aspects of any of the methods provided herein, the CAH is associated with a mutation or deletion in HSD3B2.

The present disclosure relates to methods of treating congenital adrenal hyperplasia (CAH). The methods include administering to a subject a therapeutically effective amount of an antibody or antigen-binding fragment thereof disclosed herein. In some aspects, the method includes administering to a subject a therapeutically effective amount of a pharmaceutical composition of the present disclosure that contains an antibody or antigen-binding fragment thereof disclosed herein.

Provided herein is a method of treating congenital adrenal hyperplasia (CAH) comprising administering an antibody or antigen-binding fragment thereof disclosed herein to normalize or partially normalize levels of biomarkers associated with congenital adrenal hyperplasia. In some aspects, normalizing or partially normalizing levels of biomarkers comprises reducing levels of elevated biomarkers or increasing levels of depressed biomarkers as compared to subject without CAH.

Provided herein is a method of treating congenital adrenal hyperplasia in a subject in need thereof comprising administering an antibody or antigen-binding fragment thereof disclosed herein, in an amount sufficient to reduce the level of one or more biomarkers associated with congenital adrenal hyperplasia. In some aspects, the biomarkers are selected from (a) 17-hydroxyprogesterone (17-OHP); (b) adrenocorticotropic hormone (ACTH); and (c) androstenedione in the subject.

In some aspects, the reduction in level of any of the biomarkers (e.g., any of 17-OHP, ACTH, and androstenedione) is determined by comparing the level of the biomarker as measured on a day prior to administering an antibody or antigen-binding fragment thereof disclosed herein and the level of the biomarker as measured on the day after administering an antibody or antigen-binding fragment thereof disclosed herein. A day prior to administering an antibody or antigen-binding fragment thereof disclosed herein applies to a subject that has not previously been administered an antibody or antigen-binding fragment thereof disclosed herein within at least the past 24 hours.

In some aspects of the methods provided herein, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some aspects, the level of 17-hydroxyprogesterone is reduced by at least 25%. In some aspects, the level of 17-hydroxyprogesterone is reduced by at least 50%. In some aspects of the methods provided herein, the level of 17-hydroxyprogesterone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some aspects of the methods provided herein, the level of adrenocorticotropic hormone is reduced by at least 10%, at least 15%, at least 20%, at least 25%, at least 30%, at least 35%, at least 40%, at least 50%, at least 55% or at least 60% from pre-administration levels. In some aspects, the level of adrenocorticotropic hormone is reduced by at least 25%. In some aspects, the level of adrenocorticotropic hormone is reduced by at least 40%. In some aspects, the level of adrenocorticotropic hormone is reduced by at least 50%. In some aspects of the methods provided herein, the level of adrenocorticotropic hormone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some aspects, the level of adrenocorticotropic hormone is reduced to a level within the range of adrenocorticotropic hormone expected for a subject without CAH.

In some aspects of the methods provided herein, the level of androstenedione is reduced by at least 10%, at least 15%, at least 20%, at least 25%, at least 30%, at least 35%, at least 40%, at least 50%, at least 55% or at least 60% from pre-administration levels. In some aspects, the level of androstenedione is reduced by at least 25%. In some aspects, the level of androstenedione is reduced by at least 30%. In some aspects, the level of androstenedione is reduced by at least 50%. In some aspects of the methods provided herein, the level of androstenedione is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some aspects, the level of androstenedione is reduced to a level within the range of androstenedione expected for a subject without CAH, i.e., less than 200 ng/dL.

Also provided herein is a method for reducing the severity of one or more symptoms selected from hirsutism, precocious puberty, fertility problems, acne, and growth impairment in a subject having classic congenital adrenal hyperplasia, comprising administering an antibody or antigen-binding fragment thereof disclosed herein, in an amount sufficient to reduce one or more biomarker of CAH in a subject, e.g. reduce the androstenedione in the subject. Growth impairment can refer to, e.g., accelerated height velocity, accelerated weight velocity, and/or accelerated bone age.

Provided herein is a method for reducing the level of one or more biomarkers of congenital adrenal hyperplasia in a subject having congenital adrenal hyperplasia comprising administering to the subject an antibody or antigen-binding fragment thereof disclosed herein. In some aspects, the one or more biomarkers of congenital adrenal hyperplasia are selected from (a) 17-hydroxyprogesterone (17-OHP); (b) adrenocorticotropic hormone (ACTH); and (c) androstenedione.

Therefore, provided herein is a method of treating congenital adrenal hyperplasia in a subject comprising:
(a) measuring the level of one or more biomarkers selected from (a) 17-hydroxyprogesterone (17-OHP); (b) adrenocorticotropic hormone (ACTH); and (c) androstenedione in a biological sample obtained from the subject;
(b) analyzing the level of the one or more biomarkers to determine if the level of the one or more biomarkers is elevated compared to a healthy subject not having congenital adrenal hyperplasia; and
(c) administering to the subject an antibody or antigen-binding fragment thereof disclosed herein, if the subject is determined to have elevated levels of the one or more biomarkers.

Also provided herein is a method for reducing the dosage of corticosteroid administered to a subject having congenital adrenal hyperplasia for controlling congenital adrenal hyperplasia comprising administering to the subject an antibody or antigen-binding fragment thereof disclosed herein. In some aspects, the corticosteroid is a glucocorticoid. In some aspects, the antibody or antigen-binding fragment thereof and corticosteroid are administered concurrently, either admixed as a single composition in a pharmaceutically acceptable formulation for concurrent administration, or concurrently as separate compositions with the antibody or antigen-binding fragment thereof, and the corticosteroid in pharmaceutically acceptable formulations. In some aspects, the formulations are administered sequentially, and in any order.

Also provided herein is a method of reducing the severity of one or more side effects of glucocorticoid treatment in a subject having congenital adrenal hyperplasia comprising administering to the subject an antibody or antigen-binding fragment thereof disclosed herein. The long-term effects of glucocorticoid treatment are well documented in the art (see, e.g., Gray, M. et al. (2016): Long-term effect of glucocorticoids, Expert Opinion on Drug Safety. DOI: 10.1517/14740338.2016.1140743). Such side effects are associated with every biological system, e.g., musculoskeletal (e.g., osteoporosis, avascular necrosis of bone, and myopathy), endocrine and metabolic (e.g., hyperglycemia, diabetes mellitus, dyslipidemia, weight gain, Cushing syndrome, Cushingoid features, growth suppression, adrenal suppression), gastrointestinal (e.g., gastritis, peptic ulcer, gastrointestinal bleeding, visceral perforation, hepatic steatosis, pancreatitis), cardiovascular (e.g., hypertension, coronary heart disease, ischemic heart disease, heart failure), dermatologic (e.g., dermatoprosis, skin atrophy, ecchymosis, purpura, erosions, striae, delayed wound healing, easy bruising, acne, hirsutism, and hair loss), neuropsychiatric (e.g., mood changes, depression, euphoria, mood lability, irritability, akathisia, anxiety, cognitive impairment, psychosis, dementia, and delirium), ophthalmologic (e.g., cataract, glaucoma, ptosis, mydriasis, opportunistic ocular infections, and central serous chorioretinopathy), and immunologic (e.g., suppression of cell-mediated immunity, predisposition to infections, and reactivation of latent infections).

Accordingly, in some aspects, the side effects of glucocorticoid treatment are selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections, and any combination thereof.

### 4. Glucocorticoids

Glucocorticoids are a class of corticosteroids, which are a class of steroid hormones. Glucocorticoids are corticosteroids that bind to the glucocorticoid receptor that is present in almost every vertebrate animal cell. In some aspects, the subject is concurrently receiving a dose of a glucocorticoid. In some aspects, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate. In some aspects, the glucocorticoid is cortisol (hydrocortisone). In some aspects, the glucocorticoid is cortisone. In some aspects, the glucocorticoid is prednisone. In some aspects, the glucocorticoid is dexamethasone.

In some aspects, the glucocorticoid dose is measured in hydrocortisone equivalents. In some aspects, the glucocorticoid dose is measured as a multiple of the upper limit of normal of physiologic dosing in hydrocortisone equivalents. Any glucocorticoid can be given in a dose that provides approximately the same glucocorticoid effects as normal cortisol production; this is referred to as physiologic, replacement, or maintenance dosing.

In some aspects, the glucocorticoid dose is a physiologic dose as measured after a time period of administration of an antibody or antigen-binding fragment thereof disclosed herein. In some aspects, the glucocorticoid dose concurrently given to the subject is a normal physiological dose of hydrocortisone equivalents. In some aspects, the glucocorticoid dose concurrently given to the subject is determined after a time period of administration of an antibody or antigen-binding fragment thereof disclosed herein. In some aspects, the glucocorticoid dose concurrently given to the subject is determined after a time period of administration of an antibody or antigen-binding fragment thereof disclosed herein.

In some aspects, the glucocorticoid dose concurrently given to the subject is at the upper limit of normal of a normal physiological dose of hydrocortisone equivalents. In some aspects, the glucocorticoid dose concurrently given to the subject is determined after a time period of administration of an antibody or antigen-binding fragment thereof disclosed herein.

In some aspects, the subject exhibits a decrease in glucocorticoid burden after a time period of administration of an antibody or antigen-binding fragment thereof disclosed herein, wherein the decrease in glucocorticoid burden is relative to the glucocorticoid burden prior to administration of an antibody or antigen-binding fragment thereof disclosed herein. In some aspects, one or more symptoms selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor and fertility, is improved after a time period of administration of an antibody or antigen-binding fragment thereof disclosed herein, wherein the improvement in the one or more symptoms is relative to the status of the one or more symptoms prior to administration of an antibody or antigen-binding fragment thereof disclosed herein.

In some aspects, the quality of life as measured by the EuroQol 5 Dimensions 5 Levels (EQ-5D-5L) in the subject is improved after a time period of administration of an antibody or antigen-binding fragment thereof disclosed herein, wherein the improvement in the EuroQol 5 Dimensions 5 Levels (EQ-5D-5L) is relative to the EuroQol 5 Dimensions 5 Levels (EQ-5D-5L) results prior to administration of an antibody or antigen-binding fragment thereof disclosed herein.

In some aspects, fatigue is reduced in the subject after a time period of administration of an antibody or antigen-binding fragment thereof disclosed herein, wherein the reduction in fatigue is relative to the fatigue prior to administration of an antibody or antigen-binding fragment thereof disclosed herein.

In some aspects, sleep is increased in the subject after a time period of administration of an antibody or antigen-binding fragment thereof disclosed herein, wherein the increase in sleep is relative to the sleep prior to administration of an antibody or antigen-binding fragment thereof disclosed herein.

In some aspects, insulin resistance is reduced in the subject after a time period of administration of an antibody or antigen-binding fragment thereof disclosed herein, wherein the reduction of insulin resistance is relative to the insulin resistance prior to administration of an antibody or antigen-binding fragment thereof disclosed herein.

In some aspects, glucose tolerance is reduced in the subject after a time period of administration of an antibody or antigen-binding fragment thereof disclosed herein, wherein the reduction in glucose tolerance is relative to the glucose tolerance prior to administration of an antibody or antigen-binding fragment thereof disclosed herein.

In some aspects, glucose control is increased in the subject after a time period of administration of an antibody or antigen-binding fragment thereof disclosed herein, wherein the increase in glucose control is relative to the glucose control prior to administration of an antibody or antigen-binding fragment thereof disclosed herein.

In some aspects, lipid levels reflecting dyslipidemia are reduced in the subject after a time period of administration of an antibody or antigen-binding fragment thereof disclosed herein, wherein the reduction in lipid levels is relative to the lipid levels prior to administration of an antibody or antigen-binding fragment thereof disclosed herein.

In some aspects, lipid levels reflecting hyperlipidemia are reduced in the subject after a time period of administration of an antibody or antigen-binding fragment thereof disclosed herein, wherein the reduction in lipid levels is relative to the lipid levels prior to administration of an antibody or antigen-binding fragment thereof disclosed herein.

In some aspects, bone mineral density is increased in the subject after a time period of administration of an antibody or antigen-binding fragment thereof disclosed herein, wherein the increase in bone mineral density is relative to the bone mineral density prior to administration of an antibody or antigen-binding fragment thereof disclosed herein.

In some aspects, bone turnover is increased in the subject after a time period of administration of an antibody or antigen-binding fragment thereof disclosed herein, wherein the increase in bone turnover is relative to the bone turnover prior to administration of an antibody or antigen-binding fragment thereof disclosed herein.

In some aspects, fat mass is decreased in the subject after a time period of administration of an antibody or antigen-binding fragment thereof disclosed herein, wherein the decrease in fat mass is relative to the fat mass prior to administration of an antibody or antigen-binding fragment thereof disclosed herein.

In some aspects, body weight is decreased in the subject after a time period of administration of an antibody or antigen-binding fragment thereof disclosed herein, wherein the decrease in body weight is relative to the body weight prior to administration of an antibody or antigen-binding fragment thereof disclosed herein.

In some aspects, central obesity is decreased in the subject after a time period of administration of an antibody or antigen-binding fragment thereof disclosed herein, wherein the decrease in central obesity is relative to the central obesity prior to administration of an antibody or antigen-binding fragment thereof disclosed herein.

Reduced glucocorticoid burden in growing children may result in increased statural growth and increased final adult height.

In some aspects, blood pressure is decreased in the subject after a time period of administration of an antibody or antigen-binding fragment thereof disclosed herein, wherein the decrease in blood pressure is relative to the blood pressure prior to administration of an antibody or antigen-binding fragment thereof disclosed herein.

In some aspects, the severity of hirsutism is decreased in the subject after a time period of administration of an antibody or antigen-binding fragment thereof disclosed herein, wherein the decrease in the severity of hirsutism is relative to the severity of hirsutism prior to administration of an antibody or antigen-binding fragment thereof disclosed herein.

In some aspects, menstrual cyclicity is increased in the subject after a time period of administration of an antibody or antigen-binding fragment thereof disclosed herein, wherein the increase in menstrual cyclicity is relative to the menstrual cyclicity prior to administration of an antibody or antigen-binding fragment thereof disclosed herein.

In some aspects, control of testicular adrenal rest tumor is increased in the subject after a time period of administration of an antibody or antigen-binding fragment thereof disclosed herein, wherein the increase in control of testicular adrenal rest tumor is relative to the control of testicular adrenal rest tumor prior to administration of an antibody or antigen-binding fragment thereof disclosed herein.

Antibody administration to children, by reducing androgen levels, may cause reduced maturation of the skeleton, resulting in increased final adult height.

In some aspects, fertility is increased in the subject after a time period of administration of an antibody or antigen-binding fragment thereof disclosed herein, wherein the increase in fertility is relative to the fertility prior to administration of an antibody or antigen-binding fragment thereof disclosed herein.

In some aspects, gonadotropin levels are increased in the subject after a time period of administration of an antibody or antigen-binding fragment thereof disclosed herein, wherein the increase in gonadotropin levels is relative to the gonadotropin levels prior to administration of an antibody or antigen-binding fragment thereof disclosed herein.

In some aspects, progesterone levels are increased in the subject after a time period of administration of an antibody or antigen-binding fragment thereof disclosed herein, wherein the increase in progesterone levels is relative to the progesterone levels prior to administration of an antibody or antigen-binding fragment thereof disclosed herein.

In some aspects, semen levels are increased in the subject after a time period of administration of an antibody or antigen-binding fragment thereof disclosed herein, wherein the increase in semen levels is relative to the semen levels prior to administration of an antibody or antigen-binding fragment thereof disclosed herein.

In some aspects, LH (luteinizing hormone) levels are increased in the subject after a time period of administration of an antibody or antigen-binding fragment thereof disclosed herein, wherein the increase in LH levels are relative to the LH levels prior to administration of an antibody or antigen-binding fragment thereof disclosed herein.

In some aspects, the subject is an adult subject. In some aspects, the subject is over eighteen years old. In some aspects, the subject is female. In some aspects, the subject is male. In some aspects, the subject is an adolescent under 20 years old, or a child under 13 years old.

### 5. Detection Methods

It is sometimes desirable to detect the presence or measure the amount of CRH in a sample. In this regard, the disclosure provides a method of using an antibody or antigen-binding fragment thereof described herein to measure the amount of CRH in a sample. To determine a measurement of CRH, a biological sample from a mammalian subject is contacted with an anti-CRH antibody (or antigen binding fragment thereof) described herein for a time sufficient to allow immunocomplexes to form. Immunocomplexes formed between the antibody and CRH in the sample are then detected. The amount of CRH in the biological sample is optionally quantitated by measuring the amount of the immunocomplex formed between the antibody and the CRH. For example, the antibody can be quantitatively measured if it has a detectable label, or a secondary antibody can be used to quantify the immunocomplex.

In some aspects, the biological sample comprises a tissue sample, a cell sample, or a biological fluid sample, such as blood, saliva, serum, or plasma.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats can readily be adapted to employ the antibodies (or fragments thereof) of the present disclosure. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, FL Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test sample used in the above-described method will vary based on the assay format, nature of the detection method and the tissues, cells or fluids used as the sample to be assayed.

The assay described herein may be useful in, e.g., evaluating the efficacy of a particular therapeutic treatment regime in animal studies, in clinical trials, or in monitoring the treatment of an individual patient.

In some aspects, the CRH or the anti-CRH antibody (or antigen-binding fragment thereof) is attached to a solid support, and binding is detected by detecting a complex between the CRH and the antibody (or antigen-binding fragment thereof) on the solid support. The antibody (or antigen-binding fragment thereof) optionally comprises a detectable label and binding is detected by detecting the label in the CRH-antibody complex.

Detection of the presence or absence of a CRH-antibody complex be achieved using any method known in the art. For example, the transcript resulting from a reporter gene transcription assay of a CRH peptide interacting with a target molecule (e.g., antibody) typically encodes a directly or indirectly detectable product (e.g., β-galactosidase activity and luciferase activity). For cell free binding assays, one of the components usually includes, or is coupled to, a detectable label. A wide variety of labels can be used, such as those that provide direct detection (such as radioactivity, luminescence, optical or electron density) or indirect detection (such as epitope tag such as the FLAG epitope, enzyme tag such as horseradish peroxidase). The label can be bound to the antibody, or incorporated into the structure of the antibody.

A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or streptavidin-biotin conjugates. Methods for detecting the labels are well known in the art.

### 6. Kits

Once a pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the disclosure may each contain both a first container having a dried protein and a second container having an aqueous formulation. In certain aspects, kits containing single and multi-chambered pre-filled syringes (e.g., liquid syringes and lyosyringes) are provided.

### EXAMPLES

The following Examples are provided to further illustrate aspects of the disclosure, and are not meant to constrain the disclosure to any particular application or theory of operation.

### Example 1

### Generation of CRH Knockout Mouse on H2L2 Harbour Mice® Background

Corticotropin-releasing hormone (CRH) knockout (KO) mice were generated on a H2L2 Harbour Mice® background, using the standard transgenesis method described in “Manipulating the Mouse Embryo,” a laboratory manual by Nagy et al. Cold Spring Harbor Laboratory Press. Generation of CRH KO H2L2 mice was done under animal license ADV101002016512, animal protocol 16-512-11, approved by the Dutch Central Authority for Scientific Procedures on Animals of (CCD) and EDC animal facility.

In short, super-ovulated H2L2 Harbour Mice® females and males were used for mating. Collected fertilized eggs were used for microinjection of two guide RNAs (gRNAs) at 20 ng/microliter in a microinjection buffer of 5 mM tris(hydroxymethyl)aminomethane (TRIS), 0.1 mM ethylenediaminetetraacetic acid (EDTA) in ultrapure water, adjusted to pH7.4: gRNA1 Mm.Cas9.CRH.1.AA and gRNA2 Mm.Cas9.CRH.1.AB and the CAS9 protein. The following gRNA1 sequences were used:

gRNA1 = Sequence-Mm.Cas9CHR.1.AA:
5′-AltR1/rArArC rUrCrC rArCrG rCrCrC rCrUrC rArCrC rGrCrG rUrUrU
rUrArG rArGrC rUrArU rGrCrU/altR2/-3′
gRNA2 = Mm.Cas9.CRH.1.AB:
5′-AltR1/rUrCrA rCrCrC rArUrG rCrGrG rArUrC rArGrArArCrG rUrUrU
rUrArG rArGrC rUrArU rGrCrU/altR2/-3′

Guide RNAs and CAS9 were obtained from Integrated DNA Technologies (IDT).

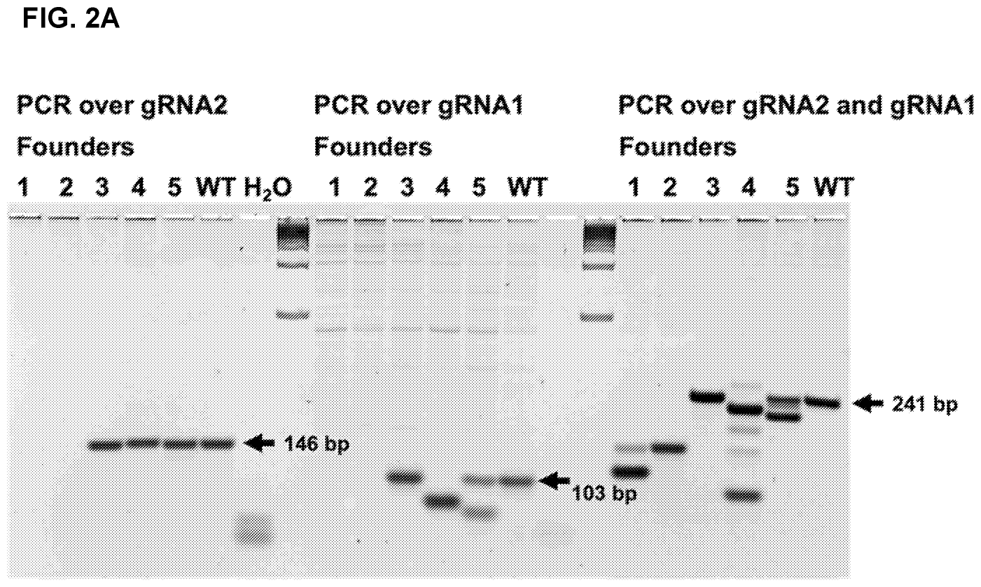

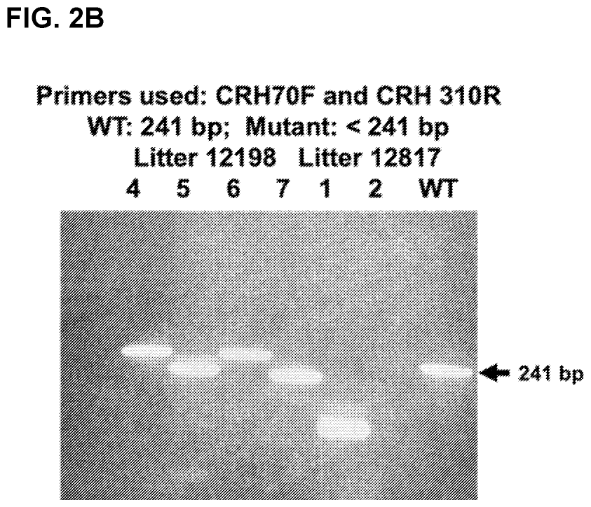

Injected eggs were transferred into foster mothers (B6CBAF1). Knockout pups were identified from toe DNA by amplification of DNA spanning the region over the gRNAs. Polymerase chain reaction (PCR) fragments smaller than these expected sizes indicated deletion in founder mice. PCR Primers CRHbp70F and CRHbp215R flanking the gRNA1 were used to amplify an expected size of 146 bp from wildtype H2L2 Harbour Mice® (FIG. 2A, left panel). PCR primers CRHbp208F and CRHbp310R were used to amplify an expected size of 103 bp from wildtype H2L2 Harbour Mice® (FIG. 2A, middle panel). PCR Primers CRHbp70F and CRHbp310R resulted in PCR fragment size of 241 bp (FIG. 2A, right panel). PCR product smaller than these expected sizes indicated mutations. Founder Mice 1, 2, 4 and 5 were identified as carrying mutations. PCR products deviating from the expected size for the wild type were further analyzed by cutting the PCR amplification products from the agarose gel, isolation of DNA and sequencing, using the same primers that were used for amplification. Founder Mouse 5 from litter 12198 in FIG. 2B carried the 11 bp deletion (5′-CAGCCGGTTCT-3′) (SEQ ID NO: 465), which was used for subsequent breedings.

### Rescue of CRH Knockout H2L2 Harbour Mice®

Rescue and immunization of CRH knockout mice were done under animal license ADV101002016512, animal working protocol 16-512-19. In short, homozygous CRH knockout H2L2 Harbour Mice® males and females were set in cages one to one. By observation of the copulation plug (0.5 d.p.c), females were taken out of the cage and separated. Ten days later, cortisol (30 μg/ml) was given in the drinking water (dark bottles) ad libitum to the pregnant females. This resulted in the birth of live knock out progenies that were used for immunization.

Immunization of H2L2 Harbour Mice® with CRH Knockout

Ten homozygous CRH knockout H2L2 Harbour Mice® of both genders at the age of about 8 weeks were immunized with subcutaneous (SC) and intraperitoneal (IP) injections of CRH-KLH conjugates on days 0, 14, 28, 42 and 50 in a maximum volume of 100 μl.

The following conjugates were used:

CRH-KLH:
(SEQ ID NO: 467)
SEEPPISLDLTFHLLREVLEMARAEQLAQQAHSNRKLMEII-
KLH(C-Terminus)(GenScript)

CRH-KLH conjugates were mixed with adjuvant Stimmune for the first injection and adjuvant Ribi for the following boosts. The last boost was IP. Blood samples were collected after the 4th injection and at the end of successful immunization experiment. Three to five days after the last IP injection, mice were euthanized, and spleens and lymph nodes were used in the fusion experiment with myeloma fusion partner for hybridoma production, according to standard protocol. Sera were screened for anti-CRH antibody titers and hybridomas later by the enzyme-linked immunosorbent assay (ELISA) with biotinylated CRH captured on streptavidin (SA)-coated plates. A detailed protocol follows.

### CRH ELISA

ELISA plates were coated with 5 μg/ml streptavidin in phosphate buffered saline (PBS) (Sigma 189730-1 mg) at 4° C. overnight or at room temperature (RT) for 2 hours. 50 μl/well was used for 96-well plates, or less for 384-well plates. After incubation, the strepavidin solution was removed and biotinylated peptide was added (ThermoFisher, biotin is at the N terminus of the peptide). Following incubation for 30 min at RT, plates were washed with 1× phosphate buffered saline (PBS)/0.1% Tween-20 and blocked with 1% milk/1% bovine serum albumin (BSA) in washing solution. Following a wash in 3×PBS/0.1% Tween-20, 50 μl of antibody was added in blocking buffer and incubated at RT for 2 hours. Plates were then washed in 3×PBS/0.1% Tween-20, and 50 μl of anti-rat IgG-HRP (horseradish peroxidase) at a 1:2,000 dilution was added and incubated at RT for 2 hours. Following a wash in 3×PBS/0.1% Tween-20, 50 μl peroxidase substrate (POD or equivalent) was added and absorbance was measured at 450 nm.

### Immunization of Balb/c Wildtype Mice

Balb/c wildtype mice were immunized with CRH-KLH protein to generate anti-CRH antibodies. The CRH and KLH conjugates are either N-terminally conjugated CRH (Peptide 1: KLH-CRH) or C-terminally conjugated CRH (Peptide 2: CRH-KLH), via an extra Cysteine (C) at either N-terminus or C-terminus of CRH, respectively. 50 μg of protein was administered to each mouse for the first prime via IP with complete Freund's adjuvant (CFA, Sigma, Cat #F5881) and 25 μg for the following boosts via IP with Ribi (Sigma S6322). Boosts were conducted bi-weekly for 4 times. At designated time points, mouse serum was sampled and titrated by indirect ELISA to test binding against human CRH.

Mice with high serum titers and specific immune responses against human CRH were selected and injected with 25 μg of protein in PBS to boost immunity. Three days later, the mice were sacrificed, and their spleen cells and lymph node cells were collected.

### Preparation of CRH-KLH Conjugate

The KLH carrier protein (Sigma, H7017-50 MG) was weighed and dialyzed against 0.1M sodium phosphate buffer, pH7.5. Sulfo-MBS (BBI, C100314-0050) solution was added to the KLH carrier protein solution at a ratio of 1:10. One hour after incubation at room temperature, the reaction mixture was applied to a G-50 desalting column (Sigma, G50150-10G) pre-equilibrated with conjugation buffer (0.1M sodium phosphate buffer, pH6.5, with 1 mM EDTA). Fractions corresponding to the first protein peak based on A280 nm were collected. KLH carrier protein-MBS and CRH peptide with an extra cysteine either at N-terminus or at C-terminus were mixed and incubated at room temperature for 2-4 hours. The reaction was terminated by adding 2-mercaptoethanol (Sigma, M3148-25ML) to a final concentration of 10 mM and incubated at room temperature for more than 30 minutes. The CRH-KLH conjugate was purified through a Sephadex G-25 desalting column to separate the unconjugated peptide from the CRH-KLH conjugate. Fractions corresponding to the first protein peak were collected. The protein concentration of CRH-KLH conjugate was determined by NanoPhotometer.

The following peptides were used for immunizing Balb/c wildtype mice:

Peptide 1 (KLH-CRH) (SEQ ID NO: 468):
KLH-C-SEEPPISLDLTFHLLREVLEMARAEQLAQQAHSNRKLMEII-
CONH2
Peptide 2 (CRH-KLH) (SEQ ID NO: 469):
SEEPPISLDLTFHLLREVLEMARAEQLAQQAHSNRKLMEII-C-KLH

And, the following peptides were used for ELISA:

Peptide 3 (Biotin-CRH) (SEQ ID NO: 470):
Biotin-K-SEEPPISLDLTFHLLREVLEMARAEQLAQQAHSNRKLMEII-
CONH2
Peptide 4 (CRH-Biotin) (SEQ ID NO: 471):
SEEPPISLDLTFHLLREVLEMARAEQLAQQAHSNRKLMEII-K-Biotin
Biotin-CRH (SEQ ID NO: 472):
Biotin-SEEPPISLDLTFHLLREVLEMARAEQLAQQAHSNRKLMEII-
COOH(ThermoFisher)

### Single B Cell Screening Based on the Beacon® Optofluidic System

A nanofluidic optoelectronic B lymphocyte antibody screening technique (NanOBlast) was used that was built around Beacon™, a commercially available (Berkeley Lights), integrated culture and imaging platform.

The NanOBlast workflow begins with generating antigen-specific antibody secreting cells (ASCs) via in vivo immunization. Selectively enriched, antigen-experienced murine ASCs were harvested from spleen and lymph nodes. ASCs are microfluidically imported into the chip and sequestered into individual nanopens for screening via OptoElectro Positioning (OEP). ASCs that secrete antigen-specific IgG are detected using a bead based, color fluorescent binding assay that produces a characteristic fluorescent bloom. Individual cells of interest are then un-penned using OEP and exported from the chip directly into 96-well plates containing cell lysis buffer. Antibody heavy chain variable domain (VH) and light chain variable domain (VL) sequences are recovered using single cell rapid amplification of cDNA ends (RACE), cloned and recombinantly expressed as canonical antibodies using standard methods. The recombinant antibodies are used for binding confirmation and eventual validation in relevant downstream assays.

### Antibody Production and Purification

Recombinant plasmids encoding target antibodies were transiently co-transfected into HEK293-6E or ExpiCHO cell cultures using polyethylenimine (PEI) (Polyscience, 24885). One day before transfection, cells were seeded in Corning Erlenmeyer Flasks. On the day of transfection, recombinant plasmids encoding target protein and transfection reagent were mixed at an optimal ratio (Heavy Chain:Light Chain=2:3) and then added to the seeded flasks for transfection. Cell culture supernatants were collected on day 6 and used for purification. Cell culture broth was centrifuged followed by filtration. Filtered cell culture supernatant was loaded onto an affinity purification column. Following washing and elution, the eluted fractions were pooled and the buffer was exchanged to storage buffer. The purified protein was analyzed by sodium dodecyl sulfate-polyacrylamide gel electrophoresis (SDS-PAGE) and size exclusion chromatography-high performance liquid chromatography (SEC-HPLC) analysis to determine molecular weight and purity. The concentration was determined by absorbance of light at 280 nm.

### Antibody Engineering

Heavy chain variable domain (VH) and light chain variable domain (VL) sequences of anti-CRH chimeric antibodies were further optimized by humanization and posttranslational modification (PTM) removal procedures. In the humanization procedure, VH and VL sequences were first aligned to the closest human germline sequence by algorithms, e.g., NCBI/Ig-BLAST, and then positions in the framework regions were reverted to human germline sequence. All key backmutation positions were scanned by Chothia numbering. If there was a mismatch, it was replaced with the mouse counterpart residue. Antibodies composed of sequence variants after humanization were then recombinantly produced by standard molecular biology techniques.

For PTM removal, VH and VL sequences were scanned for the presence of PTM motifs, e.g., isomerization motifs (e.g., DG). “Hotspot” residues (e.g., D or G in a DG motif) were mutated to either the counterpart residue in the germline sequence or another residue with similar biophysical properties. Antibodies containing sequence variants after PTM removal were then recombinantly produced by standard molecular biology techniques.

### Binding of Anti-CRH Antibodies to CRH or Human UCN1 by ELISA

Streptavidin (Thermo, Cat: 21125) was diluted in PBS to a concentration of 2 μg/ml. 100 μl of diluted streptavidin was added per well to ELISA microplates, and the plates were incubated overnight at 4° C. Plates were blocked with ELISA blocking solution (containing 2% w/v BSA, 0.05% (v/v) Tween-20, pH 7.4 PBS buffer) at 37° C. for 1 hour, and then incubated with 0.5 μg/mL CRH (NT biotin), 0.5 μg/mL CRH (CT biotin), 1 μg/mL human UCN1 (NT biotin), or 1 μg/mL human UCN1 (CT biotin) for 1 hour at 37° C. Plates were then washed and incubated with diluted anti-CRH antibody (Ab) at 15 μg/ml (100 nM, 10 diluted, 8 points) for 1 hour at 37° C. Subsequently, plates were washed and incubated with HRP-conjugated goat anti-human IgG (H+L) antibody (Jackson, Cat: 109-035-088) at 37° C. for 1 hour. 100 μL of 3,3′,5,5′-tetramethylbenzidine (TMB) substrate (Biopanda, Cat: TMB-S-003) was added, and the plates were incubated at room temperature for 15 minutes. 100 μL of ELISA stopping solution (Solarbio, Cat #: C1058) was added to terminate the reaction, and the optical density at 450 nm (OD450 nm) was determined by an ELISA plate reader (Molecular Devices, Spectra max 384 plus).

### Preparation of Plasmids and Cell Lines

The CHO-K1 (Cat #CCL-61) cell line was obtained from the American Type Culture Collection (ATCC), and pGL4.29/luc2P/CRE/Hygro (Cat #E8471) and Bio-Glo Luciferase Assay System (Cat #G7940) were obtained from Promega. Plasmids pcDNA3.1-human CRHR1 (Clone ID: OHu27188C), pcDNA3.1-human CRHR2 (Clone ID: OHu10803), pcDNA3.1-mouse CRHR1 (Clone ID: OMu00753), and pcDNA3.1-mouse CRHR2 (Clone ID: OMu23246) were synthesized in GenScript®, which are G418-resistant. Human CRH peptide was obtained from Peptide International (Cat #4136-s). Forskolin (Cat #1099) and human CRHR1 antagonist small molecule (Antalarmin, Cat #2778) were obtained from Tocris. Anti-human CRHR1 antibody (Cat #MAB3930) was obtained from R&D System. An electroporation instrument (Electro Cell Manipulator Cat #ECM630) and electroporation Cuvettes plus (2 mm Gap Cuvette, Cat #45-0125) were used from BTX. Electroporation buffer (Ingenio Solution, Cat #MIR50111) was obtained from Mirus, and sheep anti-human CRH polyclonal antibody was obtained from the Salk Institute.

### Generation of CHO-CRE-Luciferase-Human CRHR1,-Human CRHR2,-Mouse CRHR1, and -Mouse CRHR2 Reporter Cell Lines

To generate a CHO-CRE-Luciferase Reporter stable cell line, a mixture of 2 million CHO-K1 cells with 10 μg of pGL4.29/luc2P/CRE/Hygro plasmid in 100 μl of Ingenio solution was transferred into electroporation cuvettes and subjected to 1 or 2 pulses using an Electro Cell Manipulator (950 uF and 120V). The processed cells were transferred into a 6-well plate with 2 mL of Dulbecco's Modified Eagle's Medium (DMEM)/F12 with 10% fetal bovine serum (FBS). After 24 hours, selection antibiotic hygromycin at 500 μg/ml was added to the cells. This selection took about 10-14 days. Using matrix dilution, the pool of CHO-CRE cells was subcloned into 96-well plates. Individual stable clones were harvested when selected colonies became obvious. Positive clones were validated using a series of diluted forskolin. Validated positive clones were frozen for the future use.

To create CHO-CRE-hCRHR1,-hCRHR2,-mCRHR1, and -mCRHR2 reporter cell lines, pcDNA3.1-hCRHR1,-hCRHR2,-mCRHR1, and -mCRHR2 were introduced into CHO-CRE cells established above by electroporation as described above. Selection antibiotics hygromycin at 500 μg/ml and G418 at 1,000 μg/ml were employed. Individual established stable cell lines were characterized by both CRH and forskolin stimulation.

### Luciferase Reporter Assay Development

Forskolin stimulates adenylate cyclase and increases intracellular cyclic adenosine monophosphate (cAMP) levels in cells. To validate if cells respond to CAMP elevation, CHO-CRE cells were stimulated with a series of concentrations of forskolin, and cAMP-dependent luciferase expression was determined using the Bio-Glo Luciferase Assay System. CHO-CRE cells increased luciferase expression in response to forskolin stimulation in a dose-dependent manner (data not shown). CRH binds to CRHR1 and CRHR2, which are Gas coupled G-protein coupled receptors (GPCRs).

Activated GPCR proteins increase production of the intracellular cAMP. CHO-CRE-human CRHR1 stable clone 11 was generated by introduction of pcDNA3.1-human CRHR1 under the selection of G418 and hygromycin. Human CRHR1 expression in CHO-CRE-hCRHR1 Clone 11 was confirmed by flow cytometry (data not shown). To further validate that the cell line was functional, CHO-CRE-hCRHR1 Clone 11 cells were treated with forskolin and CRH peptide. CHO-CRE-hCRHR1 Clone 11 cells showed a dramatic increase of luciferase expression upon CRH treatment compared with the parental CHO-CRE, while both cell lines showed a comparable level of intracellular CAMP in response to forskolin treatment. Subsequently CHO-CRE-hCRHR2 Clone 32,-mCRHR1 Clone 22, and -mCRHR2 Clone 8 cell lines were established using similar methodology and validated with CRH treatment (data not shown).

Blocking CRHR1 by small molecules or neutralization of CRH peptide can reduce luciferase expression if the CRH-CRHR1 system works in CHO-CRE-hCRHR1 cells. Antalarmin, a small molecule CRHR1 antagonist, blocked CRH-mediated luciferase expression in CHO-CRE-hCRHR1 Clone 11 (data not shown). Furthermore, sheep anti-CRH polyclonal antibody reduced CRH-mediated luciferase expression in these cells (data not shown). Likewise, sheep anti-CRH polyclonal antibody blocked CRH-mediated luciferase expression in CHO-CRE-hCRHR2, CHO-CRE-mCRHR1, and CHO-CRE-mCRHR2 cells (data not shown).

Affinity Determination with Octet

Octet Red 96e was used for affinity measurement. Octet running buffer was prepared by diluting kinetics buffer 10× (Fortebio, Cat #18-1105) in PBS according to the manufacturer's instructions. Two columns of SA sensors (Fortebio, Cat #18-5019) were hydrated in running buffer for 10 mins, one for ligand capturing and the other for reference. Antibodies were 2-fold diluted and added into a 96-well plate. 30 nM biotinylated CRH (GL Biochem, Cat #784751) was captured by the first column of SA sensors for 20 seconds, while reference sensors were dipped into buffer wells. After a 60-second baseline step, sensors were dipped into analyte and buffer wells successively, with the association and dissociation time of 180 seconds and 800 seconds, respectively. Sensors were regenerated in glycine pH1.5 (Cytiva, Cat #BR-1003-54) before the next cycle. The equilibrium dissociation constant (KD) value was evaluated using Data Analysis Software 11.0 and the fitting model of 1:1 global fitting. Signals of reference wells and reference sensors were subtracted by selecting “Double reference” before data fitting.

### Blocking Activity of CRH Induced Signaling in Reporter Cell Lines (CHO-CRE-hCRHR1, CHO-CRE-hCRHR2, CHO-CRE-mCRHR1 and CHO-CRE-mCRHR2)

Cells were digested by trypsin-EDTA (Life technologies, Cat #: 12604-013), centrifuged at 1,000 rpm for 5 min, resuspended in growth medium, counted using a cell counter, seeded at a concentration of 5,000 cells per 100 μl in 96-well flat plates, and incubated overnight. 50 μl of 4× serially diluted antibodies was added (8 points, ¼ diluted by growth medium) to plates containing CHO-CRE-hCRHR1 cells, and 50 μl of 4×CRH (4× is 2 nM, final is 0.5 nM) was added. Similarly, 4×CRH (4× is 0.4 nM, final is 0.1 nM) was added to CHO-CRE-hCRHR2 cells and CHO-CRE-mCRHR1 cells, and 4×CRH (4× is 20 nM, final is 5 nM) was added to CHO-CRE-mCRHR2 cells. Next, plates were incubated 4-6 hours at 37° C. in a CO₂ incubator. 60 μl of culture medium was removed, to which was added 60 μl of Bio-Glo luciferase, followed by incubation for 10 min at RT. Plates were read in a luciferase plate reader (En Vision, PerkinElmer).

### Human UCN1, Human UCN2 and Human UCN3 ELISA

Streptavidin (Thermo, Cat: 21125) was diluted in PBS with a concentration of 2 μg/mL. 100 μL of diluted streptavidin was added per well to ELISA microplates, and the plates were incubated overnight at 4° C. Plates were blocked with ELISA blocking solution (containing 2% w/v BSA, 0.05% (v/v) Tween-20, pH 7.4 PBS buffer) at 37° C. for 1 hour, and then incubated with either 1 μg/mL hUCN1 (NT Biotin), hUCN1 (CT Biotin), hUCN2 (NT Biotin), hUCN2 (CT Biotin), hUCN3 (NT Biotin), or hUCN3 (CT Biotin) for 1 hour at 37° C. Plates were then washed and incubated with 100 nM and 10 nM of anti-CRH antibody for 1 hour at 37° C. Plates were subsequently washed and incubated with HRP-conjugated goat anti-human IgG (H+L) antibody (Jackson, Cat: 109-035-088) at 37° C. for 1 hour. 100 μl of TMB Substrate (Biopanda, Cat: TMB-S-003) was added, and the plates were incubated at room temperature for 15 minutes. 100 μL of ELISA stopping solution (Solarbio, Cat #: C1058) was then added to terminate the reaction, and the OD450 nm was determined by an ELISA plate reader (Molecular Devices, Spectra max 384 plus).

### Human UCN1-Induced Luciferase Reporter Assay

CHO-CRE-hCRHR1 and CHO-CRE-hCRHR2 cells were harvested with trypsin-EDTA (Life Technologies, Cat #: 12604-013). Cells were centrifuged at 1,000 rpm for 5 min and resuspended in growth medium. Cells were counted using a cell counter, seeded at 5,000 cells per 100 μl to 96-well flat plates, and incubated overnight. 50 μl of 4× serially diluted antibody (8 points, ⅓ diluted by growth medium) or CP 376395 hydrochloride (TOCRIS, Cat #: 3212) (8 points, 1/10 diluted by growth medium) was added to plates, followed by 50 μl 4× hUCN1 (4× is 4 nM, final is 1 nM) to CHO-CRE-hCRHR1 cells or CHO-CRE-hCRHR2 cells. Plates were then incubated for 4-6 hours at 37° C. in a CO₂ incubator. Following this, 60 μl of culture medium was removed, to which was added 60 μl of Bio-Glo luciferase, followed by incubation for 10 min at RT. Plates were then read in a luciferase plate reader (EnVision, PerkinElmer).

### In Vivo Efficacy Models-Wildtype Female C57BL/6 Mice and Mrap1 KO Mice

8-10 week old wildtype female C57BL/6 mice were obtained from Charles River Laboratories. Mrap1 KO mice were obtained from Queen Mary University of London, UK. SSR125543A, a small molecule CRHR1 antagonist, was obtained from Axon Medchem (Cat #Axon 1799). Mouse Corticosterone (55-Corms-E01) and adrenocorticotropic hormone (ACTH) ELISA kits (21-ACTHU-E01) were obtained from ALPCO, USA. All procedures were carried out in accordance with the Guide for the Care and Use of Laboratory Animals of the National Institutes of Health. Protocols were approved by Charles River Laboratories Institutional Animal Care and Use Committee (IACUC).

### Mrap1 KO Mice Breeding, Ear Tagging and Genotyping

To generate Mrap1 KO mice, Mrap1−/− male mice were bred with Mrap1+/− female mice. Corticosterone hormone replacement was performed by administering 50 μg/ml in drinking water to breeder cages when female mice were pregnant and their pregnancies palpable (approximately embryonic day 10). Drinking water was changed weekly, and treatment was stopped at weaning. Mice were uniquely identified with ear tags at the time of tail clipping to match future genotype with each mouse. DNA was extracted from tail tissue and used to genotype mice.

Genotyping of Mrap1−/− mice was conducted using PCR and the following PCR primers:

For the wild type allele of exon 1 of Mrap1:

Forward
(SEQ ID NO: 473)
5′-GCGTCTCTTAGTAGCCTTTGG
Reverse
(SEQ ID NO: 474)
5′-CTTGTTGGCTTTCAGCTTCT

For the mutant allele of Mrap1:

Forward
(SEQ ID NO: 475)
5′-GAACTTGCCTGGTCAACTGTTAAAAGGAC
Reverse
(SEQ ID NO: 476)
5′-TCTTTAGGCTCACTTCCTCCACTGACC

PCR band sizes: wild type allele is 345 bp and mutant allele is 459 bp.

### Physical Restraint in Wildtype Mice

To determine the effect of nonpainful physical restraint upon stress responses, mice were restrained in 50 ml polypropylene ventilated tubes in which the tip was cut off with a razor blade to allow the mouse to breathe easily for 15-20 minutes. A mouse was directed to the open tube and crawled in. A piece of paper towel was put behind the mouse so that it could not back out, and the cap was screwed on. The mouse was placed back in its cage, ventral size down on the bedding. This stressor is not considered painful, as mice are confined to a limited space (in a ventilated restraint tube) but not in an unnatural physical position. The investigator was present in the room during the entire procedure. At the end of the procedure, the mouse was removed by the tail.

### Effect of CRH Abs in Wildtype Mice in Restraint-Stress Model

On day 1, each mouse was injected intraperitoneally with 20 mg/kg of anti-CRH mAbs, hIgG1 or PBS (5 mice per group). On day 2 or 3, and 14 each mouse was subjected to physical restraint-stress described above for 15 or 20 min. Immediately after restraint-stress, 200 μl of blood was collected in EDTA tubes from the submandibular vein of each mouse. Plasma was separated using a refrigerated centrifuge (4° C. at 500×g for 10 min) and snap-frozen in dry ice for subsequent ACTH and corticosterone ELISA measurement.

### Effect of Anti-CRH Abs in Mrap1 KO Mouse Model

On day 1, each mouse was injected (IP) with 20 mg/kg of anti-CRH Ab or hIgG1 (5 or 6 mice each group). On day 2 or 3, 14 and 28, 200 μl of blood was collected in EDTA tubes from the submandibular vein of each mouse. Plasma was separated using a refrigerated centrifuge (4° C. at 500×g for 10 min) and snap-frozen in dry ice for subsequent ACTH ELISA measurement.

Parallel Comparison of Anti-CRH mAb with SSR125543A in Wildtype Mice Restraint-Stress Model

On day 1, one half of mice were injected IP with 20 mg/kg of anti-CRH Ab or hIgG1 (5 mice per group). On day 3, the other half of mice were injected IP with 30 mg/kg of SSR125543A or solvent (5 mice each group). Two hours later, four groups of mice were subjected to restraint-stress as described above for 20 min. Immediately after restraint-stress, 200 μl of blood was collected in EDTA tubes from the submandibular vein of each mouse in all four groups. Plasma was separated as described above and snap-frozen in dry ice for subsequent ACTH and corticosterone ELISA measurement. On day 4, SSR125543A or solvent-treated mice were subjected to a second round of restraint stress for 20 min and plasma was collected. Two weeks later, anti-CRH mAb- or hIgG1-treated mice were subjected to physical stress for 20 min followed by plasma collection.

### In Vivo PK Study

### Administration and Blood Collection

For each antibody tested, 6 female C57BL/6 mice with a weight of 18-22 grams were selected, and antibody was administered intravenously at a single dose of 5 mg/kg. Whole blood was collected from 3 mice before and 15 minutes, 24 hours (1 day), 4, and 10 days after administration. Whole blood from the remaining 3 mice was collected before and 5 hours, 2, 7, and 14 days after administration. Whole blood was allowed to stand for 30 minutes to coagulate and then centrifuged. Separated serum was then frozen at −80° C. until analysis.

### Analysis Method

ELISA was used to quantitatively determine the drug concentration in mouse serum. Tested antibodies (with human Fc) in mice serum were captured by goat anti-human Fc polyclonal antibody (Rockland, #609-101-017) pre-coated on a 96-well plate. HRP-labeled goat anti-human (H+L) secondary antibody (Abcam, #ab97175) was then added into the wells. Plates were then sealed and incubated at 37° C. for 30 minutes, followed by washing and adding TMB substrate and ELISA stopping solution. 96-well plates were read at 450/630 nm wavelength using SpectraMax M2. Data was processed by Soft Max Pro.

Pharmacokinetic (PK) parameters were analyzed by Phoenix WinNonlin version 8.2 with non-compartmental model (NCA).

### Determining the Binding Region of CRH for Anti-CRH Antibodies by ELISA

Streptavidin (Thermo, Cat: 21125) was diluted in PBS to a concentration of 2 μg/ml. 100 μl of diluted streptavidin was added per well to ELISA microplates, and the plates were incubated overnight at 4° C. Plates were blocked with ELISA blocking solution (containing 2% w/v BSA, 0.05% (v/v) Tween-20, pH 7.4 PBS buffer) at 37° C. for 1 hour, and then incubated with 1 μg/mL N-terminal region plus the central region (CT biotin), the central region only (NT biotin or CT biotin), or the central region plus C-terminal region (NT biotin) of CRH for 1 hour at 37° C. Plates were then washed and incubated with diluted anti-CRH antibody (Ab) at 15 μg/ml (100 nM, 10 diluted, 8 points) for 1 hour at 37° C. Subsequently, plates were washed and incubated with HRP-conjugated goat anti-human IgG (H+L) antibody (Jackson, Cat: 109-035-088) at 37° C. for 1 hour. 100 μL of 3,3′,5,5′-tetramethylbenzidine (TMB) substrate (Biopanda, Cat: TMB-S-003) was added, and the plates were incubated at room temperature for 15 minutes. 100 μL of ELISA stopping solution (Solarbio, Cat #: C1058) was added to terminate the reaction, and the optical density at 450 nm (OD450 nm) was determined by an ELISA plate reader (Molecular Devices, Spectra max 384 plus).

### Epitope Binding by Competitive ELISA

For epitope binding, a competitive ELISA was performed. Specifically, 1 μg/ml of capture antibody, 100 μl/well, was coated on a 96 well-plate at 4° C. overnight. Plates were blocked with ELISA blocking solution (containing 2% w/v BSA, 0.05% (v/v) Tween-20, pH 7.4 PBS buffer) at 37° C. for 1 hour. Then, a mixture of 50 μl/well of competitive antibody (40 μg/ml) and 50 μl/well biotin-labeled CRH with proper concentration was added to 96 well plates followed by incubation for 1 hour at 37° C. Subsequently, plates were washed and incubated with Streptavidin-Peroxidase (SIGMA, Cat: S2438-250UG) at 37° C. for 1 hour. 100 μL of 3,3′,5,5′-tetramethylbenzidine (TMB) substrate (Biopanda, Cat: TMB-S-003) was added, and the plates were incubated at room temperature for 15 minutes. 100 μL of ELISA stopping solution (Solarbio, Cat #: C1058) was added to terminate the reaction, and the optical density at 450 nm (OD450 nm) was determined by an ELISA plate reader (Molecular Devices, Spectra max 384 plus).

### Epitope Binning Using Octet

Epitope binding was performed in Octet Red 96e. Running buffer was prepared by diluting kinetics buffer 10× (Fortebio, Cat #18-1105) in PBS according to the manufacturer's instructions.

SA sensors (Fortebio, Cat #18-5019) were hydrated in running buffer for 10 mins. Performing an in-tandem epitope binding assays involved a four-step binding cycle: 1) a regeneration step was established for 30 s, 2) CRH was captured at 30 nM with the loading threshold at 0.12 nm, 3) 60 nM antibodies (1st Ab) were loaded to saturate the immobilized peptide for 200 s, 4) the mixture of 1st Ab in step 3 and competing antibodies were bound for 200 s.

Octet data were processed in Fortebio's Data Analysis Software 11.0. After exporting the signal, the inhibition ratio was calculated as below:

$$Inhibition ratio ( \% ) = ( a - b ) / a * 100 \%$$

Take antibody Abn, for example
Variable a: The signal of the binding of Abn alone to the antigen (also refers to 100% signal of Abn)
Variable b: When Abn is the 2nd Ab, the signal of it binding to antigen

### Example 2

### Generation of CRH Knockout Mice on H2L2 Harbour Mice® Background

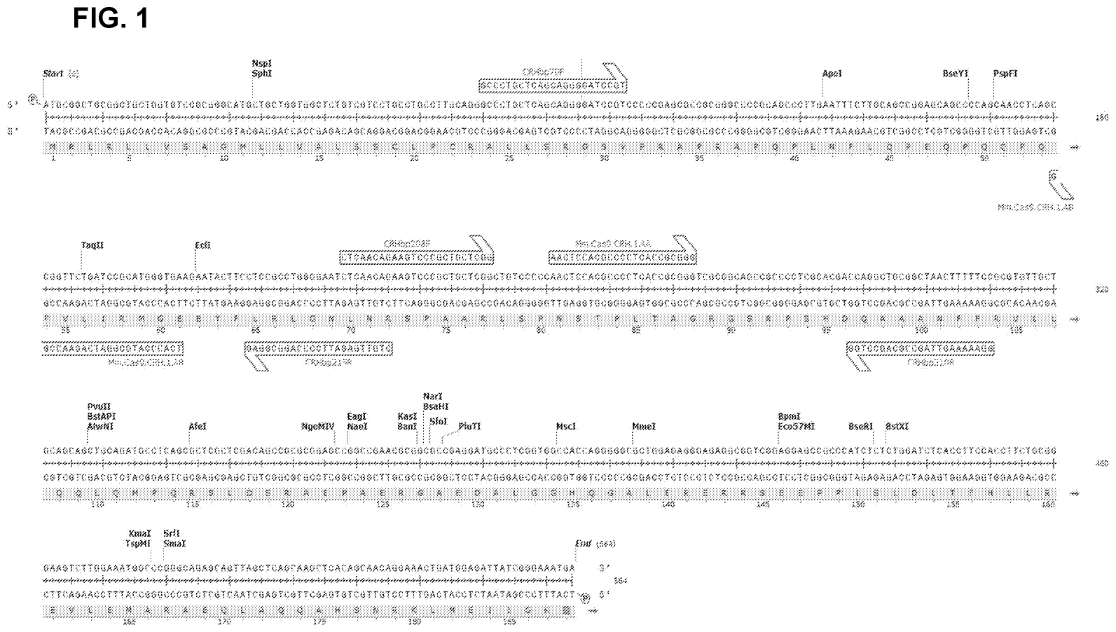

CRH knockout mice on the H2L2 Harbour Mice® background, using the materials and methods described above. FIG. 1 shows the coding sequence open reading frame of mouse CRH (GenBank NM_205769), along with the CRISPR targeting gRNAs and PCR primers used. Specifically, the following primers were used for genotyping, i.e., amplification on genomic DNA, as depicted in FIG. 1:

CRHbp70F:
(SEQ ID NO: 477)
5′-GCC CTG CTC AGC AGG GGA TCC GT-3′
CRHbp215R:
(SEQ ID NO: 478)
5′-CTG TTG AGA TTC CCC AGG CGG AG-3′
CRHbp208F:
(SEQ ID NO: 479)
5′-CTC AAC AGA AGT CCC GCT CGG-3′
CRHbp310R:
(SEQ ID NO: 480)
5′-GGA AAA AGT TAG CCG CAG CCT GG-3′

The expected PCR DNA fragment sizes from wildtype mice are 146 bp and 103 bp, respectively. PCR fragments smaller than these expected sizes indicated deletion in founder mice. FIGS. 2A-2B show the identification of CRH knockout founder mice using this method.

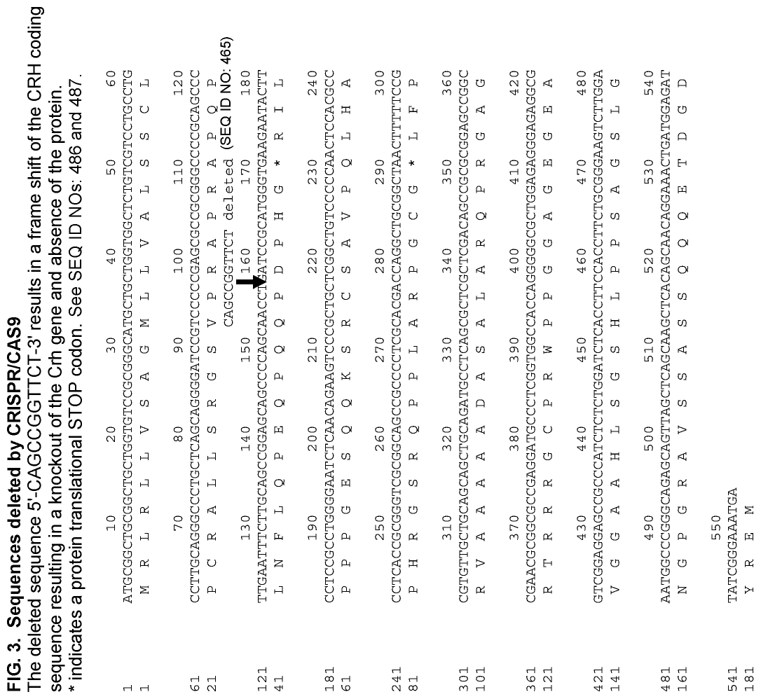

FIG. 3 shows location of an 11 bp deletion (5′-CAGCCGGTTCT-3′) (SEQ ID NO: 465) in CRH gene coding region 157-167, that leads to a downstream reading frame shift and premature stop codon. This defective CRH open reading frame is not able to synthesize CRH peptide. A founder having this deletion was chosen for further breeding to homozygosity, rescue and immunization.

### Example 3

Identification of Anti-CRH mAbs from CRH Knockout Mice on H2L2 Harbour Mice® Background

Many positive hybridoma clones were identified from multiple rounds of hybridoma fusions from immunization with CRH peptide in CRH knockout mice on a H2L2 Harbour Mice® background. Purified monoclonal antibodies (mAbs) from hybridoma supernatants bound to CRH with high affinity in ELISA. However, three functional monoclonal antibody clones, hybridoma clones 16g2, 28b3 and 52g7, were identified with blocking activity in the human CRHR1 luciferase reporter assay. These three clones were expressed recombinantly with human IgG1 wildtype Fc as PR004289, PR004290 and PR006669, respectively. PR004289 (from Hybridoma Clone 16g2), PR004290 (from Hybridoma Clone 28b3), and PR006669 (from Hybridoma Clone 52g7) share the same germline sequences of IGHV3-7 and IGKV2-28. PR004289 and PR004290 have one residue difference at IGHV position 28, being 128 in PR004289 and T28 in PR004290, respectively. Both PR004289 and PR004290 have complementarity determining regions (CDRs) different from that of PR006669. These three mAbs were designated as Series 1 (PR004289 and PR004290) and Series 2 (PR006669) mAbs. See Table 2.

TABLE 2
Identification of Anti-CRH mAbs

| | | | | | | | | | | |
|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | | [symbol] |
| [symbol] | [symbol] | [symbol] | [symbol] | | [symbol] | [symbol] | | [symbol] | [symbol] | [symbol] |
| [symbol] | [symbol] | [symbol] | [symbol] | | [symbol] | [symbol] | | [symbol] | [symbol] | [symbol] |
| [symbol] | [symbol] | [symbol] | [symbol] | | [symbol] | [symbol] | | [symbol] | [symbol] | [symbol] |
| | [symbol] | [symbol] | [symbol] | | [symbol] | [symbol] | | [symbol] | [symbol] | [symbol] |
| | [symbol] | [symbol] | [symbol] | | [symbol] | [symbol] | | [symbol] | [symbol] | [symbol] |
| | [symbol] | [symbol] | [symbol] | [symbol] | [symbol] | [symbol] | | [symbol] | [symbol] | [symbol] |
| [symbol] | [symbol] | [symbol] | [symbol] | | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| | [symbol] | [symbol] | [symbol] | | [symbol] | [symbol] | [symbol] | [symbol] | [symbol] | [symbol] |
| | [symbol] | [symbol] | [symbol] | | [symbol] | [symbol] | [symbol] | [symbol] | [symbol] | [symbol] |
| | [symbol] | [symbol] | [symbol] | | [symbol] | [symbol] | [symbol] | [symbol] | [symbol] | [symbol] |
| [symbol] | [symbol] | [symbol] | [symbol] | | [symbol] | [symbol] | [symbol] | [symbol] | [symbol] | [symbol] |
| | [symbol] | [symbol] | [symbol] | | [symbol] | [symbol] | [symbol] | [symbol] | [symbol] | [symbol] |
| [symbol] | [symbol] | [symbol] | [symbol] | | [symbol] | [symbol] | | [symbol] | [symbol] | [symbol] |
| | [symbol] | [symbol] | [symbol] | | [symbol] | [symbol] | | [symbol] | [symbol] | [symbol] |
| | [symbol] | [symbol] | [symbol] | | [symbol] | [symbol] | | [symbol] | [symbol] | [symbol] |
| | [symbol] | [symbol] | [symbol] | | [symbol] | [symbol] | | [symbol] | [symbol] | [symbol] |
| [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] |

[symbol]

indicates data missing or illegible when filed

### Example 4

Identification of Anti-CRH mAbs from Wildtype Mice

Over 200,000 single B cells from wildtype mice immunized with CRH-KLH conjugates were screened with the Beacon system, using the materials and methods described above. More than 500 monoclonal antibodies (mAbs) with binding activities in bead binding assay were identified. Over 300 of these mAbs were expressed recombinantly with human IgG1 wildtype Fc. Many of these recombinantly-expressed mAbs bound to CRH with high affinity in ELISA. Eight series of functional mAbs with blocking activities in human CRHR1 luciferase reporter assay were identified, using the materials and methods described above. mAbs from different series have different germline sequences and different CDRs. mAbs within each series share same germline sequences, but have different CDRs.

These mAbs were designated as Series 3 to Series 10 as follows.

Series 3 includes PR301306.

Series 4 includes PR301612.

Series 5 includes PR301777, PR301788, PR301790 and PR301803.

Series 6 includes PR301806.

Series 7 includes PR302309, PR302332 and PR302333.

Series 8 includes PR302315 and PR302335.

Series 9 includes PR302328, PR302331, PR302334 and PR302339.

Series 10 includes PR302312, PR302324 and PR302341.

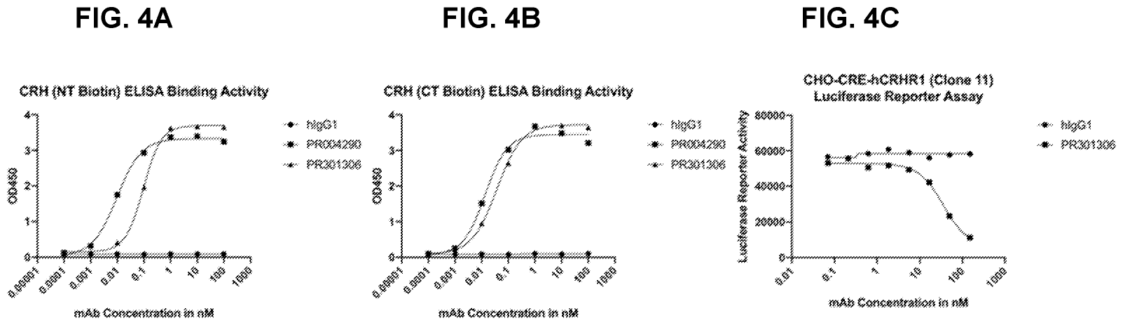

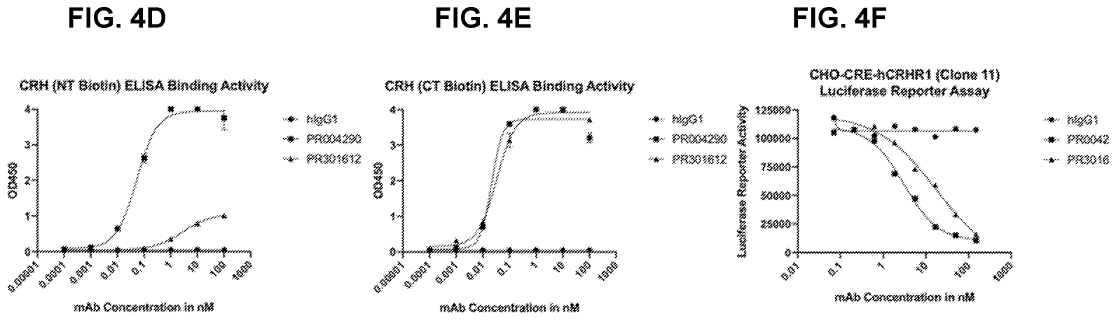

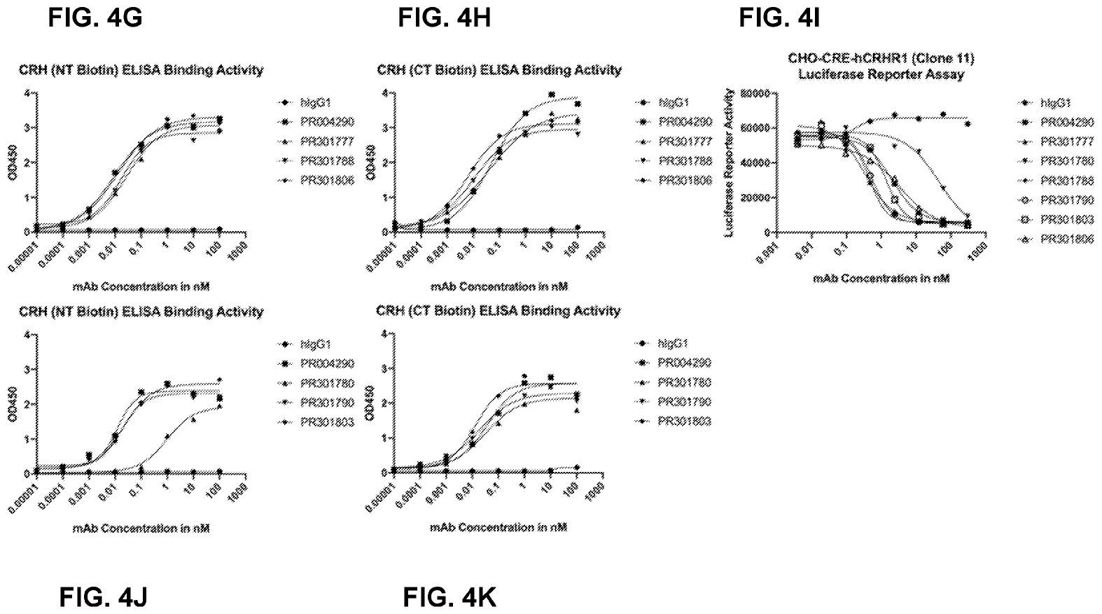

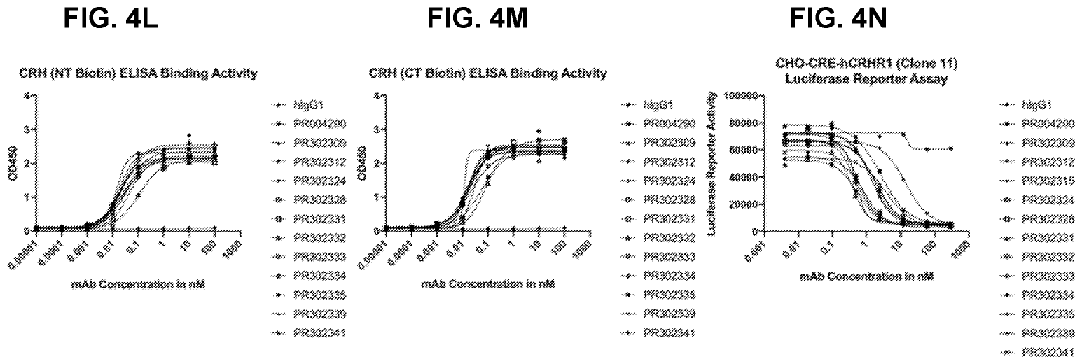

See also Table 2 and FIGS. 4A-4N.

### Example 5

### Antibody Engineering

### PTM Removal of Series 1 mAb PR004290

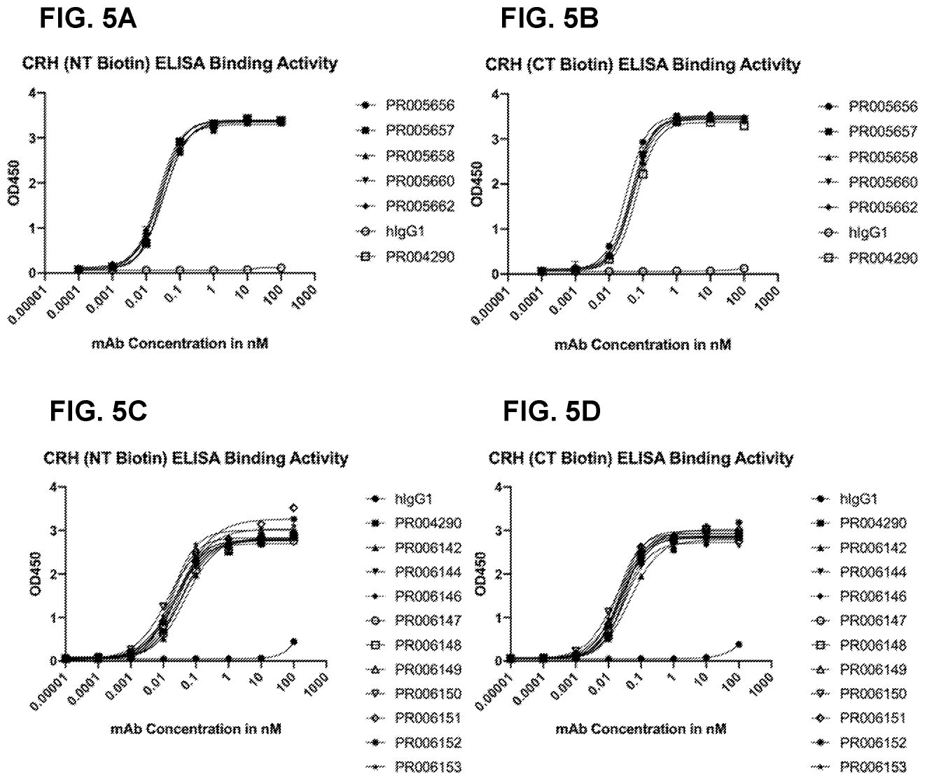

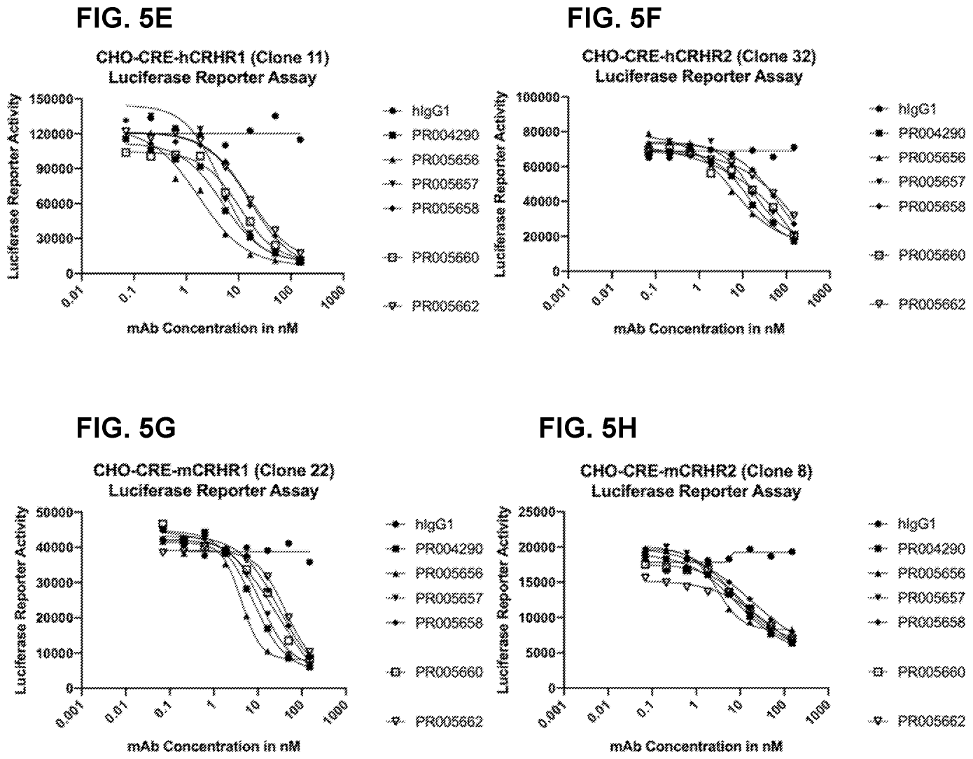

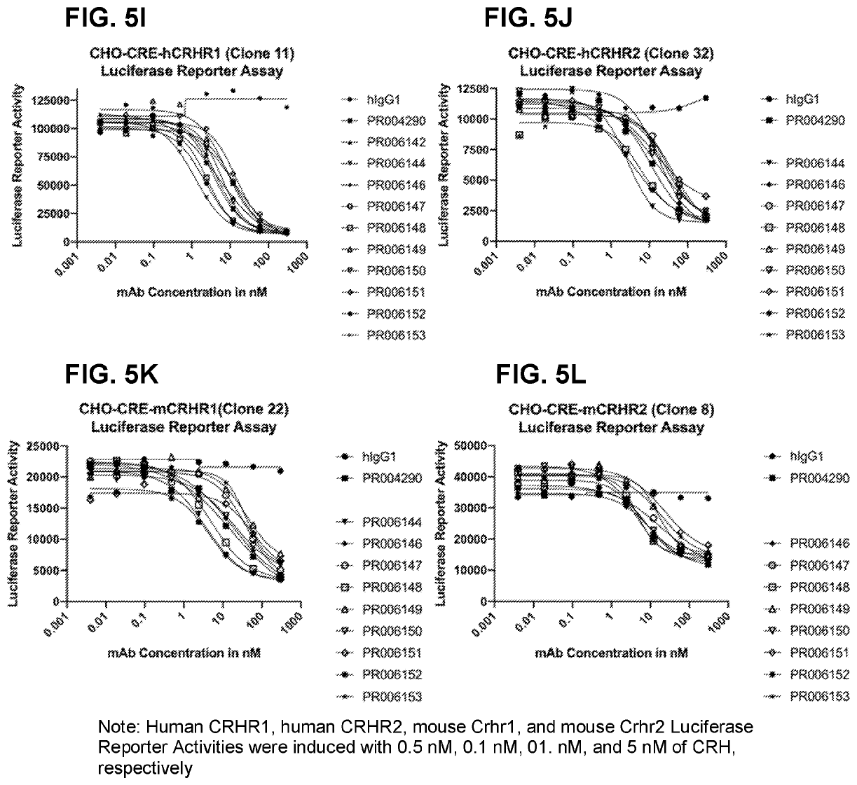

PR004290 contains two potential posttranslational modifications (PTMs), DG in heavy chain CDR2 (HCDR2) and NS in light chain CDR1 (LCDR1). PR006669 has one potential PTM of DG in HCDR2. These potential PTMs were mutated individually either in HCDR2 alone, in LCDR1 alone, or in various combinations of mutations, as detailed in Table 3. Their binding activities in CRH ELISA and functional blocking activities in human CRHR1 (hCRHR1), human CRHR2 (hCRHR2), mouse CRHR1 (mCRHR1), and mouse CRHR2 (mCRHR2) luciferase reporter assays are shown in Table 3 and FIGS. 5A-5L. Most ΔPTM variants retained functional activities. PR005660 had a 50% inhibitory concentration (IC50) of about 10 nM in the hCRHR1 reporter assay, vis-à-vis about 5 nM of parental PR004290.

TABLE 3
CRH ELISA Binding Activities and Functional Blocking Activities
of Series 1 mAb PR004290 and Its ΔPTM Variants

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| | | | | | ELISA EC50 (nM) | | | | | |
| mAb ID | IGHV | IGKV | HCDR2 | LCDR1 | CRH (NT- | CRH (CT- | Luciferase Reporter Assay IC50 (nM) | | | |
| mAb ID | IGHV | IGKV | HCDR2 | LCDR1 | Biotin) | Biotin) | hCRHR1 | hCRHR2 | mCrhr1 | mCrhr2 |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | | | |
| PR00 [symbol] | IGHV3-7 | IGKV2-28 | [symbol] | [symbol] | [symbol] | [symbol] | [symbol] | [symbol] | [symbol] | |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | [symbol] | [symbol] | [symbol] | [symbol] | [symbol] | [symbol] | [symbol] |

[symbol]

indicates data missing or illegible when filed

### PTM Removal of Series 2 mAb PR006669

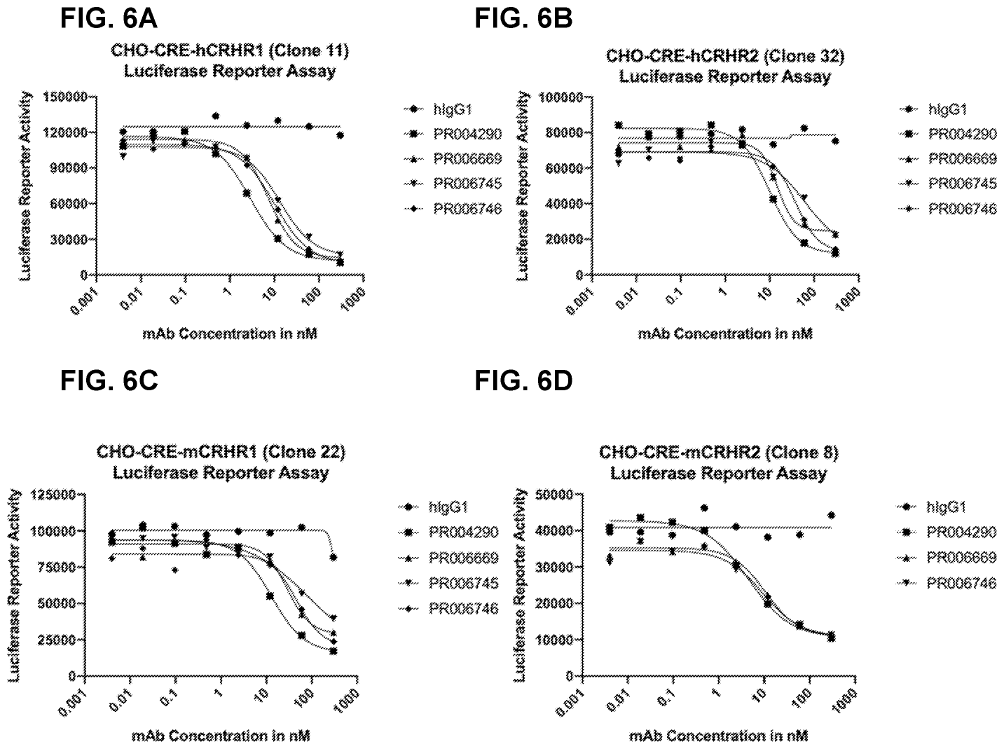

PR006745 and PR006746 are two ΔPTM variants of PR006669 and with somewhat weaker ELISA binding activities and functional blocking activities. See Table 4 and FIGS. 6A-6D.

TABLE 4
Functional Blocking Activities of Series 2 mAb PR006669 and Its ΔPTM Variants

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| PR00 [symbol] | IGHV3-7 | IGKV2-28 | [symbol] | [symbol] | [symbol] | [symbol] | [symbol] | [symbol] | [symbol] | [symbol] |
| PR00 [symbol] | IGHV3-7 | IGKV2-28 | [symbol] | | | | [symbol] | [symbol] | [symbol] | [symbol] |
| PR00 [symbol] | IGHV3-7 | IGKV2-28 | [symbol] | | | | [symbol] | [symbol] | [symbol] | |
| PR00 [symbol] | IGHV3-7 | IGKV2-28 | [symbol] | | | | [symbol] | [symbol] | [symbol] | [symbol] |

[symbol]

indicates data missing or illegible when filed

### Humanization of Series 5 mAb PR301777

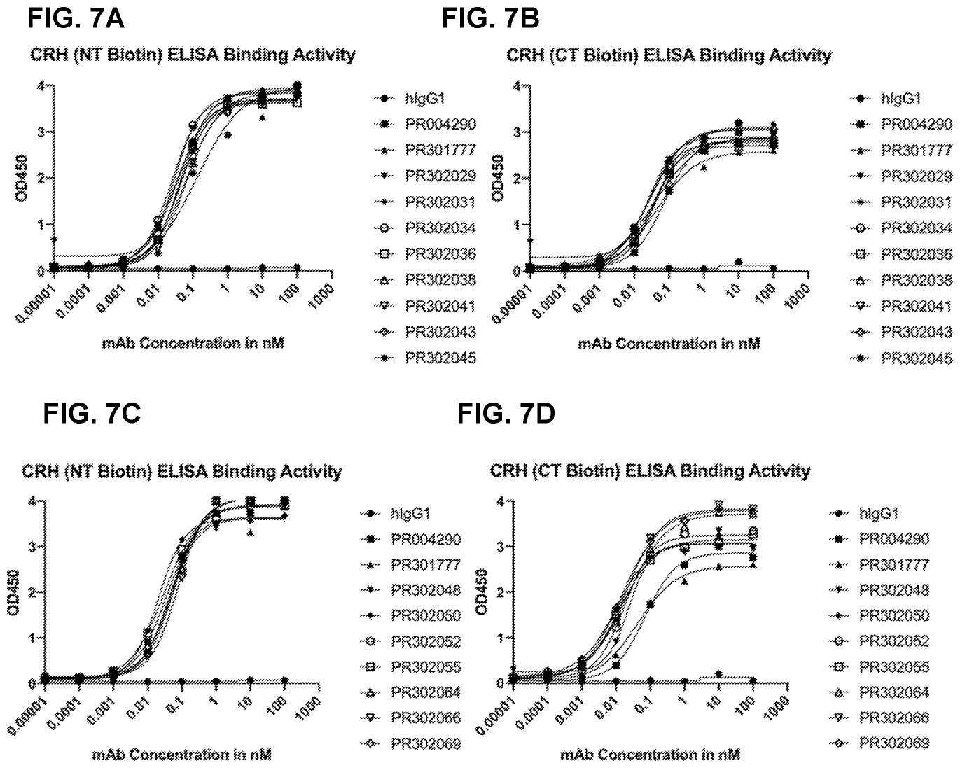

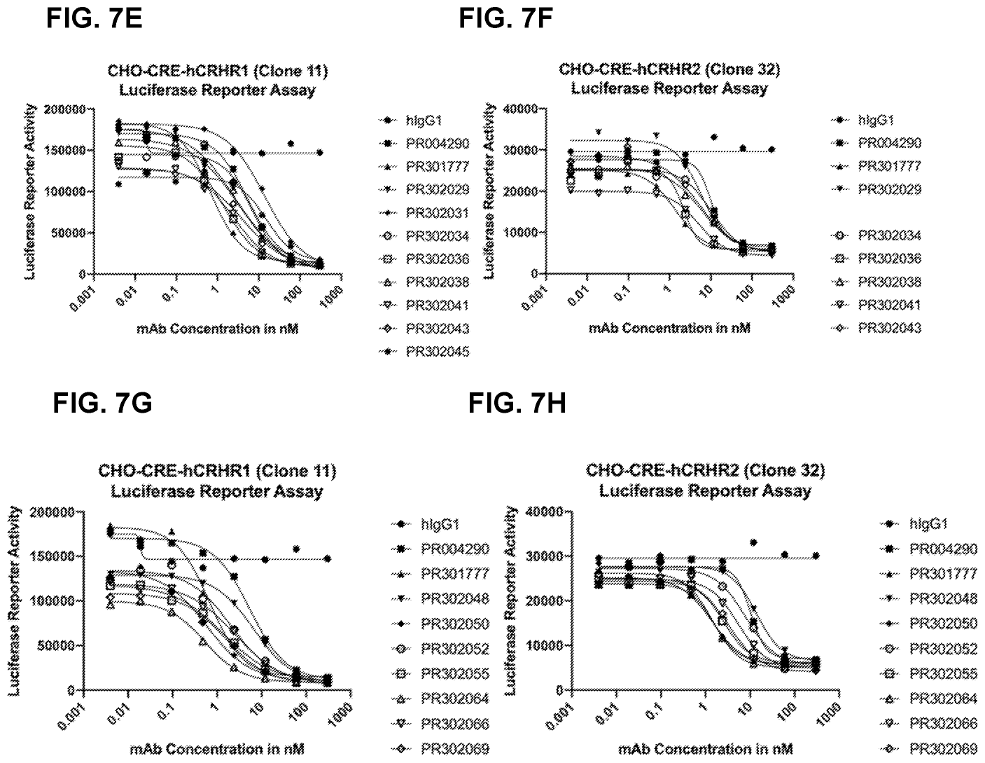

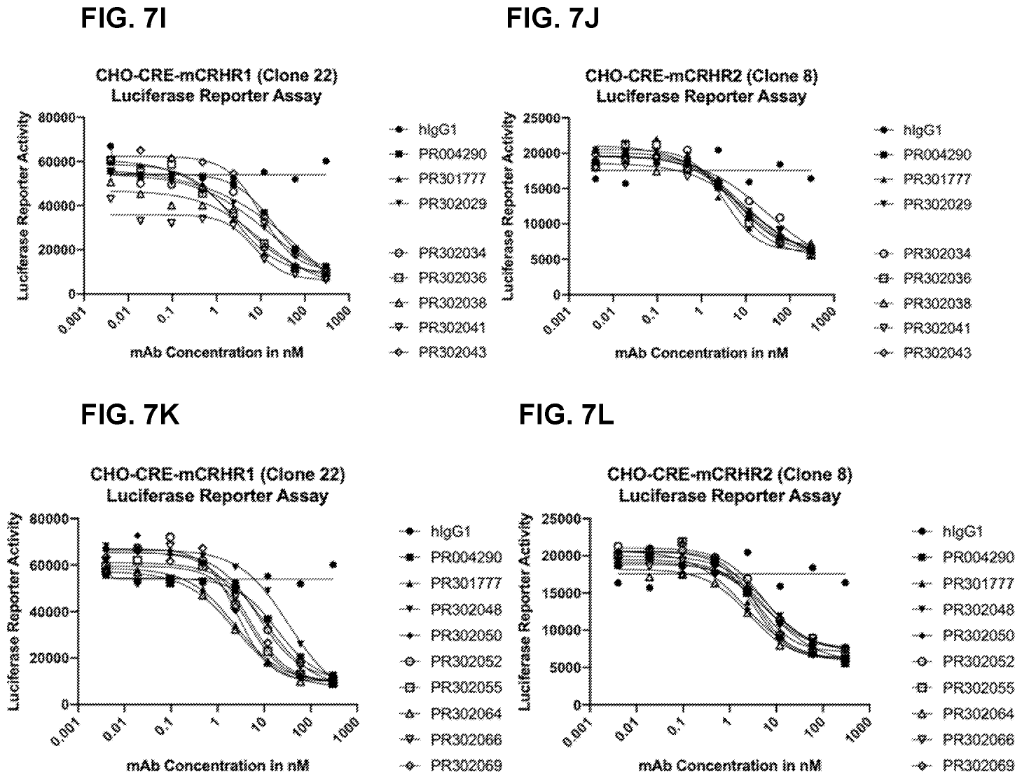

Fifteen humanized variants of PR301777 were recombinantly expressed and tested, using the materials and methods described above. Many humanization variants maintained high potency in vitro ELISA binding activities and functional blocking activities. See Table 5 and FIGS. 7A-7L.

TABLE 5
CRH ELISA Binding Activities and Functional Blocking Activities
of Series 5 mAb PR301777 and Its Humanized Variants

| | PR301777 0.674 nM | | | |
|---|---|---|---|---|
| | IGKV | IGKV4-1*01 | IGKV3-11*02 | IGKV1-39*01 |
| | VH/VL | Back Mutation | Back Mutation | Back Mutation |
| IGHV | SEQ ID NO | SEQ ID NO: 310 | SEQ ID NO: 311 | SEQ ID NO: 312 |
| IGHV3-72*01 | SEQ ID NO: 255 | PR302029 | PR302031 | PR302034 |
| | | 2.649 nM | 14.000 nM | 6.871 nM |
| IGHV3-72*01 | SEQ ID NO: 256 | PR302036 | PR302038 | PR302041 |
| Back Mutation | | 1.397 nM | 3.341 nM | 2.536 nM |
| IGHV3-73*01 | SEQ ID NO: 257 | PR302043 | PR302045 | PR302048 |
| | | 3.866 nM | 15.310 nM | 6.659 nM |
| IGHV3-73*01 | SEQ ID NO: 258 | PR302050 | PR302052 | PR302055 |
| Back Mutation | | 0.572 nM | 2.052 nM | 1.348 nM |
| IGHV3-15*07 | SEQ ID NO: 259 | PR302064 | PR302066 | PR302069 |
| Back Mutation | | 0.531 nM | 2.494 nM | 1.539 nM |

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | | | |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | | | |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |
| PR00 [symbol] | [symbol] | [symbol] | [symbol] | [symbol] | [symbol] | [symbol] | [symbol] | [symbol] | [symbol] | [symbol] |

[symbol]

indicates data missing or illegible when filed

### Humanization of Series 7 mAb PR302309

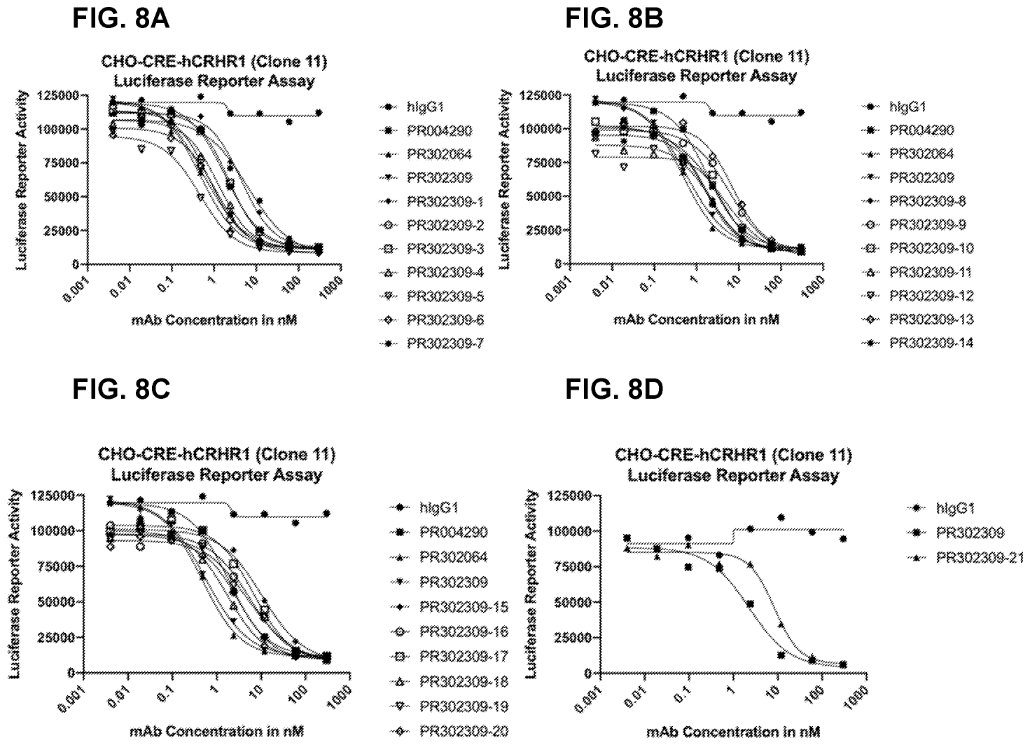

Twenty-one humanized variants of PR302309 were recombinantly expressed and tested, using the materials and methods described above. Many humanization variants maintained high potency in vitro ELISA binding activities and functional blocking activities. See Table 6 and FIGS. 8A-8D.

TABLE 6
Functional Blocking Activities of Series 7 mAb PR302309 and Its Humanized Variants

| | hCRHR1-Luc 0.5 nM CRH IC50 (nM) | | | | | | | |
|---|---|---|---|---|---|---|---|---|
| | | Original | | | | IGKV2- | | IGKV2- |
| | | MousemIGK | | IGKV2-30*02 | | 29*02 | | 24*01 |
| | | V1-117*01 | IGKV2- | Back | IGKV2- | Back | IGKV2- | Back |
| | IGKV | HCDR1 DG | 30*02 | Mutation | 29*02 | Mutation | 24*01 | Mutation |
| | VH/VL | SEQ ID NO: | SEQ ID NO: | SEQ ID NO: | SEQ ID NO: | SEQ ID NO: | SEQ ID NO: | SEQ ID NO: |
| IGHV | SEQ ID NO | 317 | 313 | 318 | 319 | 314 | 315 | 316 |
| Original | SEQ ID | PR302309 | PR302309-1 | PR302309-2 | PR302309-3 | PR302309-4 | PR302309-5 | PR302309-6 |
| Mouse | NO: 260 | (0.555/2.256 | (4.425 nM) | (0.773 nM) | (2.222 nM) | (1.261 nM) | (0.454 nM) | (1.084 nM) |
| mIGHV9-2-1*01 | | nM) | | | | | | |
| No PTM | | | | | | | | |
| IGHV7-4-1*02 | SEQ ID | PR302309-13 | | PR302309-17 | | | | |
| | NO: 262 | (7.232 nM) | | (6.687 nM) | | | | |
| IGHV7-4-1*02 | SEQ ID | PR302309-14 | PR302309-7 | PR302309-8 | PR302309-9 | PR302309-10 | PR302309-11 | PR302309- |
| Back | NO: 261 | (1.677 nM) | (6.378 nM) | (1.696 nM) | (6.081 nM) | (3.203 nM) | (2.116 nM) | 12 |
| Mutation | | | | | | | | (4.466 nM) |
| IGHV1-3*01 | SEQ ID | PR302309-15 | | PR302309-18 | | | | |
| | NO: 263 | (10.52 nM) | | (1.730 nM) | | | | |
| IGHV1-3*01 | SEQ ID | PR302309-16 | | PR302309-19 | | PR302309-20 | | PR302309- |
| Back | NO: 264 | (5.474 nM) | | (4.833 nM) | | (3.071 nM) | | 21 |
| Mutation | | | | | | | | (8.442 nM) |

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRNR1 | hCRNR2 | mCrhr1 | mCrhr2 |
| PR [symbol] | [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | | PR [symbol] | | | | |
| PR [symbol] | [symbol] | [symbol] | [symbol] | [symbol] | | PR [symbol] | | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |

[symbol]

indicates data missing or illegible when filed

### Humanization of Series 9 mAb PR302334

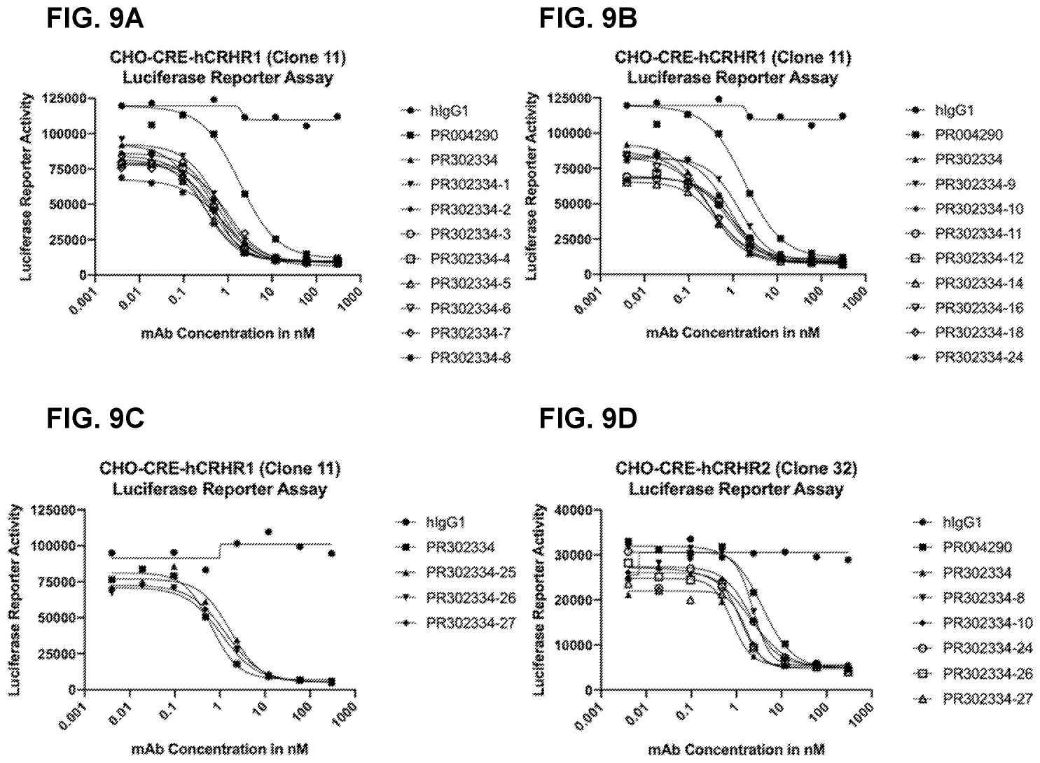

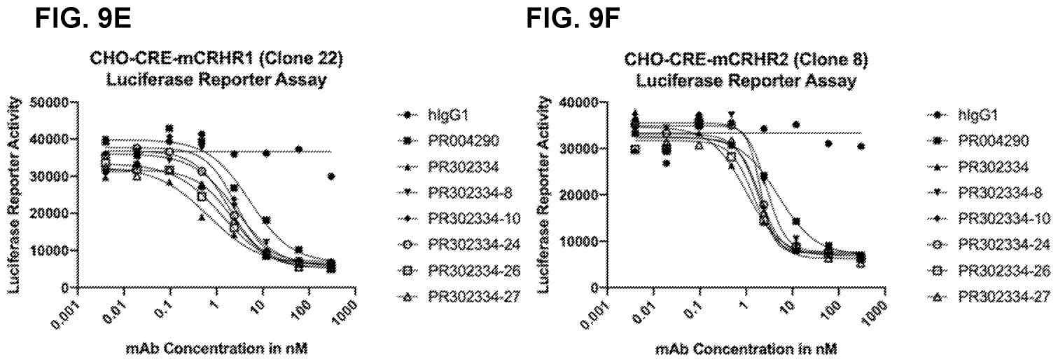

Twenty-two humanized variants of PR302334 were recombinantly expressed and tested, using the materials and methods described above. Many humanization variants maintained high potency in vitro ELISA binding activities and functional blocking activities. See Table 7 and FIGS. 9A-9F.

TABLE 7
Functional Blocking Activities of Series 9 mAb PR302334 and Its Humanized Variants

| | hCRHR1-Luc 0.5 nM CRH IC50 (nM) | | | | | | | |
|---|---|---|---|---|---|---|---|---|
| | | Original Mouse | | IGKV3- | | IGKV4- | | IGKV1- |
| | | mIGKV6- | | 15*01 | | 1*01 | | 33*01 |
| | | 32*01 | IGKV3- | Back | IGKV4- | Back | IGKV1- | Back |
| | IGKV | No PTM | 15*01 | Mutation | 1*01 | Mutation | 33*01 | Mutation |
| | VH/VL | SEQ ID NO: | SEQ ID NO: | SEQ ID NO: | SEQ ID NO: | SEQ ID NO: | SEQ ID NO: | SEQ ID NO: |
| IGHV | SEQ ID NO | 331 | 327 | 332 | 333 | 328 | 329 | 330 |
| Original | SEQ ID | PR302334 | PR302334-1 | PR302334-2 | PR302334-3 | PR302334-4 | PR302334-5 | PR302334-6 |
| Mouse | NO: 272 | (0.260/0.644 | (0.624 nM) | (0.597 nM) | (0.542 nM) | (0.553 nM) | (0.325 nM) | (0.404 nM) |
| mIGHV5- | | nM) | | | | | | |
| 9*02 | | | | | | | | |
| No PTM | | | | | | | | |
| IGHV3-23*04 | SEQ ID | PR302334-14 | PR302334-7 | PR302334-8 | PR302334-9 | PR302334-10 | PR302334-11 | PR302334-12 |
| Back Mutation | NO: 273 | (0.468 nM) | (0.996 nM) | (0.816 nM) | (1.415 nM) | (0.839 nM) | (1.003 nM) | (0.986 nM) |
| IGHV3-69- | SEQ ID | PR302334-15 | | | | | | |
| 1*01 | NO: 274 | (5.118 nM) | | | | | | |
| IGHV3-69- | SEQ ID | PR302334-16 | | | | PR302334-24 | | PR302334-25 |
| 1*01 | NO: 275 | (0.339 nM) | | | | (0.778 nM) | | (1.700 nM) |
| Back | | | | | | | | |
| Mutation | | | | | | | | |
| IGHV3-48*03 | SEQ ID | PR302334-17 | | PR302334-22 | | | | |
| | NO: 276 | (12.150 nM) | | (0.920 nM) | | | | |
| IGHV3-48*03 | SEQ ID | PR302334-18 | | | | PR302334-26 | | PR302334-27 |
| Back | NO: 277 | (0.279 nM) | | | | (1.100 nM) | | (1.364 nM) |
| Mutation | | | | | | | | |

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | | [symbol] | | [symbol] | [symbol] | [symbol] |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | [symbol] | [symbol] | [symbol] |
| [symbol] | [symbol] | [symbol] | [symbol] | [symbol] | | | [symbol] | | | |
| [symbol] | [symbol] | [symbol] | [symbol] | [symbol] | | | [symbol] | [symbol] | [symbol] | [symbol] |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | | | |
| [symbol] | [symbol] | [symbol] | [symbol] | [symbol] | | | [symbol] | [symbol] | [symbol] | [symbol] |
| [symbol] | [symbol] | [symbol] | [symbol] | [symbol] | | | [symbol] | | | |
| [symbol] | [symbol] | [symbol] | [symbol] | [symbol] | | | [symbol] | [symbol] | [symbol] | [symbol] |
| [symbol] | [symbol] | [symbol] | [symbol] | [symbol] | | | [symbol] | [symbol] | [symbol] | [symbol] |

[symbol]

indicates data missing or illegible when filed

### Humanization of Series 10 mAb PR302341

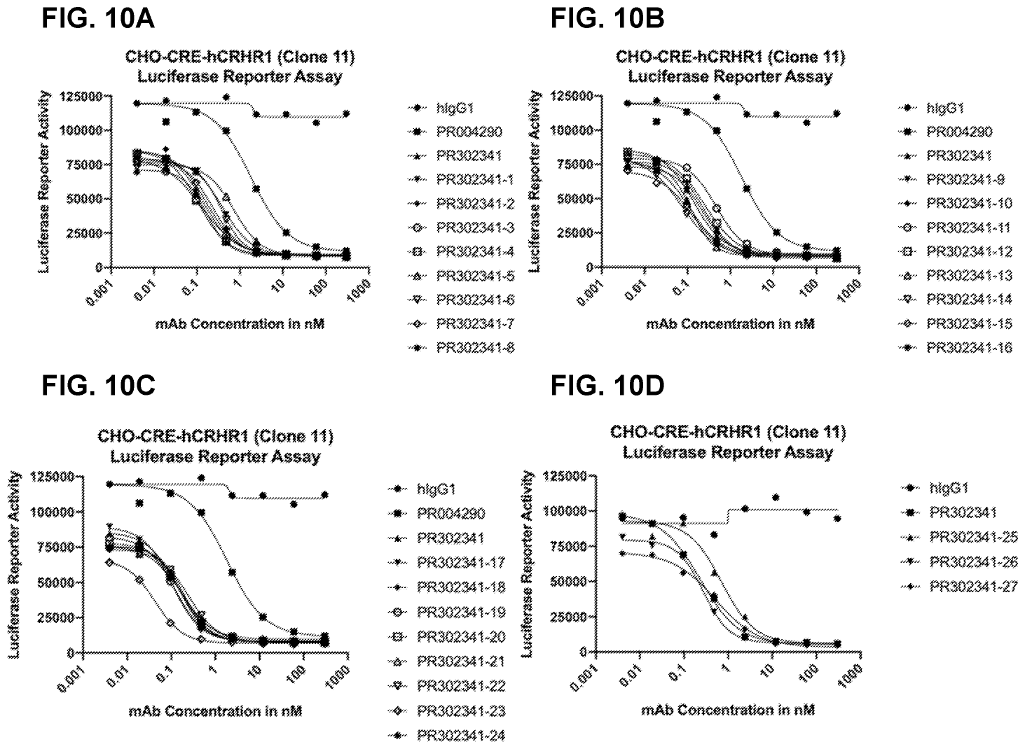

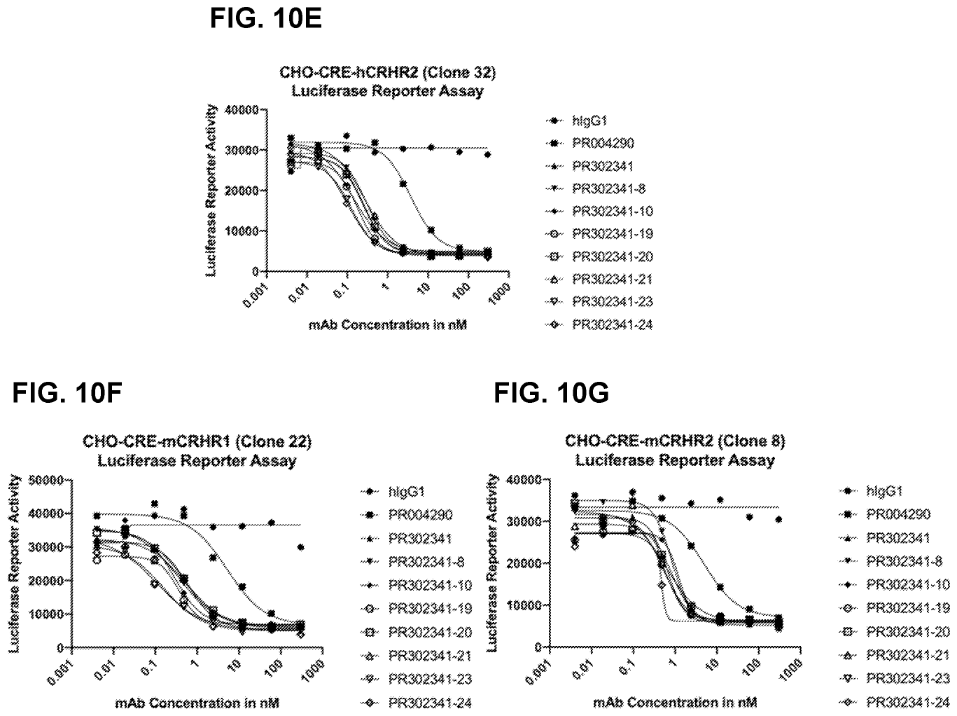

Twenty-seven humanized variants of PR302341 were recombinantly expressed and tested, using the materials and methods described above. Many humanization variants maintained high potency in vitro ELISA binding activities and functional blocking activities. See Table 8 and FIGS. 10A-10G.

TABLE 8
Functional Blocking Activities of Series 10 mAb PR302341 and Its Humanized Variants

| | hCRHR1-Luc 0.5 nM CRH IC50 (nM) | | | | | | | |
|---|---|---|---|---|---|---|---|---|
| | | Original Mouse | | IGKV2- | | IGKV2- | | IGKV2- |
| | | mIGKV1- | | 30*02 | | 24*01 | | 29*02 |
| | | 117*01 | IGKV2- | Back | IGKV2- | Back | IGKV2- | Back |
| | IGKV | LCDR1 DG | 30*02 | Mutation | 24*01 | Mutation | 29*02 | Mutation |
| | VH/VL | SEQ ID NO: | SEQ ID NO: | SEQ ID NO: | SEQ ID NO: | SEQ ID NO: | SEQ ID NO: | SEQ ID NO: |
| IGH V | SEQ ID NO | 339 | 335 | 340 | 341 | 336 | 337 | 338 |
| Original | SEQ ID | PR302341 | PR302341-1 | PR302341-2 | PR302341-3 | PR302341-4 | PR302341-5 | PR302341-6 |
| Mouse | NO: 280 | (0.139/0.231 | (0.397 nM) | (0.168 nM) | (0.128 nM) | (0.107 nM) | (0.730 nM) | (0.363 nM) |
| mIGHV1- | | nM) | | | | | | |
| S45*01 | | | | | | | | |
| HCDR2 | | | | | | | | |
| NC | | | | | | | | |
| IGHV1-46*01 | SEQ ID | PR302341-13 | | PR302341-19 | | | | |
| | NO: 282 | (0.124 nM) | | (0.121 nM) | | | | |
| IGVH1-46*01 | SEQ ID | PR302341-14 | PR302341-7 | PR302341-8 | PR302341-9 | PR302341-10 | PR302341-11 | PR302341-12 |
| Back | NO: 281 | (0.075 nM) | (0.230 nM) | (0.165 nM) | (0.234 nM) | (0.110 nM) | (0.467 nM) | (0.250 nM) |
| Mutation | | | | | | | | |
| IGHV1-3*05 | SEQ ID | PR302341-15 | | PR302341-20 | | | | |
| | NO: 283 | (0.124 nM) | | (0.162 nM) | | | | |
| IGHV1-3*05 | SEQ ID | PR302341-16 | | PR302341-21 | | PR302341-24 | | PR302341-25 |
| Back | NO: 284 | (0.225 nM) | | (0.177 nM) | | (0.192 nM) | | (0.711 nM) |
| Mutation | | | | | | | | |
| IGHV1-2*02 | SEQ ID | PR302341-17 | | PR302341-22 | | | | |
| | NO: 285 | (0.104 nM) | | (0.243 nM) | | | | |
| IGHV1-2*02 | SEQ ID | PR302341-18 | | PR302341-23 | | PR302341-26 | | PR302341-27 |
| Back | NO: 286 | (0.169 nM) | | (0.043 nM) | | (0.273 nM) | | (0.518 nM) |
| Mutation | | | | | | | | |

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| PR [symbol] | [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | | [symbol] / [symbol] | | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |

[symbol]

indicates data missing or illegible when filed

PTM Removal of Humanized Series 10 mAbs PR302341-10, PR302341-20, and PR302341-23

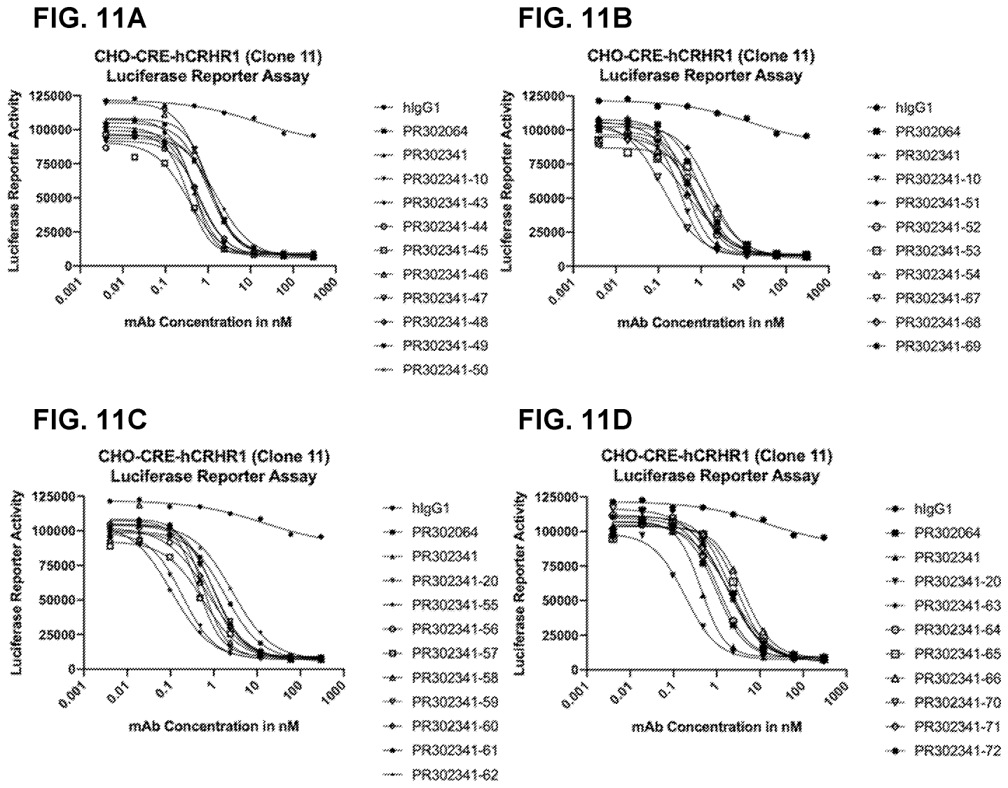

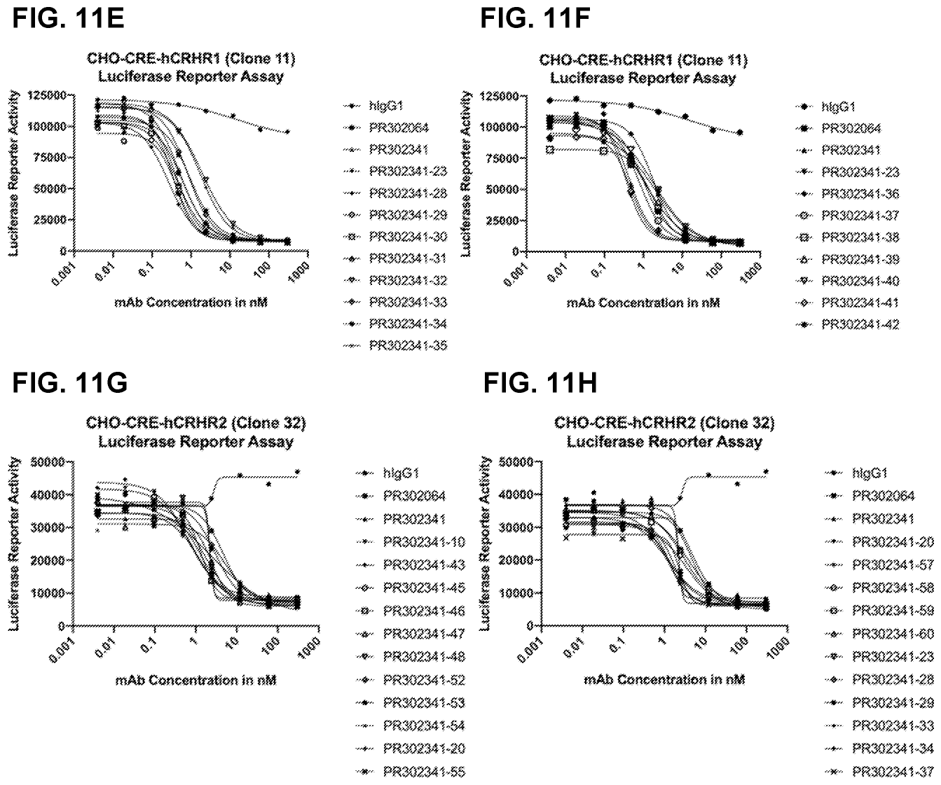

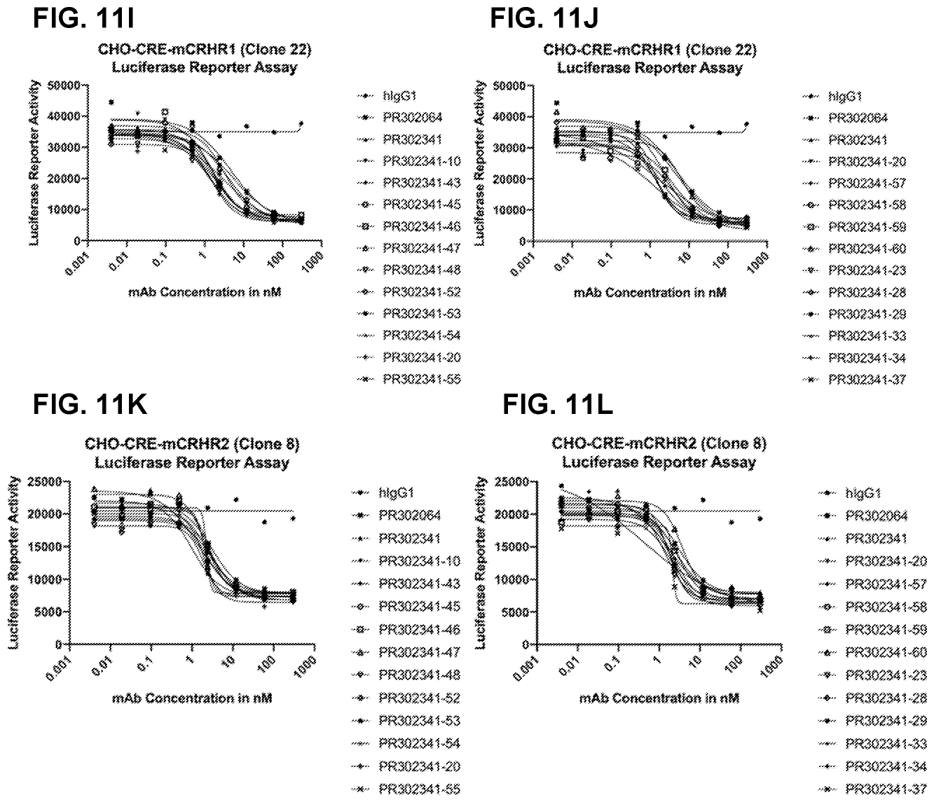

Humanized Series 10 mAbs PR302341-10, PR302341-20, and PR302341-23 were subjected to 4×4 double PTM removal mutagenesis to generate a total of 48 mAbs (3×4×4), including the above three parental humanized mAbs, using the materials and methods described above. Many humanization variants maintained high potency in vitro ELISA binding activities and functional blocking activities. See Table 9 and FIGS. 11A-11L. PR302341-34, PR302341-48, PR302341-60, PR302341-28 and PR302341-55 were scaled up for in vivo studies.

TABLE 9
Functional Blocking Activities of Series 10 Humanized mAbs PR302341-
10, PR302341-20 and PR302341-23 and their PTM Removed Variants

| mAb ID | IGKV2- [symbol] Back Mutation | | | |
|---|---|---|---|---|
| hCRHR1 Reporter IC50 (nM) | LCDR1: DG | LCDR1: DG-EG | LCDR1: DG-DA | LCDR1: DG-SG |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| mAb ID | IGKV2- [symbol] Back Mutation | | | |
| hCRHR1 Reporter IC50 (nM) | LCDR1: DG | LCDR1: DG-EG | LCDR1: DG-DA | LCDR1: DG-SG |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| mAb ID | IGKV2- [symbol] Back Mutation | | | |
| hCRHR1 Reporter IC50 (nM) | LCDR1: DG | LCDR1: DG-EG | LCDR1: DG-DA | LCDR1: DG-SG |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

| | | | | | ELISA EC50 (nM) | | | | | |
|---|---|---|---|---|---|---|---|---|---|---|
| mAb ID | IGHV | IGKV | HCDR2 | LCDR1 | CRH | CRH | Luciferase Reporter Assay IC50 (nM) | | | |
| mAb ID | IGHV | IGKV | HCDR2 | LCDR1 | NT-Biotin | CT-Biotin | hCRHR1 | hCRHR2 | mCrhr1 | mCrhr2 |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | [symbol] | [symbol] | [symbol] |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |
| PR [symbol] | [symbol] | [symbol] | [symbol] | [symbol] | | | [symbol] | | | |

[symbol]

indicates data missing or illegible when filed

### Example 6

### Selectivity Against Human UCN1, Human UCN2, and Human UCN3 Peptides

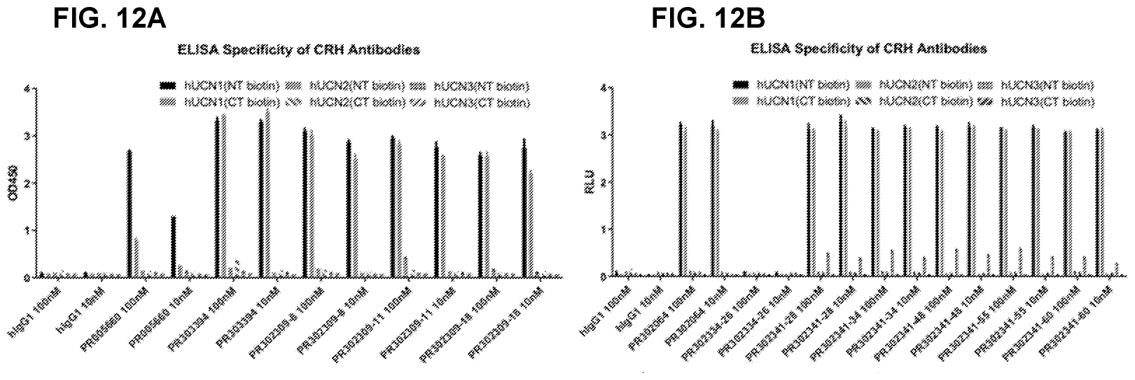

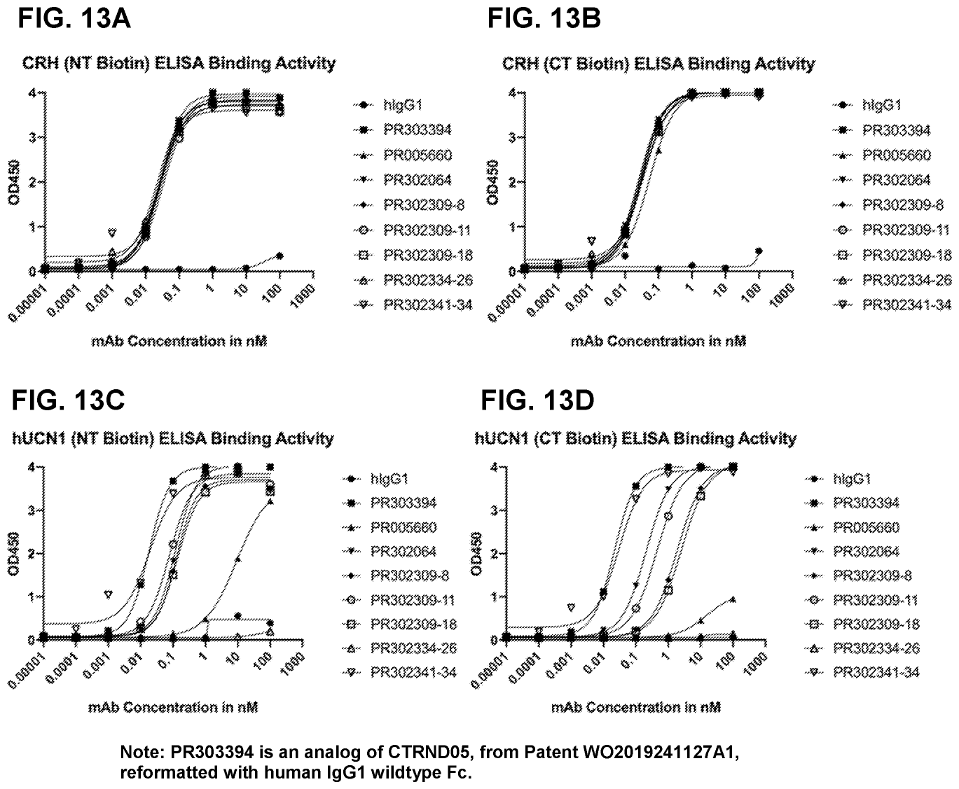

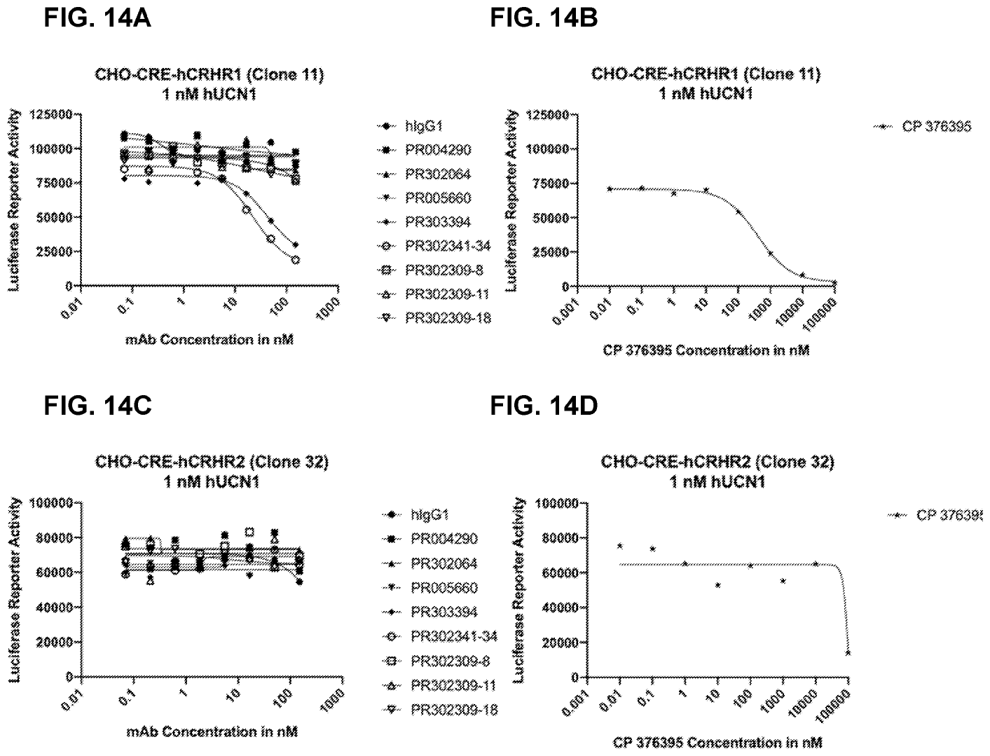

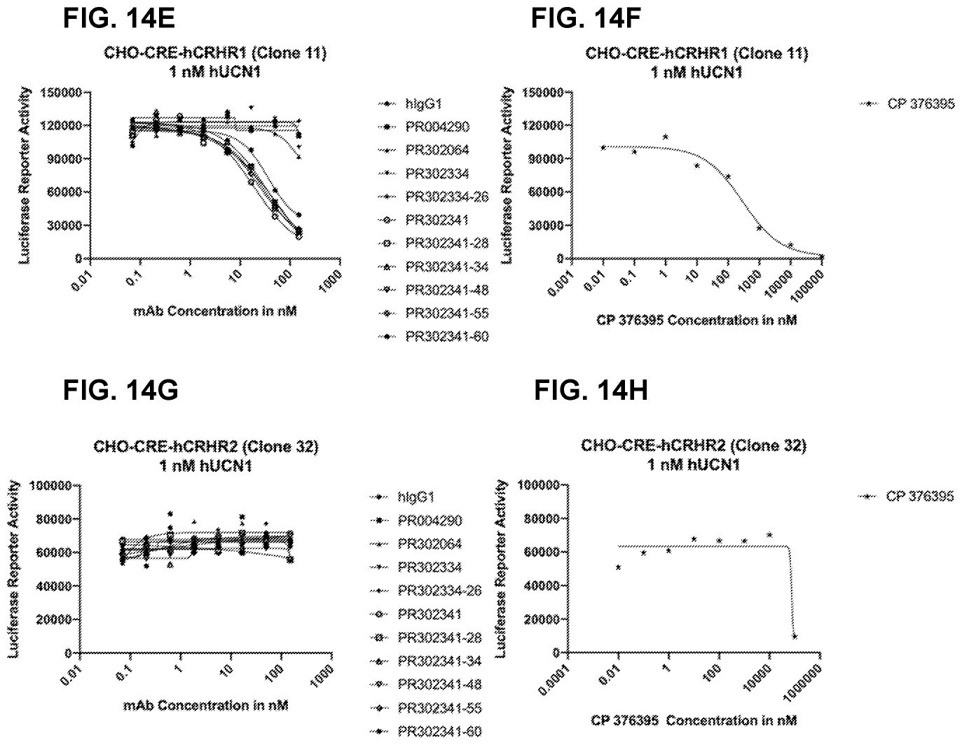

All tested mAbs, except for PR302334-26, bound to human UCN1 peptides in ELISA assays. None of the tested mAbs bound either human UCN2 or human UCN3 peptides in ELISA assays. The binding affinities of these mAbs to human UCN1 are comparable to that of CRH by ELISA. However, only mAbs from PR302341 series inhibited hUCN1-induced hCRHR1 luciferase reporter activity. In addition, these mAbs inhibited hUCN1-induced hCRHR1 reporter activity with a potency 30× to 267× lower than that of CRH-induced hCRHR1 reporter activity. None of the mAbs blocked hUCN1-induced hCRHR2 luciferase reporter activity. See Table 10 and FIGS. 12A-12B, 13A-13D and 14A-14H.

TABLE 10
Anti-CRH mAbs Block CRH-Induced Signaling with Higher Potency than that of hUCN1-Induced Signaling

| | CRH | CRH | hUCN1 | hUCN1 | | | |
|---|---|---|---|---|---|---|---|
| | (NT Biotin) | (CT Biotin) | (NT Biotin) | (CT Biotin) | CHO-CRE-hCRHR1 | CHO-CRE-hCRHR1 | |
| | ELISA EC50 | ELISA EC50 | ELISA (EC50 | ELISA EC50 | [symbol] .5 nM CRH | 1 nM hUCN1 | hUCN1 IC50/ |
| | (nM) | (nM) | nM) | nM) | IC50 (nM) | IC50 (nM) | CRH IC50 |
| Experiment 1 | | | | | | | |
| PR [symbol] | [symbol] | [symbol] | [symbol] | >1 nM | [symbol] | No Inhibition | Not Applicable |
| PR [symbol] | [symbol] | [symbol] | [symbol] | [symbol] | [symbol] | No inhibition | Not Applicable |
| PR [symbol] | [symbol] | [symbol] | [symbol] | — | [symbol] | No Inhibition | Not Applicable |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | No Inhibition | Not Applicable |
| PR [symbol] | [symbol] | [symbol] | [symbol] | [symbol] | [symbol] | No Inhibition | Not Applicable |
| PR [symbol] | [symbol] | [symbol] | [symbol] | [symbol] | [symbol] | No Inhibition | Not Applicable |
| Experiment 2 | | | | | | | |
| PR [symbol] | [symbol] | [symbol] | [symbol] | >1 nM | [symbol] | No Inhibition | Not Applicable |
| PR [symbol] | [symbol] | [symbol] | [symbol] | [symbol] | [symbol] | No Inhibition | Not Applicable |
| PR [symbol] | [symbol] | [symbol] | No Binding | No Binding | [symbol] | No Inhibition | Not Applicable |
| PR [symbol] | [symbol] | [symbol] | EC50 Not Tested | EC50 Not Tested | [symbol] | [symbol] | [symbol] |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] |

[symbol]

indicates data missing or illegible when filed

### Example 7

Anti-CRH mAbs Bind to N-Terminal Region of CRH Peptide

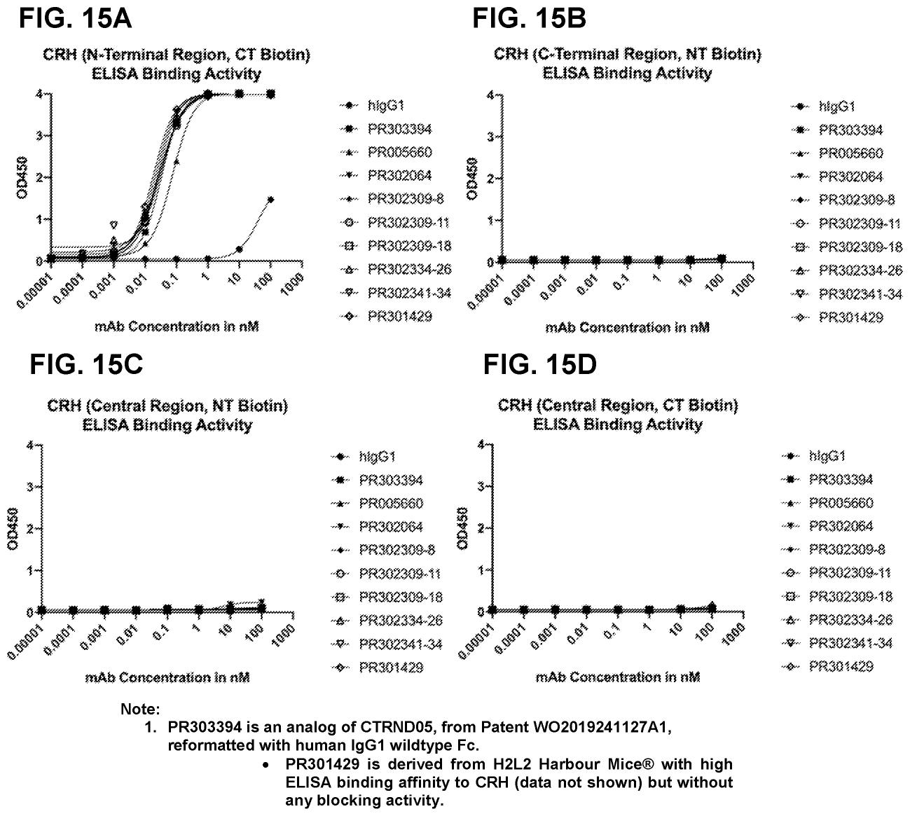

To determine the binding regions of the CRH peptide, the N-terminal region plus the central region, the central region only, and the central region plus C-terminal region of CRH peptides were used for ELISA capture of CRH, using the materials and methods described above. All mAbs bound to the N-terminal plus central region of the CRH peptide. Therefore, these mAbs likely bind to the N-terminal region of CRH. See Table 11 and FIGS. 15A-15D.

TABLE 11
Anti-CRH mAbs bind to N-terminal region of CRH peptide

| CRH Region: | N-Terminal -Central Region -C-Terminal | | | |
|---|---|---|---|---|
| Full Length CRH: | SEEPPISLDL TFHLLREVLE MARAEQLAQQ AHSNRKLMEI I-NH2 | | | |
| (SEQ ID NO: 481) | | | | |
| N-Terminal CRH: | SEEPPISLDL TFHLLREVLE MARAEQLAQQ A-K-Biotin | | | |
| (SEQ ID NO: 482) | | | | |
| C-Terminal CRH: | Biotin-K-VLE MARAEQLAQQ AHSNRKLMEI I-NH2 | | | |
| (SEQ ID NO: 483) | | | | |
| Central CRH: | Biotin-K-LREVLE MARAEQLAQQ AHSN-NH2 | | | |
| (SEQ ID NO: 484) | | | | |
| Central CRH: | LREVLE MARAEQLAQQ AHSN-K-Biotin | | | |
| (SEQ ID NO: 485) | | | | |
| | N-Ter-CRH | C-Ter-CRH | Central-CRH | Central-CRH |
| ELISA EC50 (nat) | (CT-Bion) | (CT-Bion) | (CT-Bion) | (NT-Bion) |
| hIgG1 | — | [symbol] | [symbol] | [symbol] |
| PR303394 | 0.034 | [symbol] | [symbol] | [symbol] |
| PR005660 | 0.076 | [symbol] | [symbol] | [symbol] |
| PR302064 | 0.022 | [symbol] | [symbol] | [symbol] |
| PR302309-8 | 0.025 | [symbol] | [symbol] | [symbol] |
| PR302309-11 | 0.028 | [symbol] | [symbol] | [symbol] |
| PR302309-18 | 0.028 | [symbol] | [symbol] | [symbol] |
| PR302334-26 | 0.029 | [symbol] | [symbol] | [symbol] |
| PR302341-34 | 0.034 | [symbol] | [symbol] | [symbol] |
| PR301429 | 0.019 | [symbol] | [symbol] | [symbol] |

[symbol]

indicates data missing or illegible when filed

### Example 8

Affinity Determination of mAbs with Octet

The binding affinities of anti-CRH antibodies were determined using the Octet platform, using the materials and methods described above. The identified mAbs have very high affinities, with equilibrium dissociation constant (KD) values around single digit to double digit pM. See Table 12.

TABLE 12
Affinity Determination of mAbs with Octet

| Antibody ID | KD (M) | kon (1/Ms) | kdis (1/s) | Full R\hat{ }2 | Antibody ID | KD (M) | kon (1/Ms) | kdis (1/s) | Full R\hat{ }2 |
|---|---|---|---|---|---|---|---|---|---|
| PR [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] | [symbol] | [symbol] |
| PR [symbol] | [symbol] | [symbol] | [symbol] | [symbol] | [symbol] | [symbol] | [symbol] | [symbol] | [symbol] |
| | | | | | [symbol] | [symbol] | [symbol] | [symbol] | [symbol] |
| PR004290 | [symbol] | [symbol] | [symbol] | [symbol] | [symbol] | [symbol] | [symbol] | [symbol] | [symbol] |
| PR006142 | [symbol] | [symbol] | [symbol] | [symbol] | [symbol] | [symbol] | [symbol] | [symbol] | [symbol] |
| PR006143 | [symbol] | [symbol] | [symbol] | [symbol] | [symbol] | [symbol] | [symbol] | [symbol] | [symbol] |
| PR006144 | [symbol] | [symbol] | [symbol] | [symbol] | [symbol] | [symbol] | [symbol] | [symbol] | [symbol] |
| PR006145 | [symbol] | [symbol] | [symbol] | [symbol] | [symbol] | [symbol] | [symbol] | [symbol] | [symbol] |
| PR006146 | [symbol] | [symbol] | [symbol] | [symbol] | [symbol] | [symbol] | [symbol] | [symbol] | [symbol] |
| PR006147 | [symbol] | [symbol] | [symbol] | [symbol] | [symbol] | [symbol] | [symbol] | [symbol] | [symbol] |
| PR006148 | [symbol] | [symbol] | [symbol] | [symbol] | [symbol] | [symbol] | [symbol] | [symbol] | [symbol] |
| PR006149 | [symbol] | [symbol] | [symbol] | [symbol] | [symbol] | [symbol] | [symbol] | [symbol] | [symbol] |
| PR006150 | [symbol] | [symbol] | [symbol] | [symbol] | [symbol] | [symbol] | [symbol] | [symbol] | [symbol] |
| PR006151 | [symbol] | [symbol] | [symbol] | [symbol] | [symbol] | [symbol] | [symbol] | [symbol] | [symbol] |
| PR006152 | [symbol] | [symbol] | [symbol] | [symbol] | [symbol] | [symbol] | [symbol] | [symbol] | [symbol] |
| PR006153 | [symbol] | [symbol] | [symbol] | [symbol] | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |
| | | | | | [symbol] | [symbol] | [symbol] | [symbol] | [symbol] |

[symbol]

indicates data missing or illegible when filed

### Example 9

Antibody Binding Epitope Binding with ELISA

Selected mAbs were tested for their ability to block each other for binding to biotin-CRH in a pairwise competition ELISA, using materials and methods described above. Table 13 shows that these mAbs blocked binding of each other to CRH, and therefore, likely bind to a similar epitope, possibly at the N-terminus of CRH.

TABLE 13
Antibody Binding Epitope Binding with ELISA
Competitive ELISA: Immobilized 1st mAb + Biotin-CRN + 2^{nd} mAbs + Detection StreptAvidin-HRP

| | | 2nd mAbs: Competitor mAbs at 50 μg/ml | | | | | | | | | |
|---|---|---|---|---|---|---|---|---|---|---|---|
| % Inhibition | | hIgG1 | PR004290 | PR005660 | PR006148 | PR006152 | PR301777 | PR302080 | PR302094 | PR302055 | PR302088 |
| 1^{st} mAbs: | hIgG1 | [symbol] | [symbol] | [symbol] | [symbol] | [symbol] | [symbol] | [symbol] | [symbol] | [symbol] | [symbol] |
| Coat at | PR [symbol] | [symbol] | [symbol] | [symbol] | [symbol] | [symbol] | [symbol] | [symbol] | [symbol] | [symbol] | [symbol] |
| 1 μg/ml | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |
| | PR [symbol] | [symbol] | [symbol] | [symbol] | [symbol] | [symbol] | [symbol] | [symbol] | [symbol] | [symbol] | [symbol] |

| | | |
|---|---|---|
| Key | [symbol] | [symbol] |

Note:
PR303394 is an analog of CTRND05, from Patent WO2019241127A1, reformatted with human IgG1 wildtype Fc. [symbol]
indicates data missing or illegible when filed

### Example 10

Antibody Binding Epitope Binding with Octet

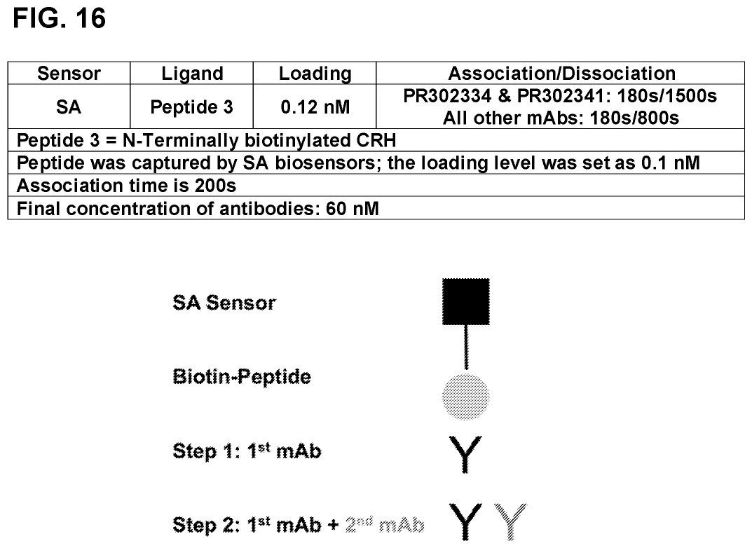

Selected mAbs were tested for their ability to block each other for binding to biotin-CRH in a pairwise competition Octet assay, using the materials and methods described above. FIG. 16 shows a diagram of the competition assay using the Octet platform. Table 14 shows that these mAbs blocked binding of each other to CRH, and therefore, likely bind to a similar epitope, possibly at the N-terminus of CRH.

TABLE 14
Antibody Binding Epitope Binding with Octet

| | | 2^{nd} mAb | | | | | | | |
|---|---|---|---|---|---|---|---|---|---|
| % Inhibition | | PR005660 | PR006669 | PR301777 | PR302309-8 | PR302334 | PR302341-34 | PR303394 | hIgG1 |
| 1^{st} | PR005660 | 95 | 94 | 93 | 93 | 88 | 90 | 91 | — |
| mAb | PR006669 | 92 | 95 | 91 | 93 | 88 | 87 | 88 | — |
| | PR301777 | 82 | 82 | 87 | 86 | 73 | 79 | 82 | — |
| | PR302309-8 | 76 | 76 | 80 | 87 | 65 | 74 | 77 | — |
| | PR302334 | 92 | 93 | 90 | 91 | 95 | 88 | 89 | — |
| | PR302341-34 | 92 | 92 | 92 | 92 | 88 | 94 | 93 | — |
| | PR303394 | 91 | 90 | 93 | 94 | 86 | 91 | 97 | — |
| | hIgG1 | 12 | 9 | 9 | 15 | 8 | 12 | 10 | — |

| | | | |
|---|---|---|---|
| Key | >80% inhibition; Same | 40%-80% Inhibition; Adjacent | <40% Inhibition; Different |
| | Epitope | Epitope | Epitope |

### Example 11

### In Vivo Efficacy Studies

### Wildtype Mice Restraint-Induced Transient Increase of ACTH and Corticosterone

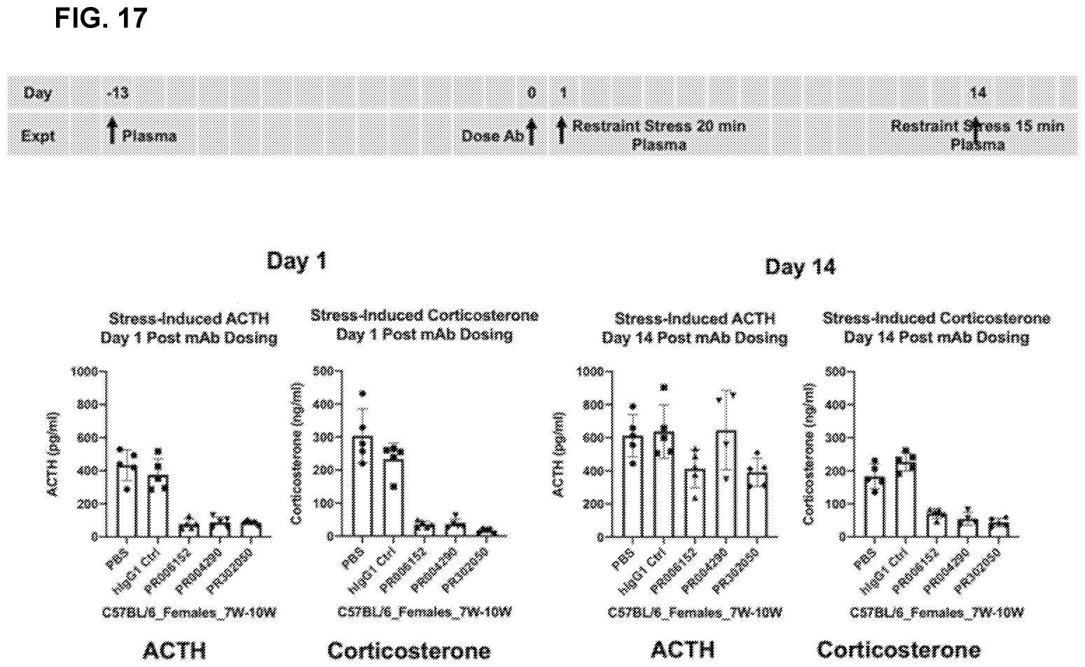

Treatment with anti-CRH mAbs PR004290 (Series 1 mAb), PR006152 (Series 1 mAb), and PR302050 (Series 5 mAb) (20 mg/kg IP) markedly inhibited adrenocorticotropic hormone (ACTH) and corticosterone concentrations in wildtype mice subjected to the restraint-induced stress model discussed above. Day 1 post-mAb dosing inhibited plasma ACTH and corticosterone concentrations down to basal unstressed levels. Fourteen days after a single dose of mAb, ACTH and corticosterone concentrations remained partially inhibited. See FIG. 17.

### Mrap1 KO Mice Constitutively High ACTH Model

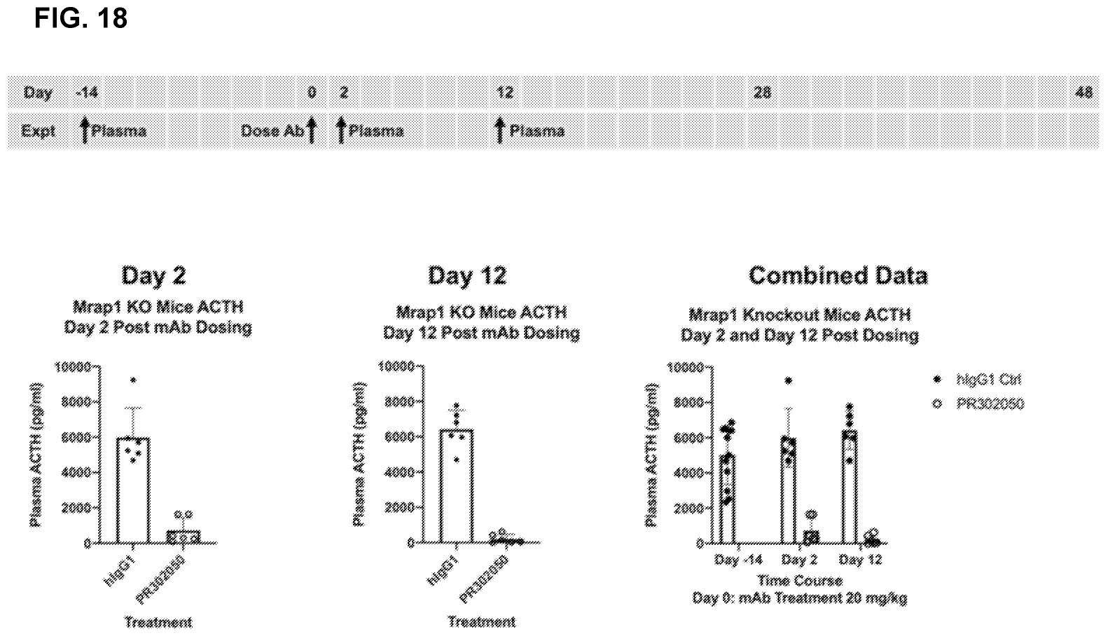

Mrap1 KO mice have constitutively high plasma ACTH concentrations of ˜5,000 μg/ml, approximately 50-fold higher than the basal ACTH in wildtype mice, and ˜10-fold higher than those observed in restraint-induced stress in wildtype mice. A single dose of anti-CRH mAb PR302050 (Series 5 mAb) at 20 mg/kg markedly reduced the constitutively high concentrations of ACTH in Mrap1 KO mice both 2 and 14 days after administration. See FIG. 18.

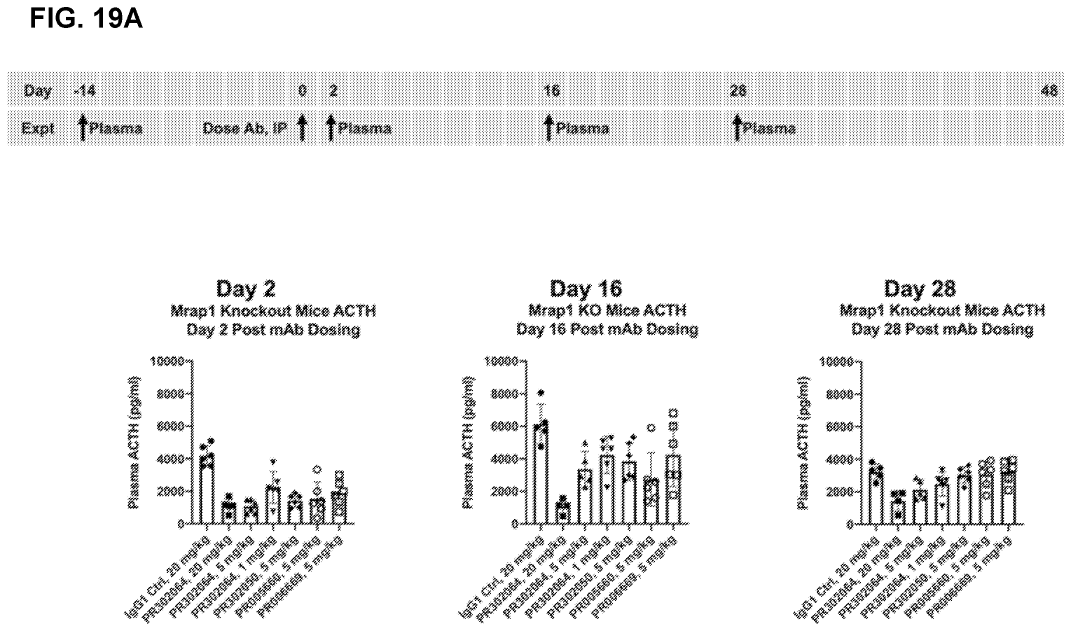

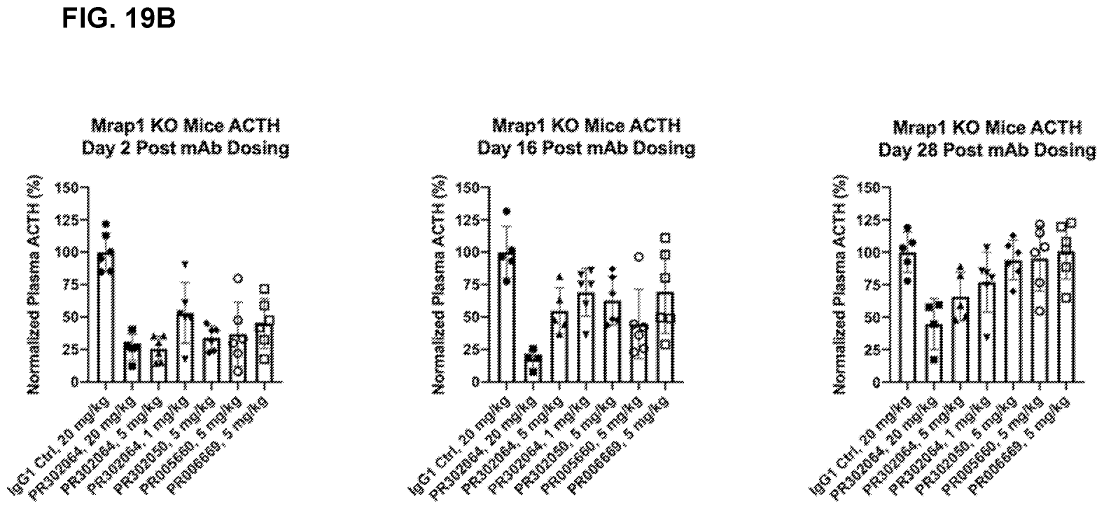

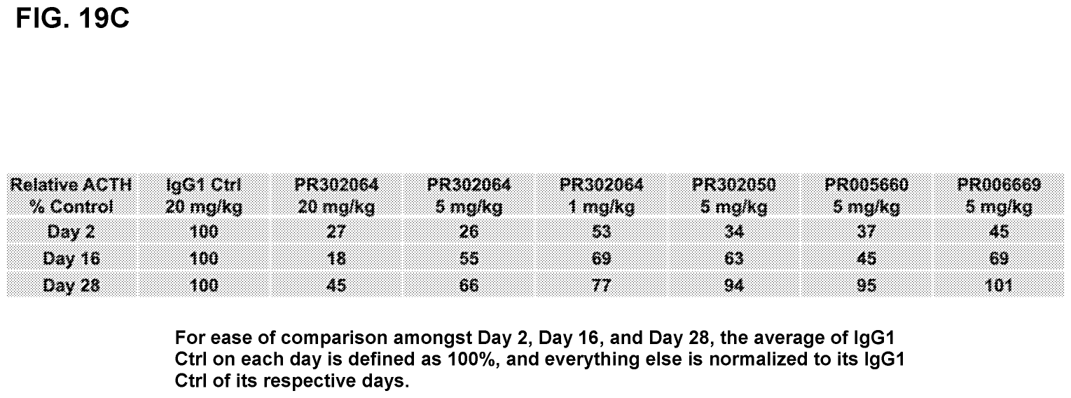

A single dose of anti-CRH mAb PR302064 (Series 5 mAb) markedly reduced constitutively high levels of ACTH in Mrap1 KO mice in a dose-dependent manner at 20 mg/kg, 5 mg/kg, and 1 mg/kg, on post-dosing Days 2, 16, and 28, respectively. PR302064 at 20 mg/kg had 73%, 82%, and 55% reduction of ACTH on Days 2, 16, and 28, respectively. PR302064 at 5 mg/kg had 74%, 45%, and 34% reduction of ACTH on Days 2, 16, and 28, respectively. PR302064 at 1 mg/kg had 47%, 31%, and 23% reduction of ACTH on Days 2, 16, and 28, respectively. Other mAbs at 5 mg/kg, including PR302050 (Series 5 mAb), PR005660 (Series 1 mAb), and PR006669 (Series 2 mAb), also significantly reduced ACTH on post-dosing days 2 and 16. See FIGS. 19A-19C.

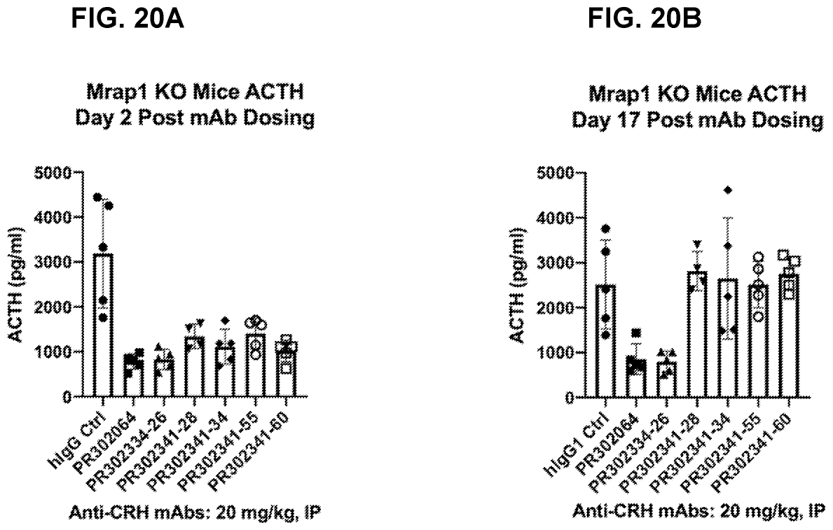

Additional anti-CRH mAbs were tested in a Mrap1 KO model. Single doses of PR302334-26 (Series 9 mAb), PR302341-28, PR302341-34, PR302341-55, and PR302341-60 (all Series 10 mAbs) at 20 mg/kg also markedly reduced plasma ACTH concentrations in Mrap1 KO mice. See FIGS. 20A-20B.

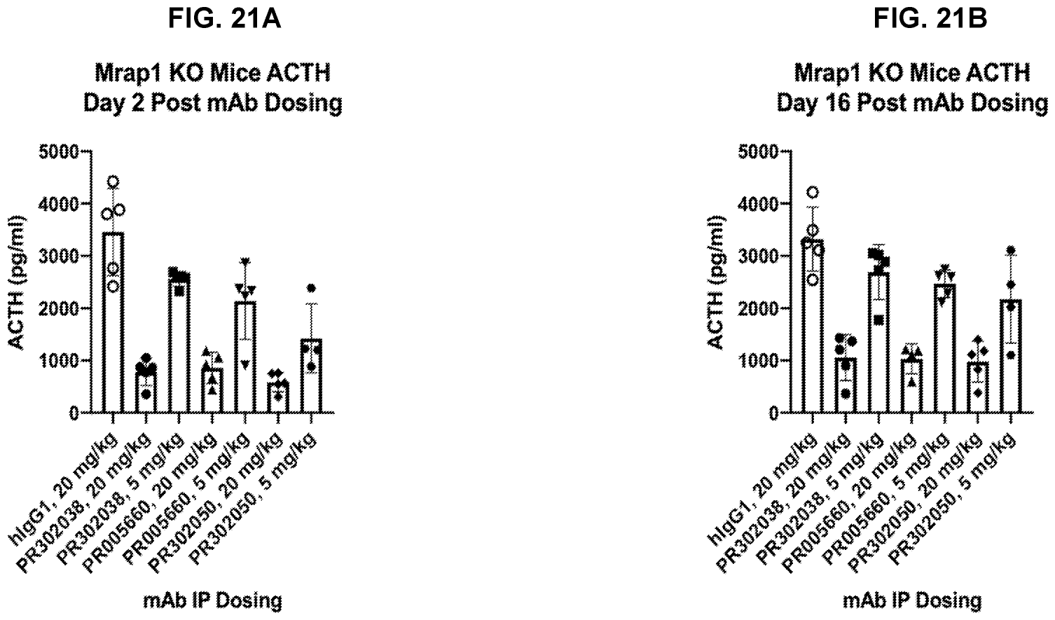

PR302038, PR005660, and PR302050 reduced plasma ACTH concentrations in Mrap1 KO mice, in a dose dependent manner, at 20 mg/kg and 5 mg/kg doses of antibody. See FIGS. 21A-21B.

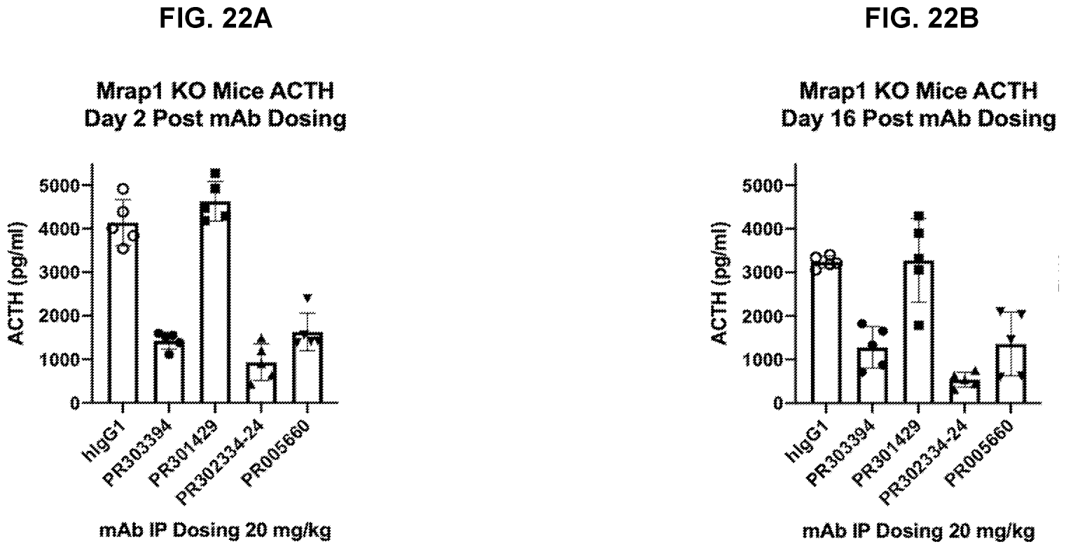

PR302334-24 and PR005660 at 20 mg/kg significantly reduced plasma ACTH concentrations in Mrap1 KO mice, whereas PR301429, an anti-CRH mAb with comparable binding affinity, but a non-blocker in vitro, did not inhibit ACTH production. PR303394, an analog of CTRND05, was used as a comparator. See FIGS. 22A-22B.

### Anti-CRH mAb PR302064 had Superior Efficacy to SSR125543A in a Wildtype Mice Restraint-Induced ACTH Model

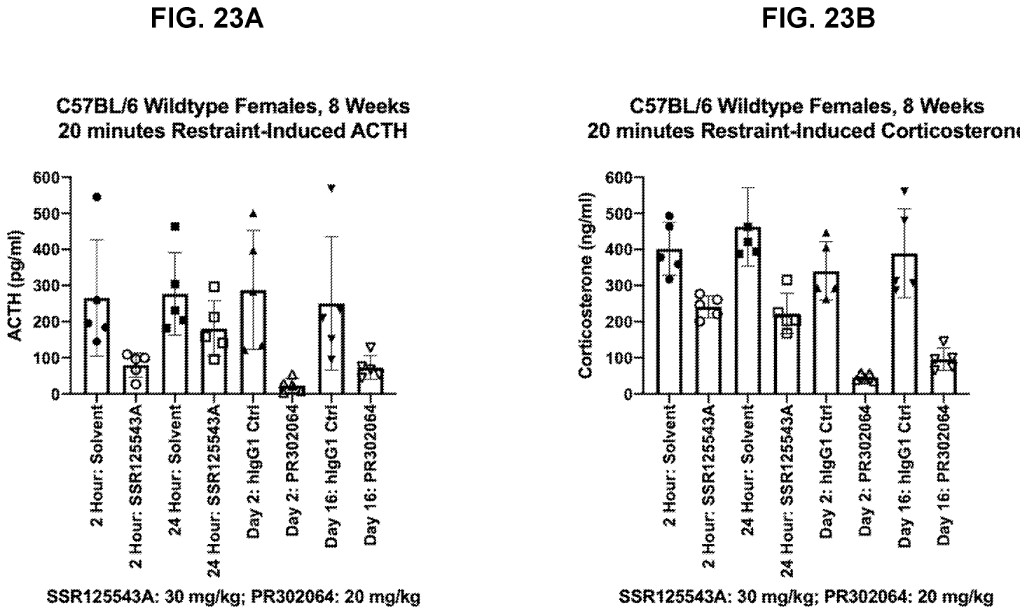

Anti-CRH mAb PR302064 (Series 5 mAb) at 20 mg/kg was more efficacious than CRHR1 small molecule antagonist SSR125543A (Crinecerfont) at 30 mg/kg in reducing restraint-induced ACTH in wildtype mice. PR302064 had a more complete inhibition of both ACTH and corticosterone, and a longer duration of action. The effects of this mAb lasted for at least 16 days. See FIGS. 23A-23B.

Anti-CRH mAbs PR302064 and PR302334-24 had Superior Efficacy to SSR125543A in the Mrap1 KO Mice Constitutively High ACTH Model

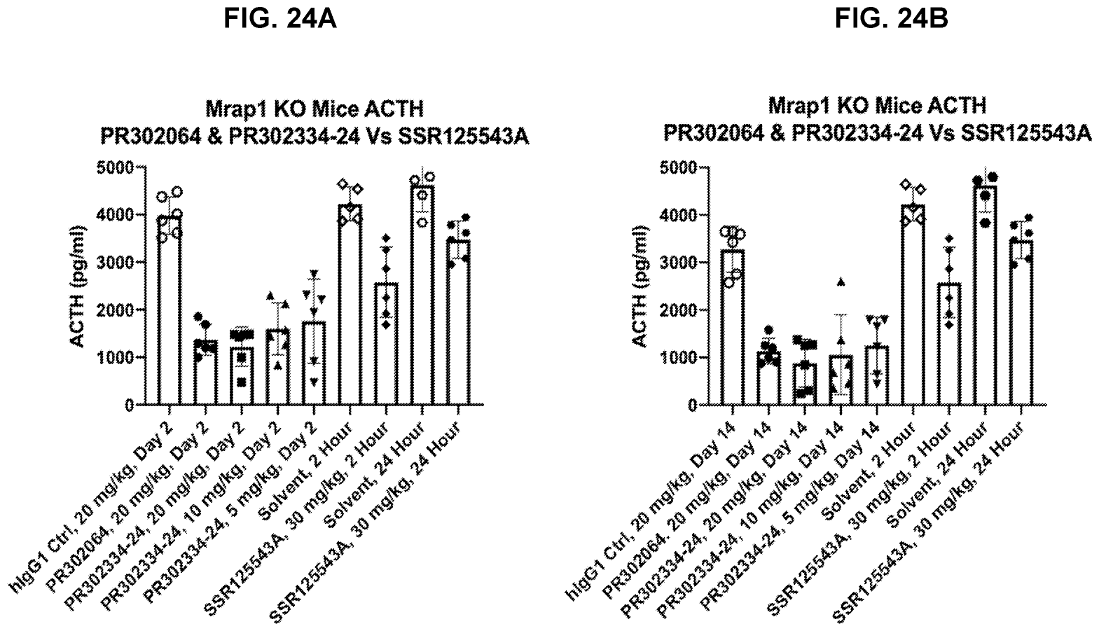

Anti-CRH mAb PR302064 at 20 mg/kg and PR302334-24 at 20 mg/kg, 10 mg/kg, and 5 mg/kg, respectively, were all more efficacious than CRHR1 small molecule antagonist SSR125543A (Crinecerfont) at 30 mg/kg in reducing plasma ACTH levels in the Mrap1 KO Mice constitutively high ACTH model. PR302064 and PR302334-24 had more complete inhibition, and more importantly had longer duration of action. The effects of these two mAbs lasted for at least 14 days. See FIGS. 24A-24B.

### Anti-CRH mAb PR302334-24 had Superior Efficacy to SSR125543A in the Wildtype Mice Restraint Stress Induced High ACTH and Corticosterone Model

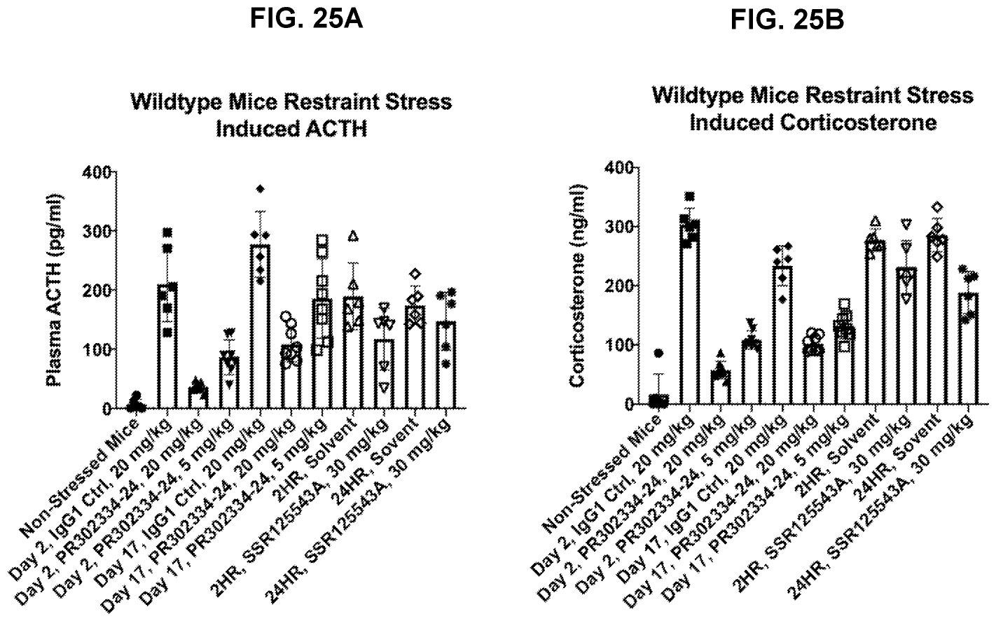

Anti-CRH mAb PR302334-24 at 20 mg/kg and 5 mg/kg were both more efficacious than CRHR1 small molecule antagonist SSR125543A (Crinecerfont) at 30 mg/kg in reducing plasma ACTH and corticosterone levels in a wildtype mice restraint stress induced high ACTH and corticosterone model. PR302334-24 had more complete inhibition, and more importantly had longer duration of action. The effects of PR302334-24 lasted for at least 17 days. See FIGS. 25A-25B.

### In Vivo PK Study

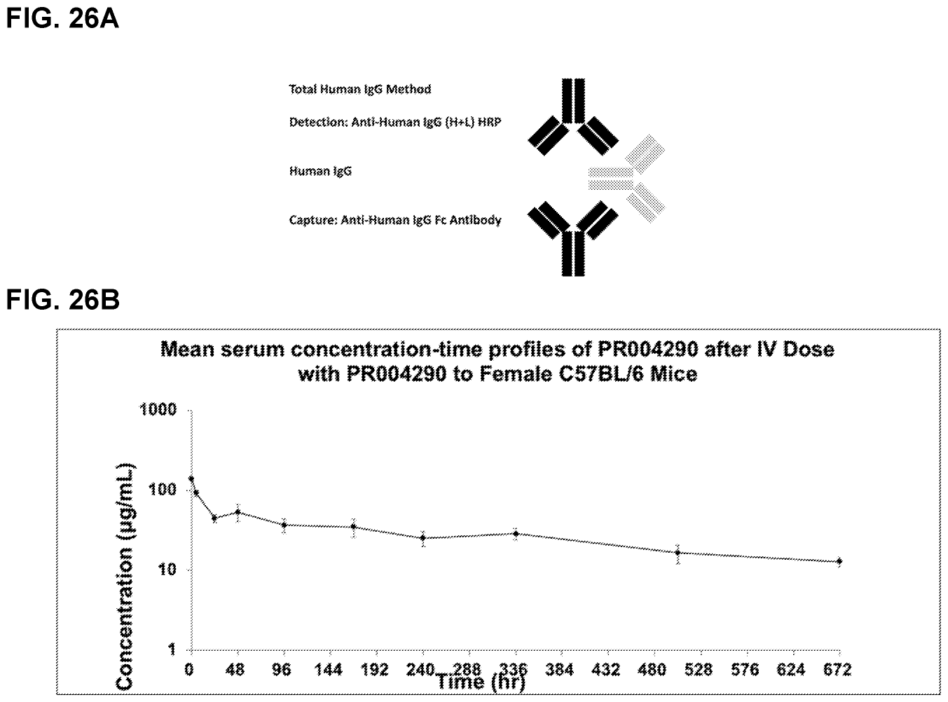

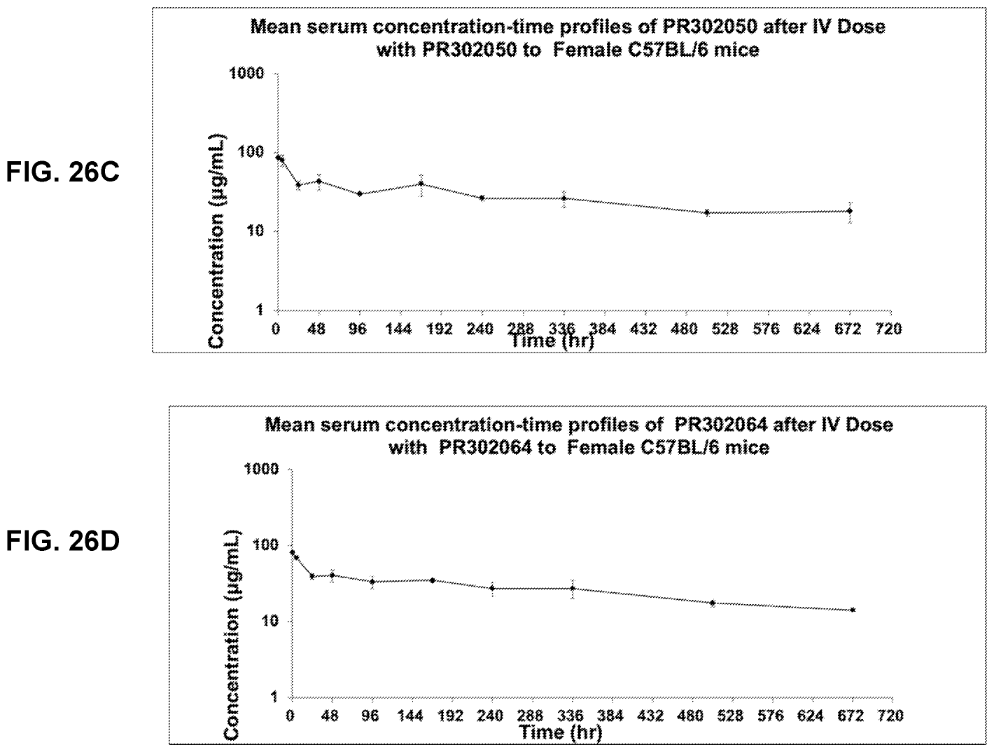

The pharmacokinetics of mAbs PR004290, PR302050, and PR302064 were tested in C57BL/6 wildtype mice in a single IV dose of 5 mg/kg. Specifically, anti-CRH mAbs in human IgG1-WT-Fc present in mouse serum were captured by goat anti-human Fc polyclonal antibody and detected with goat anti-human (H+L) secondary antibody conjugated with HRP. FIG. 26A shows a diagram of this method.

As shown in Table 15 and FIGS. 26B-26D, serum half-life (T_{1/2}) of PR004290, PR302050 and PR302064 were 14 days, 21 days and 18 days, respectively. These data indicate that these mAbs have typical human IgG1 PK profiles, with desirable T_{1/2} for clinical application.

TABLE 15
PK Analysis of Antibodies in Single IV Dose of
5 mg/kg in Female C57BL/6 Wildtype Mice (N = 6)

| PK Parameters | PR004290 | PR302050 | PR302064 |
|---|---|---|---|
| CL_pred (mL/h/kg) | 0.202 | 0.17 | 0.19 |
| Vₛₛ_pred (mL/kg) | 94.4 | 118 | 114 |
| C₀ (μg/mL) | 141 | 85.4 | 80.8 |
| T_{1/2} (h) | 338 | 502 | 426 |
| AUCₗₐₛₜ (h*μg/mL) | 18785 ± 1414 | 18300 ± 1506 | 17762 ± 1008 |

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An antibody or antigen-binding fragment thereof that specifically binds to corticotropin-releasing hormone (CRH), comprising:
(a) a heavy chain variable domain (VH) CDR1 comprising an amino acid sequence of any one of SEQ ID NOs: 22-35, a VH CDR2 comprising an amino acid sequence of any one of SEQ ID NOs: 53-78, and a VH CDR3 comprising an amino acid sequence of any one of SEQ ID NOs: 113-122 or the amino acid sequence of PDV or GID; and
(b) a light chain variable domain (VL) CDR1 comprising an amino acid sequence of any one of SEQ ID NOs: 152-174, a VL CDR2 comprising the amino acid sequence of any one of SEQ ID NOs: 193-198, and a VL CDR3 comprising an amino acid sequence of any one of SEQ ID NOs: 223-232.

2. The antibody or antigen-binding fragment thereof of claim 1, wherein the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 153, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 154, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 156 the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 54, the VH CDR3 comprises the amino acid sequence of SEQ ID NO: 113, the VL CDR1 comprises the amino acid sequence of SEQ ID NO: 157, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 154, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 156, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 55, the VH CDR3 comprises the amino acid sequence of SEQ ID NO: 113, the VL CDR1 comprises the amino acid sequence of SEQ ID NO: 157 the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 154 the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 155, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 156, the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 22, the VH CDR2 comprises the amino acid sequence of SEQ ID NO: 56, the VH CDR3 comprises the amino acid sequence of SEQ ID NO: 113, the VL CDR1 comprises the amino acid sequence of SEQ ID NO: 157 the VL CDR2 comprises the amino acid sequence of SEQ ID NO: 193, and the VL CDR3 comprises the amino acid sequence of SEQ ID NO: 223;
wherein the VH CDR1 comprises the amino acid sequence of SEQ ID NO: 23, the VH CDR2 comprises the amino acid sequence of SEQ ID NO: 57, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224;
wherein the VH CDR1 comprises the amino acid sequence of SEQ ID NO: 23, the VH CDR2 comprises the amino acid sequence of SEQ ID NO: 58, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224;
wherein the VH CDR1 comprises the amino acid sequence of SEQ ID NO: 23, the VH CDR2 comprises the amino acid sequence of SEQ ID NO: 59, the VH CDR3 comprises the amino acid sequence of SEQ ID NO: 114, the VL CDR1 comprises the amino acid sequence of SEQ ID NO: 158 the VL CDR2 comprises the amino acid sequence of SEQ ID NO: 193, and the VL CDR3 comprises the amino acid sequence of SEQ ID NO: 224;
wherein the VH CDR1 comprises the amino acid sequence of SEQ ID NO: 23, the VH CDR2 comprises the amino acid sequence of SEQ ID NO: 60, the VH CDR3 comprises the amino acid sequence of SEQ ID NO: 114, the VL CDR1 comprises the amino acid sequence of SEQ ID NO: 158, the VL CDR2 comprises the amino acid sequence of SEQ ID NO: 193, and the VL CDR3 comprises the amino acid sequence of SEQ ID NO: 224;
wherein the VH CDR1 comprises the amino acid sequence of SEQ ID NO: 24, the VH CDR2 comprises the amino acid sequence of SEQ ID NO: 61, the VH CDR3 comprises the amino acid sequence of SEQ ID NO: 115, the VL CDR1 comprises the amino acid sequence of SEQ ID NO: 159, the VL CDR2 comprises the amino acid sequence of SEQ ID NO: 194, and the VL CDR3 comprises the amino acid sequence of SEQ ID NO: 225;
wherein the VH CDR1 comprises the amino acid sequence of SEQ ID NO: 25, the VH CDR2 comprises the amino acid sequence of SEQ ID NO: 62, the VH CDR3 comprises the amino acid sequence of PDV, the VL CDR1 comprises the amino acid sequence of SEQ ID NO: 160, the VL CDR2 comprises the amino acid sequence of SEQ ID NO: 195, and the VL CDR3 comprises the amino acid sequence of SEQ ID NO: 226;
wherein the VH CDR1 comprises the amino acid sequence of SEQ ID NO: 24, the VH CDR2 comprises the amino acid sequence of SEQ ID NO: 63, the VH CDR3 comprises the amino acid sequence of SEQ ID NO: 115, the VL CDR1 comprises the amino acid sequence of SEQ ID NO: 161, the VL CDR2 comprises the amino acid sequence of SEQ ID NO: 196, and the VL CDR3 comprises the amino acid sequence of SEQ ID NO: 225;
wherein the VH CDR1 comprises the amino acid sequence of SEQ ID NO: 26, the VH CDR2 comprises the amino acid sequence of SEQ ID NO: 64, the VH CDR3 comprises the amino acid sequence of SEQ ID NO: 116, the VL CDR1 comprises the amino acid sequence of SEQ ID NO: 162, the VL CDR2 comprises the amino acid sequence of SEQ ID NO: 197, and the VL CDR3 comprises the amino acid sequence of SEQ ID NO: 227;
wherein the VH CDR1 comprises the amino acid sequence of SEQ ID NO: 24, the VH CDR2 comprises the amino acid sequence of SEQ ID NO: 63, the VH CDR3 comprises the amino acid sequence of SEQ ID NO: 115, the VL CDR1 comprises the amino acid sequence of SEQ ID NO: 163, the VL CDR2 comprises the amino acid sequence of SEQ ID NO: 196, and the VL CDR3 comprises the amino acid sequence of SEQ ID NO: 225;
wherein the VH CDR1 comprises the amino acid sequence of SEQ ID NO: 24, the VH CDR2 comprises the amino acid sequence of SEQ ID NO: 65, the VH CDR3 comprises the amino acid sequence of SEQ ID NO: 115, the VL CDR1 comprises the amino acid sequence of SEQ ID NO: 161, the VL CDR2 comprises the amino acid sequence of SEQ ID NO: 196, and the VL CDR3 comprises the amino acid sequence of SEQ ID NO: 225;
wherein the VH CDR1 comprises the amino acid sequence of SEQ ID NO: 27, the VH CDR2 comprises the amino acid sequence of SEQ ID NO: 66, the VH CDR3 comprises the amino acid sequence of SEQ ID NO: 117, the VL CDR1 comprises the amino acid sequence of SEQ ID NO: 164, the VL CDR2 comprises the amino acid sequence of SEQ ID NO: 197, and the VL CDR3 comprises the amino acid sequence of SEQ ID NO: 228;
wherein the VH CDR1 comprises the amino acid sequence of SEQ ID NO: 28, the VH CDR2 comprises the amino acid sequence of SEQ ID NO: 67, the VH CDR3 comprises the amino acid sequence of SEQ ID NO: 118, the VL CDR1 comprises the amino acid sequence of SEQ ID NO: 165, the VL CDR2 comprises the amino acid sequence of SEQ ID NO: 197, and the VL CDR3 comprises the amino acid sequence of SEQ ID NO: 227;
wherein the VH CDR1 comprises the amino acid sequence of SEQ ID NO: 29, the VH CDR2 comprises the amino acid sequence of SEQ ID NO: 68, the VH CDR3 comprises the amino acid sequence of SEQ ID NO: 119, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 227;
wherein the VH CDR1 comprises the amino acid sequence of SEQ ID NO: 30, the VH CDR2 comprises the amino acid sequence of SEQ ID NO: 69, the VH CDR3 comprises the amino acid sequence of SEQ ID NO: 120, the VL CDR1 comprises the amino acid sequence of SEQ ID NO: 167, the VL CDR2 comprises the amino acid sequence of SEQ ID NO: 197, and the VL CDR3 comprises the amino acid sequence of SEQ ID NO: 229;
wherein the VH CDR1 comprises the amino acid sequence of SEQ ID NO: 29, the VH CDR2 comprises the amino acid sequence of SEQ ID NO: 70, the VH CDR3 comprises the amino acid sequence of SEQ ID NO: 121, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 31, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231;
wherein the VH CDR1 comprises the amino acid sequence of SEQ ID NO: 32, the VH CDR2 comprises the amino acid sequence of SEQ ID NO: 72, the VH CDR3 comprises the amino acid sequence of SEQ ID NO: 118, the VL CDR1 comprises the amino acid sequence of SEQ ID NO: 166, the VL CDR2 comprises the amino acid sequence of SEQ ID NO: 197, and the VL CDR3 comprises the amino acid sequence of SEQ ID NO: 227;
wherein the VH CDR1 comprises the amino acid sequence of SEQ ID NO: 33, the VH CDR2 comprises the amino acid sequence of SEQ ID NO: 73, the VH CDR3 comprises the amino acid sequence of SEQ ID NO: 118, the VL CDR1 comprises the amino acid sequence of SEQ ID NO: 169, the VL CDR2 comprises the amino acid sequence of SEQ ID NO: 197, and the VL CDR3 comprises the amino acid sequence of SEQ ID NO: 227;
wherein the VH CDR1 comprises the amino acid sequence of SEQ ID NO: 34, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231;
wherein the VH CDR1 comprises the amino acid sequence of SEQ ID NO: 30, the VH CDR2 comprises the amino acid sequence of SEQ ID NO: 74, the VH CDR3 comprises the amino acid sequence of SEQ ID NO: 122, the VL CDR1 comprises the amino acid sequence of SEQ ID NO: 170, the VL CDR2 comprises the amino acid sequence of SEQ ID NO: 197, and the VL CDR3 comprises the amino acid sequence of SEQ ID NO: 232;
wherein the VH CDR1 comprises the amino acid sequence of SEQ ID NO: 35, the VH CDR2 comprises the amino acid sequence of SEQ ID NO: 71, the VH CDR3 comprises the amino acid sequence of GID, the VL CDR1 comprises the amino acid sequence of SEQ ID NO: 168, the VL CDR2 comprises the amino acid sequence of SEQ ID NO: 198, and the VL CDR3 comprises the amino acid sequence of SEQ ID NO: 231;
wherein the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 172 the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 172, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 173, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 76, the VH CDR3 comprises the amino acid sequence of SEQ ID NO: 121, the VL CDR1 comprises the amino acid sequence of SEQ ID NO: 174 the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 77, the VH CDR3 comprises the amino acid sequence of SEQ ID NO: 121, the VL CDR1 comprises the amino acid sequence of SEQ ID NO: 174 the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 78, the VH CDR3 comprises the amino acid sequence of SEQ ID NO: 121, the VL CDR1 comprises the amino acid sequence of SEQ ID NO: 171, the VL CDR2 comprises the amino acid sequence of SEQ ID NO: 197, and the VL CDR3 comprises the amino acid sequence of SEQ ID NO: 230;
wherein the VH CDR1 comprises the amino acid sequence of SEQ ID NO: 29, the VH CDR2 comprises the amino acid sequence of SEQ ID NO: 75, the VH CDR3 comprises the amino acid sequence of SEQ ID NO: 121, the VL CDR1 comprises the amino acid sequence of SEQ ID NO: 174, the VL CDR2 comprises the amino acid sequence of SEQ ID NO: 197, and the VL CDR3 comprises the amino acid sequence of SEQ ID NO: 230; or
wherein the VH CDR1 comprises the amino acid sequence of SEQ ID NO: 22, the VH CDR2 comprises the amino acid sequence of SEQ ID NO: 53, the VH CDR3 comprises the amino acid sequence of SEQ ID NO: 113, the VL CDR1 comprises the amino acid sequence of SEQ ID NO: 152, the VL CDR2 comprises the amino acid sequence of SEQ ID NO: 193, and the VL CDR3 comprises the amino acid sequence of SEQ ID NO: 223.

3-56. (canceled)

57. The antibody or antigen-binding fragment thereof of claim 1, comprising:
(a) a VH comprising an amino acid sequence at least 90% identical to any one of SEQ ID NOs: 240-295, 459 and 461; and/or
(b) a VL comprising an amino acid sequence at least 90% identical to any one of SEQ ID NOs: 296-347 and 462.

58. The antibody or antigen-binding fragment thereof of claim 57, comprising:
(a) a VH comprising an amino acid sequence at least 95% identical to any one of SEQ ID NOs: 240-295, 459 and 461; and/or
(b) a VL comprising an amino acid sequence at least 95% identical to any one of SEQ ID NOs: 296-347 and 462.

59. The antibody or antigen-binding fragment thereof of claim 58, comprising:
(a) a VH comprising an amino acid sequence of any one of SEQ ID NOs: 240-295, 459 and 461; and/or
(b) a VL comprising an amino acid sequence of any one of SEQ ID NOs: 296-347 and 462.

60. The antibody or antigen-binding fragment thereof of claim 59,
wherein the VH comprises the amino acid sequence of SEQ ID NO: 240, and/or the VL comprises the amino acid sequence of SEQ ID NO: 296;
wherein the VH comprises the amino acid sequence of SEQ ID NO: 241, and/or the VL comprises the amino acid sequence of SEQ ID NO: 296;
wherein the VH comprises the amino acid sequence of SEQ ID NO: 242, and/or the VL comprises the amino acid sequence of SEQ ID NO: 296;
wherein the VH comprises the amino acid sequence of SEQ ID NO: 240, and/or the VL comprises the amino acid sequence of SEQ ID NO: 297;
wherein the VH comprises the amino acid sequence of SEQ ID NO: 241, and/or the VL comprises the amino acid sequence of SEQ ID NO: 297;
wherein the VH comprises the amino acid sequence of SEQ ID NO: 242, and/or the VL comprises the amino acid sequence of SEQ ID NO: 297;
wherein the VH comprises the amino acid sequence of SEQ ID NO: 241, and/or the VL comprises the amino acid sequence of SEQ ID NO: 298;
wherein the VH comprises the amino acid sequence of SEQ ID NO: 241, and the VL comprises the amino acid sequence of SEQ ID NO: 299;
wherein the VH comprises the amino acid sequence of SEQ ID NO: 241, and the VL comprises the amino acid sequence of SEQ ID NO: 300;
wherein the VH comprises the amino acid sequence of SEQ ID NO: 241, and/or the VL comprises the amino acid sequence of SEQ ID NO: 301;
wherein the VH comprises the amino acid sequence of SEQ ID NO: 242, and/or the VL comprises the amino acid sequence of SEQ ID NO: 298;
wherein the VH comprises the amino acid sequence of SEQ ID NO: 242, and/or the VL comprises the amino acid sequence of SEQ ID NO: 299;
wherein the VH comprises the amino acid sequence of SEQ ID NO: 242, and/or the VL comprises the amino acid sequence of SEQ ID NO: 300;
wherein the VH comprises the amino acid sequence of SEQ ID NO: 242, and/or the VL comprises the amino acid sequence of SEQ ID NO: 301;
wherein the VH comprises the amino acid sequence of SEQ ID NO: 243, and/or the VL comprises the amino acid sequence of SEQ ID NO: 298;
wherein the VH comprises the amino acid sequence of SEQ ID NO: 243, and/or the VL comprises the amino acid sequence of SEQ ID NO: 299;
wherein the VH comprises the amino acid sequence of SEQ ID NO: 243, and/or the VL comprises the amino acid sequence of SEQ ID NO: 300;
wherein the VH comprises the amino acid sequence of SEQ ID NO: 243, and/or the VL comprises the amino acid sequence of SEQ ID NO: 301;
wherein the VH comprises the amino acid sequence of SEQ ID NO: 244, and/or the VL comprises the amino acid sequence of SEQ ID NO: 302;
wherein the VH comprises the amino acid sequence of SEQ ID NO: 245, and/or the VL comprises the amino acid sequence of SEQ ID NO: 302;
wherein the VH comprises the amino acid sequence of SEQ ID NO: 246, and/or the VL comprises the amino acid sequence of SEQ ID NO: 302;
wherein the VH comprises the amino acid sequence of SEQ ID NO: 247, and/or the VL comprises the amino acid sequence of SEQ ID NO: 302;
wherein the VH comprises the amino acid sequence of SEQ ID NO: 248, and/or the VL comprises the amino acid sequence of SEQ ID NO: 303;
wherein the VH comprises the amino acid sequence of SEQ ID NO: 249, and/or the VL comprises the amino acid sequence of SEQ ID NO: 304;
wherein the VH comprises the amino acid sequence of SEQ ID NO: 250, and/or the VL comprises the amino acid sequence of SEQ ID NO: 305;
wherein the VH comprises the amino acid sequence of SEQ ID NO: 251, and/or the VL comprises the amino acid sequence of SEQ ID NO: 306;
wherein the VH comprises the amino acid sequence of SEQ ID NO: 252, and/or the VL comprises the amino acid sequence of SEQ ID NO: 305;
wherein the VH comprises the amino acid sequence of SEQ ID NO: 252, and/or the VL comprises the amino acid sequence of SEQ ID NO: 307;
wherein the VH comprises the amino acid sequence of SEQ ID NO: 253 and/or the VL comprises the amino acid sequence of SEQ ID NO: 308;
wherein the VH comprises the amino acid sequence of SEQ ID NO: 254, and/or the VL comprises the amino acid sequence of SEQ ID NO: 309;
wherein the VH comprises the amino acid sequence of SEQ ID NO: 255, and/or the VL comprises the amino acid sequence of SEQ ID NO: 310;
wherein the VH comprises the amino acid sequence of SEQ ID NO: 255, and/or the VL comprises the amino acid sequence of SEQ ID NO: 311;
wherein the VH comprises the amino acid sequence of SEQ ID NO: 255, and/or the VL comprises the amino acid sequence of SEQ ID NO: 312;
wherein the VH comprises the amino acid sequence of SEQ ID NO: 256, and/or the VL comprises the amino acid sequence of SEQ ID NO: 310;
wherein the VH comprises the amino acid sequence of SEQ ID NO: 256, and/or the VL comprises the amino acid sequence of SEQ ID NO: 311;
wherein the VH comprises the amino acid sequence of SEQ ID NO: 256, and/or the VL comprises the amino acid sequence of SEQ ID NO: 312;
wherein the VH comprises the amino acid sequence of SEQ ID NO: 257, and/or the VL comprises the amino acid sequence of SEQ ID NO: 310;
wherein the VH comprises the amino acid sequence of SEQ ID NO: 257, and/or the VL comprises the amino acid sequence of SEQ ID NO: 311;
wherein the VH comprises the amino acid sequence of SEQ ID NO: 257, and/or the VL comprises the amino acid sequence of SEQ ID NO: 312;
wherein the VH comprises the amino acid sequence of SEQ ID NO: 258, and/or the VL comprises the amino acid sequence of SEQ ID NO: 310;
wherein the VH comprises the amino acid sequence of SEQ ID NO: 258, and/or the VL comprises the amino acid sequence of SEQ ID NO: 311;
wherein the VH comprises the amino acid sequence of SEQ ID NO: 258, and/or the VL comprises the amino acid sequence of SEQ ID NO: 312;
wherein the VH comprises the amino acid sequence of SEQ ID NO: 259, and/or the VL comprises the amino acid sequence of SEQ ID NO: 310;
wherein the VH comprises the amino acid sequence of SEQ ID NO: 259, and/or the VL comprises the amino acid sequence of SEQ ID NO: 311;
wherein the VH comprises the amino acid sequence of SEQ ID NO: 259, and/or the VL comprises the amino acid sequence of SEQ ID NO: 312;
wherein the VH comprises the amino acid sequence of SEQ ID NO: 260, and/or the VL comprises the amino acid sequence of SEQ ID NO: 317;
wherein the VH comprises the amino acid sequence of SEQ ID NO: 260, and/or the VL comprises the amino acid sequence of SEQ ID NO: 313;
wherein the VH comprises the amino acid sequence of SEQ ID NO: 261, and/or the VL comprises the amino acid sequence of SEQ ID NO: 314;
wherein the VH comprises the amino acid sequence of SEQ ID NO: 261, and/or the VL comprises the amino acid sequence of SEQ ID NO: 315;
wherein the VH comprises the amino acid sequence of SEQ ID NO: 261, and/or the VL comprises the amino acid sequence of SEQ ID NO: 316;
wherein the VH comprises the amino acid sequence of SEQ ID NO: 262, and/or the VL comprises the amino acid sequence of SEQ ID NO: 317;
wherein the VH comprises the amino acid sequence of SEQ ID NO: 261, and/or the VL comprises the amino acid sequence of SEQ ID NO: 317;
wherein the VH comprises the amino acid sequence of SEQ ID NO: 263, and/or the VL comprises the amino acid sequence of SEQ ID NO: 317;
wherein the VH comprises the amino acid sequence of SEQ ID NO: 264, and/or the VL comprises the amino acid sequence of SEQ ID NO: 317;
wherein the VH comprises the amino acid sequence of SEQ ID NO: 262, and/or the VL comprises the amino acid sequence of SEQ ID NO: 318;
wherein the VH comprises the amino acid sequence of SEQ ID NO: 263, and/or the VL comprises the amino acid sequence of SEQ ID NO: 318;
wherein the VH comprises the amino acid sequence of SEQ ID NO: 264, and/or the VL comprises the amino acid sequence of SEQ ID NO: 318;
wherein the VH comprises the amino acid sequence of SEQ ID NO: 260, and/or the VL comprises the amino acid sequence of SEQ ID NO: 318;
wherein the VH comprises the amino acid sequence of SEQ ID NO: 264, and/or the VL comprises the amino acid sequence of SEQ ID NO: 314;
wherein the VH comprises the amino acid sequence of SEQ ID NO: 264, and/or the VL comprises the amino acid sequence of SEQ ID NO: 316;
wherein the VH comprises the amino acid sequence of SEQ ID NO: 260, and/or the VL comprises the amino acid sequence of SEQ ID NO: 319;
wherein the VH comprises the amino acid sequence of SEQ ID NO: 260, and/or the VL comprises the amino acid sequence of SEQ ID NO: 314;
wherein the VH comprises the amino acid sequence of SEQ ID NO: 260, and/or the VL comprises the amino acid sequence of SEQ ID NO: 315;
wherein the VH comprises the amino acid sequence of SEQ ID NO: 260, and/or the VL comprises the amino acid sequence of SEQ ID NO: 316;
wherein the VH comprises the amino acid sequence of SEQ ID NO: 261, and/or the VL comprises the amino acid sequence of SEQ ID NO: 313;
wherein the VH comprises the amino acid sequence of SEQ ID NO: 261, and/or the VL comprises the amino acid sequence of SEQ ID NO: 318;
wherein the VH comprises the amino acid sequence of SEQ ID NO: 261, and/or the VL comprises the amino acid sequence of SEQ ID NO: 319;
wherein the VH comprises the amino acid sequence of SEQ ID NO: 265, and/or the VL comprises the amino acid sequence of SEQ ID NO: 320;
wherein the VH comprises the amino acid sequence of SEQ ID NO: 266, and/or the VL comprises the amino acid sequence of SEQ ID NO: 321;
wherein the VH comprises the amino acid sequence of SEQ ID NO: 267, and/or the VL comprises the amino acid sequence of SEQ ID NO: 322;
wherein the VH comprises the amino acid sequence of SEQ ID NO: 268, and/or the VL comprises the amino acid sequence of SEQ ID NO: 323;
wherein the VH comprises the amino acid sequence of SEQ ID NO: 269, and/or the VL comprises the amino acid sequence of SEQ ID NO: 324;
wherein the VH comprises the amino acid sequence of SEQ ID NO: 270, and/or the VL comprises the amino acid sequence of SEQ ID NO: 325;
wherein the VH comprises the amino acid sequence of SEQ ID NO: 271, and/or the VL comprises the amino acid sequence of SEQ ID NO: 326;
wherein the VH comprises the amino acid sequence of SEQ ID NO: 272, and/or the VL comprises the amino acid sequence of SEQ ID NO: 331;
wherein the VH comprises the amino acid sequence of SEQ ID NO: 272, and/or the VL comprises the amino acid sequence of SEQ ID NO: 327;
wherein the VH comprises the amino acid sequence of SEQ ID NO: 273, and/or the VL comprises the amino acid sequence of SEQ ID NO: 328;
wherein the VH comprises the amino acid sequence of SEQ ID NO: 273, and/or the VL comprises the amino acid sequence of SEQ ID NO: 329;
wherein the VH comprises the amino acid sequence of SEQ ID NO: 273, and/or the VL comprises the amino acid sequence of SEQ ID NO: 330;
wherein the VH comprises the amino acid sequence of SEQ ID NO: 273, and/or the VL comprises the amino acid sequence of SEQ ID NO: 331;
wherein the VH comprises the amino acid sequence of SEQ ID NO: 274, and/or the VL comprises the amino acid sequence of SEQ ID NO: 331;
wherein the VH comprises the amino acid sequence of SEQ ID NO: 275, and/or the VL comprises the amino acid sequence of SEQ ID NO: 331;
wherein the VH comprises the amino acid sequence of SEQ ID NO: 276, and/or the VL comprises the amino acid sequence of SEQ ID NO: 331;
wherein the VH comprises the amino acid sequence of SEQ ID NO: 277, and/or the VL comprises the amino acid sequence of SEQ ID NO: 331;
wherein the VH comprises the amino acid sequence of SEQ ID NO: 272, and/or the VL comprises the amino acid sequence of SEQ ID NO: 332;
wherein the VH comprises the amino acid sequence of SEQ ID NO: 276, and/or the VL comprises the amino acid sequence of SEQ ID NO: 332;
wherein the VH comprises the amino acid sequence of SEQ ID NO: 275, and/or the VL comprises the amino acid sequence of SEQ ID NO: 328;
wherein the VH comprises the amino acid sequence of SEQ ID NO: 275, and/or the VL comprises the amino acid sequence of SEQ ID NO: 330;
wherein the VH comprises the amino acid sequence of SEQ ID NO: 277, and/or the VL comprises the amino acid sequence of SEQ ID NO: 328;
wherein the VH comprises the amino acid sequence of SEQ ID NO: 277, and/or the VL comprises the amino acid sequence of SEQ ID NO: 330;
wherein the VH comprises the amino acid sequence of SEQ ID NO: 272, and/or the VL comprises the amino acid sequence of SEQ ID NO: 333;
wherein the VH comprises the amino acid sequence of SEQ ID NO: 272, and/or the VL comprises the amino acid sequence of SEQ ID NO: 328;
wherein the VH comprises the amino acid sequence of SEQ ID NO: 272, and/or the VL comprises the amino acid sequence of SEQ ID NO: 329;
wherein the VH comprises the amino acid sequence of SEQ ID NO: 272 and/or the VL comprises the amino acid sequence of SEQ ID NO: 330;
wherein the VH comprises the amino acid sequence of SEQ ID NO: 273, and/or the VL comprises the amino acid sequence of SEQ ID NO: 327;
wherein the VH comprises the amino acid sequence of SEQ ID NO: 273, and/or the VL comprises the amino acid sequence of SEQ ID NO: 332;
wherein the VH comprises the amino acid sequence of SEQ ID NO: 273, and/or the VL comprises the amino acid sequence of SEQ ID NO: 333;
wherein the VH comprises the amino acid sequence of SEQ ID NO: 278, and/or the VL comprises the amino acid sequence of SEQ ID NO: 334;
wherein the VH comprises the amino acid sequence of SEQ ID NO: 279, and/or the VL comprises the amino acid sequence of SEQ ID NO: 323;
wherein the VH comprises the amino acid sequence of SEQ ID NO: 280, and/or the VL comprises the amino acid sequence of SEQ ID NO: 339;
wherein the VH comprises the amino acid sequence of SEQ ID NO: 280, and/or the VL comprises the amino acid sequence of SEQ ID NO: 335;
wherein the VH comprises the amino acid sequence of SEQ ID NO: 281, and/or the VL comprises the amino acid sequence of SEQ ID NO: 336;
wherein the VH comprises the amino acid sequence of SEQ ID NO: 281, and/or the VL comprises the amino acid sequence of SEQ ID NO: 337;
wherein the VH comprises the amino acid sequence of SEQ ID NO: 281, and/or the VL comprises the amino acid sequence of SEQ ID NO: 338;
wherein the VH comprises the amino acid sequence of SEQ ID NO: 282, and/or the VL comprises the amino acid sequence of SEQ ID NO: 339;
wherein the VH comprises the amino acid sequence of SEQ ID NO: 281, and/or the VL comprises the amino acid sequence of SEQ ID NO: 339;
wherein the VH comprises the amino acid sequence of SEQ ID NO: 283, and/or the VL comprises the amino acid sequence of SEQ ID NO: 339;
wherein the VH comprises the amino acid sequence of SEQ ID NO: 284, and/or the VL comprises the amino acid sequence of SEQ ID NO: 339;
wherein the VH comprises the amino acid sequence of SEQ ID NO: 285, and/or the VL comprises the amino acid sequence of SEQ ID NO: 339;
wherein the VH comprises the amino acid sequence of SEQ ID NO: 286, and/or the VL comprises the amino acid sequence of SEQ ID NO: 339;
wherein the VH comprises the amino acid sequence of SEQ ID NO: 282, and/or the VL comprises the amino acid sequence of SEQ ID NO: 340;
wherein the VH comprises the amino acid sequence of SEQ ID NO: 280, and/or the VL comprises the amino acid sequence of SEQ ID NO: 340;
wherein the VH comprises the amino acid sequence of SEQ ID NO: 283, and/or the VL comprises the amino acid sequence of SEQ ID NO: 340;
wherein the VH comprises the amino acid sequence of SEQ ID NO: 284, and/or the VL comprises the amino acid sequence of SEQ ID NO: 340;
wherein the VH comprises the amino acid sequence of SEQ ID NO: 285, and/or the VL comprises the amino acid sequence of SEQ ID NO: 340;
wherein the VH comprises the amino acid sequence of SEQ ID NO: 286, and/or the VL comprises the amino acid sequence of SEQ ID NO: 340;
wherein the VH comprises the amino acid sequence of SEQ ID NO: 284, and/or the VL comprises the amino acid sequence of SEQ ID NO: 336;
wherein the VH comprises the amino acid sequence of SEQ ID NO: 284, and/or the VL comprises the amino acid sequence of SEQ ID NO: 338;
wherein the VH comprises the amino acid sequence of SEQ ID NO: 286, and/or the VL comprises the amino acid sequence of SEQ ID NO: 336;
wherein the VH comprises the amino acid sequence of SEQ ID NO: 286, and/or the VL comprises the amino acid sequence of SEQ ID NO: 338;
wherein the VH comprises the amino acid sequence of SEQ ID NO: 287, and/or the VL comprises the amino acid sequence of SEQ ID NO: 340;
wherein the VH comprises the amino acid sequence of SEQ ID NO: 288, and/or the VL comprises the amino acid sequence of SEQ ID NO: 340;
wherein the VH comprises the amino acid sequence of SEQ ID NO: 280, and/or the VL comprises the amino acid sequence of SEQ ID NO: 341;
wherein the VH comprises the amino acid sequence of SEQ ID NO: 286, and/or the VL comprises the amino acid sequence of SEQ ID NO: 342;
wherein the VH comprises the amino acid sequence of SEQ ID NO: 286, and/or the VL comprises the amino acid sequence of SEQ ID NO: 343;
wherein the VH comprises the amino acid sequence of SEQ ID NO: 287, and/or the VL comprises the amino acid sequence of SEQ ID NO: 342;
wherein the VH comprises the amino acid sequence of SEQ ID NO: 288, and/or the VL comprises the amino acid sequence of SEQ ID NO: 343;
wherein the VH comprises the amino acid sequence of SEQ ID NO: 287, and/or the VL comprises the amino acid sequence of SEQ ID NO: 343;
wherein the VH comprises the amino acid sequence of SEQ ID NO: 288, and/or the VL comprises the amino acid sequence of SEQ ID NO: 342;
wherein the VH comprises the amino acid sequence of SEQ ID NO: 289, and/or the VL comprises the amino acid sequence of SEQ ID NO: 342;
wherein the VH comprises the amino acid sequence of SEQ ID NO: 289, and/or the VL comprises the amino acid sequence of SEQ ID NO: 343;
wherein the VH comprises the amino acid sequence of SEQ ID NO: 287, and/or the VL comprises the amino acid sequence of SEQ ID NO: 344;
wherein the VH comprises the amino acid sequence of SEQ ID NO: 288, and/or the VL comprises the amino acid sequence of SEQ ID NO: 344;
wherein the VH comprises the amino acid sequence of SEQ ID NO: 280, and/or the VL comprises the amino acid sequence of SEQ ID NO: 336;
wherein the VH comprises the amino acid sequence of SEQ ID NO: 289, and/or the VL comprises the amino acid sequence of SEQ ID NO: 344;
wherein the VH comprises the amino acid sequence of SEQ ID NO: 289, and/or the VL comprises the amino acid sequence of SEQ ID NO: 340;
wherein the VH comprises the amino acid sequence of SEQ ID NO: 286, and/or the VL comprises the amino acid sequence of SEQ ID NO: 344;
wherein the VH comprises the amino acid sequence of SEQ ID NO: 290, and/or the VL comprises the amino acid sequence of SEQ ID NO: 336;
wherein the VH comprises the amino acid sequence of SEQ ID NO: 291, and/or the VL comprises the amino acid sequence of SEQ ID NO: 336;
wherein the VH comprises the amino acid sequence of SEQ ID NO: 292, and/or the VL comprises the amino acid sequence of SEQ ID NO: 336;
wherein the VH comprises the amino acid sequence of SEQ ID NO: 290, and/or the VL comprises the amino acid sequence of SEQ ID NO: 345;
wherein the VH comprises the amino acid sequence of SEQ ID NO: 291, and/or the VL comprises the amino acid sequence of SEQ ID NO: 345;
wherein the VH comprises the amino acid sequence of SEQ ID NO: 292, and/or the VL comprises the amino acid sequence of SEQ ID NO: 345;
wherein the VH comprises the amino acid sequence of SEQ ID NO: 290, and/or the VL comprises the amino acid sequence of SEQ ID NO: 346;
wherein the VH comprises the amino acid sequence of SEQ ID NO: 280, and/or the VL comprises the amino acid sequence of SEQ ID NO: 337;
wherein the VH comprises the amino acid sequence of SEQ ID NO: 291, and/or the VL comprises the amino acid sequence of SEQ ID NO: 346;
wherein the VH comprises the amino acid sequence of SEQ ID NO: 292, and/or the VL comprises the amino acid sequence of SEQ ID NO: 346;
wherein the VH comprises the amino acid sequence of SEQ ID NO: 290, and/or the VL comprises the amino acid sequence of SEQ ID NO: 347;
wherein the VH comprises the amino acid sequence of SEQ ID NO: 291, and/or the VL comprises the amino acid sequence of SEQ ID NO: 347;
wherein the VH comprises the amino acid sequence of SEQ ID NO: 292, and/or the VL comprises the amino acid sequence of SEQ ID NO: 347;
wherein the VH comprises the amino acid sequence of SEQ ID NO: 293, and/or the VL comprises the amino acid sequence of SEQ ID NO: 340;
wherein the VH comprises the amino acid sequence of SEQ ID NO: 294, and/or the VL comprises the amino acid sequence of SEQ ID NO: 340;
wherein the VH comprises the amino acid sequence of SEQ ID NO: 295, and/or the VL comprises the amino acid sequence of SEQ ID NO: 340;
wherein the VH comprises the amino acid sequence of SEQ ID NO: 293, and/or the VL comprises the amino acid sequence of SEQ ID NO: 343;
wherein the VH comprises the amino acid sequence of SEQ ID NO: 294, and/or the VL comprises the amino acid sequence of SEQ ID NO: 343;
wherein the VH comprises the amino acid sequence of SEQ ID NO: 280, and/or the VL comprises the amino acid sequence of SEQ ID NO: 338;
wherein the VH comprises the amino acid sequence of SEQ ID NO: 295, and/or the VL comprises the amino acid sequence of SEQ ID NO: 343;
wherein the VH comprises the amino acid sequence of SEQ ID NO: 293, and/or the VL comprises the amino acid sequence of SEQ ID NO: 342;
wherein the VH comprises the amino acid sequence of SEQ ID NO: 294, and/or the VL comprises the amino acid sequence of SEQ ID NO: 342;
wherein the VH comprises the amino acid sequence of SEQ ID NO: 295 and/or the VL comprises the amino acid sequence of SEQ ID NO: 342;
wherein the VH comprises the amino acid sequence of SEQ ID NO: 293, and/or the VL comprises the amino acid sequence of SEQ ID NO: 344;
wherein the VH comprises the amino acid sequence of SEQ ID NO: 294, and/or the VL comprises the amino acid sequence of SEQ ID NO: 344;
wherein the VH comprises the amino acid sequence of SEQ ID NO: 295, and/or the VL comprises the amino acid sequence of SEQ ID NO: 344;
wherein the VH comprises the amino acid sequence of SEQ ID NO: 281, and/or the VL comprises the amino acid sequence of SEQ ID NO: 345;
wherein the VH comprises the amino acid sequence of SEQ ID NO: 281, and/or the VL comprises the amino acid sequence of SEQ ID NO: 346;
wherein the VH comprises the amino acid sequence of SEQ ID NO: 281, and/or the VL comprises the amino acid sequence of SEQ ID NO: 347;
wherein the VH comprises the amino acid sequence of SEQ ID NO: 281, and/or the VL comprises the amino acid sequence of SEQ ID NO: 335;
wherein the VH comprises the amino acid sequence of SEQ ID NO: 283, and/or the VL comprises the amino acid sequence of SEQ ID NO: 343;
wherein the VH comprises the amino acid sequence of SEQ ID NO: 283, and/or the VL comprises the amino acid sequence of SEQ ID NO: 342;
wherein the VH comprises the amino acid sequence of SEQ ID NO: 283, and/or the VL comprises the amino acid sequence of SEQ ID NO: 344;
wherein the VH comprises the amino acid sequence of SEQ ID NO: 281, and/or the VL comprises the amino acid sequence of SEQ ID NO: 340;
wherein the VH comprises the amino acid sequence of SEQ ID NO: 281, and/or the VL comprises the amino acid sequence of SEQ ID NO: 341;
wherein the VH comprises the amino acid sequence of SEQ ID NO: 461, and/or the VL comprises the amino acid sequence of SEQ ID NO: 462; or
wherein the VH comprises the amino acid sequence of SEQ ID NO: 459, and/or the VL comprises the amino acid sequence of SEQ ID NO: 296.

61-231. (canceled)

232. The antibody or antigen-binding fragment thereof of claim 1, comprising:
(a) a heavy chain comprising an amino acid sequence at least 90% identical to any one of SEQ ID NOs: 348-403, 460 and 463; and/or
(b) a light chain comprising an amino acid sequence least 90% identical to any one of SEQ ID NOs: 404-455 and 464.

233. The antibody or antigen-binding fragment thereof of claim 232, comprising:
(a) a heavy chain comprising an amino acid sequence at least 95% identical to any one of SEQ ID NOs: 348-403, 460 and 463; and/or
(b) a light chain comprising an amino acid sequence at least 95% identical to any one of SEQ ID NOs: 404-455 and 464.

234. The antibody or antigen-binding fragment thereof of claim 233, comprising:
(a) a heavy chain comprising an amino acid sequence of any one of SEQ ID NOs: 348-403, 460 and 463; and/or
(b) a light chain comprising an amino acid sequence of any one of SEQ ID NOs: 404-455 and 464.

235. The antibody or antigen-binding fragment thereof of claim 234,
wherein the heavy chain comprises the amino acid sequence of SEQ ID NO: 348, and/or the light chain comprises the amino acid sequence of SEQ ID NO: 404;
wherein the heavy chain comprises the amino acid sequence of SEQ ID NO: 349, and/or the light chain comprises the amino acid sequence of SEQ ID NO: 404;
wherein the heavy chain comprises the amino acid sequence of SEQ ID NO: 350, and/or the light chain comprises the amino acid sequence of SEQ ID NO: 404;
wherein the heavy chain comprises the amino acid sequence of SEQ ID NO: 348, and/or the light chain comprises the amino acid sequence of SEQ ID NO: 405;
wherein the heavy chain comprises the amino acid sequence of SEQ ID NO: 349, and/or the light chain comprises the amino acid sequence of SEQ ID NO: 405;
wherein the heavy chain comprises the amino acid sequence of SEQ ID NO: 350, and/or the light chain comprises the amino acid sequence of SEQ ID NO: 405;
wherein the heavy chain comprises the amino acid sequence of SEQ ID NO: 349, and/or the light chain comprises the amino acid sequence of SEQ ID NO: 406;
wherein the heavy chain comprises the amino acid sequence of SEQ ID NO: 349, and/or the light chain comprises the amino acid sequence of SEQ ID NO: 407;
wherein the heavy chain comprises the amino acid sequence of SEQ ID NO: 349, and/or the light chain comprises the amino acid sequence of SEQ ID NO: 408;
wherein the heavy chain comprises the amino acid sequence of SEQ ID NO: 349, and/or the light chain comprises the amino acid sequence of SEQ ID NO: 409;
wherein the heavy chain comprises the amino acid sequence of SEQ ID NO: 350, and/or the light chain comprises the amino acid sequence of SEQ ID NO: 406;
wherein the heavy chain comprises the amino acid sequence of SEQ ID NO: 350, and/or the light chain comprises the amino acid sequence of SEQ ID NO: 407;
wherein the heavy chain comprises the amino acid sequence of SEQ ID NO: 350, and/or the light chain comprises the amino acid sequence of SEQ ID NO: 408;
wherein the heavy chain comprises the amino acid sequence of SEQ ID NO: 350, and/or the light chain comprises the amino acid sequence of SEQ ID NO: 409;
wherein the heavy chain comprises the amino acid sequence of SEQ ID NO: 351, and/or the light chain comprises the amino acid sequence of SEQ ID NO: 406;
wherein the heavy chain comprises the amino acid sequence of SEQ ID NO: 351, and/or the light chain comprises the amino acid sequence of SEQ ID NO: 407;
wherein the heavy chain comprises the amino acid sequence of SEQ ID NO: 351, and/or the light chain comprises the amino acid sequence of SEQ ID NO: 408;
wherein the heavy chain comprises the amino acid sequence of SEQ ID NO: 351, and/or the light chain comprises the amino acid sequence of SEQ ID NO: 409;
wherein the heavy chain comprises the amino acid sequence of SEQ ID NO: 352, and/or the light chain comprises the amino acid sequence of SEQ ID NO: 410;
wherein the heavy chain comprises the amino acid sequence of SEQ ID NO: 353, and/or the light chain comprises the amino acid sequence of SEQ ID NO: 410;
wherein the heavy chain comprises the amino acid sequence of SEQ ID NO: 354, and/or the light chain comprises the amino acid sequence of SEQ ID NO: 410;
wherein the heavy chain comprises the amino acid sequence of SEQ ID NO: 355, and/or the light chain comprises the amino acid sequence of SEQ ID NO: 410;
wherein the heavy chain comprises the amino acid sequence of SEQ ID NO: 356, and/or the light chain comprises the amino acid sequence of SEQ ID NO: 411;
wherein the heavy chain comprises the amino acid sequence of SEQ ID NO: 357, and/or the light chain comprises the amino acid sequence of SEQ ID NO: 412;
wherein the heavy chain comprises the amino acid sequence of SEQ ID NO: 358, and/or the light chain comprises the amino acid sequence of SEQ ID NO: 413;
wherein the heavy chain comprises the amino acid sequence of SEQ ID NO: 359, and/or the light chain comprises the amino acid sequence of SEQ ID NO: 414;
wherein the heavy chain comprises the amino acid sequence of SEQ ID NO: 360, and/or the light chain comprises the amino acid sequence of SEQ ID NO: 413;
wherein the heavy chain comprises the amino acid sequence of SEQ ID NO: 360, and/or the light chain comprises the amino acid sequence of SEQ ID NO: 415;
wherein the heavy chain comprises the amino acid sequence of SEQ ID NO: 361, and/or the light chain comprises the amino acid sequence of SEQ ID NO: 416;
wherein the heavy chain comprises the amino acid sequence of SEQ ID NO: 362, and/or the light chain comprises the amino acid sequence of SEQ ID NO: 417;
wherein the heavy chain comprises the amino acid sequence of SEQ ID NO: 363, and/or the light chain comprises the amino acid sequence of SEQ ID NO: 418;
wherein the heavy chain comprises the amino acid sequence of SEQ ID NO: 363, and/or the light chain comprises the amino acid sequence of SEQ ID NO: 419;
wherein the heavy chain comprises the amino acid sequence of SEQ ID NO: 363, and/or the light chain comprises the amino acid sequence of SEQ ID NO: 420;
wherein the heavy chain comprises the amino acid sequence of SEQ ID NO: 364, and/or the light chain comprises the amino acid sequence of SEQ ID NO: 418;
wherein the heavy chain comprises the amino acid sequence of SEQ ID NO: 364, and/or the light chain comprises the amino acid sequence of SEQ ID NO: 419;
wherein the heavy chain comprises the amino acid sequence of SEQ ID NO: 364, and/or the light chain comprises the amino acid sequence of SEQ ID NO: 420;
wherein the heavy chain comprises the amino acid sequence of SEQ ID NO: 365, and/or the light chain comprises the amino acid sequence of SEQ ID NO: 418;
wherein the heavy chain comprises the amino acid sequence of SEQ ID NO: 365, and/or the light chain comprises the amino acid sequence of SEQ ID NO: 419;
wherein the heavy chain comprises the amino acid sequence of SEQ ID NO: 365, and/or the light chain comprises the amino acid sequence of SEQ ID NO: 420;
wherein the heavy chain comprises the amino acid sequence of SEQ ID NO: 366, and/or the light chain comprises the amino acid sequence of SEQ ID NO: 418;
wherein the heavy chain comprises the amino acid sequence of SEQ ID NO: 366, and/or the light chain comprises the amino acid sequence of SEQ ID NO: 419;
wherein the heavy chain comprises the amino acid sequence of SEQ ID NO: 366, and/or the light chain comprises the amino acid sequence of SEQ ID NO: 420;
wherein the heavy chain comprises the amino acid sequence of SEQ ID NO: 367, and/or the light chain comprises the amino acid sequence of SEQ ID NO: 418;
wherein the heavy chain comprises the amino acid sequence of SEQ ID NO: 367, and/or the light chain comprises the amino acid sequence of SEQ ID NO: 419;
wherein the heavy chain comprises the amino acid sequence of SEQ ID NO: 367, and/or the light chain comprises the amino acid sequence of SEQ ID NO: 420;
wherein the heavy chain comprises the amino acid sequence of SEQ ID NO: 368, and/or the light chain comprises the amino acid sequence of SEQ ID NO: 425;
wherein the heavy chain comprises the amino acid sequence of SEQ ID NO: 368, and/or the light chain comprises the amino acid sequence of SEQ ID NO: 421;
wherein the heavy chain comprises the amino acid sequence of SEQ ID NO: 369, and/or the light chain comprises the amino acid sequence of SEQ ID NO: 422;
wherein the heavy chain comprises the amino acid sequence of SEQ ID NO: 369, and/or the light chain comprises the amino acid sequence of SEQ ID NO: 423;
wherein the heavy chain comprises the amino acid sequence of SEQ ID NO: 369, and/or the light chain comprises the amino acid sequence of SEQ ID NO: 424;
wherein the heavy chain comprises the amino acid sequence of SEQ ID NO: 370, and/or the light chain comprises the amino acid sequence of SEQ ID NO: 425;
wherein the heavy chain comprises the amino acid sequence of SEQ ID NO: 369, and/or the light chain comprises the amino acid sequence of SEQ ID NO: 425;
wherein the heavy chain comprises the amino acid sequence of SEQ ID NO: 371, and/or the light chain comprises the amino acid sequence of SEQ ID NO: 425;
wherein the heavy chain comprises the amino acid sequence of SEQ ID NO: 372, and/or the light chain comprises the amino acid sequence of SEQ ID NO: 425;
wherein the heavy chain comprises the amino acid sequence of SEQ ID NO: 370, and/or the light chain comprises the amino acid sequence of SEQ ID NO: 426;
wherein the heavy chain comprises the amino acid sequence of SEQ ID NO: 371, and/or the light chain comprises the amino acid sequence of SEQ ID NO: 426;
wherein the heavy chain comprises the amino acid sequence of SEQ ID NO: 372, and/or the light chain comprises the amino acid sequence of SEQ ID NO: 426;
wherein the heavy chain comprises the amino acid sequence of SEQ ID NO: 368, and/or the light chain comprises the amino acid sequence of SEQ ID NO: 426;
wherein the heavy chain comprises the amino acid sequence of SEQ ID NO: 372, and/or the light chain comprises the amino acid sequence of SEQ ID NO: 422;
wherein the heavy chain comprises the amino acid sequence of SEQ ID NO: 372, and/or the light chain comprises the amino acid sequence of SEQ ID NO: 424;
wherein the heavy chain comprises the amino acid sequence of SEQ ID NO: 368, and/or the light chain comprises the amino acid sequence of SEQ ID NO: 427;
wherein the heavy chain comprises the amino acid sequence of SEQ ID NO: 368, and/or the light chain comprises the amino acid sequence of SEQ ID NO: 422;
wherein the heavy chain comprises the amino acid sequence of SEQ ID NO: 368, and/or the light chain comprises the amino acid sequence of SEQ ID NO: 423;
wherein the heavy chain comprises the amino acid sequence of SEQ ID NO: 368, and/or the light chain comprises the amino acid sequence of SEQ ID NO: 424;
wherein the heavy chain comprises the amino acid sequence of SEQ ID NO: 369, and/or the light chain comprises the amino acid sequence of SEQ ID NO: 421;
wherein the heavy chain comprises the amino acid sequence of SEQ ID NO: 369, and/or the light chain comprises the amino acid sequence of SEQ ID NO: 426;
wherein the heavy chain comprises the amino acid sequence of SEQ ID NO: 369, and/or the light chain comprises the amino acid sequence of SEQ ID NO: 427;
wherein the heavy chain comprises the amino acid sequence of SEQ ID NO: 373, and/or the light chain comprises the amino acid sequence of SEQ ID NO: 428;
wherein the heavy chain comprises the amino acid sequence of SEQ ID NO: 374, and/or the light chain comprises the amino acid sequence of SEQ ID NO: 429;
wherein the heavy chain comprises the amino acid sequence of SEQ ID NO: 375, and/or the light chain comprises the amino acid sequence of SEQ ID NO: 430;
wherein the heavy chain comprises the amino acid sequence of SEQ ID NO: 376, and/or the light chain comprises the amino acid sequence of SEQ ID NO: 431;
wherein the heavy chain comprises the amino acid sequence of SEQ ID NO: 377, and/or the light chain comprises the amino acid sequence of SEQ ID NO: 432;
wherein the heavy chain comprises the amino acid sequence of SEQ ID NO: 378, and/or the light chain comprises the amino acid sequence of SEQ ID NO: 433;
wherein the heavy chain comprises the amino acid sequence of SEQ ID NO: 379, and/or the light chain comprises the amino acid sequence of SEQ ID NO: 434;
wherein the heavy chain comprises the amino acid sequence of SEQ ID NO: 380, and/or the light chain comprises the amino acid sequence of SEQ ID NO: 439;
wherein the heavy chain comprises the amino acid sequence of SEQ ID NO: 380, and/or the light chain comprises the amino acid sequence of SEQ ID NO: 435;
wherein the heavy chain comprises the amino acid sequence of SEQ ID NO: 381, and/or the light chain comprises the amino acid sequence of SEQ ID NO: 436;
wherein the heavy chain comprises the amino acid sequence of SEQ ID NO: 381, and/or the light chain comprises the amino acid sequence of SEQ ID NO: 437;
wherein the heavy chain comprises the amino acid sequence of SEQ ID NO: 381, and/or the light chain comprises the amino acid sequence of SEQ ID NO: 438;
wherein the heavy chain comprises the amino acid sequence of SEQ ID NO: 381, and/or the light chain comprises the amino acid sequence of SEQ ID NO: 439;
wherein the heavy chain comprises the amino acid sequence of SEQ ID NO: 382, and/or the light chain comprises the amino acid sequence of SEQ ID NO: 439;
wherein the heavy chain comprises the amino acid sequence of SEQ ID NO: 383, and/or the light chain comprises the amino acid sequence of SEQ ID NO: 439;
wherein the heavy chain comprises the amino acid sequence of SEQ ID NO: 384, and/or the light chain comprises the amino acid sequence of SEQ ID NO: 439;
wherein the heavy chain comprises the amino acid sequence of SEQ ID NO: 385, and/or the light chain comprises the amino acid sequence of SEQ ID NO: 439;
wherein the heavy chain comprises the amino acid sequence of SEQ ID NO: 380, and/or the light chain comprises the amino acid sequence of SEQ ID NO: 440;
wherein the heavy chain comprises the amino acid sequence of SEQ ID NO: 384, and/or the light chain comprises the amino acid sequence of SEQ ID NO: 440;
wherein the heavy chain comprises the amino acid sequence of SEQ ID NO: 383, and/or the light chain comprises the amino acid sequence of SEQ ID NO: 436;
wherein the heavy chain comprises the amino acid sequence of SEQ ID NO: 383, and/or the light chain comprises the amino acid sequence of SEQ ID NO: 438;
wherein the heavy chain comprises the amino acid sequence of SEQ ID NO: 385, and/or the light chain comprises the amino acid sequence of SEQ ID NO: 436;
wherein the heavy chain comprises the amino acid sequence of SEQ ID NO: 385, and/or the light chain comprises the amino acid sequence of SEQ ID NO: 438;
wherein the heavy chain comprises the amino acid sequence of SEQ ID NO: 380, and/or the light chain comprises the amino acid sequence of SEQ ID NO: 441;
wherein the heavy chain comprises the amino acid sequence of SEQ ID NO: 380, and/or the light chain comprises the amino acid sequence of SEQ ID NO: 436;
wherein the heavy chain comprises the amino acid sequence of SEQ ID NO: 380, and/or the light chain comprises the amino acid sequence of SEQ ID NO: 437;
wherein the heavy chain comprises the amino acid sequence of SEQ ID NO: 380, and/or the light chain comprises the amino acid sequence of SEQ ID NO: 438;
wherein the heavy chain comprises the amino acid sequence of SEQ ID NO: 381, and/or the light chain comprises the amino acid sequence of SEQ ID NO: 435;
wherein the heavy chain comprises the amino acid sequence of SEQ ID NO: 381, and/or the light chain comprises the amino acid sequence of SEQ ID NO: 440;
wherein the heavy chain comprises the amino acid sequence of SEQ ID NO: 381, and/or the light chain comprises the amino acid sequence of SEQ ID NO: 441;
wherein the heavy chain comprises the amino acid sequence of SEQ ID NO: 386, and/or the light chain comprises the amino acid sequence of SEQ ID NO: 442;
wherein the heavy chain comprises the amino acid sequence of SEQ ID NO: 387, and/or the light chain comprises the amino acid sequence of SEQ ID NO: 431;
wherein the heavy chain comprises the amino acid sequence of SEQ ID NO: 388, and/or the light chain comprises the amino acid sequence of SEQ ID NO: 447;
wherein the heavy chain comprises the amino acid sequence of SEQ ID NO: 388, and/or the light chain comprises the amino acid sequence of SEQ ID NO: 443;
wherein the heavy chain comprises the amino acid sequence of SEQ ID NO: 389, and/or the light chain comprises the amino acid sequence of SEQ ID NO: 444;
wherein the heavy chain comprises the amino acid sequence of SEQ ID NO: 389, and/or the light chain comprises the amino acid sequence of SEQ ID NO: 445;
wherein the heavy chain comprises the amino acid sequence of SEQ ID NO: 389, and/or the light chain comprises the amino acid sequence of SEQ ID NO: 446;
wherein the heavy chain comprises the amino acid sequence of SEQ ID NO: 390, and/or the light chain comprises the amino acid sequence of SEQ ID NO: 447;
wherein the heavy chain comprises the amino acid sequence of SEQ ID NO: 389, and/or the light chain comprises the amino acid sequence of SEQ ID NO: 447;
wherein the heavy chain comprises the amino acid sequence of SEQ ID NO: 391, and/or the light chain comprises the amino acid sequence of SEQ ID NO: 447;
wherein the heavy chain comprises the amino acid sequence of SEQ ID NO: 392, and/or the light chain comprises the amino acid sequence of SEQ ID NO: 447;
wherein the heavy chain comprises the amino acid sequence of SEQ ID NO: 393, and/or the light chain comprises the amino acid sequence of SEQ ID NO: 447;
wherein the heavy chain comprises the amino acid sequence of SEQ ID NO: 394, and/or the light chain comprises the amino acid sequence of SEQ ID NO: 447;
wherein the heavy chain comprises the amino acid sequence of SEQ ID NO: 390, and/or the light chain comprises the amino acid sequence of SEQ ID NO: 448;
wherein the heavy chain comprises the amino acid sequence of SEQ ID NO: 388, and/or the light chain comprises the amino acid sequence of SEQ ID NO: 448;
wherein the heavy chain comprises the amino acid sequence of SEQ ID NO: 391, and/or the light chain comprises the amino acid sequence of SEQ ID NO: 448;
wherein the heavy chain comprises the amino acid sequence of SEQ ID NO: 392, and/or the light chain comprises the amino acid sequence of SEQ ID NO: 448;
wherein the heavy chain comprises the amino acid sequence of SEQ ID NO: 393, and/or the light chain comprises the amino acid sequence of SEQ ID NO: 448;
wherein the heavy chain comprises the amino acid sequence of SEQ ID NO: 394, and/or the light chain comprises the amino acid sequence of SEQ ID NO: 448;
wherein the heavy chain comprises the amino acid sequence of SEQ ID NO: 392, and/or the light chain comprises the amino acid sequence of SEQ ID NO: 444;
wherein the heavy chain comprises the amino acid sequence of SEQ ID NO: 392, and/or the light chain comprises the amino acid sequence of SEQ ID NO: 446;
wherein the heavy chain comprises the amino acid sequence of SEQ ID NO: 394, and/or the light chain comprises the amino acid sequence of SEQ ID NO: 444;
wherein the heavy chain comprises the amino acid sequence of SEQ ID NO: 394, and/or the light chain comprises the amino acid sequence of SEQ ID NO: 446;
wherein the heavy chain comprises the amino acid sequence of SEQ ID NO: 395, and/or the light chain comprises the amino acid sequence of SEQ ID NO: 448;
wherein the heavy chain comprises the amino acid sequence of SEQ ID NO: 396, and/or the light chain comprises the amino acid sequence of SEQ ID NO: 448;
wherein the heavy chain comprises the amino acid sequence of SEQ ID NO: 388, and/or the light chain comprises the amino acid sequence of SEQ ID NO: 449;
wherein the heavy chain comprises the amino acid sequence of SEQ ID NO: 394, and/or the light chain comprises the amino acid sequence of SEQ ID NO: 450;
wherein the heavy chain comprises the amino acid sequence of SEQ ID NO: 394, and/or the light chain comprises the amino acid sequence of SEQ ID NO: 451;
wherein the heavy chain comprises the amino acid sequence of SEQ ID NO: 395, and/or the light chain comprises the amino acid sequence of SEQ ID NO: 450;
wherein the heavy chain comprises the amino acid sequence of SEQ ID NO: 396, and/or the light chain comprises the amino acid sequence of SEQ ID NO: 451;
wherein the heavy chain comprises the amino acid sequence of SEQ ID NO: 395, and/or the light chain comprises the amino acid sequence of SEQ ID NO: 451;
wherein the heavy chain comprises the amino acid sequence of SEQ ID NO: 396, and/or the light chain comprises the amino acid sequence of SEQ ID NO: 450;
wherein the heavy chain comprises the amino acid sequence of SEQ ID NO: 397, and/or the light chain comprises the amino acid sequence of SEQ ID NO: 450;
wherein the heavy chain comprises the amino acid sequence of SEQ ID NO: 397, and/or the light chain comprises the amino acid sequence of SEQ ID NO: 451;
wherein the heavy chain comprises the amino acid sequence of SEQ ID NO: 395, and/or the light chain comprises the amino acid sequence of SEQ ID NO: 452;
wherein the heavy chain comprises the amino acid sequence of SEQ ID NO: 396, and/or the light chain comprises the amino acid sequence of SEQ ID NO: 452;
wherein the heavy chain comprises the amino acid sequence of SEQ ID NO: 388, and/or the light chain comprises the amino acid sequence of SEQ ID NO: 444;
wherein the heavy chain comprises the amino acid sequence of SEQ ID NO: 397, and/or the light chain comprises the amino acid sequence of SEQ ID NO: 452;
wherein the heavy chain comprises the amino acid sequence of SEQ ID NO: 397, and/or the light chain comprises the amino acid sequence of SEQ ID NO: 448;
wherein the heavy chain comprises the amino acid sequence of SEQ ID NO: 394, and/or the light chain comprises the amino acid sequence of SEQ ID NO: 452;
wherein the heavy chain comprises the amino acid sequence of SEQ ID NO: 398, and/or the light chain comprises the amino acid sequence of SEQ ID NO: 444;
wherein the heavy chain comprises the amino acid sequence of SEQ ID NO: 399, and/or the light chain comprises the amino acid sequence of SEQ ID NO: 444;
wherein the heavy chain comprises the amino acid sequence of SEQ ID NO: 400, and/or the light chain comprises the amino acid sequence of SEQ ID NO: 444;
wherein the heavy chain comprises the amino acid sequence of SEQ ID NO: 398, and/or the light chain comprises the amino acid sequence of SEQ ID NO: 453;
wherein the heavy chain comprises the amino acid sequence of SEQ ID NO: 399, and/or the light chain comprises the amino acid sequence of SEQ ID NO: 453;
wherein the heavy chain comprises the amino acid sequence of SEQ ID NO: 400, and/or the light chain comprises the amino acid sequence of SEQ ID NO: 453;
wherein the heavy chain comprises the amino acid sequence of SEQ ID NO: 398, and/or the light chain comprises the amino acid sequence of SEQ ID NO: 454;
wherein the heavy chain comprises the amino acid sequence of SEQ ID NO: 388, and/or the light chain comprises the amino acid sequence of SEQ ID NO: 445;
wherein the heavy chain comprises the amino acid sequence of SEQ ID NO: 399, and/or the light chain comprises the amino acid sequence of SEQ ID NO: 454;
wherein the heavy chain comprises the amino acid sequence of SEQ ID NO: 400, and/or the light chain comprises the amino acid sequence of SEQ ID NO: 454;
wherein the heavy chain comprises the amino acid sequence of SEQ ID NO: 398, and/or the light chain comprises the amino acid sequence of SEQ ID NO: 455;
wherein the heavy chain comprises the amino acid sequence of SEQ ID NO: 399, and/or the light chain comprises the amino acid sequence of SEQ ID NO: 455;
wherein the heavy chain comprises the amino acid sequence of SEQ ID NO: 400, and/or the light chain comprises the amino acid sequence of SEQ ID NO: 455;
wherein the heavy chain comprises the amino acid sequence of SEQ ID NO: 401, and/or the light chain comprises the amino acid sequence of SEQ ID NO: 448;
wherein the heavy chain comprises the amino acid sequence of SEQ ID NO: 402, and/or the light chain comprises the amino acid sequence of SEQ ID NO: 448;
wherein the heavy chain comprises the amino acid sequence of SEQ ID NO: 403, and/or the light chain comprises the amino acid sequence of SEQ ID NO: 448;
wherein the heavy chain comprises the amino acid sequence of SEQ ID NO: 401, and/or the light chain comprises the amino acid sequence of SEQ ID NO: 451;
wherein the heavy chain comprises the amino acid sequence of SEQ ID NO: 402, and/or the light chain comprises the amino acid sequence of SEQ ID NO: 451;
wherein the heavy chain comprises the amino acid sequence of SEQ ID NO: 388, and/or the light chain comprises the amino acid sequence of SEQ ID NO: 446;
wherein the heavy chain comprises the amino acid sequence of SEQ ID NO: 403, and/or the light chain comprises the amino acid sequence of SEQ ID NO: 451;
wherein the heavy chain comprises the amino acid sequence of SEQ ID NO: 401, and/or the light chain comprises the amino acid sequence of SEQ ID NO: 450;
wherein the heavy chain comprises the amino acid sequence of SEQ ID NO: 402, and/or the light chain comprises the amino acid sequence of SEQ ID NO: 450;
wherein the heavy chain comprises the amino acid sequence of SEQ ID NO: 403, and/or the light chain comprises the amino acid sequence of SEQ ID NO: 450;
wherein the heavy chain comprises the amino acid sequence of SEQ ID NO: 401, and/or the light chain comprises the amino acid sequence of SEQ ID NO: 452;
wherein the heavy chain comprises the amino acid sequence of SEQ ID NO: 402, and/or the light chain comprises the amino acid sequence of SEQ ID NO: 452;
wherein the heavy chain comprises the amino acid sequence of SEQ ID NO: 403, and/or the light chain comprises the amino acid sequence of SEQ ID NO: 452;
wherein the heavy chain comprises the amino acid sequence of SEQ ID NO: 389, and/or the light chain comprises the amino acid sequence of SEQ ID NO: 453;
wherein the heavy chain comprises the amino acid sequence of SEQ ID NO: 389, and/or the light chain comprises the amino acid sequence of SEQ ID NO: 454;
wherein the heavy chain comprises the amino acid sequence of SEQ ID NO: 389, and/or the light chain comprises the amino acid sequence of SEQ ID NO: 455;
wherein the heavy chain comprises the amino acid sequence of SEQ ID NO: 389, and/or the light chain comprises the amino acid sequence of SEQ ID NO: 443;
wherein the heavy chain comprises the amino acid sequence of SEQ ID NO: 391, and/or the light chain comprises the amino acid sequence of SEQ ID NO: 451;
wherein the heavy chain comprises the amino acid sequence of SEQ ID NO: 391, and/or the light chain comprises the amino acid sequence of SEQ ID NO: 450;
wherein the heavy chain comprises the amino acid sequence of SEQ ID NO: 391, and/or the light chain comprises the amino acid sequence of SEQ ID NO: 452;
wherein the heavy chain comprises the amino acid sequence of SEQ ID NO: 389, and/or the light chain comprises the amino acid sequence of SEQ ID NO: 448;
wherein the heavy chain comprises the amino acid sequence of SEQ ID NO: 389, and/or the light chain comprises the amino acid sequence of SEQ ID NO: 449;
wherein the heavy chain comprises the amino acid sequence of SEQ ID NO: 463, and/or the light chain comprises the amino acid sequence of SEQ ID NO: 464, or wherein the heavy chain comprises the amino acid sequence of SEQ ID NO: 460, and/or the light chain comprises the amino acid sequence of SEQ ID NO: 404.

236-406. (canceled)

407. The antibody or antigen-binding fragment thereof of claim 1, wherein the antibody is polyclonal, monoclonal, human, humanized, or chimeric.

408. The antibody or antigen-binding fragment thereof of claim 1, wherein the antigen-binding fragment is a Fab, scFab, Fab′, F(ab′)₂, Fv, scFv, diabody, or triabody.

409. A pharmaceutical composition, comprising the antibody or antigen-binding fragment thereof of claim 1 and a pharmaceutically acceptable carrier.

410. (canceled)

411. A method of treating CAH in a subject in need thereof, comprising administering to the subject the antibody or antigen-binding fragment thereof of claim 1.

412-413. (canceled)

414. A method of reducing androgen concentration in a subject in need thereof, comprising administering to the subject the antibody or antigen-binding fragment of claim 1.

415-418. (canceled)

419. The method of claim 414, wherein the androgen is testosterone, free testosterone, androstenedione, an 11-oxygenated androgen, or a combination thereof.

420. The method of claim 419, wherein the 11-oxygenated androgen is 11-hydroxyandrostenedione (110HA4), 11-hydroxytestosterone (11OHT), 11-ketoandrostenedione (11KA4), 11-ketotestosterone (11KT), or a combination thereof.

421. (canceled)

422. A method for reducing the severity of one or more symptoms or signs in a subject having CAH, comprising administering to the subject the antibody or antigen-binding fragment thereof of claim 1.

423-424. (canceled)

425. The method of claim 422, wherein the one or more symptoms or signs is virilization, acne, oily skin or hair, bone age advancement, precocious puberty, short height, hirsutism, male-pattern baldness, enlarged thyroid cartilage, fusion of the labia minora, fusion of the labia majora, hyperpigmentation of the labia majora or minora, rugation of the labia majora, clitoralmegaly, testicular adrenal rest tumors, fertility problems, or irregular or absent menstrual periods.

426-437. (canceled)

438. A method of inhibiting the hypothalamic pituitary adrenal (HPA) axis in a subject in need thereof, comprising administering to the subject the antibody or antigen-binding fragment thereof of claim 1.

439-440. (canceled)

441. The method of claim 411, wherein the CAH is classic CAH.

442. The method of claim 411, wherein the CAH is nonclassic CAH.

443-444. (canceled)

445. The method of claim 411, further comprising administering to the subject a mineralocorticoid.

446. The method of claim 411, further comprising administering to the subject a glucocorticoid.

447. (canceled)

448. The method of claim 411, wherein the CAH is associated with a mutation or deletion in the 21-hydroxylase gene (CYP21A2), a mutation or deletion in the 11-beta-hydroxylase gene (CYP11B1), or a mutation or deletion in the 3-beta-hydroxysteroid dehydrogenase gene (HSD3B2).

449-453. (canceled)
